(19) United States
(12) Patent Application Publication
(10) Pub. No.: US 2025/0154477 A1
(43) Pub. Date: May 15, 2025

(54) NUDIX OVEREXPRESSING ENGINEERED PLANTS AND USES THEREOF

(71) Applicant: VIRGINIA TECH INTELLECTUAL PROPERTIES, INC., Blacksburg, VA (US)

(72) Inventors: Catherine Freed, Blacksburg, VA (US); Glenda Gillaspy, Blacksburg, VA (US)

(21) Appl. No.: 18/730,449

(22) PCT Filed: Jan. 19, 2023
(86) PCT No.: PCT/US23/60894

§ 371 Date: Jul. 19, 2024

Related U.S. Application Data
(60) Provisional application No. 63/301,026, filed on Jan. 19, 2022.

(51) Int. Cl. C12N 9/14; B09C 1/10; C02F 3/32; C02F 101/10; C05D 9/00; C10B 53/02; C12N 15/82

(52) U.S. Cl. CPC C12N 9/14; B09C 1/105; C02F 3/327; C05D 9/00; C10B 53/02; C12N 15/8259; B09C 2101/00; C02F 2101/105

(57) ABSTRACT

Described in several example embodiments herein are NUDIX overexpressing plants and uses thereof. In some embodiments, the NUDIX overexpressing plants are used for phytoremediation.

### CROSS-REFERENCE TO RELATED APPLICATIONS

This application claims the benefit of and priority to U.S. Provisional Patent Application No. 63/301,026, filed on Jan. 19, 2022, entitled “NUDIX Overexpressing Engineered Plants and Uses Thereof,” the contents of which is incorporated by reference herein in its entirety.

### STATEMENT REGARDING FEDERALLY SPONSORED RESEARCH

This invention was made with government support under Grant No. MCB 161038 awarded by National Science Foundation. The government has certain rights in the invention.

### SEQUENCE LISTING

This application contains a sequence listing filed in electronic form entitled VTIP-0365WP_ST26.xml, created on Jan. 19, 2023, and having a size of 145,852 bytes. The content of the sequence listing is incorporated herein in its entirety.

### TECHNICAL FIELD

The subject matter disclosed herein is generally directed to engineered plants useful for e.g., phytoremediation.

### BACKGROUND

Phosphate (Pi) is crucial for genetic maintenance, cellular function, and energy metabolism in plants as intra- and extracellular Pi pools regulate numerous plant signaling pathways (Kanno et al. Plant Cell Physiol 2016, 57, 690-706 and Kuo et al., The Plant Journal 2018, 95, 613-630). Pi is arguably the greatest plant growth-limiting macronutrient, making it the foundation for food production and security worldwide (Kuo et al., The Plant Journal 2018, 95, 613-630; Sattari et al., Proc Natl Acad Sci USA 2012, 109, 6348-6353; and Song et al., Front. Plant Sci. 2015, 6, doi: 10.3389/fpls.2015.00796). While critically important, Pi is, unfortunately, scarce in most soils. There is a phosphate crises, with at most an estimated 300 year supply remaining in minable reserves. Ironically, there is simultaneously a phosphorous pollution is a widespread problem with where excessive application of fertilizer to agricultural land and urban areas leaches into aquatic environments. Thus, there exists a need for improved compositions, methods, and/or techniques for improved Pi utilization and management.

Citation or identification of any document in this application is not an admission that such a document is available as prior art to the present invention.

### SUMMARY

Described in certain example embodiments herein are engineered plant comprising (a) increased NUDIX gene expression as compared to a suitable control; (b) increased amount of one or more NUDIX gene products as compared to a suitable control; (c) increased activity of one or more NUDIX enzymes as compared to a suitable control; or (d) any combination of (a) to (c).

In certain example embodiments, the engineered plant comprises one or more modified NUDIX genes and/or gene products thereby producing an engineered plant that overexpresses one or more NUDIX polypeptides as compared to a suitable control.

In certain example embodiments, the one or more NUDIX gene products are NUDIX mRNA, NUDIX polypeptide(s), or both.

In certain example embodiments, the engineered plant comprising increased NUDIX gene expression (a) comprises one or more exogenous NUDIX genes or portion thereof that encodes a NUDIX polypeptide; (b) comprises one or more modified endogenous NUDIX genes; (c) comprises a modified epigenome thereby increasing NUDIX gene expression; or (d) any combination of (a) to (c).

In certain example embodiments, (a) the one or more modified endogenous NUDIX genes comprise one or more mutations, insertions, deletions, substitutions, or any combination thereof in the coding or non-coding region of the one or more modified endogenous NUDIX genes such that expression of the one or more modified endogenous NUDIX genes are increased as compared to an unmodified control; (b) the modified epigenome comprises decreased DNA methylation of one or more NUDIX genes and/or histone methylation of a histone associated with the one or more NUDIX genes thereby increasing gene expression of the one or more NUDIX genes; (c) the modified epigenome comprises increased histone acetylation of a histone associated with the one or more NUDIX genes thereby increasing gene expression of the one or more NUDIX genes; or (d) any combination of (a) to (c).

In certain example embodiments, the engineered plant comprising increased amount of one or more NUDIX gene products as compared to a suitable control comprises (a) a modified NUDIX mRNA, wherein the mRNA has been modified as compared to an unmodified control to increase protein translation, mRNA stability, or both; (b) a modified NUDIX polypeptide, wherein the polypeptide has been modified as compared to an unmodified control to increase protein stability; or both (a) and (b).

In certain example embodiments, the modified NUDIX mRNA comprises (a) one or more mutations, insertions, deletions, substitutions, or any combination thereof such that translation and/or stability of the NUDIX mRNA is increased; (b) one or more nucleic acid modifications that increases translation and/or mRNA stability; or both (a) and (b).

In certain example embodiments, the engineered plant comprising increased activity of one or more NUDIX enzymes comprises (a) one or more modified NUDIX polypeptides comprises one or more amino acid mutations, insertions, deletions, substitutions, or any combination thereof such that activity of the one or more modified NUDIX polypeptides is increased as compared to a control; (b) comprises one or more post-translational modifications such that activity of the one or more modified NUDIX polypeptides is increased as compared to a control.

In certain example embodiments, the engineered plant comprises a CRISPR-Cas system or component(s) thereof.

In certain example embodiments, the NUDIX gene and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof. In certain example embodiments, the NUDIX gene and/or gene products are selected from NUDIX 4, NUDIX 12, NUDIX 13, NUDIX 14, NUDIX 16, NUDIX 17, NUDIX 18, NUDIX 21, or any combination thereof. In certain example embodiments, the NUDIX gene and/or gene products are selected from NUDIX 4, NUDIX 12, NUDIX, 13, NUDIX 16, NUDIX 21, or any combination thereof.

In certain example embodiments, the exogenous or endogenous NUDIX gene and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof. In certain example embodiments, the exogenous or endogenous NUDIX gene and/or gene products are selected from NUDIX 4, NUDIX 12, NUDIX 13, NUDIX 14, NUDIX 16, NUDIX 17, NUDIX 18, NUDIX 21, or any combination thereof. In certain example embodiments, the exogenous or endogenous NUDIX gene and/or gene products are selected from NUDIX 4, NUDIX 12, NUDIX, 13, NUDIX 16, NUDIX 21, or any combination thereof.

Described in certain example embodiments herein are methods of producing an engineered plant having increased expression of a NUDIX gene and/or gene product, the method comprising overexpressing a NUDIX gene and/or gene products in one or more cells of the engineered plant. In certain example embodiments, the method comprises (a) expressing one or more exogenous NUDIX genes and/or gene products in one or more cells of the engineered plant; (b) modifying one or more endogenous NUDIX genes and/or gene products in one or more cells of the engineered plant, wherein the one or more modifications increase expression of the one or more modified endogenous NUDIX genes and/or gene products as compared to an unmodified control; or both (a) and (b).

In certain example embodiments, the one or more NUDIX genes and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof. In certain example embodiments, the one or more NUDIX genes and/or gene products are selected from NUDIX 4, NUDIX 12, NUDIX 13, NUDIX 14, NUDIX 16, NUDIX 17, NUDIX 18, NUDIX 21, or any combination thereof. In certain example embodiments, the one or more NUDIX genes and/or gene products are selected from NUDIX 4, NUDIX 12, NUDIX, 13, NUDIX 16, NUDIX 21, or any combination thereof.

Described in certain example embodiments herein are methods comprising growing, propagating, harvesting, and/or cultivating an engineered plant of the present description herein.

Described in certain example embodiments herein are methods of phytoremediation and/or reducing the amount of inorganic phosphate in a soil, water, or other aqueous environment, the method comprising planting, growing, harvesting, cultivating, or any combination thereof an engineered plant of the present description herein in a soil and/or aqueous environment. In certain example embodiments, the soil and/or aqueous environment is in need of phytoremediation or inorganic phosphate reduction. In certain example embodiments, the method further comprises producing biochar, fertilizer, or both from the engineered plant.

Described in certain example embodiments herein is use of an engineered plant of the present description herein to reduce the amount of inorganic phosphate in a soil, water, or other aqueous environment in which the engineered plant is grown or cultivated in.

Described in certain example embodiments herein is use of the engineered plant of the present description herein to produce biochar, fertilizer, or both.

Described in certain example embodiments herein are methods of producing biochar, the method comprising growing, propagating, harvesting, and/or cultivating an engineered plant of the present description herein in a soil, water, or other aqueous environment, optionally one in need of phytoremediation or reduction of inorganic phosphate; and carbonizing biomass from the engineered plant by a suitable process to form the biochar.

Described in certain example embodiments herein is biochar and/or fertilizer produced from an engineered plant of the present description herein.

Described in certain example embodiments is biochar produced by the method of the present description herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Maximum likelihood phylogenetic tree representing relationships of amino acid sequences between 27 NUDIX enzymes (Arabidopsis thaliana) and DDP1 (Saccharomyces cerevisiae). The number above each branch is the bootstrap support percentage of n=1000 bootstrapreplications. This phylogenetic tree was rooted on DDP1. Divided into 9 groups based on structural annotations made in (Gunawardana et al., 2009). Blue boxes show annotations based on (Gunawardana et al., 2009), orange boxes show empirically verified substrates from (Ogawa et al., 2008), purple boxes show overlap between Gunawardana et al annotations and Ogawa et al verified substrates, the green box shows annotations from UniProt (The UniProt Consortium, 2021), and the gray box shows empirically verified substrates from (Olejnik et al., 2007).

FIG. 2A-2D—NUDIX12 and NUDIX13 overexpressing (OX) transgenics. (FIG. 2A) Western blot of leaf tissue from heterozygous NUDIX13 OX and NUDIX12 OX Arabidopsis transgenics. DDP1 OX samples were included as a positive control. Tissue used in this analysis came from 4-week-old WT and DDP1 OX plants and 5-week-old NUDIX12 and NUDIX13 OX plants. Ponceau staining shows RuBisCo accumulation in all plants as a positive control (˜50-60 kDa). (FIG. 2B) Rosette growth oer the course of 6 weeks, each image representative of n=3 independent experiments containing 12 or more plants per genotype; scale bar=1 cm. (FIG. 2C) Close up view of ipk1, DDP1-I, and NUDIX13 OX rosettes; scale bar=1 cm, arrowheads mark leaftip necrosis and chlorosis. (FIG. 2D) Average rosette diameter growth over time. Each point represents n=3 independent experiments with over 12 plants per experiment; error bars show standard deviation (SD). Different letters indicate statistically significant means (Tukey HSD, α=0.05).

FIG. 3A-3C—NUDIX12 and NUDIX13 OX InsP and PP-InsP Profiles. (FIG. 3A) All genotypes were grown for 14 days on semi-solid ½× MS media with 0.2% agar then 100 μCi [3H]-myo-inositol was added for 4 days. All InsPs were extracted, separated using anion exchange HPLC, and data analyzed, as described in Experimental Procedures. These InsP profiles are representative of 2-3 independent replicates per genotype; see FIG. 9 for all profiles. (FIG. 3B) Quantification of InsP₆, InsP₇, and InsP₈ from HPLC traces in (FIG. 3A). (FIG. 3C) Quantification of (FIG. 3B) expressed as ratios; InsP₇/InsP₆, InsP₈/InP₆, and InsP₈/InP₇. For FIG. 3B-3C, asterisks show significant differences from WT; analyzed using Student's t-test; **P<0.005, error bars show SD of n=2.

FIG. 4A-4K—Subcellular localization of NUDIX12-GFP in Arabidopsis epidermal cells. Mature leaves of 4-week-old soil grown Arabidopsis plants were imaged. NUDIX12-I (FIG. 4A-4C, 4J-4K), NUDIX12-N (FIG. 4D-4F), and WT (FIG. 4G-4I). Images (FIG. 4A, 4D, 4G, 4J, 4K) were imaged using the GFP channel and chlorophyll autofluorescence was imaged in (FIG. 4B, 4E, 4H). Image (FIG. 4J) highlights NUDIX12-GFP localized in guard cell nuclei. Image (FIG. 4K) is an enlargement of the region outlined in image (FIG. 4J). All scale bars=50 μm.

FIG. 5A-5K—Subcellular localization of NUDIX13-GFP in Arabidopsis epidermal cells. Mature leaves of 10-week-old soil grown Arabidopsis plants were imaged. NUDIX13-O (FIG. 5A-5C, 5J-5K), NUDIX13-H (FIG. 5D-5F), and WT (FIG. 45-5I). Images (FIG. 5A, 5D, 5G, 5J, 5K) were imaged using the GFP channel and chlorophyll autofluorescence was imaged in (FIG. 5B, 5E, 5H). Image (FIG. 5J) highlights NUDIX13-GFP localized in guard cell nuclei. Image (FIG. 5K) is an enlargement of the region outlined in image (FIG. 5J). All scale bars=50 μm.

FIG. 6A-D—Pi accumulation in NUDIX12 and NUDIX13 OX lines. Leaf Pi accumulation from 3-4 week-old soil-grown plants. Each point represents pooled plant tissue from n=2-3 independent experiments (except for NUDIX12-I n=1); error bars show SD. Asterisks show significant differences from WT; analyzed using Student's t-test; **P<0.005; ***P<0.0005 (FIG. 6A). Cauline leaf Pi accumulation from 6 week-old soil-grown plants. Each point represents pooled plant cauline leaves from n=2-3 independent experiments; error bars show SD (FIG. 6B). Pi accumulation in pooled leaf tissue from 2-6-week-old soil-grown plants. Each point represents pooled leaf tissue from n=1 independent experiment; letters “a” and “b” denote independent replicates (FIG. 6C-D).

FIG. 7A-7H-Structures of DDP1, NUDIX12, and NUDIX13 superimposed with 5-InsP₇ DDP1 (teal, as represented in greyscale) resolved crystal structure (FIG. 7A); NUDIX12 (purple, as represented in greyscale) (FIG. 7B) and NUDIX13 (pink, as represented in greyscale) (FIG. 7C) homology models. Mg²⁺ (green, as represented in greyscale) shown in active site; 5-InsP₇. Multiple sequence alignment (MSA) of DDP1 and Group 9 Arabidopsis NUDIX enzymes (SEQ ID NOS: 63-70) (FIG. 7D). All sequences were aligned using Schrödinger-Maestro's Multiple Sequence Viewer. The percent amino acid similarity(S) and identity (I) are shown on the right with DDP1 as the reference. All sequences are displayed using a single letter for each amino acid and conserved residues are colored based on side-chain properties. The conserved NUDIX box is indicated by the black box and the “SSA” from DDP1 is indicated by the grey box. Overlay of all three enzyme models (FIG. 7E). DDP1 (FIG. 7F), NUDIX12 (FIG. 7G), and NUDIX13 (FIG. 7H) interacting amino acids within 4 Å of 5-InsP₇. Dotted lines represent interactions within 4.5 Å of 5-InsP₇ and amino acid molecules; red box locates amino acids within red box from the MSA alignment on (FIG. 7D).

FIG. 8—Five different InsP₇ binding modes (BM) within DDP1 active site. White arrow heads in BM-C and BM-D indicate that the PPi could be found on either of these locations and would be considered part of this BM group. The sphere represents the Mg²⁺ in the active site.

FIG. 9—Multiple sequence alignment (MSA) of DDP1 and 27 predicted Arabidopsis NUDIX enzymes (SEQ ID NOS: 71-98). All amino acid sequences were obtained from UniProt (The UniProt Consortium, 2021) and aligned using Schrödinger-Maestro's Multiple Sequence Viewer (Schrödinger, 2021). The percent amino acid similarity(S) and identity (I) are shown on the right with DDP1 as the reference. All sequences are displayed using a single letter for each amino acid and conserved residues are shaded based on side-chain properties. The conserved NUDIX box is indicated by the black box and the “SSA” from DDP1 is indicated by the box.

FIG. 10A-10E-Structural validation of energy minimized NUDIX4 homology model. (FIG. 10A) The Ramachandran plot shows that 98.1% of residues are in favorable regions and 0.5% in outlier regions generated using SWISS-MODEL structural assessment tools (Kiefer et al., 2009, p.). (FIG. 10B) The Protein Structure Analysis (ProSA) indicates Applicant's homology model has a Z-score of −5.4 (black dot) and its similarity to other resolved NMR and crystal structures (Wiederstein and Sippl, 2007). (FIG. 10C) Verify3D analysis displays side chain favorability with 80.7% of residues above the line (Bowie et al., 1991). (FIG. 10D) Structure of NUDIX4 homology model superimposed with 5-InsP₇; visualized using PyMol (Schrödinger, LLC, 2021). (FIG. 10E) The QMEAN Z-scores of the NUDIX4 homology model.

FIG. 11A-11E-Structural validation of energy minimized NUDIX12 homology model. (FIG. 11A) The Ramachandran plot shows that 96. % of residues are in favorable regions and 0% in outlier regions generated using SWISS-MODEL structural assessment tools (Kiefer et al., 2009, p.). (FIG. 11B) The ProSA indicates Applicant's homology model has a Z-score of −6.11 (black dot) and its similarity to other resolved NMR and crystal structures (Wiederstein and Sippl, 2007). (FIG. 11C) Verify3D analysis displays side chain favorability with 92.1% of residues above the line (Bowie et al., 1991). (FIG. 11D) Structure of NUDIX12 homology model superimposed with 5-InsP₇; visualized using PyMol (Schrödinger, LLC, 2021). (FIG. 11E) The QMEAN Z-scores of the NUDIX12 homology model.

FIG. 12A-12E—Structural validation of energy minimized NUDIX13 homology model. (FIG. 12A) The Ramachandran plot shows that 96% of residues are in favorable regions and 1% in outlier regions generated using SWISS-MODEL structural assessment tools (Kiefer et al., 2009, p.). (FIG. 12B) The Protein Structure Analysis (ProSA) indicates Applicant's homology model has a Z-score of −6.67 (black dot) and its similarity to other resolved NMR and crystal structures (Wiederstein and Sippl, 2007). (FIG. 12C) Verify3D analysis displays side chain favorability with 91.6% of residues above the line (Bowie et al., 1991). (FIG. 12D) Structure of NUDIX13 homology model superimposed with 5-InsP₇; visualized using PyMol (Schrödinger, LLC, 2021). (FIG. 12E) The QMEAN Z-scores of the NUDIX13 homology model.

FIG. 13A-13E—Structural validation of energy minimized NUDIX16 homology model. (FIG. 13A) The Ramachandran plot shows that 98.9% of residues are in favorable regions and 0% in outlier regions generated using SWISS-MODEL structural assessment tools (Kiefer et al., 2009, p.). (FIG. 13B) The Protein Structure Analysis (ProSA) indicates Applicnt's homology model has a Z-score of −6.25 (black dot) and its similarity to other resolved NMR and crystal structures (Wiederstein and Sippl, 2007). (FIG. 13C) Verify3D analysis displays side chain favorability with 100% of residues above the line (Bowie et al., 1991). (FIG. 13D) Structure of NUDIX16 homology model superimposed with 5-InsP₇; visualized using PyMol (Schrödinger, LLC, 2021). (FIG. 13E) The QMEAN Z-scores of the NUDIX16 homology model.

FIG. 14A-14E—Structural validation of energy minimized NUDIX17 homology model. (FIG. 14A) The Ramachandran plot shows that 96.7% of residues are in favorable regions and 0.6% in outlier regions generated using SWISS-MODEL structural assessment tools (Kiefer et al., 2009, p.). (FIG. 14B) The Protein Structure Analysis (ProSA) indicates Applicant's homology model has a Z-score of −6.82 (black dot) and its similarity to other resolved NMR and crystal structures (Wiederstein and Sippl, 2007). (FIG. 14C) Verify3D analysis displays side chain favorability with 82.4% of residues above the line (Bowie et al., 1991). (FIG. 14D) Structure of NUDIX17 homology model superimposed with 5-InsP₇; visualized using PyMol (Schrödinger, LLC, 2021). (FIG. 14E) The QMEAN Z-scores of the NUDIX17 homology model.

FIG. 15A-15E-Structural validation of energy minimized NUDIX18 homology model. (FIG. 15A) The Ramachandran plot shows that 99.4% of residues are in favorable regions and 0.6% in outlier regions generated using SWISS-MODEL structural assessment tools (Kiefer et al., 2009, p.). (FIG. 15B) The Protein Structure Analysis (ProSA) indicates Applicant's homology model has a Z-score of −6.32 (black dot) and its similarity to other resolved NMR and crystal structures (Wiederstein and Sippl, 2007). (FIG. 15C) Verify3D analysis displays side chain favorability with 84.1% of residues above the line (Bowie et al., 1991). (FIG. 15D) Structure of NUDIX18 homology model superimposed with 5-InsP₇; visualized using PyMol (Schrödinger, LLC, 2021). (FIG. 15E) The QMEAN Z-scores of the NUDIX18 homology model.

FIG. 16A-16E—Structural validation of energy minimized NUDIX21 homology model. (FIG. 16A) The Ramachandran plot shows that 94.4% of residues are in favorable regions and 1% in outlier regions generated using SWISS-MODEL structural assessment tools (Kiefer et al., 2009, p.). (FIG. 16B) The Protein Structure Analysis (ProSA) indicates Applicant's homology model has a Z-score of −5.48 (black dot) and its similarity to other resolved NMR and crystal structures (Wiederstein and Sippl, 2007). (FIG. 16C) Verify3D analysis displays side chain favorability with 71.2% of residues above the yellow line (Bowie et al., 1991). (FIG. 16D) Structure of NUDIX21 homology model superimposed with 5-InsP₇; visualized using PyMol (Schrödinger, LLC, 2021). (FIG. 16E) The QMEAN Z-scores of the NUDIX21 homology model.

FIG. 17—Pairwise sequence alignment of NUDIX12 and NUDIX13 (SEQ ID NOS: 99-100). Sequences were aligned using Schrödinger-Maestro's Multiple Sequence Viewer. The percent amino acid similarity(S) and identity (I) are shown on the right with NUDIX12 as the reference. Sequences are displayed using a single letter for each amino acid and conserved residues are shaded based on side-chain properties. The conserved NUDIX box is indicated by the black box and the “SSA” motif from DDP1 is indicated by the box.

FIG. 18—Structure of the ligands used in molecular modeling experiments. Position of the PPi groups on InsP₇ and InsP₈ molecules denoted by a blue arrow. Oxygens are depicted in red, as represented in greyscale, Phosphorus in orange as represented in greyscale, and Carbon as represented in various shades of grey.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further embodiment. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. Biologic entities include animals, plants, and the like. A biologic sample can contain whole cells and/or live cells and/or cell debris, and/or cell products, and/or virus particles. A biological sample can be or be from a product produced by a biologic entity, such as a bodily fluid, fruit, nut, pollen, and/or the like, whether still part of, connected to, associated with, or separated from the biologic entity that produced it. The biological sample can be obtained from an environment (e.g., water source, soil, air, and the like). Such samples are also referred to herein as environmental samples.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen, and microspores. The class of plants which can be used in the methods of the invention is generally as broad as the class of higher plants amenable to transformation techniques, including both monocotyledonous and dicotyledonous plants.

As used herein, “engineered plant” includes reference to a plant that comprises within its genome a polynucleotide that has been modified to impart some functional change in the sequence, expression, gene product function, phenotype, and/or the like in the engineered plant. It will be appreciated that the engineered plant may be or may not a “genetically modified organism” defined by the U.S. Department of Agriculture, Federal Drug Administration, or other appropriate regulatory authority and that any such classification does impact its standing as an engineered plant in the context of the present description. Generally, the modified polynucleotide is stably integrated within the genome such that the polynucleotide is passed on to successive generations. The modified polynucleotide may be integrated into the genome alone or as part of a recombinant expression cassette. “Engineered” is used herein to include any cell, cell line, callus, tissue, plant part or plant, the genotype of which has been altered by the presence of a modified nucleic acid including those engineered plants initially so altered as well as those created by sexual crosses or asexual propagation from the initial transgenic. The term “engineered” as used in this context herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

The terms “subject,” “individual,” are used interchangeably herein to refer to a plant, cell thereof, and/or progeny thereof.

Various embodiments are described hereinafter. It should be noted that the specific embodiments are not intended as an exhaustive description or as a limitation to the broader embodiments discussed herein. One embodiment described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, “control” can refer to an alternative subject or sample used in an experiment for comparison purpose and included to minimize or distinguish the effect of variables other than an independent variable.

As used herein with reference to the relationship between DNA, cDNA, RNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “culturing” can refer to maintaining cells under conditions in which they can proliferate and avoid senescence as a group of cells. “Culturing” can also include conditions in which the cells also or alternatively differentiate.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “gene product” refers to a polynucleotide and/or polypeptide products produced from a transcribed and optionally translated gene. Gene products include products produced from post-transcriptional and post-translational modifications of the polynucleotide and/or polypeptide directly transcribed and optionally translated from the gene, including but not limited to splice variants and polypeptide products produced by cleavage of one or more portions of a pre- or prepro-protein.

As used herein, “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, in embodiments modulation may encompass an increase in the value of the measured variable by about 10 to 500 percent or more. In embodiments, modulation can encompass an increase in the value of at least 10%, 20%, 30%, 40%, 50%, 75%, 100%, 150%, 200%, 250%, 300%, 400% to 500% or more, compared to a reference situation or suitable control without said modulation. In embodiments, modulation may encompass a decrease or reduction in the value of the measured variable by about 5 to about 100%. In some embodiments, the decrease can be about 5%, 10%, 20%, 30%, 40%, 50%, 60%, 70%, 80%, 90%, 95%, 96%, 97%, 98%, 99% to about 100%, compared to a reference situation or suitable control without said modulation. In embodiments, modulation may be specific or selective, hence, one or more desired phenotypic embodiments of a cell or cell population may be modulated without substantially altering other (unintended, undesired) phenotypic embodiment(s).

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more greater than the normal, unmodified, or control cell. “increased expression” or “overexpression” are both used to refer to an increased expression of a gene, such as a transgene or gene product thereof in a sample as compared to the expression of said gene or gene product in a suitable control. The term “increased expression” refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control in some embodiments herein.

As used herein, “plasmid” refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “substantial” and “substantially,” specify an amount of between 95% and 100%, inclusive, between 96% and 100%, inclusive, between 97% and 100%, inclusive, between 98% and 100%, inclusive, or between 99% and 100%, inclusive.

As used herein, the term “vector” or is used in reference to a vehicle used to introduce an exogenous nucleic acid sequence into a cell. A vector may include a DNA molecule, linear or circular (e.g. plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### Overview

As previously discussed, there is a paradoxical phosphate crises and simultaneous phosphorous pollution problem plaguing modern agriculture. Despite regulations, management plans, and other approaches to address this issue over the years, it is still a growing problem worldwide. Thus, there still exists an urgent need for improved compositions, methods, and/or techniques for improved Pi utilization and management.

With that said, embodiments disclosed herein provide, among other embodiments, engineered plants that overexpress one or more NUDIX enzymes and/or have increased NUDIX enzyme activity. In some embodiments, the engineered NUDIX overexpressing plants can accumulate more Pi than unmodified plants. Also provided herein are their use for inorganic phosphate reuptake or phytoremediation. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered NUDIX Overexpressing Plants

Described in exemplary embodiments engineered plant having (a) increased NUDIX gene expression as compared to a suitable control; (b) increased amount of one or more NUDIX gene products as compared to a suitable control; (c) increased activity of one or more NUDIX enzymes as compared to a suitable control; or (d) any combination of (a) to (c).

In some embodiments, the engineered plant has about 0.01-100 fold or more increase in the expression of one or more NUDX genes, amount of one or more NUDIX gene products, and/or activity of one or more NUDIX enzymes as compared to a suitable control. In some embodiments, the engineered plant has about 0.01, to/or 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 fold or more in the expression of one or more NUDX genes, amount of one or more NUDIX gene products, and/or activity of one or more NUDIX enzymes as compared to a suitable control.

In some embodiments, the engineered plant has about 1-1,000 percent or more increase in the expression of one or more NUDX genes, amount of one or more NUDIX gene products, and/or activity of one or more NUDIX enzymes as compared to a suitable control. In some embodiments, the engineered plant has about a 1%, to/or 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more increase in the expression of one or more NUDX genes, amount of one or more NUDIX gene products, and/or activity of one or more NUDIX enzymes as compared to a suitable control.

In some embodiments, the one or more NUDIX gene products are one or more NUDIX mRNA, one or more NUDIX polypeptides, or both.

In some embodiments, the engineered plant contains one or more modified NUDIX genes and/or gene products such that the engineered plant overexpresses one or more NUDIX polypeptides as compared to a suitable control.

In some embodiments, the engineered NUDIX overexpressing plant accumulates more Pi than an unmodified plant or other suitable control. In some embodiments, the engineered NUDIX overexpressing plant accumulates about 1%-1,000% or more Pi as compared to an unmodified plant or other suitable control. In some embodiments, the engineered NUDIX overexpressing plant accumulates about 1%, to/or 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more Pi as compared as compared to an unmodified plant or other suitable control.

### NUDIX Genes and Gene Products

NUDIX genes and gene products as the terms are used herein refers to a NUDIX gene and products produced therefrom by any native or synthetic synthesis, including but not limited to RNA (e.g., mRNA, and any other RNAs transcribed from a NUDIX gene), polypeptides translated from a NUDIX RNA, and any intermediates, splice variants, proproteins, pre-proproteins, glycosylation variants, and/or the like. The term “NUDIX gene” as used herein refers to the gene encoding NUDIX (Nucleoside Diphosphate compounds linked to a moiety X) polypeptides or regions thereof capable of performing NUDIX enzymatic activities. NUDIX enzymes are a highly diverse set of pyrophosphatases found in all domains of life. The term “NUDIX polypeptide” refers to a polypeptide that is the full length NUDIX enzyme or is a functional fragment thereof that is capable of performing one or more enzymatic activities. NUDIX polypeptides are encoded by NUDIX genes. In some embodiments, the functional fragment of a NUDIX polypeptide is or at least contains a NUDIX box (SEQ ID NO: 55). In some embodiments, the functional fragment of a NUDIX polypeptide at least hydrolysis activity. In some embodiments, the functional fragment of a NUDIX polypeptide is capable of hydrolyzing non-nucleotide-based molecules, such as inositol pyrophosphates (PP-InsPs), polyphosphates (polyP), 5-phosphoribosyl 1-diphosphate, thiamine pyrophosphate, or dihydroneopterin triphosphate.

NUDIX enzymes share a highly conserved motif of 23 amino acids known as the NUDIX box or MutT: GX₅EX₇REUXEEXGU where X is any amino acid and U is a hydrophobic residue (SEQ ID NO: 55) (Sheikh et al., 1998; Xu et al., 2002; Kraszewska, 2008). The Nudix box motif is essential for NUDIX hydrolysis of a variety of substrates (Gunawardana et al., 2009; Márquez-Moñino et al., 2021). Structurally, this sequence is found in a loop-helix-loop motif within the NUDIX enzyme (McLennan, 2006). NUDIX catalytic activity is dependent on the two underlined glutamates, REUXEE (SEQ ID NO: 62), for hydrolysis activity and binding to metal cofactors (Mildvan et al., 2005; McLennan, 2006). NUDIX enzymes have been found to hydrolyze a wide range of substrates. These include nucleotide-based molecules such as dinucleotides, dNTPs, along with capped mRNAs, diadenosine polyphosphates (ApₙAs), and other modified nucleotides, as well as non-nucleotide-based molecules: inositol pyrophosphates (PP-InsPs), polyphosphates (polyP), 5-phosphoribosyl 1-diphosphate, thiamine pyrophosphate, and dihydroneopterin triphosphate (McLennan, 2006; Kraszewska, 2008). While the structures of multiple NUDIX enzymes have been resolved across organisms (Massiah et al., 2003; Zong et al., 2021; Márquez-Moñino et al., 2021; Srouji et al., 2017), there remain many unknowns within the plant NUDIX enzyme family.

A total of 27 NUDIX enzymes (referred to herein as NUDIX 1-NUDIX 27) have been identified in Arabidopsis thaliana (Kraszewska, 2008; Williams, 2015). Of these 27 enzymes, only the structures of NUDIX1 (Liu et al., 2018) and NUDIX7 (Tang et al., 2015) have been resolved. Further, the enzymatic activities of about half of these enzymes have been empirically explored but the roles of many of these NUDIX enzymes remain unelucidated (Kraszewska, 2008).

In some embodiments, the NUDIX gene and/or gene products are selected from a NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof. In some embodiments, the NUDIX gene and/or gene products are selected from NUDIX 4, NUDIX 12, NUDIX 13, NUDIX 14, NUDIX 16, NUDIX, 17, NUDIX 18, NUDIX 21, or any combination thereof. In some embodiments, the NUDIX gene and/or gene products are selected from NUDIX 4, NUDIX 12, NUDIX, 13, NUDIX 16, NUDIX 21, or any combination thereof. In some embodiments, the NUDIX gene and/or gene products are selected from any one of those presented in Tables 1-2 below, or a homologue, orthologue, and/or structural or functional variant thereof. In some embodiments, the homologue, orthologue, and/or structural or functional variant thereof of a NUDIX gene and/or gene products selected from any one of those presented in Tables 1-2 below is from a plant or other eukaryotic organism (e.g., a mammal such as a human, dog, mouse, cat, etc.). In some embodiments, the homologue, orthologue, and/or structural or functional variant thereof of a NUDIX gene and/or gene products selected from any one of those presented in Tables 1-2 below is from a yeast. In some embodiments, the NUDIX gene or gene product is not DDP1. In some embodiments, the NUDIX polypeptide is not DDP1. A reference DDP1 sequence is provided in SEQ ID NO: 101, which corresponds to YOR163W [Saccharomyces cerevisiae] DDP1 (GenBank Accession No. AAS56762.1). A reference encoding DDP1 sequence will be appreciated by one of ordinary skill in the art. An exemplary reference sequence encoding a DDP1 can be found at GenBank Accession No: AY558436. In some embodiments, the DDP1 polypeptide sequence is 70-100% identical to SEQ ID NO: 101. In some embodiments, the NUDIX polypeptide is not a DDP1 polypeptide sequence that is 70-100% identical to SEQ ID NO: 101. In some embodiments, the NUDIX polypeptide is not a DDP1 polypeptide sequence that is 70-100% identical to a coding sequence available at GenBank Accession No. AY558436).

The Tables below provides reference sequences for NUDIX genes and/or gene products. All homologues, orthologues, and structural and functional variants of a NUDIX gene and/or gene product described herein are within the scope of the present description and will be appreciated by those of skill in the art in view of the description herein. Where a mutation is described or presented in the context of a reference NUDIX sequence (DNA, RNA, or protein), one of ordinary skill in the art will, using generally available techniques and methodology known in the art, appreciate and understand corresponding mutations in homologues and orthologues of the reference NUDIX sequence.

TABLE 1
Reference NUDIX Encoding polynucleotides from Arabidopsis thaliana

| | mRNA (GenBank | NCBI | | SEQ |
|---|---|---|---|---|
| NUDIX | Accession No.) | Gene ID | Sequence | ID NO: |
| 1 | NM_105549 | 843207 | AAATTTTTTAATAAATAAAATGAAAACAAGTGCAAT | 1 |
| | | | GGAAAAAGAAACAGCGATAATTGTGTACAGCCAA | |
| | | | TCACACCGACACGGCGACACATTTGCCAACACGTAT | |
| | | | ATAAACGCATAACCGATACGACATCGTTTTAAAC | |
| | | | TCAAATTAAGTCAACAAAACTAAAACACGACAAGA | |
| | | | AAGGGTTTCACTGTGTTCGATAATAATGTCGACAG | |
| | | | GAGAAGCGATACCAAGAGTCGCCGTCGTCGTTTTCA | |
| | | | TTCTCAACGGAAACTCAATCTTATTAGGTCGCCG | |
| | | | CCGTTCCTCAATCGGCAACTCCACTTTCGCTCTTCCC | |
| | | | GGTGGCCACCTCGAATTCGGAGAGAGCTTTGAA | |
| | | | GAATGTGCAGCGAGAGAAGTAATGGAGGAAACAGG | |
| | | | TCTAAAGATTGAAAAGATGAAGCTTTTGACTGTTA | |
| | | | CAAACAATGTCTTCAAAGAAGCACCAACGCCATCAC | |
| | | | ACTACGTCTCTGTTTCGATACGTGCGGTGTTGGT | |
| | | | GGATCCAAGTCAAGAACCGAAGAATATGGAACCAG | |
| | | | AGAAGTGTGAAGGATGGGATTGGTATGATTGGGAG | |
| | | | AATCTACCAAAGCCTTTGTTTTGGCCACTTGAGAAA | |
| | | | TTGTTTGGAAGTGGTTTCAATCCTTTCACTCATG | |
| | | | GTGGTGGAGACTAATAGATGTAAGAGTTAATGATTG | |
| | | | ATTTGGGATTGAATGTTGCAAATTGGGCATTTGG | |
| | | | TCTAGTGGTATGATTCTCGCTTAGGGTGCGAGAGGT | |
| | | | CCCGAGTTCAATTCTCGGAATGCCCCTCTCTTTT | |
| | | | ACTCTTTTTTATTGTATTTTTGGTTTCTGTTTTCCTAA | |
| | | | TTAGTAATAAACTTATCATGTGTCCTATCGAT | |
| | | | TTTATTTCTC | |
| 2 | NM_124139 | 834816 | GAGAAACTATTCCACGTACGTATTAACCTTTGACCT | 2 |
| | | | TAACCTTCTTAAACAGAAAATATTGAAGTTTTTA | |
| | | | TGTTCTTGTGATAGAAATATACAGTAACAGAATATT | |
| | | | TTCTTTGGCCTTTATTGACATATTTTAGAAGAAA | |
| | | | AATAGATTACTTCCTTGTTTTATTATTATTTCACTCA | |
| | | | TCATACGTTGTAGAAAGGGAACCGCAAAGTGAA | |
| | | | AAAACAGTTGCTCCACCACGAGGAACAGATCCATGT | |
| | | | CTGCTTCTAGTTCCTCCACAAATCCCATGTCCCG | |
| | | | TGAGGACGCAACAACACTGCTTCCGTCTGTTCAAGA | |
| | | | CAAGTACGGTGGCGTGATGACCGAGATGACTCAT | |
| | | | CCCATGGATCCTTCTCTCTTCTCCACACTTCTCCGAT | |
| | | | CCTCTCTATCTACCTGGACTCTTCAGGGCAAAA | |
| | | | AGGGAGTGTGGATCAAGTTGCCAAAGCAGCTTATTG | |
| | | | GTCTTGCTGAAACTGCTGTTAAGGAGGGATTCTG | |
| | | | GTTTCACCACGCGGAGAAAGACTACTTGATGCTTGT | |
| | | | CTATTGGATTCCCAAAGAAGACGATACGCTTCCT | |
| | | | GCCAATGCATCTCACCGTGTTGGCATAGGTGCCTTT | |
| | | | GTCATTAACCACAACAAAGAGGTGCTGGTGGTTC | |
| | | | AAGAGAAGACAGGGAGATTTCAAGGTCAAGGTATC | |
| | | | TGGAAGTTCCCCACCGGAGTTGTGAATGAGGGTGA | |
| | | | AGACATTCATGATGGCTCAGTCAGAGAGGTGAAAG | |
| | | | AAGAGACCGGAGTGGATACAGAATTCGACCAAATA | |
| | | | TTGGCCTTCAGACAAACACACAAAGCGTTCTTTGGA | |
| | | | AAGTCAGATTTGTTCTTCGTGTGCATGTTAAAGC | |
| | | | CCCTTTCTTTGGAGATCAACGCACAGGAGTCAGAGA | |
| | | | TTGAGGCAGCTCAGTGGATGCCTTGGGAGGAATA | |
| | | | CATAAATCAACCATTTGTACAAAATTATGAGCTATT | |
| | | | GAGGTATATGACAGACATTTGCTCTGCTAAAACC | |
| | | | AACGGAGACTACGAAGGCTTCACTCCTCTCCGCGTC | |
| | | | TCTGCACCTGATCAACAAGGCAACTTGTACTACA | |
| | | | ACACCCGCGACTTACATTCGCGCAATTGACCTTCTTT | |
| | | | CTCAACAAACAGAGCTTTCCTCACAGATTCTAA | |
| | | | CGAAGCTGGTTTCTTTGCTGCCTCTCTATGTAAGCAT | |
| | | | TTTGACACTTGAATCACTACATAACTCTCACCA | |
| | | | AAACATAAAAGCATCCCAAATGAAAACATTTGCATT | |
| | | | CAATACAATGGCTTTAGTTACATACAAGATTCAA | |
| | | | TGAAGCAGATTCGTTAGT | |
| 3 | NM_106618 | 844308 | CAACTAACATGAACTAACTGCTGACATAATCGTCTT | 3 |
| | | | AACATTAAAAAAAAATGAAATGAATGCATGCATT | |
| | | | GCATATAAATGTACAAATGTTGGCTTGATCTCTTGA | |
| | | | CCCCTACCTAAGACGTAAGAAGGTATTTTAGAGC | |
| | | | TGTGAAATGTGTCAAGCTGAGTCTCGGAGTGAAAAG | |
| | | | GGTCCGGTTTAGTGAAGCCAAGTAAGCAAAGCCC | |
| | | | CTTGTTTTTGACGGACATTATATCTCCTCCTCTTCTC | |
| | | | TTCTATAAATTGGGCACCTTTTTCCAATTTTAC | |
| | | | GCACAGTCACGGAGATAGATAGTAAGAGAGAGCTA | |
| | | | TGGCGGAGGAGCACTTCGACGTTCTGACTAAATCC | |
| | | | GGTGAGAAGACCGGTGTGTCCAAACCCAGGGGAGA | |
| | | | GGTTCACAGAGATGGAGATTACCATCGTGCTGTTC | |
| | | | ATGTCTGGATTTTTGTGGAGACTACTCAACAGCTAC | |
| | | | TTCTCCAACTTCGATCTGATGATAAAGATTCTTG | |
| | | | GCCTGGTCAATGGGACATCTCTAGTGCTGGCCATAT | |
| | | | ATCAGCTGGTGATACATCGCTTCTATCTGCACAG | |
| | | | CGGGAGCTTGAGGAAGAGCTTGGCGTAAAACTCCC | |
| | | | AAAGGATGCATTTGAAAAGATCTTCGTTTTTCTGC | |
| | | | AGGAATGTGTCACAAACGATGGCAAATTCATAAAT | |
| | | | AATGAGTTCAATGATGTGTATCTTGTTACTATTCT | |
| | | | GCATCCAATCCCATTAGAAGCCTTTACTCTTCAAAA | |
| | | | AGAGGAAGTCTCTGCTGTAAAGTACGTACCTTAC | |
| | | | GAAGAATACAGAAACTTTCTGTCTAAGGAAGATCCT | |
| | | | GCTTATGTGCCATATGATGTTAATGGAGAGTATG | |
| | | | GAAAACTATTTGACATAATCAGACAGAGGTGCCAA | |
| | | | GTGAACACTGAGGCCCGTAGTTTATCTCTGCAGAA | |
| | | | GCAACTCCAGAGATATTCTCCAGTCACCCTCGAAGC | |
| | | | CAAGTTGACAGAACTTTCTGAAGCAGATCAGAAA | |
| | | | GCGTTGGGTCTAATTGTGAAGGCTGCAAAAATTATG | |
| | | | GACGATATATTCTATGAGCAGGTTTGGAACAGCA | |
| | | | ATCCAGCCCTAAGAGATTGGCTGAAGGATCATGCCA | |
| | | | ATGCATCAAAACTGGACAAACTAAAATGGGATTA | |
| | | | CTTCACGATCAACAAAAGTCCATGGTCGTCTCTTGA | |
| | | | TGAAAATGAAGCATTCTTGAGTACTGCGGATTCA | |
| | | | GCTGTAAAGTTGCTTCCTGGAGCAACAAAAGCCATA | |
| | | | GCTGGCTGGAAGGGTCTTGAATATAGAGCAGCCT | |
| | | | TTCCAGTGACTAAACCACCTGGTGCCAATTTCTATC | |
| | | | CTCCCGATATGGATAAAATGGAGTTCACCTTATG | |
| | | | GCTCAATGGCCTTACGGAAGAGCAAAAGCATGCTG | |
| | | | CAACAGGATTTTTCAGTGTTATAAAAAGGCGAAGT | |
| | | | GAAGCTAATTTAGATGCTTCTGATCATCTTGCCAGC | |
| | | | AGCACAAAGAAGTTACCAGACTCTAATTCCGATC | |
| | | | TGTATAGTATTCCTTATTCAGAGATATATCGACCTTT | |
| | | | CCTCAAGAAAGCTTCTGAATTCTTGCAGAAAGC | |
| | | | TGGGGACTTGGTCAGTTCGCCAAGTTTGAAGAAGCT | |
| | | | ACTGCACAGCAAAGCTGAAGCCTTCCTTTCAAAT | |
| | | | GAGTATTATGAGTCAGACATCGCATGGATGGACTTG | |
| | | | GATTCAAAGTTAGATATTACCATTGGTCCATATG | |
| | | | AGACGTACGAGGATGAAATCTTTGGGTATAAGGCTA | |
| | | | CATTTGAAACCTTCATCGGTATTCGAGATGATAA | |
| | | | AGCCACAGCTGATCTGAAACTCTTTGGTGACAATCT | |
| | | | AAAGCTATTGGAAGATAATCTCCCGCTTGAGAGT | |
| | | | GTATACAAATCGACAGATGTATCTGCTGCTCCTATT | |
| | | | CGGGTCATCCAGTTAATTTACAATTCCGGGGATG | |
| | | | TGAAGGGTCCTCAGACTGTTGCTTACAATTTGCCAA | |
| | | | ACGATGAAAAAATCGTGAAGGATAGAGGAACATC | |
| | | | AATGGTTATGCTCAAGAACGTCCAAGAGGCAAAGTT | |
| | | | TGAACATATTTTAAAGCCTATAGCTGAAATCACC | |
| | | | ATCAGCAAGGAACAGAGAGGATTAGTTGATTTTGAT | |
| | | | TCTTTCTTCACTCACACCATTTGCCATGAATGTT | |
| | | | GCCATGGCATTGGTCCCCATACCATAACCCTTCCTG | |
| | | | GTGGTCAAACCTCAACAGTGAGAAAGGAACTACA | |
| | | | AGAAGTCCATTCTGCAATGGAAGAAGCAAAGGCAG | |
| | | | ATATAGTTGGTCTTTGGGCACTGAAGTTTCTCATC | |
| | | | ACTAAGGGGTTGCTTTCAAAGAGTATGGTGGAGAGT | |
| | | | ATGTATGTTTCGTTCCTAGCTGGTTGCTTTAGAT | |
| | | | CGATCCGGTTTGGTCTCACAGAAGCTCATGGGAAAG | |
| | | | GGCAAGCCTTGCAATTCAACTATTTATATGAAAA | |
| | | | AGGAGCCTTCGTGTTCCACGAGGACTCTACATTCTC | |
| | | | GGTTGATTTCGCTAAGATCGAAGGTGCAGTTGAG | |
| | | | AGCCTGAGCCATGAGATACTAACTATTCAAGGAAA | |
| | | | AGGTGACAAAAACGCAGCAACATTGCTTCTAAATA | |
| | | | AGTACTGCACTATCACCGGACCATTGAAAACCGCTC | |
| | | | TGGAGAATCTCGAACGCGTTAAGGTACCGGTGGA | |
| | | | TATATCTCCGACATTTCCATTAGCAGAAGCATTGAT | |
| | | | GAACTGAGGAAAGTTTCAGTTTCAGCAAGACTCT | |
| | | | GGTTTTAACTTTTAATAAATAAATAAAACTCAGATG | |
| | | | ATCCATCATCATAAACCAAATTTGGGATTTAAAA | |
| | | | GAGTTATCTGAATGGTGTTGTGTTCTTATCATAATAA | |
| | | | AGTTGCTACCTTTACATCTTTATTCCATAGAAG | |
| | | | AAATTTTTGAGAGCTTTAAGATAGAATGTTATCATC | |
| | | | ATCACTAAGAAATA | |
| 4 | NM_101688 | 838410 | AAAACCAATAACGCTCTTAACAAATTCACATCTCCC | 4 |
| | | | TCTCTCTTATCCATTTCCTCTCTAGACTTCCTTA | |
| | | | AATCGATTCTCCCGAATTCGAGAACAGAGCCTTTTT | |
| | | | TTCTTTAATTCCAACTCTTTCTCTCTTGTTCTGA | |
| | | | ATTCGTGGTTCGTCGGTATTATAAAAAAAAACATGA | |
| | | | CAGGGTTCTCTGTGTCTCTTTTTGTGTCAAACTT | |
| | | | GTCAAATGTGGCCTCGTATTTGTCGCCCATCTTCGA | |
| | | | GAACATCCCCTCCACCAAGGTTGTTCCCGCACAG | |
| | | | ATCGAGAAAGTTGTCTCTTTGGTCTCTCGTACCGGTC | |
| | | | GCGATTTGCAGCGTTACGATCACGCTGGCTATC | |
| | | | GTCAAGTCGTCGGATGTGTACCGTATAGATACAAGA | |
| | | | AACAAGAAGTCAATGGAGTTGAAACCCAAGTAAT | |
| | | | CCAAGTTCTTCTTGTCAGTGCTCAAAAGGGCAAAGG | |
| | | | AATGTTATTTCCAAAAGGAGGTTGGGAGACGGAT | |
| | | | GAATCAATGGAGGAAGCTGCTTTGAGAGAGACGAT | |
| | | | CGAAGAAGCGGGTGTAACAGGAGAGCTCGAAGAAA | |
| | | | AGCTTGGGAAATGGCAATACAAAAGCAAAAGACAT | |
| | | | AGCATAATTCACGATGGGTATATGTTTGCTTTGCT | |
| | | | TGTCAGTCAAGAGTTCGAGCGATGGCCTGAGGCGG | |
| | | | AAATGAGACAACGCAGATGGGTAAGTTTGGATGAA | |
| | | | GCAAGAGAAGTATGTCAGAATTGGTGGATGAGAGA | |
| | | | AGCTCTTGAAGCATTCATTAACCTGAAATGTCTAG | |
| | | | CTGACGATGATGAAAGTGGGAACTGAATTAGTAGC | |
| | | | ACTCAGAAGAAAAATACGATGTTTATTTAGAGTTG | |
| | | | GTGAAACAGAGCTTTTGACTTAGATAACCTCTACAT | |
| | | | GGAGCTGAAGCTTCCTAAGGGATACAGTTGGTCA | |
| | | | TAGCTTCTCTCTGTCTATTGAGAGGTGTGGAAAAGT | |
| | | | TTTGATTGTTCGATGAAAGTCAACTTTTGTCTTC | |
| | | | TAATTTTATTCATTCTTTTGTATTTTGATGATTCATTC | |
| | | | ATTCTTTTATTGTGAATAAAATGCTAGACAGG | |
| | | | TTCCTTTATAAAAACGTTTACTAACTTAATCACACAT | |
| | | | GTCGCTTTCATGATCATATGTCTAATCATTCCA | |
| | | | AAGGATTTTACTGCAG | |
| 5 | NM_126476 | 814983 | CTAACATTTACTTTTGGGTTCTGTTTTGATTGATTGA | 5 |
| | | | AGCGACATGGGTTTGACAAAAAACTTTGCTTTT | |
| | | | CTTGTGTTTTGTTGTCAAAGATACTGTAGCATGGAC | |
| | | | GGTGAAGCTTTTGAAATCTCATTACTTGATGGAG | |
| | | | AAGAAGACAGATTTGGAGGTACTGTGGTGAACTTG | |
| | | | ATGGAAGTGGAATCCATGACTATTGGTGATTTTGA | |
| | | | TTCAAAGCTTGATGTTTCACTTAAGGCTTGGAAGGA | |
| | | | TCAGGGGAAGAAGGGGATTTGGATAAAGCTGCCT | |
| | | | AGTGAGCTATCTAGTCTTGTTGATACTGCAATTAAG | |
| | | | AAAGGGTTTACATACCACCATGCCGAGAATGAGT | |
| | | | ATGTGATGCTTACATTTTGGCTTCCAGAGCCACCTA | |
| | | | GCACTCTTCCTTGCAATGCTTCTCATCGTATTGG | |
| | | | GATCGGTGCTTTTGTCCTAAACAAGAACGGAGAGAT | |
| | | | GCTTGTGGTTCAGGAGAATAGTGGATATTTCAAA | |
| | | | GATAAAAATGTCTGGAAGGTGCCTACCGGTACTATT | |
| | | | AAAGAGGGTGAGAGTATCTGGGCTGGAGCAGTTA | |
| | | | GGGAAGTGAAAGAAGAAACTGATATTGATGCAGAG | |
| | | | TTTGTAGAAGTCTTGTCTTTCATGGAAAGTCACCA | |
| | | | AGCAGTGTGGCAAAGAAAAACGGATATATTTTTCGT | |
| | | | CTGCGAGTTGGAAGCAAGAACTTTTGAAATCCAG | |
| | | | AAACAAGATTCTGAGATCCATGCTGCTAAGTGGATG | |
| | | | CCGGTTGAGGAATACGTAAACCAACCCTATCACA | |
| | | | ACAAGGAGGGGAACGAGATGTTCAAGTTAATAGCT | |
| | | | AACATCTGCTTAAAAAGGTCGCGGGAGAAGTACAC | |
| | | | GGGCTTCGTTCTTACTACGAATTCAGCCAAAAAGAG | |
| | | | CCTTTACTGCAGCGTCGATCACGCCAACCTCCTG | |
| | | | AAGGAAACAGCTGACCAAGCCTCCACCTCTCTCTCT | |
| | | | GACTAGTCGGAAAGTGTTTCTGCTTCTTTGTTTC | |
| | | | ACGTCTTGTGTATTCAATTAAAGTTCACCAGTCTTGT | |
| | | | GTGTCCAAAGACTGGAGACTTGCCATTAGTAAT | |
| | | | TCACCATGGTTTCCTTCTAAATAAAATGTACTGAAG | |
| 6 | NM_126478 | 814985 | AAGAAACATTTGCTTGCGAACGGCTTTTGAGAAA | 6 |
| | | | GGCTTTGTTTTGGTTGATTTAATTCTG | |
| | | | AAATGTGCCTCTTTGGTTCCAAAGTCGCTCTGTAACT | |
| | | | TAAGAGAGAGTGACTGTTCCGTTGACAGAGCAA | |
| | | | AATGGCCCAAACCTATTGAAAACCCCAAAACCCTTA | |
| | | | GTTTGGAATCTCATCTCATTTTGCTTCTGTTCTT | |
| | | | TTCCATGTAATTTCAACAAAGTTTGAAACTTTGTTTC | |
| | | | TATAGCAACTCAATTACATGGAAATTCTTAAAA | |
| | | | GTCTACTCTTTAAGTCGCCCTCTTGGCTGAAACTCTT | |
| | | | AGCTTCTTGTTATAAAACCTGAAGTTCGAAATC | |
| | | | ATTCCAATACCACAAATAACTCAACTTTCTTCAAGC | |
| | | | ATGGACAATGAAGATCAGGAGTCCCTTTTACTTC | |
| | | | AAGGGGTTCCAGATAATTATGGAGGTGTTAAGGTGA | |
| | | | ACTTGACAGAACCAATGACTATTGAGGATTTTGT | |
| | | | CCCAAAGCTGAGAGCTTCACTTGTGTATTGGAGCAA | |
| | | | TCAGGGGACGAAGGGGATATGGTTAAAGCTTGCC | |
| | | | GATGGGCTTGACAATCTTATTGCACCTGCAAAAGCA | |
| | | | GAAGGGTTTGTGTGCCACCATGCCGAGAGGGAAT | |
| | | | ACACCATGCTTACATCTTGGATCGCTGATGTTCCTA | |
| | | | GTACTCTTCCTGCCAATGCGTCTCATCGTATTGG | |
| | | | AGTTGGTGCCTTTGTCCTAAACAAGAAGACCAAAGA | |
| | | | GGTGCTTGTGGTCCAGGAGATTGATGGTCATTTC | |
| | | | AAAGGTACAGGAGTGTGGAAACTGCCTACCGGTGTT | |
| | | | GTTAAAGAGGGTGAGAATATTTGGGAAGGAGCAC | |
| | | | TTAGGGAAGTGGAAGAAGAAACTGGTATTAAGACA | |
| | | | AAATTTGTAGAAGTCCTGGCTTTCAGGGAAAGCCA | |
| | | | CCAAGCATTCTTGGAAATAAAAACGGATATATTTTT | |
| | | | CCTCTGTGAGTTAGAACCAACCACTTTTGAAATC | |
| | | | AAGAAACAAGACTCTGAGATCTTAGCTGCTAAGTGG | |
| | | | ATGCCAATTGAAGAATACGTAAACCAACCCTGGA | |
| | | | ACCAGAAGAAAGAACTGTTTAGGTTCATGGCGAAC | |
| | | | ATCTGCTTGAAGAGATTGCAGGAGATGGAATACAT | |
| | | | GGGTTTCTCCAAAGTCCTTACTACGACTTCATCTGG | |
| | | | GAAAGAGAGCTATCTTTACTGCAACACCGATCAC | |
| | | | GCCAACCTCCTCAACGCAACGCGTGGCCTAGCCTCC | |
| | | | ACCTCTGGTTGAAAAACCCTTCTTCAGAACGTGT | |
| | | | TTATGTTTCGTTGTTTCATGTCCATTGTTCAATAAAA | |
| | | | GCACCAGTCTCGTGTGTACAACGATTGGAGACT | |
| | | | TCTTTATGTAATTTACCATCATTTCCTCACTACAACT | |
| | | | TCATATATAATATATTTCTTTGGCTACCAAAAA | |
| | | | GAGGTATCCTTTTTATTCTAGTTTCAATAAGAGTGTG | |
| | | | AACCCATAGATGCTGAG | |
| 7 | NM_179036 | 826884 | CTAACACAAAGAATAAACGACTACAATTTATAATTT | 7 |
| | | | TTCATTTATAAAATAAAATAAAAATTGGAATATA | |
| | | | TTGTGCGGAATTCCTATATATCAGAACTCATTGATCT | |
| | | | GCTGCTTCTTCTGAAAGTTCTTAAGTGGTTCCA | |
| | | | CGATGTTTCCATTTTGTTCTGACTTCCGAGGCCAACG | |
| | | | AGATGAGTCCAATAAACAAAGGGCACGGAAACA | |
| | | | TTCTTTTTAAATTAGGTTTGTTTTTTTATTTTTGGTTT | |
| | | | TTGGTTTTTCCCGGAAATTGTATGAAGTCTTC | |
| | | | GTTACAGTTTCTCTCTCTCACCTTCTCTGTATAAAGT | |
| | | | CTTCTTGAGATCGAAGGATAACTTCAATTCAAT | |
| | | | TCTCTCCAAGGTACACACAGCTGATTTTTGTGATAA | |
| | | | GAAGATTGATTGAGATGGGTACTAGAGCTCAGCA | |
| | | | GATTCCTTTACTTGAAGGTGAGACTGATAATTACGA | |
| | | | TGGTGTTACTGTAACCATGGTGGAACCTATGGAT | |
| | | | TCTGAGGTTTTTACTGAAAGTCTTAGGGCTTCTCTTT | |
| | | | CGCATTGGAGAGAAGAGGGGAAGAAGGGAATTT | |
| | | | GGATAAAGCTGCCTCTTGGATTGGCTAATCTTGTGG | |
| | | | AGGCTGCAGTTAGTGAAGGATTTAGATATCACCA | |
| | | | CGCGGAGCCTGAGTACTTGATGCTTGTATCTTGGAT | |
| | | | CTCTGAAACTCCTGATACAATCCCAGCCAATGCT | |
| | | | TCTCATGTTGTAGGTGCTGGTGCTTTGGTCATCAACA | |
| | | | AAAATACTAAAGAGGTCCTCGTTGTCCAGGAGA | |
| | | | GGAGTGGGTTTTTCAAAGATAAAAATGTGTGGAAGC | |
| | | | TGCCTACTGGTGTTATCAACGAGGGCGAGGATAT | |
| | | | ATGGACTGGAGTAGCTAGGGAAGTGGAAGAAGAAA | |
| | | | CTGGAATTATTGCAGATTTTGTCGAAGTACTGGCT | |
| | | | TTCAGGCAAAGCCACAAAGCCATCTTAAAAAAGAA | |
| | | | AACAGATATGTTTTTCCTGTGTGTCTTAAGTCCGC | |
| | | | GCTCTTACGATATTACTGAACAAAAATCTGAGATCT | |
| | | | TGCAAGCTAAGTGGATGCCGATCCAAGAGTATGT | |
| | | | AGACCAACCATGGAACAAGAAGAACGAGATGTTCA | |
| | | | AGTTCATGGCTAACATTTGCCAAAAGAAGTGTGAG | |
| | | | GAAGAATACTTGGGATTCGCCATTGTGCCAACTACC | |
| | | | ACATCATCTGGTAAAGAGAGCTTTATCTACTGCA | |
| | | | ATGCGGATCATGCCAAGCGCCTTAAAGTATCGCGTG | |
| | | | ACCAAGCCTCTGCTTCTCTCTGAAAACATTCTCA | |
| | | | TCTGATTCTGTTTGATCTCGGCATGTGTGTGTCCAGT | |
| | | | GTGTACAAAACAGGAAACTTTCTTCTATATTAT | |
| | | | TCCGATTCTACAAAATTGCGTCTAAATATTTAAGTTT | |
| | | | ATACCATCTCTATGAGCCACTGTAAAACTTGCC | |
| | | | ATCGACTTCTAGTCTATTTCCAGTGATGGTTTGATTC | |
| | | | TCCCAACGCTTACGTGCTTTTTAAAGATATGAG | |
| | | | GCTTAAGAGTGTTACCATACACTTGGTTTATTCTTCC | |
| | | | AAAATATCTTCTCGTAACCGGTTTTAAGTTCTC | |
| | | | AGCTGTATCGTCTCTTGACCATTTACTACACTGGTAA | |
| | | | ATGGTGTGAATCCAAGAAAGTGTTAATGCCATT | |
| | | | AAAAAGGTTCACATTACTTTGTATATGTACACCAAT | |
| | | | TTTTGCGACTCGTTTGTACTGTGTGCCTAACGTG | |
| | | | AAAAAGATAAGGCATATATGGGCTTTAGCCCCAAA | |
| | | | AAAACTTTATAGGCCCCATATTAAAATTATAGTGT | |
| | | | ATTTATTTTTCCAGAAATAGAAAATTT | |
| 8 | NM_124095 | 834771 | AACCACTCGATCCACTTTAAACCACACATTATAAGT | 8 |
| | | | GTGCCTCAAAAAAATCTAACATTACTCTCTCAAC | |
| | | | ACATCTCTCTTTCTTCTCCATTTCCTTCTTCTCTTTCA | |
| | | | TTCTCTTATTTAAGATTTAAATCTGCTCAAAT | |
| | | | GCTTGTAAACAAATTTCAAGTTCTTTAAACGATTTCC | |
| | | | TTTTCAAGAAAAATCCTTCTCCTTCTTCTTTTT | |
| | | | ACCACATAGATCTTCTTGATCTACAATGGATTCTGTT | |
| | | | TCTCTTTCTGAGGTTACTGTCATTAAAGGAACA | |
| | | | ACACATTTGGGTTTTATGCACAGTTTCAGACAGCCA | |
| | | | TTTTGTGGTGTCAAAATATCTCCAAAGTTCTATT | |
| | | | TATCCAAAGTTGATGGACCAAAGGCAATATCTTCGA | |
| | | | GCAGCAACACAAAGAGTCAATTTGTTTATGGAGG | |
| | | | AGGAAGCATAGCTGCAACTTCAGATTCAGGTTATAA | |
| | | | GATGAATGGAGTAAACCTAAAAAGCAGAACATTG | |
| | | | ATGAGTTCTGCAGTTAAAGAAAGATCATTACTTGAT | |
| | | | GCTTATGATGATGAGTATGGTGGAGTAATAGTAG | |
| | | | ATCATGGAAAATTACCATCAAACCCTTATGCTTTCG | |
| | | | CTTCTATGCTTCGAGCTTCTCTATCCGATTGGAG | |
| | | | AAGAAAGGGAAAGAAAGGAGTTTGGTTAAAGTTAC | |
| | | | CTGTGGAACAATCAGAATTAGTCCCAATAGCTATA | |
| | | | AAGGAAGGTTTTGAGTATCATCATGCAGAGAAAGG | |
| | | | ATATGTAATGTTAACATATTGGATACCAGAGGAGG | |
| | | | AACCTAGTATGCTTCCTGCAAATGCTTCACATCAAG | |
| | | | TTGGTGTTGGAGGTTTTGTATTAAATCAACATAA | |
| | | | AGAGGTGCTTGTGGTACAAGAAAAGTATTGTGCTCC | |
| | | | TTCGATTACTGGTCTATGGAAGTTACCAACAGGG | |
| | | | TTTATTAATGAATCTGAAGAGATTTTCTCTGGTGCTG | |
| | | | TAAGAGAAGTCAAGGAAGAAACTGGGGTAGATA | |
| | | | CAGAGTTCTCAGAGGTTATAGCTTTCAGACATGCTC | |
| | | | ACAACGTTGCATTTGAGAAATCTGATCTGTTCTT | |
| | | | CATCTGTATGTTGAGACCACTCTCTGATAAGATAAT | |
| | | | CATCGATGCTCTTGAGATCAAAGCCGCAAAGTGG | |
| | | | ATGCCATTGGCTGAATTTGTGGAGCAACCGATGATA | |
| | | | AGAGGAGACAAAATGTTTAAAAGAGTGATTGAAA | |
| | | | TATGCGAGGCGAGATTAAGCCATCGGTACTGCGGTC | |
| | | | TTTCTCCTCATCGACTTGTCTCTACTTTTGATGG | |
| | | | CAAACCTTCTTCTCTCTATTACAACGTTGTTGATGAT | |
| | | | GATCATGATCCTTCCCACTCCAATTGTAGCACT | |
| | | | GAGTTTTATAGATAGACGGGTAAAACAGAACCAAA | |
| | | | TCAAGTGGTTCGATTCTGTATATACTTATTCTCAT | |
| | | | GTAATGTATGTTGTTATTTGATACTCTCTCTTCAGTA | |
| | | | TAGTAAATAGTAATAGATCCTCTGCAAAATCCA | |
| | | | TGTAGCATCGAACCGGTACTCTCAGTAGACTATACA | |
| | | | ATAAAAAGATCTGTTC | |
| 9 | NM_114489 | 823765 | ATAATAGTAATCATTCACAAATCCAAACCAAAAATA | 9 |
| | | | CCTAAACTTACTCTCAAATCAAATATATATATGC | |
| | | | TTCTTTCAAAATGTTCTTTCAACTATTCTCACTTCTCT | |
| | | | TCTTAACTCTATTTGTTACTTCCGTTACTTCC | |
| | | | AGCCGTTTCATCTACGACTTTGATCCTGTCCCATCAT | |
| | | | CTGCCACCACGCCACTTGAAACTAACCCAGTTG | |
| | | | GCTTCAGCGGAGCCGTCATCAAAGTCGGGGCAAAT | |
| | | | ATGGCCGTCAGTGGAAGTCAGATTCCGTTAGCTAG | |
| | | | CCTTTTGATACGTAATGGCGCCATTTTGACCAGTAA | |
| | | | CAAAGACGAAGTCCCTCTCATTCACCATCACCAT | |
| | | | ATCAGTGGTACAATATCCAACATAATCAAATGATCA | |
| | | | GCTCGTTGGTACAACTAGTCAACAAAACCATAAT | |
| | | | TTTTAAGGAATTGTTTTTCTCAATACATGCAACGTTT | |
| | | | TCTTCTAGAGTGAATTCATTATCGACTTGGAAG | |
| | | | ATTGAACATGTATCATCAAGAGTTGTATCTGTAATC | |
| | | | ATATCAGTTGACAGTTTGAATGGTCTTATTGAAC | |
| | | | TAAGGGGTGTTAACAAAGCCAAAGCAAGTAGATTT | |
| | | | ATGCTCCAATGAGCTTATTAAGATAGAAATAAACT | |
| | | | AGGGACTTGAATAGCTCATGCAGAGCTTTATTGTGC | |
| | | | AAAGAAGTAAAGAGAGGAAGAACAACTTATCAAA | |
| | | | AGATTAAAGCGTTGACAAAAATATAAAATAGATTTA | |
| | | | AGCTTCGGCCCATTAGTTCCCTTCTTGGGATTAA | |
| | | | TGGTTTATTTATCGGCCCATTTAGTGATTTCAGCCCA | |
| | | | TTAATGGTTTCATTCCTCTCTCATTTTTTTCTT | |
| | | | TCTGTGTTCGAGCTTCTTCCATGGCGAAACTAGCAG | |
| | | | AAGAGATGAATCCTGAAGGTTCACGATACCAGCT | |
| | | | TCTTCTCTCATGTCCTTCTGGCCTCTCTCCGTCTCAG | |
| | | | GTCTCAGTGGATTTCTCGAAATCGCATGATCGG | |
| | | | ATTCCTCGTCAAGATCCTGGTCTAGAAGATTCTATTT | |
| | | | CACAGGTTTGGGAGCAGAGATCACAGGGAAATT | |
| | | | CATCATTGTTCAATGGCCAAAAATTTAGGTATGGAG | |
| | | | GATATTGTTTAGATGATGATGATGGTAGTACCAA | |
| | | | TGAAGTGCCACACGTTTGTCTTCGTCTTGGCCTGAC | |
| | | | AGATTACAGGACTTTTGTAGGAACGAATTTGAGT | |
| | | | TCTCTGTGGGAGAAGTTCCTTGTTACATCAGAAGAT | |
| | | | GACTCTGTTAGATGTCGACACACCTCAAGTCCAT | |
| | | | TGGGAAATGGTGCAGTTATCGAAACTTCTGATAAGA | |
| | | | AGATTATTGTGTTACGTCGGAGTAATAATGTTGG | |
| | | | GGAATTTCCAGGACACTATGTATTCCCTGGGGGCCA | |
| | | | TCCAGAGCCAACAGCAGTAGGTATTGATTATCAT | |
| | | | CAGCTTGAAAACAATGTTCAAACCGGTGAGGTTTTA | |
| | | | AACAAGAAGGTAACTCAAGAAATGTTTGATAGCA | |
| | | | TTATCTGTGAAGTTGTCGAAGAAACTGGAATACCAG | |
| | | | CATCATCACTTAGCTCGCCTCTCTTCATCGGTAT | |
| | | | ATCTCGAAGGGAGTTGAATGTAAGACCAGCTATGTT | |
| | | | TTTCTACCTCAAGTGTAGTCATCATTCAGATGAC | |
| | | | ATTCAAAGATTATACTCTAGCGCTGAAGATGGGTTC | |
| | | | GAGTCAACACAGCTCCACACTGTCTCATTGGATG | |
| | | | AGCTGAAGATGATGACATCGAGAATGCCAGGTTGC | |
| | | | CATCATGGCGGATTCGCTCTCTACGAACTGATGCT | |
| | | | TCAACGTCTCAAGAACACTAAGGAAACGTCTTTAAT | |
| | | | AGCAACGTAAGAGGAAGATACTTAAACTTTACAC | |
| | | | CTAGTTGTGAATTATTGTTTGCTAATGATCATGTCAT | |
| | | | TTTCGAGTAACTTTTATTAGATTAACCGTATTA | |
| | | | ATTTGTAAAATCCATGATAATGAGTAACTATCATGT | |
| | | | AATTCAGGGATAGAAAGCTCTAAGTTATGTTGGA | |
| | | | TAACAGGTTCATTTGTTCACATATGTAAATGGTAAG | |
| | | | GGTTTCGTTTCTGTAGAACCTACAACATGGGGAA | |
| | | | TATGAAAGCTTACAAATCTCACGCAAATACCATTAT | |
| | | | GATTCATGCGTTTACCCTGTTTTGGATGAAAATT | |
| 10 | NM_179112 | 828648 | CGTGATTGTTCTCCTTTTGAAATCATTTGCTTCGTTG | 10 |
| | | | AAACCACACTCTCCTTTTACTTTATCTTCTCCT | |
| | | | TTTCGTTGAATACTTGAAATCTTCCAATCTTCCTCCA | |
| | | | AATTTTTTCAGAGTTTGAAGCATGTCAGACCAA | |
| | | | GAGGCTCCTCTGCGTAATGGAGTAGAACACAAGATT | |
| | | | TTCGAAGTACTTCCGTTTGTGGATGATGATTACG | |
| | | | GTGGAGTCATCGTGGAGATGAAAACTCCTATGGATA | |
| | | | CTAAGAATTTCGTTGCAGCGCTTAGAGATTCATT | |
| | | | CGAGCAGTGGCGCTTACAGGGTAAAAAGGGAGTCT | |
| | | | GGTTGAACTTGCCTCTCTCACACGTAAACCTCGTG | |
| | | | GAACCCGCGGTTAAGGAAGGTTTCCGGTATCACCAT | |
| | | | GCTGAGCCAACTTACCTGATGCTAGTTTATTGGA | |
| | | | TTCCGGAAGCCGAGAGTACTATTCCTTTGAATGCTT | |
| | | | CCCATCGTGTTCGTGTAGGTGCTGTTGTCTTGAA | |
| | | | CCATAACAAAGAGGAAAAATACGGTTCATTATGCG | |
| | | | GATCAGGTATCTGGAAGATCCCAACAGGAGTTGTT | |
| | | | GACGAGGGTGAGGAGATTTTCGCCGCAGCCATTAG | |
| | | | AGAAGTAAAAGAAGAAACAGGCATCGACACTGAGT | |
| | | | TTTTGGAGATTCTAGCTTTCTGCCAAACCCACGAGT | |
| | | | CATTTTTTGCAAAGTCAGATTTATTCTTCGTTTG | |
| | | | TCTCTTGAGACCTACCTCCTTCGATATTCAAAAACA | |
| | | | GGACTTAGAGATCGAAGCAGCTCAATGGATGCGG | |
| | | | TTCGAGGACTCTGCCTCTCAACCTATCACACACAAG | |
| | | | AACGATCTCTTCAAAGATATTCACCACATTTGCT | |
| | | | CAATGAAAATGGAGAAGAGCTATAGTGGTTTTTCAA | |
| | | | AAAAACCTATTACAACTTTCTTTGACGACAAGTT | |
| | | | AGGTTATCTCTACTTGAATAAACAAGAAGATATGGA | |
| | | | ACAACCTATTTCTTAAACTTCTTCCAAGAACATC | |
| | | | GTCATTAAGAGATCATATTCTGATCTAAAACAATCA | |
| | | | ATCGATCTGAATGAAAAACAAATGCATGAGTTTC | |
| | | | AGATTTGTTATCCTAAAAGAGATGCTGAATTCTTCT | |
| | | | CCAGCAAGCAAAAATACAAATACAGTAAGAAGAG | |
| | | | AGGGAGCTTGTGACGAGGAGGAATTAAGAAGTGAA | |
| | | | GATCTTGTTTTTATTTGATGATTTGAAATCACTAG | |
| | | | AATCCACTAATACAATAATTAAATAAATAAATACAT | |
| | | | ACATACCGAAGCACAATTATTTGATTGCACTCAA | |
| | | | ATGAATCATTGTCTTCAAGACTCCTTCATCCATTTTT | |
| | | | CTTTAACGTTCCATTCTAAACTCGACTCTTTGA | |
| | | | ATGTTGATGTATTTGTTTTAAGTAATTCATTTTGATT | |
| | | | ATTCCATCATTAATTACCACTTTTTCTAATAAC | |
| | | | TTGGAAATTGATGTTGATGTGCTTTGTCTTCCAGTTT | |
| 11 | NM_123962 | 834634 | AAATAAAGATATGGATCTGTTACTAAAACAAAGTCG | 11 |
| | | | CTTTATTCAGATCTGAAGAAGAAGAAGAAGAAGA | |
| | | | AACATAGTTGAAGTCTCTGTTAATATGTCTTCAACA | |
| | | | ACAACAGATTCTACAGAATTGCAAAATCTGATCA | |
| | | | AACTGTTTCAAAATTGCCAAACTCATCCTCGTCAAC | |
| | | | ATTTTCCTGCAAAATCTTCAGCTGTATTAGTTTG | |
| | | | TCTATACCAAGAACAAAGAGAAGACAAAAACGAGC | |
| | | | TACGCGTAATCTTAACCAAACGATCTACTACTCTC | |
| | | | TCTTCTCATCCTGGGGAAGTAGCATTGCCTGGAGGA | |
| | | | AAAAGAGACCAAGAAGATAAAGATGATATAGCTA | |
| | | | CTGCGTTGAGAGAAGCTCGTGAAGAAATCGGTTTAG | |
| | | | ATCCTTCTCTTGTTACAATCATTTCTGTTCTTGA | |
| | | | ACCATTTGTTAACAAGAAGGGAATGTCGGTAGCACC | |
| | | | AGTTATCGGTTTTCTACACGATAAGAAAGCGTTT | |
| | | | AAACAGTTACCAAATCCAGCTGAAGTGGAAGAGAT | |
| | | | CTTTGATGTGCCATTAGAGATGTTCCTCAAGGACA | |
| | | | GAAACAGGCGAGCAGAGGAACGAGAGCATGAAGG | |
| | | | AGAGAGATATCTTCTTCAATACTTTGATTACTATTC | |
| | | | CGAAGATAAAGAGAGAAGCTTTATCATATGGGCAC | |
| | | | TCACTGCTGGTATTCTGATCAGAGTTGCCTCCATT | |
| | | | GTTTATCAGAGATTACCGGAGTTTCAAGAACGTAAA | |
| | | | CCGAGTTTCTGGAATCAGCCTAACTGATTCTTGA | |
| | | | CTTGATCTGATTGAGAAAATTGAAACTTTTATGTAT | |
| | | | AATAAGCGATACAGATTGAATCTTCTTTAAATGT | |
| | | | GTTTCAAGAATCTATCTTTACTCTAA | |
| 12 | NM_101159 | 837845 | CAAAAATAATTTATTTATTTTCATGTATAAATATTAA | 12 |
| | | | AACTGATTATATAGAGAGACAGGTGATAGTTAG | |
| | | | CAGTGAATGCTCGTTGGTACAACTCCTCATTCTTCTC | |
| | | | CGCAAAAATAGCAAGTTATTTAACAAACAAAAA | |
| | | | AATTCATATAAACGATTTTATTTTTTTGTGACTTTTG | |
| | | | AGATTCGTTAATTCTGGAAAATTTGTTTATTTT | |
| | | | CAAAACAAGAATATGGATTCGTCATAAAAGCTAGG | |
| | | | CTATGATCAACATGTTTCATACAAACAAGAACAGG | |
| | | | GCTTTTCAAGAAAGTAGCATTCTCTGTTTCTGTTGTC | |
| | | | TCAATCCCTTTTGATTTTTTTTGTGTTGGCAGA | |
| | | | TTCATCAAATGTCGGTTCTTTCTTCTCGGACAGGACG | |
| | | | AGATCGTCAACGTTACGACAACAACTTCCGTCT | |
| | | | CGTTTCTGGATGCATTCCTTATAGGCTGATGAAAGC | |
| | | | TGACGAAACTGAAGAAGACTCAGGTGTTGACTTT | |
| | | | GTAAACAAACTTGAAGTTCTTATGGTTTCATCTCCTA | |
| | | | ATCGTCATGATCTTGTCTTCCCAAAGGGAGGAT | |
| | | | GGGAAGATGATGAGACTGTTCTTGAAGCTGCTTCTC | |
| | | | GTGAAGCCATTGAAGAAGCAGGAGTTAAAGGAAT | |
| | | | ACTTAGAGAACTTCCTTTAGGAGTTTGGGAATTTAG | |
| | | | AAGCAAAAGTAGCACAGTGGAGGATGAATGTTTA | |
| | | | GGTGGATGTAAAGGATATATGTTTGCATTAAAAGTT | |
| | | | ACCGAAGAACTCGAGGATTGGCCAGAGCGAAAGA | |
| | | | ATCGTGAACGAAGATGGTTGACTGTTAAAGAAGCGT | |
| | | | TGGAGCTTTGTCGGTATGAGTGGATGCAGAGAGC | |
| | | | ACTTGAAGAGTTTTTGAGAGTAATGGAAGATGAAA | |
| | | | GAAGATTGAGAACAGAGGAAGAGACAGTGCATGAT | |
| | | | TCTTCAAAGCTAGAGGAGGAGAGTCAAATAGATCCT | |
| | | | TGGTACTGTTTTGTAGTTAACTAGTTTTTCATAT | |
| | | | TCTTTAGCTAACGTAAACAAATTGAAAAAATGAGAA | |
| | | | GTAAAGTGACTGCAACACATGTGACATGATATAA | |
| | | | TCTCAAATCATCTGTCTTGAATCTTGATATCTGTCTG | |
| | | | TTTGAAGTGATAAAGATAGAGTTGTCTTTATAT | |
| | | | GCAATTGGGACACCAAATGCAAAAAGACAAAGGAC | |
| | | | ATAGAAGAAATTACCTAATCACACACAAATCAATG | |
| | | | CTTAAGGCTTGACTTCAAAATCAACTGAATACACTT | |
| | | | AAGAATCCAAATCCCTAAGCTTATGTCACATGTT | |
| | | | TAGTTAGG | |
| 13 | NM_180305 | 822281 | CTTATTATATATGTCCCCGCAAAAATCTCATCTTTTT | 13 |
| | | | CCTTCTTCCTTCCTTCCTATATGTGCCAACAAC | |
| | | | AAACCAAAACCGTTCCTTTCTTTTTCCATTTTTCTTG | |
| | | | TTTTTTTTTGTTTTGTTTTTGGAGTTGTGTTTC | |
| | | | ACCTGTTGTTGGTTCTTTAGTTCGTCTTCTGGGTTAA | |
| | | | ACAAAGAAGATCGGTTCTGTTTTTATTTTTATT | |
| | | | TTGAAACAAGAAGAAGAAAGCAAGCTACGATGATG | |
| | | | TTTCTTGCTGACGCCAATAGATAAAGACACGACTT | |
| | | | TTACAAAGCTTAGGACCTGCATTCTCACCGTAATTA | |
| | | | GCTCTTCTTCTCAATTTCTCCAAATAAATTTGTG | |
| | | | TGTTTTTAATGTGTGTGTTGCAGGTTATCAAAAATGT | |
| | | | CGAATCTTTCTGCAAGAACAGGACGAGACCATC | |
| | | | AACGTTATGACAACAACTTCCGTCTTGTTTCTGGAT | |
| | | | GCATTCCGTATAGGCTGGTGAAAGATGAAGAAGA | |
| | | | GGACTCAACAAGTGTTGACTTTGAAAACAAGCTTCA | |
| | | | AGTTCTTATGATTTCATCTCCTAATCGTCACGAT | |
| | | | CTTGTCTTCCCAAAGGGAGGATGGGAAGATGATGA | |
| | | | AACTGTTCTTGAAGCTGCTTCTCGTGAAGCTATGG | |
| | | | AAGAAGCAGGAGTTAAAGGAATACTAAGAGAAGAT | |
| | | | CCATTAGGAGTTTGGGAATTTAGAAGTAAGAGTAG | |
| | | | CAGTGTGGAAGCTGATTGCTGCTTAGGTGGTGGATG | |
| | | | CAAAGGTTATATGTTTGCGTTAGAGGTGAAGGAA | |
| | | | GAACTTGCGATATGGCCTGAGCAAGATGACCGCGA | |
| | | | GAGAAGATGGCTGAATGTAAAAGAAGCCTTGGAGC | |
| | | | TGTGTCGGTATGAGTGGATGCAGAGCGCTCTTGAAG | |
| | | | AGTTTCTACGAGTAATGGCAGAAGAAGGAAGCAC | |
| | | | AAAGGAAGACTCTCTTGCCATTTCCAGCATTTCAAA | |
| | | | TCGAGGAGAGCGTCAAATTGATCCTCGCTACTGC | |
| | | | TTTGTAGTCTAACTAGTTTCGTTTCTCTAGGCGTATC | |
| | | | AAACCTCTCTCTCAATTGTTATATATTATGGAA | |
| | | | AGACTTCTTCTGTAAACAAAGTTAAAACATGAGAAG | |
| | | | CAAATGCTTTCAGACCCTGTGACTTGAATGTGTT | |
| | | | TCTCATAAGCAAAAGATATATAGAGATTATATTAAC | |
| | | | T | |
| 14 | NM 117268 | 826805 | GTGATTTGCTTGCTTTCACGTTTTTCATTGATGGCTG | 14 |
| | | | GCTTTACTCTTCTTCCCTCGCGGTTACTCGCGTTTCC | |
| | | | TTCACGCGCTTTACCTCGTCGCCTTCATCACCACCAT | |
| | | | GCAAAGCTGATTCTTCGCTGCAAAATGTCTTCTTCTT | |
| | | | CTTCCTCTCTTACTCAATCCATCACTCTCCCGAGCCA | |
| | | | ACCCAACGAGCCTGTCCTTGTCTCTGCAACCGCTGG | |
| | | | AATCTCTTCTTCCGATTTCAGGGATGCGATTGATTCA | |
| | | | TCTCTGTTTAGGAATTGGCTAAGGAATTTGGAATCA | |
| | | | GAGAGTGGAATTTTAGCTGATGGTTCAATGACATTA | |
| | | | AAGCAAGTTCTTATCCAGGGAGTTGATATGTTTGGC | |
| | | | AAAAGAATTGGATTTCTCAAATTCAAAGCGGATATT | |
| | | | TTCGACAAGGAAACTGGTCAAAAGGTTCCAGGTATT | |
| | | | GTATTTGCACGAGGACCAGCTGTAGCTGTGCTTATT | |
| | | | CTCTTGGAGTCAGATGGTGAGACTTATGCGGTTCTC | |
| | | | ACTGAACAGGTTCGGGTTCCTACTGGGAAGATTGTT | |
| | | | CTGGAATTACCTGCTGGAATGTTGGATGATGATAAA | |
| | | | GGTGACTTTGTTGGTACTGCAGTTCGCGAGGTCGAA | |
| | | | GAGGAGATTGGTATAAAACTGAAAAAAGAAGATAT | |
| | | | GGTTGATCTCACTGCTTTTCTTGACCCATCTACTGGT | |
| | | | TACCGGATCTTCCCTTCTCCTGGAGGCTGTGATGAA | |
| | | | GAGATGAGCGTGTTTCTTTACAGAGGACAAGTCGAA | |
| | | | AAGGAAACAATTAGACAGTTGCAAGGCAAAGAGAC | |
| | | | AGGACTCCGTGAACACGGTGAGTTCATCAAAGTCCG | |
| | | | ACTCATCCCATATAGAGAGCTCTGGCGCAAAACAGC | |
| | | | TGACGCTAAGGTTCTTATGAGCATTGGTCTCTATGA | |
| | | | AATGGCTCAGCGAGAGGGTCTTGTGTCCAGCCAGAG | |
| | | | ACTGAAACCCAACTCTTGATCTCCTACAATTCTCTG | |
| | | | AAATATACAATAACTGGATTCAGTCAACCAGATGAG | |
| | | | ATAGATTGAGATTAGACGTTCTTATGTTGTAAAAGG | |
| | | | AGTTTTTAGATAAATGTTATCGCTTTATAACTTGGTA | |
| | | | CCATGACAACTTGGGAACTTATCTTTAGAACAAAAG | |
| | | | ATTTTGCAAACCCAATTTAGGGTTTAGGTTTCC | |
| 15 | NM_001036027 | 839773 | AAAAAAGGAGAAACACGTGGACACTCACACGAGGA | 15 |
| | | | GGTGTCTGATTTTGTCTAATTAAATTTGAGTTTTT | |
| | | | TTTTTCTTCTTTGAAATCATATGATTGATCTACACAG | |
| | | | AATCCATTTCCGCAAATCGGATTCATGTTTTTG | |
| | | | CTTTATCGTAGGCTTCCTTCATTTGCACGAACAACA | |
| | | | ACAACAACCCTTCTCTGCAAATCTATGGAGCCTG | |
| | | | CGATAACAGCGACTTCTTCTTCTTCTTTCGGTGGTGG | |
| | | | CTCTTCTCGTCTCGCCGCTTTAGCCCAGCAACT | |
| | | | TCGCCAATACAAGCCCCCACCTTCTTCATCGTTCGAT | |
| | | | GACTCCGAGGAGATGCAGACAGATCAGGAGACC | |
| | | | GCTGGGAAAGTCGTTTCTCAGGTTGGGTTTCAGGAA | |
| | | | TCTATTGCTCCTCTTTCGAAAGACCCTGATAGGT | |
| | | | TTAAACCCAAGAGAGCCGCTGTGTTGATCTGTCTCT | |
| | | | TTGAAGGAGATGATGGTGATTTGCGTGTTATTCT | |
| | | | TACTAAGAGATCTTCCAAATTGTCTACTCACTCTGG | |
| | | | AGAAGTTTCATTGCCAGGTGGTAAAGCGGAGGAG | |
| | | | GATGATAAAGATGATGGGATGACTGCTACCAGAGA | |
| | | | GGCTGAGGAAGAGATTGGATTGGACCCTTCTCTTG | |
| | | | TTGATGTTGTTACTTCTCTTGAACCATTTCTGTCTAA | |
| | | | GCATCTTCTTAGAGTAATTCCTGTGATAGGCAT | |
| | | | CTTGAGGGACAAAAATAAATTCAATCCGATACCAA | |
| | | | ATCCTGGGGAAGTGGAAGCTGTGTTTGATGCACCC | |
| | | | TTGGAAATGTTCCTTAAGGATGAGAATCGAAGATCT | |
| | | | GAAGAGAGAGAGTGGATGGGTGAAAAGTATTTGA | |
| | | | TCCATTACTTTGACTACAGAACAGGAGATAAGGATT | |
| | | | ATATGATATGGGGTTTAACTGCTGGGATTTTGAT | |
| | | | CAGAGCTGCATCTGTGACTTATGAAAGACCACCTGC | |
| | | | TTTTATCGAGCAGTGCCCGAAGTTTAAGTACCCT | |
| | | | AAAATGGTAGAAAAACATACTTGTATGCCTTAATGA | |
| | | | ACATAATTTTTCGAATGATGCTTTCCTAGACTAG | |
| | | | TCTTCTTCCACTATTTGATAACTTGTACCAGACAACA | |
| | | | AGACAATAACATTCCATACAACTGATTCTCTGT | |
| | | | ACCAGTGAATGGCTGAATGCAGAACTTGGCTACTTG | |
| | | | CTTATATATATGCAGACGCAGTAGCTGAAGCTCC | |
| | | | AATTCATTTCCTGCTGGGGCATCTCATAACAGGCTT | |
| | | | CATTTCTGAGTTGTTCCCACAGGATGCTCAATGG | |
| | | | TGATTCAGAGAAAAGTTGGCTTGTTATGATTCTAGG | |
| | | | CAAACATCACCCTCAATTGTGAATTTGGAGGAAT | |
| | | | ATTTGTACTCAGTATTTACCTTCAAACGCTCATTTTT | |
| | | | CATTGTATACTTGTTATCTTCTATTTGTATACT | |
| | | | GAACCAATTTCAAAGCAAACTTGTCTTATGAGGCTT | |
| | | | ATGA | |
| 16 | NM_001084673 | 820440 | ATAGACAGACACACCCAAACCCTAGATCTAACCCCA | 16 |
| | | | GCCGTCAGATCAGATTGATCGGGCAATCGCTTCT | |
| | | | AGACCGTTGGAATCAAGCAATGGTAGAGCAGCGGT | |
| | | | ACGAGGATGGCTCTCGTCTCGTCGCCGGGTGTATT | |
| | | | CCGTTTAGGTATGTAAATTCTGACAAAGATGGTAAT | |
| | | | TCTGAATCTGGGAAAGTGATTCAAGTGTTGATGA | |
| | | | TCAGCTCGTCTAGTGGACCTGGACTTTTGTTTCCCAA | |
| | | | GGGAGGATGGGAGAATGATGAGACAGTCAGGGA | |
| | | | GGCTGCAGCGAGGGAAGCAGTGGAAGAAGCAGGAG | |
| | | | TCCGTGGGATTCTTATGGATTTTTTGGGGAATTAT | |
| | | | GAGTTCAAGAGCAAATCACACCAAGATGAGTTTAG | |
| | | | CCCAGAAGGTTTATGTAAAGCGGCAATGTACGCGT | |
| | | | TGTATGTGAAAGAAGAGCTAGCGACGTGGCCGGAA | |
| | | | CATGAGACGAGAACACGTAAATGGCTGACAATTGA | |
| | | | AGAAGCGGTAGAGAGTTGCAGGCACCCTTGGATGA | |
| | | | AGGACGCATTGGTGGAAGGATTCTGTAAATGGCAT | |
| | | | AAGGAGAAGATGGTTAAAGGAGAGGAGATAACTGG | |
| | | | TGAACATTGAAGCAAATGTGCATAAACCTCTGAGC | |
| | | | GTTGGTTTCATTCCTTTCGATTTTGTTTTTACCTTTTT | |
| | | | GTTTTTGTTTTTGAAGTCTTATGCATGGATAA | |
| | | | TCTCTTGTGACTGCGAAACGTAATAGTTTCGAAGAT | |
| | | | TTAAAGTTCAATTATTGCCCTATTTTCACATAGC | |
| | | | TGTCTTCACCATTTTCAATAATTATGGAAAATGACA | |
| | | | GC | |
| 17 | NM_126228 | 814696 | GCTTCTCGACCAATGATGAAAATTCCAAGAAAAGA | 17 |
| | | | AAGAAACATTCATAATTGTTTTTTTTTCAAACAAA | |
| | | | AAAAAAGAGCGGCCGTTTTATTATAAAAAGAAGAT | |
| | | | AAGCTATTGGAGGTTGATACAACGCTTTTTGTCTC | |
| | | | ATCTCCTTCTTCTTCTCCTTCTCAAATCTTTTTTTTCA | |
| | | | GGCGTAACGAATCGAATCATAAACACTTACGC | |
| | | | AGAAACAAAAACTAAACTTTGGATCTTTTAACCGAC | |
| | | | CATAGAGATTCGTGGGTTCTTCTTAGTTTCGACA | |
| | | | GATTCTTGATCCGTTTTAGTTGTTACGCTCATCTTTG | |
| | | | CTTTTATACATCAGACCCTTCTCTTCTCTTCCT | |
| | | | TCTTTATCTAAGAAACATTAGGGTTCTTTGAAATCTT | |
| | | | GCTAAAAAAAATGGGTGTTGAGAAAATGGTGTG | |
| | | | TTTAGCTTCTCGTACGGGTCGTCAGTTTCAGAGATA | |
| | | | CAACAAAGGTCGTCGTCAAGTCGTTGGATGTGTT | |
| | | | CCTTACAGATTCAAGCTATCTAATGATGGTAAAATA | |
| | | | AGTGATGAAGTAGAAGTTCTTGTTATCTCTTCCC | |
| | | | AAAAAGGCCACGCCTTAATGTTCCCCAAGGGTGGTT | |
| | | | GGGAGCTTGATGAATCTGTAGAAGAAGCTGCATC | |
| | | | AAGAGAATGTCTTGAAGAAGCTGGAGTTCTTGGCAA | |
| | | | CGTTGAGCACCAACTTGGAAAATGGGATTTTTTA | |
| | | | AGCAAAAGCAGAGGAACATACTATGAAGGACTAAT | |
| | | | GTTCCCTATGCTTGTTACAGAACAACTTGAGCTTT | |
| | | | GGCCTGAACAACATGTTAGGCAAAGAATCTGGATG | |
| | | | AATGTAACCGAAGCAAGAGAAGCTTGTAGAGATTG | |
| | | | GTGGATGAAAGAAGCATTAGATGTTTTGGTTGAGAG | |
| | | | ACTTTCATCACCAATGAATCAACCAAAGGAAGAG | |
| | | | AAGACTATGTCAATCTCTATTGAAACAATGTGTTGA | |
| | | | TGATGAAGCTGATGATTGCTAAATTGCTGATTGT | |
| | | | TGATGGGAGTTTTTATGAGCTAATATGGATCTCTGT | |
| | | | AGGTATTAAATAGATGGGTCTCAATTAGTTATTG | |
| | | | AGACGGCTAAAAAAATCTATTTCGAAAAGCCGATG | |
| | | | CAGAGAGCATGGACAAAGAAAAAGATGGTAGAGTT | |
| | | | TTAAGTTTTGGATCAGAAAGATTAACATCATGTGAT | |
| | | | TTAAGAAGTCTTGTGTTTGTGTTTATAATGTTTC | |
| | | | TGCAAGAACTTCTTAAGATATGATTAAGAGGGAAG | |
| | | | ATGATAAACTTAGAGTGCAAGCAAAACCTGTATAA | |
| | | | TTGTGTTTCCCTCTGTTTATTTTTCCTCTCTCTTTGAA | |
| | | | TTTGCAATGGAGACATTTGATTTCATCATGAT | |
| | | | GCTTTTGTGATTCAGAGATACTTAAATTATGCAAGT | |
| | | | AGATGATACGTGGCGTGATGTGATTGATTTACAA | |
| | | | GAAACAAAGCAAGAATAAGTTACTCTAATCATATG | |
| | | | AAAAAAAAAACATGTCATTTTCAAAATTTATTATG | |
| 18 | NM_101355 | 838051 | AGAGCAAAAAAGAGTTTAGAAAATGTATGTCGTTTT | 18 |
| | | | TCTATAAAAAGGATAAGCCGCAAGGAGGCAAAAG | |
| | | | ACAACGCTTTTTGTCTTATCTCAAATCTTTTGTTCAC | |
| | | | TCACCCCATCATAACTTCATAACTCCTATCCAA | |
| | | | TCTTTGCTTATAACTATTATCAAACAATTCTTAACTT | |
| | | | CGATCTCTAACCTGCAAAAACATTCATTACACT | |
| | | | CTTCAGATCCTCCTTCCCTTTAGCCGCCAAAGCAAA | |
| | | | ACCGTCATCGATCTCTCTTCTTTTATACAGTCAA | |
| | | | AAATTAGATTCAGATTCTTCAAGAACTCTGTTTTTCT | |
| | | | AGTAGCATGGTGTGTTTGGTCTCCCGTACGGGT | |
| | | | CGTCAATCCCAAAGATACAACAAGGGACGGCGTCA | |
| | | | AGTCGTCGGGTGTATTCCATACAGACTCAAGATTT | |
| | | | CGAGTGATGGCACAATTAGCGACGAATTTGAGGTTC | |
| | | | TTGTTATTTCATCACAAAAGGGTCACGCTCTGAT | |
| | | | GTTCCCAAAGGGTGGTTGGGAGCTTGATGAATCTGT | |
| | | | AGAAGAAGCTGCGTCAAGAGAATCTCTAGAAGAA | |
| | | | GCTGGAGTTGTTGGAAACGTTGAGAGACAACTTGGA | |
| | | | AAATGGGATTTCTTAAGCAAAAGCAAAGGAACAT | |
| | | | TCTATGAAGGATTCATGTTCCCAATGCTTGTGAAAG | |
| | | | AAGAGCTTGAGCTTTGGCCTGAACAACATCTTCG | |
| | | | TCAAAGAATTTGGATGAAAGTTGATGAAGCAAGAG | |
| | | | ACGCTTGTAGAGATTGGTGGATGAAGGAAGCGTTA | |
| | | | GATGTTTTGGTCCAAAGGCTTTCTTTACTATCATTGA | |
| | | | AGCCTATGGAAGAAGACGAGAACCTTCCACTGA | |
| | | | TCTCTATCTACTGATCAAAAAAAAAACAATCAGACG | |
| | | | TGTTCTTGATCACTTTGTATATAACATCTTACGC | |
| | | | CTGCCTTTCCATGAAGAGTCTACTCTACTGCTGCAG | |
| | | | GAAAAGTATGTACAGTGAAACTTTAGGTGTGAAC | |
| | | | TTGGCCGAGATTTGTTTGGATTTCGGTTTGGTTTAAC | |
| | | | AAAAAGGTTTTGTAATTGTTGTCATGATGTATC | |
| | | | CGCAACTGCTTAGGATGGGAGGAAGAAGACAGAAG | |
| | | | CAAAGAGTGCAAACAAAAAACTGTACAATAACGTT | |
| | | | TCCTCTCTTTGCATCTCTCCTCATCTTCAAATGAATC | |
| | | | TATGATCTAAATGAAACTAATAAAGAGAATTTA | |
| | | | CAGAAAATCATATTCACAAGACTATTATACAACTTA | |
| | | | ATTCTGTTTTCTTAACTGCATATATTTGTTATTT | |
| | | | TGAATTTCATGATAAGTTAAAAAAGAAAAAAAGAA | |
| | | | CCCAACAGCTACTAAATACGAATATATGCTATTT | |
| 19 | NM_122014 | 832129 | CTCATTTCTCACTCTGTGTGGGAGCCTAAACAATGG | 19 |
| | | | GGAGACGAAACCAGAGATGCTTGCTCTCTTCCTC | |
| | | | TCTTCTTCTTCGTATCCAACTCTCTCTTTCCTCTCCAG | |
| | | | ATCAGTAACTCTAAACTTAGCCAGAACAACAA | |
| | | | CACTCTCAGCTCTCACAATGTCGATGAACCTAAAGA | |
| | | | CTCATGCCTTCGCAGGCAATCCCTTGAAGTCGAA | |
| | | | AACTCCGAAATCTACCGATCCATTCTCACCCACCTC | |
| | | | CGCTTTCGAATCTCTGAAAACCCTAATTCCAGTA | |
| | | | ATCCCTAATCACTCAACACCTTCCCCTGATTTTAAAG | |
| | | | TCCTTCCCTTTAGCAAGGGTCGTCCACTGGTGT | |
| | | | TCTCAAGCGGTGGAGATGCTAATACGACGCCGATTT | |
| | | | GGCATCTGGGTTGGGTCAGCTTGGCTGATTGTAA | |
| | | | GGTTTTGTTAGCAAGTTGTGGGGTTGATCTGAATGA | |
| | | | GGACTCACTCGTGTATTTAGGACCCAAGTTGGAA | |
| | | | GAAGATTTGGTGTATTGGGCTGTTGATTTGGCAGAG | |
| | | | GATGGTTTCGTTTCTGAATTGGGAGGTAGGAAGT | |
| | | | TGTGTTTTGTTGAGCTTCGAACCCTAATGGTAGCTGC | |
| | | | TGATTGGGCGGATCAACGTGCTATGGATGAGTT | |
| | | | GGCTATTGCCGGCAATGCCAGGGCGTTACTTGAATG | |
| | | | GCACAACGTGTCACAATTTTGTGGATCATGTGGA | |
| | | | AGTAAAACTTTTCCAAAGGAAGCAGGAAGGAGAAA | |
| | | | ACAATGCTCAGACGAAACATGCAGAAAGAGGGTCT | |
| | | | ATCCCCGTGTTGATCCGGTGGTTATAATGTTAGTTAT | |
| | | | TGATCGAGAAAATGATCGCGCACTTTTAAGCAG | |
| | | | GCAATCTAGATATGTACCGAGAATGTGGAGTTGTTT | |
| | | | AGCTGGATTTATTGAGCCAGGGGAAAGCTTAGAA | |
| | | | GAGGCTGTGAGGCGGGAAACATGGGAAGAGACAGG | |
| | | | CATTGAAGTAGGAGATGTTGTATACCACAGCTCTC | |
| | | | AGCCTTGGCCTGTGGGACCAAGTAGCATGCCATGCC | |
| | | | AATTGATGCTCGGTTTCTTTGCTTTCGCCAAGAC | |
| | | | GCTCGACATAAACGTAGACAAAGAAGAGTTAGAAG | |
| | | | ATGCTCAATGGCACAGTAGAGAAGAGGTGAAGAAA | |
| | | | GCATTGGCGGTTGCAGAATACAGGAAGGCTCAAAG | |
| | | | AACAGCAGCTGCAAAGGTAGAGCAGATCTGTAAAG | |
| | | | GTGTGGAGAGAAGCCAGAGTCTATCAACTGATTTCA | |
| | | | ATCTTGAAAGCGGTGAGCTTGCTCCTATGTTTAT | |
| | | | CCCGGGGCCATTTGCCATCGCGCACCACCTGATCTC | |
| | | | AGCATGGGTAAATCAGGCTCCTGACGATGTTCAC | |
| | | | TCAAAACAACAAGCGGGTGTCTCTCTCTCAAGTTTG | |
| | | | TAGCTAGAAAGATGAACTTAGATGCTTAGAAACC | |
| | | | AAACGTTTGCTATGGCTTCTATGCTTATTTTGAATTG | |
| | | | CTGAGTCCTAAGTGTGTGGACTTCGCTCGAAGT | |
| | | | TATTTCTTTGCAACGACGATTTAGATCAATAAAAAT | |
| | | | TAATCAAAGATTGACCTCAATAAATTTGAGTTTT | |
| | | | ATAGTTCGTTTTTCTTTAGTGG | |
| 20 | NM_121951 | 832066 | CACGTTTATCAAATTCATAAATAAAAACTACTACTA | 20 |
| | | | CTATTTAACTATAAATATTTTTCCAAACAACACA | |
| | | | AAATAATAGCAATAGGACGTATACTAATAGAGTAA | |
| | | | TAGGTGGGAGCAATTGTTAGATTGTTACCAATCAA | |
| | | | ACGAGCCGTGACCAAAACTATAGCGCGGAAAGACA | |
| | | | AAACTATTTAACGAAAACGATATTGCCATTTGCGT | |
| | | | TTGTTAATAATCACTACTTACCAGTGCTTTTTACCGA | |
| | | | ACAGTACCGAGCCGTGACTAAAAATCAAGCATT | |
| | | | TCAATATCGAGTATACTCGATTCCGATTTTGGTCATC | |
| | | | CGTATATAATAACTCTTGATTTCCAAACTCGAC | |
| | | | ACAGCCATCGAAAATGGCATCTGGGTTTTGCTCACT | |
| | | | GGCTCTAACTGTCACCACCTCCTTATTCTCGTCC | |
| | | | CACGCGATTACACGGCGCGTTCTACCCATTCTACGG | |
| | | | TGGCGTTCCAGTTCTATGTCCCTTTCTCCGCTGC | |
| | | | GTCATTCCCGCGCTTTATCAGCTGCAACAACTGTTCC | |
| | | | GATAAGCTCATCCTTCACGTGGGACGACGTAAT | |
| | | | CGAGACCGGTCGGGCAGAGTACAACTCTTCCGATCT | |
| | | | CACCGGATTTTTCGAGAAAATCAACCGCTGCAAT | |
| | | | CGCGGATCCGAAAAGTTAGGTGAGTTCATTCCATTT | |
| | | | GTTATAGAGGAACAAATCGTTGGTTATATTCATA | |
| | | | AAAGATTTACGGAGTACTTGAGGGAGTTTCATGATA | |
| | | | TCTTTACATTTTCACAAAATGGTTCTTGTCCTGA | |
| | | | CCGTGTTGATGGCTATGTAACGCTTAATCTGATGCTT | |
| | | | CAAAAGCCTGAAGATAGAACCAGAGCAGTTGCA | |
| | | | GATGTGATCAAAATATTGGGTGACAAAGGAATCATT | |
| | | | CCAGGAATACGTAATGAGCTGTATCCTGTGAAAC | |
| | | | CATCGTTTAATGCCCCGGTCTTTTTTTCTCTAGAGCG | |
| | | | TGCTGCTGCTCCTTATTTCGGAATTAAGGGTTA | |
| | | | CGGAGTTCACATGAATGGGTATGTAGAAAGAGATG | |
| | | | GACAGAAATTACTGTGGATAGGTAAGAGAAGTCTC | |
| | | | TCAAAATCCACGTATCCAGGAATGCTTGATCATCTG | |
| | | | GTTGCTGGAGGATTGCCTCACGGGATTAGCTGCG | |
| | | | GTGGAAATCTAGTAAAGGAATGTGAAGAGGAAGCT | |
| | | | GGGATTTCCAGAGCCATTGCTGATAGGGCAATAGC | |
| | | | GGTTGGTGCTGTCTCATATTTGGACATCGATCAGTA | |
| | | | CTGTTTCAAACGTGATGTGCTGTTTTGTTATGAT | |
| | | | TTGGAACTCCCTGAAGATTTTGTTCCCAAAAATCAA | |
| | | | GATGGAGAAGTTGAGAGCTTCAAGTTGATTCCAG | |
| | | | TAGCCCAAGTTGCTAGTGTGATTAAGAAAACTAGTT | |
| | | | TCTTCAAGGCGAACTGTTCCCTTGTCATTATTGA | |
| | | | CTTCTTGTTTCGGCACGGGTTTATCAGACCAGAATC | |
| | | | ATCGGGTTACTTGGATCTATACCAACGCCTGAGG | |
| | | | AATAGAGATTGCTCATAGGGATGTTACTATCTGATG | |
| | | | ATTAGTCTGCTGGGTCACTTCAGGTGCCTTCATG | |
| | | | AACATAATGAAGTCCAAAGAATACAATTTTTTATAC | |
| | | | AGAGAGCGAGCAAAACAAACTCACGTTTAGAGTT | |
| | | | GTACATTTTAGCTCCTGTTCTGTTCTAGACTATGAGA | |
| | | | CTACGAGGGAAAATGCTTCTCTATTAGTTGTGA | |
| | | | TTTCTGGTTTGAGCTAACCGGAAGCTAATGAGCTCA | |
| | | | CATGGAATTGAAGGCTAGCTTAGTGAATTGTTGG | |
| | | | AAACCTAGCCGAGATTAGGGGATCACACACTGTAAT | |
| | | | TGCTTAACTAGTAAATCGCACTGTGATCATTATC | |
| | | | TTCTTTCTCGATCAATATACAAGTTTCTCGATTAATG | |
| | | | TTCCATACTTTGACTGTGAAATCATGGTTGATG | |
| | | | CAAAGATCGCAACATTCTCTCTACGGATCAGCTCTA | |
| | | | ATGTGAGCCTTG | |
| 21 | NM_106012 | 843688 | GTGAGATTTTCCATATAAAGACGCTCGCAAACCCAA | 21 |
| | | | TCCTCTTTCACAAATCACAAATCACATCTCTTCA | |
| | | | TCTTCCTCGGTTTATCCAAATCCTCATTCAGACTTCA | |
| | | | TCAAATCGTTTCTTGCTTCCGATCCGAAATTTG | |
| | | | CGGCGGGAAAAGAAGATAACATCAACAAAAAAATG | |
| | | | ATTTCTCTATTCATCTCAAACTTTTCAAACTTATC | |
| | | | AAACTTATCTCCGACCTTTGATAACATGAACATGAA | |
| | | | CATCCCATCGAAGAAGATCGTACCGGTACCGACA | |
| | | | CCGAGTGAGAAAGTTGTCTCTTTGGTATCTCGAACC | |
| | | | GGACGAGATCTCCAACGTTACAACACCGCAGGGT | |
| | | | ATCGTCAAGTCGTCGGATGTGTACCATATAGATACA | |
| | | | AGAAACACGGAGGAGGAGAAATTGAAGTGCTTTT | |
| | | | GATAAGTGCACAGAAGAAAGGGAAAGGAATGTTAT | |
| | | | TACCAAAAGGAGGTTGGGAGATTGATGAATCAATA | |
| | | | GAAGAAGCTGCTTTAAGAGAGACAATTGAAGAAGC | |
| | | | TGGTGTTACGGGTCAGCTTGAAGAATCACTTGGGA | |
| | | | AATGGCAATACAAAAGCAAAAGACATACTATGATT | |
| | | | CATGATGGTCATATGTTTCCTTTGCTTGTTAGTCA | |
| | | | GCAGTTTGAGATATGGCCTGAATCTGAATTCAGACA | |
| | | | ACGCAAATGGGTTTCTTTGTCTGAAGCGATTGAG | |
| | | | TTGTGTCAGAATTCGTGGATGAGAGAAGCTTTGGAA | |
| | | | GCTTTCATTAACCGGAAATGCCAGACCCAATAAG | |
| | | | GATAATGTAGCATCTCTGGGAACAAAGAGCTCTTCA | |
| | | | TTTGGAAGCAGATGCATATAATTGAAGAGCAGGT | |
| | | | CCTCAAGTTTAGTGTTCTGATTTGATTCATTCATTTG | |
| | | | TTAATGATGTTAGTAGACTAATTGATAATAACT | |
| | | | GATTGTTCTGAATGATTGTCTTCACAACATGTAATGT | |
| | | | GTACATATATAAAATGGATCAATCTCATAGATG | |
| | | | TAATAACGACTTGGTCTTCACTTTTGATTTCTTATGA | |
| | | | TTTTCAATTTGTTGGTGTTATTTTTTGAAAAAA | |
| | | | ATGCAGATTAGGCGGTGATGAATAATTATTGACGAG | |
| | | | ACCAAGGAAAA | |
| 22 | NM_128949 | 817959 | TTGGTCTGCCTTTCAAGCGAGCCTTGGCTAGCGGAC | 22 |
| | | | AACTGTTGTAATTCACTGCAATTCCATGAAGTCG | |
| | | | GGAGCATCAGCCGCGTCGCCAACGGCGAAAAGCTT | |
| | | | CAATTTTGGATCTTCCAGACTCTTAGCCTTGGCGC | |
| | | | AGCAGCTGCGTGTATACAAGCCGCCGCTTTCGTCAT | |
| | | | CCTTCGACGAGAGGGAAGAGCTGCTTGCGTACAA | |
| | | | GGAAAGCACCAGGAAATCGATAACCCACGTTGGGT | |
| | | | TTCAGGAATCTATGGCTCCGGTGAGGTTCAGACCG | |
| | | | AAGAAAGCTGCTGTCTTGATATGTCTCTTCGAAGGA | |
| | | | GACGATGGTGATTTGCGTGTGATTCTTACTAAGA | |
| | | | GGTCTTCCACATTGTCTACTCACTCAGGAGAAGTTT | |
| | | | CTTTGCCAGGTGGTAAAGCAGAAGAGCATGATAA | |
| | | | GGATGATGGGATTACTGCAACTAGAGAGGCAGAGG | |
| | | | AAGAGATTGGATTAGACCCTTCGCTTGTTGATGTT | |
| | | | GTTGCTTTCCTCGAACCATTTCTATCTCAGCATCTGC | |
| | | | TTAGAGTAATTCCTGTGGTAGGAATATTATGGG | |
| | | | ACAGAAAAGCATTCAATCCAACACCAAATCCTGCA | |
| | | | GAAGTGGAAGCCGTGCTTGATGCACCCTTTGAAAT | |
| | | | GTTTCTGAAGGATGAGAACCGGAGATCCGAGGAGT | |
| | | | TTGATTGGATGGGAGAAAAGCATTTGGTCCACTTC | |
| | | | TTTGATTACAAAACAGGAGATAGTGATTATGTTATA | |
| | | | TGGGGCTTAACTGCAAGAATCTTGATTAGAGCTG | |
| | | | CAACAGTGGTTTATCAACGACCACCTGCTTTCATTG | |
| | | | AACAGAAACCTAACTTGAAGTACTCCAAAATGAA | |
| | | | CCAGGCTACTCGTTTATATGGATGGCTGAAGCCGCT | |
| | | | GGCAAATTTACATGAATCTTGTGAAAATGGAAGA | |
| | | | TTACTAGGTCATGTCCCAGCATACTGCTTGCGTTATC | |
| | | | TAGTTGGAATATCATCAAGATGATCGTGTTTGT | |
| | | | GCATATCTCAATCGTTTGTGCTGAAAGACAAGCTCA | |
| | | | TTCTTGGAAAGTCTTTCAGCATGCTTTTTGGATG | |
| | | | ACTCTGGTATCGGAAATCAACAGATTTAAGTTATCT | |
| | | | AATTCATCTGATGCAAGAGCAAAGGTTAAAAAGC | |
| | | | TAAAGGGAAGGCTGCTGCGTAGATCCATCTTCTAGA | |
| | | | TGCTTTGTTTAGCTCTTTCATGTTCTAATGTCTC | |
| | | | AAGAGCCACTGATGCAGGTAATAGGTTTAGAGCCAT | |
| | | | TTCTGATTGATTTGCATCTAGGAAGGTGGTTTTG | |
| | | | ATTCCGTGAAACTAAGCCAGTTGATCTGATGACAAG | |
| | | | GATACAATTTGGATGTGGTTTTCCTAGTGGACCT | |
| | | | TTACCACATAATCAAATATTGGATGTGCTCCTTGAA | |
| | | | AACATGGGTAGCTCCCGTTTTGCAAGAAGTGTGT | |
| | | | TTTTTGGTGAAACCCCAACCATTTGACCTGAAAGTA | |
| | | | AAGCTCGTTCTTGAAAAATCTGCCAGCAGGTGGG | |
| | | | TAGTGCTTGTGCTGCTTTCTGATGTTTTCCGGTTTTT | |
| | | | CTGGTGGAACAAATTAATACATCTGATGCGAAA | |
| | | | GAAAAGCTCGTTCTTGCCTTCTTGGAGAATCTGGCA | |
| | | | GCATAGCGCTTTGTCATGGTCAATGTGTGTCTGA | |
| | | | TATTATCTGGTTGGAAATCTCAATCATATGACCTGA | |
| | | | TAGCAAAGGAGCATTTGACTTCAGTTAAAAGAGC | |
| | | | TACAGACACGCTGTTCTAATAGGTCTGAGATGAAAT | |
| | | | TCCGTGTAACTTTGGTCTCCTGAAGAAGCCAAGT | |
| | | | GGTCGATAAAGCCATCACTCGTCTTATGGAACAAAG | |
| | | | GAGAATGCCTAAATATAATGAATCAACGTTGCCA | |
| | | | AAACTAAGATGGATGATAAACCTTATAAGTTAGGG | |
| | | | ATCAAATTCAATAAAAAATAAACAATGAGAAAAGT | |
| | | | AAAACAGTACATGTATGAAGTGACACACGAGTGAA | |
| | | | AAAGAAGACTAAATCGTCTTATTGTACTCCAAACA | |
| | | | TTGATCCTCTACATTTCTTTAGGTTATATGTAATTAT | |
| | | | GCATGCTACAACTTAAGCATATTGTCGTAATTT | |
| | | | AGTGATATATTGGAGCCACAT | |
| 23 | NM_129770 | 818807 | ATTGCCCTGTGATTCTTTAATGTTTGTTTTCTCTTTAG | 23 |
| | | | TTTACAAGTACATGTGACTCTATAACACTCAC | |
| | | | TCAGCTCTGCTTTTGTGTCTGAACCGGTTAATTTGGT | |
| | | | CAAATTAAACCGGTTGTTGGGTCTTAGTGAAAC | |
| | | | CAATAATGCCGGACCTTTGGCTAAGTTAAAGAGTCA | |
| | | | CTTCATTCAACAAATCTCCTCAAATGTCAAATTT | |
| | | | TTGGAACGAAATAGATTGGCCATCGAGAGAGGAGC | |
| | | | TTCATTGACAAAGAGTCCTATAGCCGGAAGTGAAG | |
| | | | AAATTTCCGGTGCGTATAATGCTAAAGGCCGTCCAG | |
| | | | ATCTTGGGTTGGTCATCAGGGTTAACCATCTCAC | |
| | | | AGAGATTGACCAAGACTCGCAAGTCCTCTACCGTCT | |
| | | | CTTTCATCTCGTCATCTCTCAATCTCTCCTCTGT | |
| | | | CACTTCGAGCTCTCCTCGGAGAATCTTCTCTTTCAAG | |
| | | | CCAACCAGAATGTCTTCCTCACTTCCCGGGTCT | |
| | | | GACCCAGTTGCAAATTCCCCGACTTTCGTCTCTGTTC | |
| | | | AATCAGCTGGAGATGTTCGTAAGATAAAGTTCT | |
| | | | GTCAATGGTGTGGAGGACCAACCAAGCATGAAATA | |
| | | | CCTGACGGTGAAGAGAAATTGAGAGCTATTTGCAC | |
| | | | ACATTGCGGCAAAATCGCTTATCAGAATCCTAAGAT | |
| | | | GGTTGTTGGTTGCCTTATAGAGCATGAAGGAAAA | |
| | | | GTTTTACTTTGCAAACGTAACATTCAACCTTCACAC | |
| | | | GGTTTATGGACTCTTCCTGCTGGTTATCTAGAAG | |
| | | | TTGGAGAATCAGCTGCACAAGGAGCAATGAGGGAA | |
| | | | ACTTGGGAAGAAGCAGGAGCTACTGTGGAAGTTAT | |
| | | | TTCACCTTTTGCTCAACTTGATATTCCTCTTATTGGC | |
| | | | CAAACTTATGTTATATTCTTAGCCAAACTGAAG | |
| | | | AACCTGCATTTTGCACCTGGTCCTGAATCATTGGAG | |
| | | | TGCCGTCTTTTTGCACTAGATGAGATACCGTTTG | |
| | | | ATTCCTTGGCTTTTTCATCTATATATGTTACCTTAAA | |
| | | | TTTGTACTTGGAAGATCTGAAAAAAGGGAAACT | |
| | | | AAAGTTTCACTATGGTACGATAAATAAAAGGCCTGG | |
| | | | AAGTAGTCCTTCAGATATTCGCGCCTTTAGTCTT | |
| | | | GATTACCATCTGCAACCGTAACTCAAGTTTACAGAA | |
| | | | GCAGCTTCTGGATTGGCTTTGTTCTTCCATGTCG | |
| | | | AGTCTTTTCATTTCGCAACTCGATGTTACTAAAAGA | |
| | | | AAAGGAATGATGAAGTTGCTTATGTGAGAGATTC | |
| | | | CTTGGACAGATGAGAAAGAACTCGGAAGAAGATAA | |
| | | | TTTGGACAGAGTTTTGTTCTGTATTTGTGGTGGAG | |
| | | | AAATAGGTTTGGATGACTTTTAGGTCTCTCATGTCA | |
| | | | GATTTTATGGCTGAACAATGTGTATCTTTATCTG | |
| | | | TTTCTGGTTTCAGTTGCCTCAAATGTAAAAGGAAAA | |
| | | | AAATCATATGTGGATGCAACATGTTGATCAATCT | |
| | | | TTGTGTAATTAAGTCACCGTGTTACTGTTACTTCAAG | |
| | | | GTCTTCAGTGAAAGAAAGATAAACCAAATAGAT | |
| | | | AGGATATATTTATTC | |
| 24 | NM_121952 | 832067 | GAGATTTTTTTTCAAAAAACTTAATCATCTGTATTTA | 24 |
| | | | TAACGCCTTGCATCCCAAGCTAGAACAGCATTG | |
| | | | TGTCTCCTCCACAGACATGGCATCTGCATTTTGCTCA | |
| | | | CTTTGTCCCACTCCCACCTCCTTATTCTCTTCC | |
| | | | CACGCGCTTATACCCACTCTACAGTGGCGTTCGAGT | |
| | | | TCGAGTTCGAGGTCTCCTCCGCTACATATTTCCC | |
| | | | GCGTTTTATCAGTTGAAACTGTTCCTTTAAGCCCATC | |
| | | | ATTCACCTGGAACGATGTTTTTGAGAACAGTCG | |
| | | | AAAAGAATACGTGCCTCAGAACTCCTCCGATCTCAC | |
| | | | CGGATTTCTCGAGAAAGTCGACCGCTGTAATCGT | |
| | | | GGATTAGAGAAGTTAGGTGAGTTCATTCCATTTGTT | |
| | | | ATAGAGGAACAAATCGTTGGTTATATTCACAAGG | |
| | | | GATTTACAAAGTACTTGAGGGACTTTAATGATATCT | |
| | | | TTACATTTTCACAATATGGTGGCCATGTAACGCT | |
| | | | TAACATGATGCTTGACAAGCCTGAAGAAAGAACCA | |
| | | | GAGCAGTTGCACATGTGATCAAAATATTGGGTAAC | |
| | | | AAAGGAATCATCCCTGGGATACGAAATGAGCTATAT | |
| | | | CCTGTGAAGCCATCGTTTAATGCTCCTGCCTTTT | |
| | | | TTTCTATAGAGCGTGCTGCTGCTCCTTATTTTGGATT | |
| | | | GAAGGGTTACGCAATTCATGTGAATGGGTATGT | |
| | | | AGAAAGAGATGGACAAAAATTTCTATGGATAGGTA | |
| | | | AAAGAAGTCTAGCAAAATCCACTTATCCAGGAAAA | |
| | | | CTTGATCATCTGGTTGCTGGAGGATTGCCTCACGGG | |
| | | | ATTAGCGTTTGTGAGAATCTAGTAAAGGAATGCG | |
| | | | AAGAGGAAGCTGGGATTTCCAAAGTCTTGGCTGATA | |
| | | | GGGCGATTGCGGTCGGTGTTGTTTCCTACATGGA | |
| | | | TATCGATCGGTACTGTTTCACACGTGATGTGCTGTTT | |
| | | | TGTTATGATTTGGAACTCCCTCAAGATTTTGTT | |
| | | | CCCACAAATCAAGATGGAGAAGTTGACAGCTTCAG | |
| | | | GTTGATTCCAGTCGCTCAAGTTGCTAATGTGGTTC | |
| | | | GGAAGACTAGTTTTTTTAAGGACAGTTGTTCCCTGG | |
| | | | TCATTATTGACTTCTTGTTTCGGCACGGGTTAAT | |
| | | | CAGACCAGAGTCACCGGGTTACTTGGATCTATACCG | |
| | | | ACGCCTGAGGAATGGAGATTGCTCATAAGGACGT | |
| | | | TACTTTCTATCTAATGTTTTAAAGAAAAAGCTCACTC | |
| | | | TCTAATAAATTAATATTAGATACTCTGACTATG | |
| | | | TAATAAGACTCATGCATGTATATAATATTCAGAGTC | |
| | | | TTCATGTGCCTTCATGAACATATATATACCAATC | |
| | | | AAGGAAGTTATAAAACTTCATTTATAC | |
| 25 | NM_001198185 | 839890 | ACATTAGAAGTTAAAACAAATTTTGTCAACTAATTT | 25 |
| | | | GAAAATTACAACCAGGTCATATAAGTATAAAAAA | |
| | | | ACAAAAAAAAAACAAAACACACACACACACACATG | |
| | | | TTCTTCGTTGCTGCTTCGTTGGGTTAAAAACTTTT | |
| | | | CTTTTGTTTCTTGCTTTTTGCAAAGTCAAAGATAACA | |
| | | | AATAAATAAGAAAAATGGAGAATCTACCACCTG | |
| | | | GTTATCGTCCCAATGTTGGTGTTTGTCTAATCAACTC | |
| | | | GGATAATCTGGTATTTGTAGCTTCTAGATTGAA | |
| | | | TGTTCCAGGAGCATGGCAGATGCCACAGGGAGGCA | |
| | | | TTGAAGATGGGGAGGATCCAAAGTCAGCAGCCATG | |
| | | | AGAGAGTTACAAGAAGAAACTGGTGTTGTTTCAGCT | |
| | | | GAAATCGTCTCTGAGGTCCCAAATTGGTTGACAT | |
| | | | ATGATTTTCCACCAGCAGTAAAAGCAAAAGTTAACC | |
| | | | GTCTTTGGGGCGGTGAATGGCATGGTCAAGCTCA | |
| | | | GAAATGGTATTTAGTGAGACTGAGGAACGATGAGG | |
| | | | ACGAGAAAGAGATCAATCTAGCGAACAACGAAGCG | |
| | | | GATTCAGAGTTTGCGGAGTGGAAATGGGCGAAGCC | |
| | | | AGAAGAAGTGGTAGAGCAAGCAGTGGATTACAAAA | |
| | | | GGCCAACCTATGAAGAAGTCATCAAGACTTTTGGTT | |
| | | | CGTTCTTAAACGACACAGGAAGAGCTGCTAAATG | |
| | | | TAAATCAGCCAAGTGGTGATATAAGATACCAAGAA | |
| | | | CCAATGTTTGTCTTTTTATTTTTTTTGTTAACTGT | |
| | | | TGTTTTTCTTTTTTTGGGCGGTTCTTTTTAGTAGAAC | |
| | | | CCTTTTGGAATGCAAAAGCTTTCTCTAGGGGTT | |
| | | | TTTTACT | |
| 26 | NM_111898 | 820231 | GTGTAGTCTTTGTACGTAATATTCATGATGAGTCAG | 26 |
| | | | GGGCATAACGGTCCAGTTACTTATTAATTTCCCG | |
| | | | AATACGTGGAAAAATAGGTTTTGGCTCGAAATGGCA | |
| | | | CTGTACCGACCCCTTCTCTTGCATCATCCAACTT | |
| | | | CTCCGTCTGTAACTACGTTTCTACGAAATTACCCCTC | |
| | | | GAAACCGATCAAATTCTCCAGCCTGCCCTTCCT | |
| | | | TCATCGCTGCCGTAAGTCTCGGGTCTCCTCCTCCTCC | |
| | | | GCTCGGTGTTGTTCTTCCATGGAATCTCCTCCT | |
| | | | GAAGGATACAGAAGAAATGTCGGCGTCTGCCTCAT | |
| | | | GAATTCTTCCAAAAAGATTTTCACTGCCTCGAGGT | |
| | | | TGGATATTCCCAGTGCTTGGCAAATGCCTCAGGGTG | |
| | | | GAATCGACGAAGGTGAAGATCCAAGAGTTGCAGT | |
| | | | CATGAGAGAGCTTAAAGAAGAGACTGGAGTTCACT | |
| | | | CTGCTGAGATTCTCGCTGAGGCACCTCACTGGATA | |
| | | | ACCTATGACTTCCCTCCTGATGTTAGGGAGAAGCTT | |
| | | | AAGGTCCGATGGGGATCCGATTGGAAGGGTCAAG | |
| | | | CACAGAAATGGTTTCTTCTGAAATTTACTGGGAAAG | |
| | | | ATGAAGAAATCAATCTTCTTGGCGATGGTACTGA | |
| | | | GAAACCTGAGTTTGGTGAATGGTCCTGGACTTCTCC | |
| | | | TGATCAAGTCGTTGAAAATGCAGTGGAGTTCAAG | |
| | | | AAGCCAGTGTACAAGGAAGTCATGTCAGCTTTCGCT | |
| | | | TCTCATCTCCAGTAGCAAAATAAATCGAAAACTA | |
| | | | AACTCTCTGTTTTAAGTTTTTCGTAAGAAACTCTGAG | |
| | | | AATAAGCTCTTCTTGACATATTTGCTCTTTTTT | |
| | | | GAACAATCTTTGAATCAAAATACAATTCTTGTTCCC | |
| | | | TCTCTTGATAAAAGTAAAGTGTGAGATCCTGTCA | |
| 27 | NM_120717.4 | 830522 | GTCTAAGCGGGTAAGGGTAGTGTCGTAATAATATAT | 27 |
| | | | TTTATCACTAGTGAGAAAATTGAAACCAATCACC | |
| | | | GATTGTTCTGAGCCGGACGAAGGAGAGGCAGCCGC | |
| | | | CTAAGAAGTTGCCGTCGTGCGCCATGGCCGTGAAG | |
| | | | GCTTCTGGATTCATTGGGAAATCAGCAATCTCTGTT | |
| | | | CATCTAGATTTCTCTTCGTTTCCTGTGAAATTTT | |
| | | | CATGCTTGAAACAGTTTTCTGTATCTTCACCAAAGC | |
| | | | CACTGGTCGTTTTATCCGTTGCCTTATCGTCGCC | |
| | | | GGCAAGAACAGTTGAGTCTCCGCCTGTTGGTTACCG | |
| | | | GAAGAATGTTGGGATTTGTCTCGTTAGCCCATGT | |
| | | | AGAAAGATTTTTACGGCGTCAAAGATTCACATTCCG | |
| | | | GATACATGGCAGATGCCTCAGGGTGGTGCTGATG | |
| | | | AGGGAGAAGATTTGAGGAACGCTGCCTTTAGAGAA | |
| | | | CTGAGAGAAGAAACAGGGGTTACTTCGGCAGAATT | |
| | | | CATTGCGGAGATACCAAACTGGCTGACCTATGATTT | |
| | | | TCCCCGAGAAGTAAAGGACAAACTAAACCGGAAA | |
| | | | TGGCGAACAAGTTACAAAGGCCAAGCTCAGAAGTG | |
| | | | GTTTCTCTTTAAGTTCACGGGCAAGGAAGAAGAGA | |
| | | | TAAATCTCCTTGGAGACGGTACTGCAAAACCAGAGT | |
| | | | TTAAGGTGTGGTCGTGGATGCTACCGGAACAAGT | |
| | | | TATTGAACATGCTGTATATTTCAAGAGACCTGTTTAT | |
| | | | GAGCATGTCATAAAGCAATTCAACCCTTATTTT | |
| | | | GTGGATGAAGAAAAGGATTCCATGAACTCGTCTAA | |
| | | | GGATTGAATGTGCACAAAGATTCACCAAGTGAATA | |
| | | | CTGATTAGAAGCAAATCAATCAATTCATTATACTGG | |
| | | | TTTCTCTTGAATCTCATCAAAAATAAAAATTCAT | |
| | | | GAATCTCAGACATTGATCTTGTAGTTTGATGCTATAT | |
| | | | ATAAATCCTCACGAATTAAATTCACGAACTAAA | |
| | | | TTACCACATTAATAAATAATTAATTTACCCATAAAT | |
| | | | TACAACATTATGCTGATAC | |

TABLE 2
Reference NUDIX proteins from Arabidopsis thaliana

| | Protein (GenBank | | SEQ |
|---|---|---|---|
| NUDIX | Accession No.) | Reference Sequence | ID NO: |
| 1 | NP_177044 | MSTGEAIPRVAVVVFILNGNSILLGRRRSSIGNSTFALPGGHL | 28 |
| | | EFGESFEECAAREVMEETGLKIEKMKLLTVINNVFKEAPTPS | |
| | | HYVSVSIRAVLVDPSQEPKNMEPEKCEGWDWYDWENLPKP | |
| | | LFWPLEKLFGSGFNPFTHGGGD | |
| 2 | NP_568687 | MSASSSSTNPMSREDATTLLPSVQDKYGGVMTEMTHPMDP | 29 |
| | | SLFSTLLRSSLSTWTLQGKKGVWIKLPKQL | |
| | | IGLAETAVKEGFWFHHAEKDYLMLVYWIPKEDDTLPANAS | |
| | | HRVGIGAFVINHNKEVLVVQEKTGRFQGQG | |
| | | IWKFPTGVVNEGEDIHDGSVREVKEETGVDTEFDQILAFRQT | |
| | | HKAFFGKSDLFFVCMLKPLSLEINAQES | |
| | | EIEAAQWMPWEEYINQPFVQNYELLRYMTDICSAKTNGDY | |
| | | EGFTPLRVSAPDQQGNLYYNTRDLHSRN | |
| 3 | NP_565218 | MAEEHFDVLTKSGEKTGVSKPRGEVHRDGDYHRAVHVWIF | 30 |
| | | VETTQQLLLQLRSDDKDSWPGQWDISSAGHISAGDTSLLSA | |
| | | QRELEEELGVKLPKDAFEKIFVFLQECVTNDGKFINNEFNDV | |
| | | YLVTILHPIPLEAFTLQKEEVSAVKYVPYEEYRNFLSKEDPA | |
| | | YVPYDVNGEYGKLFDIIRQRCQVNTEARSLSLQKQLQRYSP | |
| | | VTLEAKLTELSEADQKALGLIVKAAKIMDDIFYEQVWNSNP | |
| | | ALRDWLKDHANASKLDKLKWDYFTINKSPWSSLDENEAFL | |
| | | STADSAVKLLPGATKAIAGWKGLEYRAAFPVTKPPGANFYP | |
| | | PDMDKMEFTLWLNGLTEEQKHAATGFFSVIKRRSEANLDAS | |
| | | DHLASSTKKLPDSNSDLYSIPYSEIYRPFLKKASEFLQKAGDL | |
| | | VSSPSLKKLLHSKAEAFLSNEYYESDIAWMDLDSKLDITIGP | |
| | | YETYEDEIFGYKATFETFIGIRDDKATADLKLFGDNLKLLED | |
| | | NLPLESVYKSTDVSAAPIRVIQLIYNSGDVKGPQTVAYNLPN | |
| | | DEKIVKDRGTSMVMLKNVQEAKFEHILKPIAEITISKEQRGL | |
| | | VDFDSFFTHTICHECCHGIGPHTITLPGGQTSTVRKELQEVHS | |
| | | AMEEAKADIVGLWALKFLITKGLLSKSMVESMYVSFLAGCF | |
| | | RSIRFGLTEAHGKGQALQFNYLYEKGAFVFHEDSTFSVDFA | |
| | | KIEGAVESLSHEILTIQGKGDKNAATLLLNKYCTITGPLKTAL | |
| | | ENLERVKVPVDISPTFPLAEALMN | |
| 4 | NP_173266 | MTGFSVSLFVSNLSNVASYLSPIFENIPSTKVVPAQIEKVVSL | 31 |
| | | VSRTGRDLQRYDHAGYRQVVGCVPYRYKKQEVNGVETQVI | |
| | | QVLLVSAQKGKGMLFPKGGWETDESMEEAALRETIEEAGV | |
| | | TGELEEKLGKWQYKSKRHSIIHDGYMFALLVSQEFERWPEA | |
| | | EMRQRRWVSLDEAREVCQNWWMREALEAFINLKCLADDD | |
| | | ESGN | |
| 5 | NP_178524 | MGLTKNFAFLVFCCQRYCSMDGEAFEISLLDGEEDRFGGTV | 32 |
| | | VNLMEVESMTIGDFDSKLDVSLKAWKDQG | |
| | | KKGIWIKLPSELSSLVDTAIKKGFTYHHAENEYVMLTFWLPE | |
| | | PPSTLPCNASHRIGIGAFVLNKNGEMLV | |
| | | VQENSGYFKDKNVWKVPTGTIKEGESIWAGAVREVKEETDI | |
| | | DAEFVEVLSFMESHQAVWQRKTDIFFVCE | |
| | | LEARTFEIQKQDSEIHAAKWMPVEEYVNQPYHNKEGNEMF | |
| | | KLIANICLKRSREKYTGFVLTTNSAKKSLY | |
| | | CSVDHANLLKETADQASTSLSD | |
| 6 | NP_178526 | MDNEDQESLLLQGVPDNYGGVKVNLTEPMTIEDFVPKLRAS | 33 |
| | | LVYWSNQGTKGIWLKLADGLDNLIAPAKAEGFVCHHAERE | |
| | | YTMLTSWIADVPSTLPANASHRIGVGAFVLNKKTKEVLVVQ | |
| | | EIDGHFKGTGVWKLPTGVVKEGENIWEGALREVEEETGIKT | |
| | | KFVEVLAFRESHQAFLEIKTDIFFLCELEPTTFEIKKQDSEILA | |
| | | AKWMPIEEYVNQPWNQKKELFRFMANICLKRLQEMEYMG | |
| | | FSKVLTTTSSGKESYLYCNTDHANLLNATRGLASTSG | |
| 7 | NP_849367 | MGTRAQQIPLLEGETDNYDGVTVTMVEPMDSEVFTESLRAS | 34 |
| | | LSHWREEGKKGIWIKLPLGLANLVEAAVSEGFRYHHAEPEY | |
| | | LMLVSWISETPDTIPANASHVVGAGALVINKNTKEVLVVQE | |
| | | RSGFFKDKNVWKLPTGVINEGEDIWTGVAREVEEETGIIADF | |
| | | VEVLAFRQSHKAILKKKTDMFFLCVLSPRSYDITEQKSEILQ | |
| | | AKWMPIQEYVDQPWNKKNEMFKFMANICQKKCEEEYLGF | |
| | | AIVPTTTSSGKESFIYCNADHAKRLKVSRDQASASL | |
| 8 | NP_568680 | MDSVSLSEVTVIKGTTHLGFMHSFRQPFCGVKISPKFYLSKV | 35 |
| | | DGPKAISSSSNTKSQFVYGGGSIAATSD | |
| | | SGYKMNGVNLKSRTLMSSAVKERSLLDAYDDEYGGVIVDH | |
| | | GKLPSNPYAFASMLRASLSDWRRKGKKGVW | |
| | | LKLPVEQSELVPIAIKEGFEYHHAEKGYVMLTYWIPEEEPSM | |
| | | LPANASHQVGVGGFVLNQHKEVLVVQEK | |
| | | YCAPSITGLWKLPTGFINESEEIFSGAVREVKEETGVDTEFSE | |
| | | VIAFRHAHNVAFEKSDLFFICMLRPLS | |
| | | DKIIIDALEIKAAKWMPLAEFVEQPMIRGDKMFKRVIEICEA | |
| | | RLSHRYCGLSPHRLVSTFDGKPSSLYYN | |
| | | VVDDDHDPSHSNCSTEFYR | |
| 9 | NP_190206.2 | MAKLAEEMNPEGSRYQLLLSCPSGLSPSQVSVDFSKSHDRIP | 36 |
| | | RQDPGLEDSISQVWEQRSQGNSSLFNGQKFRYGGYCLDDD | |
| | | DGSTNEVPHVCLRLGLTDYRTFVGTNLSSLWEKFLVTSEDD | |
| | | SVRCRHTSSPLGNGAVIETSDKKIIVLRRSNNVGEFPGHYVFP | |
| | | GGHPEPTAVGIDYHQLENNVQTGEVLNKKVTQEMFDSIICE | |
| | | VVEETGIPASSLSSPLFIGISRRELNVRPAMFFYLKCSHHSDDI | |
| | | QRLYSSAEDGFESTQLHTVSLDELKMMTSRMPGCHHGGFA | |
| | | LYELMLQRLKNTKETSLIAT | |
| 10 | NP_849443 | MSDQEAPLRNGVEHKIFEVLPFVDDDYGGVIVEMKTPMDT | 37 |
| | | KNFVAALRDSFEQWRLQGKKGVWLNLPLSHVNLVEPAVKE | |
| | | GFRYHHAEPTYLMLVYWIPEAESTIPLNASHRVRVGAVVLN | |
| | | HNKEEKYGSLCGSGIWKIPTGVVDEGEEIFAAAIREVKEETG | |
| | | IDTEFLEILAFCQTHESFFAKSDLFFVCLLRPTSFDIQKQDLEI | |
| | | EAAQWMRFEDSASQPITHKNDLFKDIHHICSMKMEKSYSGF | |
| | | SKKPITTFFDDKLGYLYLNKQEDMEQPIS | |
| 11 | NP_199406.1 | MSSTTTDSTELQNLIKLFQNCQTHPRQHFPAKSSAVLVCLYQ | 38 |
| | | EQREDKNELRVILTKRSTTLSSHPGEVALPGGKRDQEDKDDI | |
| | | ATALREAREEIGLDPSLVTIISVLEPFVNKKGMSVAPVIGFLH | |
| | | DKKAFKQLPNPAEVEEIFDVPLEMFLKDRNRRAEEREHEGE | |
| | | RYLLQYFDYYSEDKERSFIIWALTAGILIRVASIVYQRLPEFQ | |
| | | ERKPSFWNQPN | |
| 12 | NP_563919 | MSVLSSRTGRDRQRYDNNFRLVSGCIPYRLMKADETEEDSG | 39 |
| | | VDFVNKLEVLMVSSPNRHDLVFPKGGWED | |
| | | DETVLEAASREAIEEAGVKGILRELPLGVWEFRSKSSTVEDE | |
| | | CLGGCKGYMFALKVTEELEDWPERKNRE | |
| | | RRWLTVKEALELCRYEWMQRALEEFLRVMEDERRLRTEEE | |
| | | TVHDSSKLEEESQIDPWYCFVVN | |
| 13 | NP_850636 | MSNLSARTGRDHQRYDNNFRLVSGCIPYRLVKDEEEDSTSV | 40 |
| | | DFENKLQVLMISSPNRHDLVFPKGGWEDD | |
| | | ETVLEAASREAMEEAGVKGILREDPLGVWEFRSKSSSVEAD | |
| | | CCLGGGCKGYMFALEVKEELAIWPEQDDR | |
| | | ERRWLNVKEALELCRYEWMQSALEEFLRVMAEEGSTKEDS | |
| | | LAISSISNRGERQIDPRYCFVV | |
| 14 | NP_567384.1 | MAGFTLLPSRLLAFPSRALPRRLHHHHAKLILRCKMSSSSSS | 41 |
| | | LTQSITLPSQPNEPVLVSATAGISSSDFRDAIDSSLFRNWLRN | |
| | | LESESGILADGSMTLKQVLIQGVDMFGKRIGFLKFKADIFDK | |
| | | ETGQKVPGIVFARGPAVAVLILLESDGETYAVLTEQVRVPTG | |
| | | KIVLELPAGMLDDDKGDFVGTAVREVEEEIGIKLKKEDMVD | |
| | | LTAFLDPSTGYRIFPSPGGCDEEMSVFLYRGQVEKETIRQLQ | |
| | | GKETGLREHGEFIKVRLIPYRELWRKTADAKVLMSIGLYEM | |
| | | AQREGLVSSQRLKPNS | |
| 15 | NP_001031104 | MFLLYRRLPSFARTTTTTLLCKSMEPAITATSSSSFGGGSSRL | 42 |
| | | AALAQQLRQYKPPPSSSFDDSEEMQTD | |
| | | QETAGKVVSQVGFQESIAPLSKDPDRFKPKRAAVLICLFEGD | |
| | | DGDLRVILTKRSSKLSTHSGEVSLPGGK | |
| | | AEEDDKDDGMTATREAEEEIGLDPSLVDVVTSLEPFLSKHLL | |
| | | RVIPVIGILRDKNKFNPIPNPGEVEAVF | |
| | | DAPLEMFLKDENRRSEEREWMGEKYLIHYFDYRTGDKDYM | |
| | | IWGLTAGILIRAASVTYERPPAFIEQCPKF | |
| | | KYPKMVEKHTCMP | |
| 16 | NP_001078142 | MVEQRYEDGSRLVAGCIPFRYVNSDKDGNSESGKVIQVLMI | 43 |
| | | SSSSGPGLLFPKGGWENDETVREAAAREA | |
| | | VEEAGVRGILMDFLGNYEFKSKSHQDEFSPEGLCKAAMYAL | |
| | | YVKEELATWPEHETRTRKWLTIEEAVESC | |
| | | RHPWMKDALVEGFCKWHKEKMVKGEEITGEH | |
| 17 | NP_565273 | MGVEKMVCLASRTGRQFQRYNKGRRQVVGCVPYRFKLSN | 44 |
| | | DGKISDEVEVLVISSQKGHALMFPKGGWELDESVEEAASRE | |
| | | CLEEAGVLGNVEHQLGKWDFLSKSRGTYYEGLMFPMLVTE | |
| | | QLELWPEQHVRQRIWMNVTEAREACRDWWMKEALDVLV | |
| | | ERLSSPMNQPKEEKTMSISIETMC | |
| 18 | NP_172939 | MVCLVSRTGRQSQRYNKGRRQVVGCIPYRLKISSDGTISDEF | 45 |
| | | EVLVISSQKGHALMFPKGGWELDESVEEAASRESLEEAGVV | |
| | | GNVERQLGKWDFLSKSKGTFYEGFMFPMLVKEELELWPEQ | |
| | | HLRQRIWMKVDEARDACRDWWMKEALDVLVQRLSLLSLK | |
| | | PMEEDENLPLISIY | |
| 19 | NP_197507 | MLALFLSSSSYPTLSFLSRSVTLNLARTTTLSALTMSMNLKT | 46 |
| | | HAFAGNPLKSKTPKSTDPFSPTSAFESL | |
| | | KTLIPVIPNHSTPSPDFKVLPFSKGRPLVFSSGGDANTTPIWH | |
| | | LGWVSLADCKVLLASCGVDLNEDSLVY | |
| | | LGPKLEEDLVYWAVDLAEDGFVSELGGRKLCFVELRTLMV | |
| | | AADWADQRAMDELAIAGNARALLEWHNVSQ | |
| | | FCGSCGSKTFPKEAGRRKQCSDETCRKRVYPRVDPVVIMLV | |
| | | IDRENDRALLSRQSRYVPRMWSCLAGFIE | |
| | | PGESLEEAVRRETWEETGIEVGDVVYHSSQPWPVGPSSMPC | |
| | | QLMLGFFAFAKTLDINVDKEELEDAQWHS | |
| | | REEVKKALAVAEYRKAQRTAAAKVEQICKGVERSQSLSTDF | |
| | | NLESGELAPMFIPGPFAIAHHLISAWVNQ | |
| | | APDDVHSKQQAGVSLSSL | |
| 20 | NP_197447 | MASGFCSLALTVTTSLFSSHAITRRVLPILRWRSSSMSLSPLR | 47 |
| | | HSRALSAATTVPISSSFTWDDVIETGRAEYNSSDLTGFFEKIN | |
| | | RCNRGSEKLGEFIPFVIEEQIVGYIHKRFTEYLREFHDIFTFSQ | |
| | | NGSCPDRVDGYVTLNLMLQKPEDRTRAVADVIKILGDKGIIP | |
| | | GIRNELYPVKPSFNAPVFFSLERAAAPYFGIKGYGVHMNGY | |
| | | VERDGQKLLWIGKRSLSKSTYPGMLDHLVAGGLPHGISCGG | |
| | | NLVKECEEEAGISRAIADRAIAVGAVSYLDIDQYCFKRDVLF | |
| | | CYDLELPEDFVPKNQDGEVESFKLIPVAQVASVIKKTSFFKA | |
| | | NCSLVIIDFLFRHGFIRPESSGYLDLYQRLRNRDCS | |
| 21 | NP_177495 | MISLFISNFSNLSNLSPTFDNMNMNIPSKKIVPVPTPSEKVVSL | 48 |
| | | VSRTGRDLQRYNTAGYRQVVGCVPYR | |
| | | YKKHGGGEIEVLLISAQKKGKGMLLPKGGWEIDESIEEAAL | |
| | | RETIEEAGVTGQLEESLGKWQYKSKRHTM | |
| | | IHDGHMFPLLVSQQFEIWPESEFRQRKWVSLSEAIELCQNSW | |
| | | MREALEAFINRKCQTQ | |
| 22 | NP_565776 | MKSGASAASPTAKSFNFGSSRLLALAQQLRVYKPPLSSSFDE | 49 |
| | | REELLAYKESTRKSITHVGFQESMAPVR | |
| | | FRPKKAAVLICLFEGDDGDLRVILTKRSSTLSTHSGEVSLPG | |
| | | GKAEEHDKDDGITATREAEEEIGLDPSL | |
| | | VDVVAFLEPFLSQHLLRVIPVVGILWDRKAFNPTPNPAEVEA | |
| | | VLDAPFEMFLKDENRRSEEFDWMGEKHL | |
| | | VHFFDYKTGDSDYVIWGLTARILIRAATVVYQRPPAFIEQKP | |
| | | NLKYSKMNQATRLYGWLKPLANLHESCE | |
| | | NGRLLGHVPAYCLRYLVGISSR | |
| 23 | NP_565965 | MLKAVQILGWSSGLTISQRLTKTRKSSTVSFISSSLNLSSVTS | 50 |
| | | SSPRRIFSFKPTRMSSSLPGSDPVANS | |
| | | PTFVSVQSAGDVRKIKFCQWCGGPTKHEIPDGEEKLRAICTH | |
| | | CGKIAYQNPKMVVGCLIEHEGKVLLCKR | |
| | | NIQPSHGLWTLPAGYLEVGESAAQGAMRETWEEAGATVEV | |
| | | ISPFAQLDIPLIGQTYVIFLAKLKNLHFAP | |
| | | GPESLECRLFALDEIPFDSLAFSSIYVTLNLYLEDLKKGKLKF | |
| | | HYGTINKRPGSSPSDIRAFSLDYHLQP | |
| 24 | NP_197448 | MASAFCSLCPTPTSLFSSHALIPTLQWRSSSSSRSPPLHISRVL | 51 |
| | | SVETVPLSPSFTWNDVFENSRKEYVPQNSSDLTGFLEKVDRC | |
| | | NRGLEKLGEFIPFVIEEQIVGYIHKGFTKYLRDFNDIFTFSQY | |
| | | GGHVTLNMMLDKPEERTRAVAHVIKILGNKGIIPGIRNELYP | |
| | | VKPSFNAPAFFSIERAAAPYFGLKGYAIHVNGYVERDGQKF | |
| | | LWIGKRSLAKSTYPGKLDHLVAGGLPHGISVCENLVKECEE | |
| | | EAGISKVLADRAIAVGVVSYMDIDRYCFTRDVLFCYDLELP | |
| | | QDFVPTNQDGEVDSFRLIPVAQVANVVRKTSFFKDSCSLVII | |
| | | DFLFRHGLIRPESPGYLDLYRRLRNGDCS | |
| 25 | NP_001185114 | MENLPPGYRPNVGVCLINSDNLVFVASRLNVPGAWQMPQG | 52 |
| | | GIEDGEDPKSAAMRELQEETGVVSAEIVSE | |
| | | VPNWLTYDFPPAVKAKVNRLWGGEWHGQAQKWYLVRLR | |
| | | NDEDEKEINLANNEADSEFAEWKWAKPEEVVE | |
| | | QAVDYKRPTYEEVIKTFGSFLNDTGRAAKCKSAKW | |
| 26 | NP_187673 | MALYRPLLLHHPTSPSVTTFLRNYPSKPIKFSSLPFLHRCRKS | 53 |
| | | RVSSSSARCCSSMESPPEGYRRNVGVC | |
| | | LMNSSKKIFTASRLDIPSAWQMPQGGIDEGEDPRVAVMREL | |
| | | KEETGVHSAEILAEAPHWITYDFPPDVRE | |
| | | KLKVRWGSDWKGQAQKWFLLKFTGKDEEINLLGDGTEKPE | |
| | | FGEWSWTSPDQVVENAVEFKKPVYKEVMSA | |
| | | FASHLQ | |
| 27 | NP_196252 | MAVKASGFIGKSAISVHLDFSSFPVKFSCLKQFSVSSPKPLVV | 54 |
| | | LSVALSSPARTVESPPVGYRKNVGICL | |
| | | VSPCRKIFTASKIHIPDTWQMPQGGADEGEDLRNAAFRELRE | |
| | | ETGVTSAEFIAEIPNWLTYDFPREVKDK | |
| | | LNRKWRTSYKGQAQKWFLFKFTGKEEEINLLGDGTAKPEF | |
| | | KVWSWMLPEQVIEHAVYFKRPVYEHVIKQF | |
| | | NPYFVDEEKDSMNSSKD | |

In some embodiments, the NUDIX gene or gene product contains or is composed completely of a polynucleotide. In some embodiments, the NUDIX gene or gene product contains or is completely composed of a sequence that is about 70% to 100% identical to any one of SEQ ID NO: 1-27 and/or a sequence complementary thereto. In some embodiments, the NUDIX gene or gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 1-27 and/or a sequence complementary thereto. In some embodiments, the NUDIX gene or gene product contains or is completely composed of a sequence that is about 70% to 100% identical to any one of SEQ ID NO: 4, 12, 13, 14, 16, 17, 18, and 21 and/or a sequence complementary thereto. In some embodiments, the NUDIX gene or gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 4, 12, 13, 14, 16, 17, 18, and 21 and/or a sequence complementary thereto. In some embodiments, the NUDIX gene or gene product contains or is completely composed of a sequence that is about 70% to 100% identical to any one of SEQ ID NO: 4, 12, 13, 16, and 21 and/or a sequence complementary thereto. In some embodiments, the NUDIX gene or gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 4, 12, 13, 16, and 21 and/or a sequence complementary thereto.

In some embodiments, the NUDIX gene product contains or is composed completely of a polypeptide. In some embodiments, the NUDIX gene product contains or is completely composed of a sequence that is about 70% to 100% identical to any one of SEQ ID NO: 28-55. In some embodiments, the NUDIX gene product contains or is completely composed of a sequence that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 29-55. In some embodiments, the NUDIX gene product contains or is completely composed of a sequences that is about 70% to 100% identical to any one of SEQ ID NO: 31, 39, 40, 41, 43, 44, 45, and 48. In some embodiments, the NUDIX gene product contains or is completely composed of a sequences that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 31, 39, 40, 41, 43, 44, 45, and 48. In some embodiments, the NUDIX gene product contains or is completely composed of a sequences that is about 70% to 100% identical to any one of SEQ ID NO: 31, 39, 40, 43, and 48. In some embodiments, the NUDIX gene product contains or is completely composed of a sequences that is about 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 31, 39, 40, 43, and 48.

### Vectors and Vector Systems

Also provided herein are vectors that can contain one or more of the NUDIX encoding polynucleotides described herein. The vectors can be useful in producing, for example, bacterial, plant cells, and/or transgenic (engineered) plants that can contain, replicate, and/or express a NUDIX gene and/or gene product described herein. Within the scope of this disclosure are vectors containing one or more of the polynucleotide sequences described herein. One or more of the polynucleotides that are part of the NUDIX encoding polynucleotides described herein can be included in a vector or vector system. The vectors and/or vector systems can be used, for example, to express one or more of the polynucleotides in a cell, such as a plant cell or agrobacterium or to produce particles such as viral particles that can be used to generate transgenic cells and/or plants. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g. a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for expression one or more NUDIX or other (e.g., reporter) polynucleotides and/or one or more regions or domains thereof described herein.

### Cell-Based Vector Amplification and Expression

Vectors can be designed for amplification, propagation, and expression of one or more elements of the NUDIX encoding polynucleotides described herein (e.g. nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, mammalian cells, and more particularly plant cells. The vectors can be viral-based or non-viral based. In some embodiments, the suitable host cell is a eukaryotic cell. In some embodiments, the suitable host cell is a suitable bacterial cell. Suitable bacterial cells include, but are not limited to bacterial cells from the bacteria of the species Escherichia coli. Many suitable strains of E. coli are known in the art for expression of vectors. These include, but are not limited to Pir1, Stb12, Stb13, Stb14, TOP10, XLI Blue, and XL10 Gold. In some embodiments, the host cell is a suitable insect cell. Suitable insect cells include those from Spodoptera frugiperda. Suitable strains of S. frugiperda cells include, but are not limited to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSecl (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, where one or more NUDIX polynucleotides, functional domains thereof, and/or one or more additional polynucleotides are included and/or expressed (e.g. a reporter polynucleotide), each be operably linked to separate regulatory elements on the same or separate vectors. In some embodiments, two or more of the elements expressed from the same or different regulatory element(s), can be combined in a single vector, with one or more additional vectors providing any components of the system not included in the first vector. Encoding polynucleotides (NUDIX or others) that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more NUDIX proteins and/or functionals domains thereof and/or one or more other genes (e.g., reporter gene), embedded within one or more intron sequences (e.g., each in a different intron, two or more in at least one intron, or all in a single intron). In some embodiments, the NUDIX proteins and/or functionals domains thereof and/or one or more other genes (e.g., reporter gene) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

Where a vector or vector system is provided that contains the NUDIX encoding polynucleotide, the encoding polynucleotide can be operatively coupled to one or more regulatory elements. In some embodiments, the regulatory element can drive ubiquitous expression. In some embodiments, the regulatory element can drive or control cell or tissue specific expression. In some embodiments, the regulatory element can drive conditional or inducible expression. In some embodiments, the NUDIX encoding polynucleotide is operatively coupled to a plant promoter.

In embodiments, the polynucleotides and/or vectors thereof described herein (such as the NUDIX encoding polynucleotides) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as leaves, stem, fruit, flower, etc., or particular cell types. Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g. promoters, that direct the transcription of the gene and/or translation of the encoded protein in a cell.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the NUDIX encoding polynucleotides described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the NUDIX encoding polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expression of the NUDIX encoding polynucleotides are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Further exemplary plant promoters suitable to drive expression of the heterologous DDP1 encoding polynucleotides include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more NUDIX encoding polynucleotides described herein, a light-responsive cytochrome heterodimer (e.g. from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g. embodiments of inducible DNA binding proteins and methods of use and can be adapted for use with the present invention.

In some embodiments, transient or inducible expression can be achieved by including, for example, chemical-regulated promotors, i.e. whereby the application of an exogenous chemical induces gene expression. Modulation of gene expression can also be obtained by including a chemical-repressible promoter, where application of the chemical represses gene expression. Chemical-inducible promoters include, but are not limited to, the maize In2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

Where transient expression of an encoding polynucleotide described herein is desired, transient expression may be achieved using suitable vectors. Exemplary vectors that may be used for transient expression include a PEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7): 682-93; and Yin K et al., Scientific Reports volume 5, Article number: 14926 (2015).

A plant promoter is capable of initiating transcription in plant cells, whether or not its origin is a plant cell. The use of different types of promoters is envisaged.

In some embodiments, the vector or system thereof can include one or more elements capable of translocating and/or expressing a NUDIX encoding polynucleotide to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the NUDIX encoding polynucleotides can be operably linked, fused to, or otherwise modified to include a polynucleotide that encodes or is a selectable marker or tag, which can be a polynucleotide or polypeptide. In some embodiments, the polynucleotide encoding a polypeptide selectable marker can be incorporated with the NUDIX encoding polynucleotides polynucleotide such that the selectable marker polypeptide, when translated, is inserted between two amino acids between the N- and C-terminus of the NUDIX polypeptide or at the N- and/or C-terminus of the NUDIX. In some embodiments, the selectable marker or tag is a polynucleotide barcode or unique molecular identifier (UMI).

It will be appreciated that the polynucleotide encoding such selectable markers or tags can be incorporated into a NUDIX encoding polynucleotide described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more NUDIX polypeptides described herein via suitable linker, such as a glycine or glycine serine linkers which are known in the art.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organelles, etc. In some embodiments, the targeting moiety encoding polynucleotides can be included in the vector or vector system such that the NUDIX polynucleotides and/or products expressed therefrom include the targeting moiety and can be targeted to specific cells, tissues, organelles, etc. In some embodiments, such as non-viral carriers, the targeting moiety can be attached to the carrier (e.g. polymer, lipid, inorganic molecule etc.) and can be capable of targeting the carrier and any attached or associated NUDIX polynucleotide(s) to specific cells, tissues, organelles, etc.

### Cell-Free Vector and Polynucleotide Expression

In some embodiments, the polynucleotide encoding a NUDIX gene product is expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g. translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g. 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg₂₊, K+, etc.). As previously mentioned, in vitro translation can be based on RNA or DNA starting material. Some translation systems can utilize an RNA template as starting material (e.g. reticulocyte lysates and wheat germ extracts). Some translation systems can utilize a DNA template as a starting material (e.g. E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the NUDIX gene product encoding polynucleotides described herein can be codon optimized. In some embodiments, one or more polynucleotides contained in a vector (“vector polynucleotides”) described herein that are in addition to an optionally codon optimized the NUDIX gene product encoding polynucleotides described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with a heterologous NUDIX encoding polynucleotide and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid, polynucleotide molecule, or composition that be attached to or otherwise interact with, encapsulate, and/or associate with a polynucleotide to be delivered, such as a NUDIX gene product encoding polynucleotide of the present invention.

### Naked Polynucleotides

In some embodiments, one or more NUDIX gene product encoding polynucleotides described elsewhere herein can be included in and/or delivered as a naked polynucleotide. The term of art “naked polynucleotide” as used herein refers to polynucleotides that are not associated with another molecule (e.g. proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the NUDIX gene product encoding polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g. plasmids and artificial chromosomes), molecules that contain portions that are single stranded and portions that are double stranded (e.g. ribozymes), and the like. In some embodiments, the naked polynucleotide contains only the NUDIX gene product encoding polynucleotide(s) described herein. In some embodiments, the naked polynucleotide can contain other nucleic acids and/or polynucleotides in addition to the NUDIX gene product encoding polynucleotide(s) described herein. The naked polynucleotides can include one or more elements of a transposon system. Transposons and system thereof are described in greater detail elsewhere herein.

### Non-Viral Polynucleotide Vectors

In some embodiments, one or more of the NUDIX gene product encoding polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, agrobacterium vectors (Ti or Ri vectors), PSK systems (post-segregationally killing systems), ORT (operator repressor titration) plasmids, and the like. See e.g. Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g. Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more NUDIX gene product encoding polynucleotides described herein) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the NUDIX gene product encoding polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the NUDIX gene product encoding polynucleotide(s)) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the NUDIX gene product encoding polynucleotide(s)) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tc1/mariner superfamily) (see e.g. Ivics et al. 1997. Cell. 91(4): 501-510), piggyBac (piggyBac superfamily) (see e.g. Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31 (23): 6873-6881) and variants thereof.

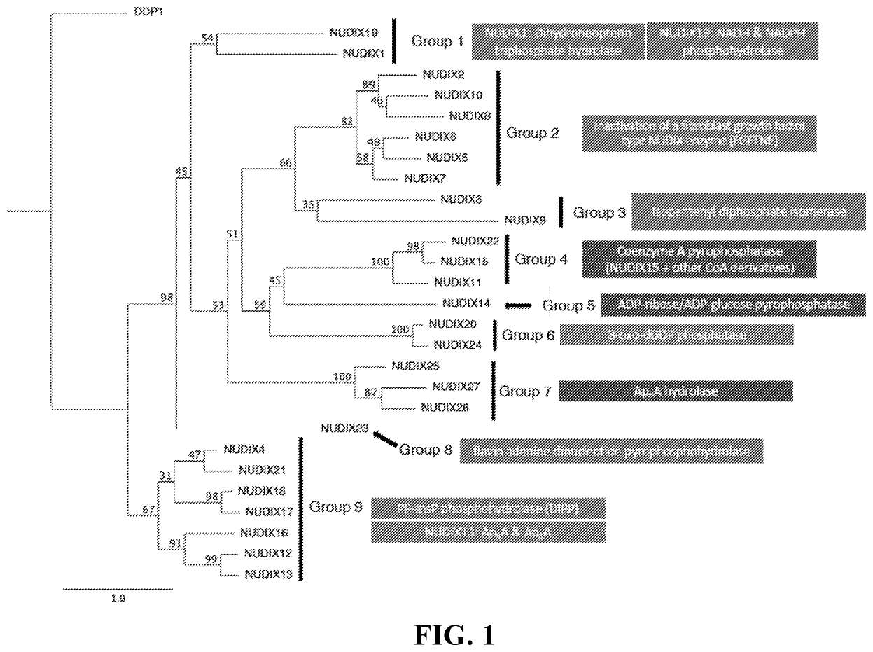

In some embodiments, the non-viral vector or vector system is an agrobacterium vector or vector system. In some embodiments, the NUDIX gene product encoding polynucleotide(s) is included in a T-DNA (or Ti) vector or an Ri vector (See e.g., Gelvin, S. 2003. Microbiol Mol Biol Rev. 2003 March; 67(1): 16-37, particularly at FIG. 1A; Lee and Gelvin. Plant Physiol. 2008 February; 146(2): 325-332 and as described elsewhere herein.

### Chemical Carriers

In some embodiments, the NUDIX gene product encoding polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the NUDIX gene product encoding polynucleotide(s)), (2) those capable of targeting specific cells, (3) those capable of increasing delivery of the polynucleotide (such as the NUDIX gene product encoding polynucleotide(s)) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the NUDIX gene product encoding polynucleotide(s) described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticuloendothelial system. In some embodiments, the inorganic particles can be optimized to protect an entrapped molecule from degradation. The suitable inorganic particles that can be used as non-viral carriers in this context can include, but are not limited to, calcium phosphate, silica, metals (e.g. gold, platinum, silver, palladium, rhodium, osmium, iridium, ruthenium, mercury, copper, rhenium, titanium, niobium, tantalum, and combinations thereof), magnetic compounds, particles, and materials, (e.g. supermagnetic iron oxide and magnetite), quantum dots, fullerenes (e.g. carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g. such as a NUDIX gene product encoding polynucleotide). In some embodiments, chemical non-viral carrier systems can include a polynucleotide such as the NUDIX gene product encoding polynucleotide(s)) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g. the NUDIX gene product encoding polynucleotide(s)). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g. polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g. US Pat. Pub. 2017/0079916 whose techniques and compositions can be adapted for use with the NUDIX gene product encoding polynucleotide(s)), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g. calcium, NaCl, and the like), pressure and the like. In some embodiments, the non-viral carrier can be a particle that is configured includes one or more of the NUDIX gene product encoding polynucleotide(s) described herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the NUDIX gene product encoding polynucleotide(s)).

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a NUDIX gene product encoding polynucleotide, into a virus particle and producing said virus particle when used alone or with one or more other viral vectors (such as in a viral vector system). Viral vectors and systems thereof can be used for producing viral particles for delivery of and/or expression of one or more components of the NUDIX gene product encoding polynucleotide described herein. The viral vector can be part of a viral vector system involving multiple vectors. In some embodiments, systems incorporating multiple viral vectors can increase the safety of these systems. Suitable viral vectors can include any plant viral vector or system such as any of those set forth in e.g., K. Hefferon. Biomedicines. Zaidi* and Mansoor. 2017 September; 5(3): 44, Front. Plant Sci., 11 Apr. 2017. https://doi.org/10.3389/fpls.2017.00539, and Abrahamian et al. 2020. Ann. Rev. Virol. 7:513-535, particularly those based on tobacco mosaic virus (TMV), Potexviruses, and Comovirus Cowpea mosaic virus (CPMV). Other embodiments of viral vectors and viral particles produce therefrom are described elsewhere herein. In some embodiments, the viral vectors are configured to produce replication incompetent viral particles for improved safety of these systems.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Virus Particle Production from Viral Vectors

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g. pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g. a NUDIX gene product encoding polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., NUDIX transcripts, proteins, enzymes, mutant forms thereof, fusion proteins thereof, etc.), and virus particles (such as from viral vectors and systems thereof).

One or more NUDIX gene product encoding polynucleotides can be delivered using an engineered plant virus particle containing the one or more NUDIX gene product encoding polynucleotides using appropriate formulations and doses.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

### Engineered Plants Having Increased NUDIX Gene Expression

As previously described in some embodiments the engineered plant has increased NUDIX gene expression. In some embodiments, the engineered plant contains one or more exogenous NUDIX genes or portion thereof that encodes a NUDIX polypeptide, contains one or more modified endogenous NUDIX genes; contains a modified epigenome thereby increasing NUDIX gene expression; or any combination thereof. Methods and techniques of producing the engineered plants are discussed elsewhere herein.

### Engineered Plants Expressing Exogenous NUDIX Gene(s)

In some embodiments, the engineered plant contains one or more exogenous NUDIX genes or portion thereof that encodes a NUDIX polypeptide. The one or more exogenous NUDIX genes or portion thereof that encodes a NUDIX polypeptide can be stably integrated into the genome of one or more cells in the plant. The one or more exogenous NUDIX genes or portion thereof that encodes a NUDIX polypeptide can be transiently expressed of one or more cells in the plant. It will be appreciated that the exogenous NUDIX gene can be a NUDIX gene that is not natively expressed in the plant into which it is introduced or be an extra copy that is identical to or a modified (e.g., mutant) variant of a NUDIX gene that is natively expressed in the plant into which it is introduced. In some embodiments, the engineered plant having increased NUDIX enzyme activity has and/or expresses one or more exogenous NUDIX genes.

In some embodiments, the exogenous NUDIX gene expression construct that is contained and/or expressed in the one or more cells of the engineered plant comprises one or more NUDIX genes or portions thereof encoding the one or more NUDIX polypeptides operatively coupled to one or more exogenous promoters and/or regulator elements that drive and/or regulate expression of the one or more NUDIX genes and/or portions thereof encoding one or more NUDIX polypeptides. In some embodiments, the one or more exogenous promoters is a constitutive promoter. In some embodiments, the one or more exogenous promoters is a cell or tissue specific promoter. In some embodiments, the one or more exogenous promoters is a temporal promoter, i.e., a promoter that expressed only during certain time (e.g., times of day, year, or moth, or during certain developmental or production stages). In some embodiments, the promoter is an inducible promoter. Exemplary promoters and other regulatory elements that can be coupled to the one or more exogenous NUDIX genes are described elsewhere herein.

In some embodiments, the exogenous NUDIX gene expression construct that is contained and/or expressed in the one or more cells of the engineered plant does not contain a promoter and/or regulator elements. In some embodiments one or more exogenous NUDIX genes or portion thereof that encodes a NUDIX polypeptide are introduced into a genome of one or more cells in a location such that the expression of the exogenous NUDIX gene is driven and/or otherwise regulated by promoter(s) and/or other regulatory elements endogenous to the engineered plant. For example, in some embodiments, The one or more exogenous NUDIX genes or portion thereof that encodes a NUDIX polypeptide are introduced such that the exogenous NUDIX gene expression is driven off of and/or regulated by an endogenous NUDIX promoter and/or endogenous regulator elements. In such embodiments, the exogenous NUDIX gene can replace or be integrated in-frame with one or more endogenous NUDIX genes. In embodiments, where the one or more exogenous NUDIX genes or portion thereof that encodes a NUDIX polypeptide are introduced such they are driven off a non-NUDIX promoter and/or other regulator elements, the one or more exogenous NUDIX genes or portion thereof that encodes a NUDIX polypeptide can be introduced such that they replace one or more non-NUDIX genes or are integrated in-frame with the one or more non-NUDIX genes such that the expression of the exogenous NUDIX gene or portion thereof that encodes a NUDIX polypeptide is driven off of and/or regulated by the promoter and/or other regulator elements that drive and/or regulate expression of the non-NUDIX endogenous gene.

### Engineered Plants Expressing Modified Endogenous NUDIX Gene(s)

In some embodiments, the engineered plant contains one or more modified endogenous NUDIX genes comprise one or more mutations, insertions, deletions, substitutions, or any combination thereof in the coding or non-coding region of the one or more modified endogenous NUDIX genes such that expression of the one or more modified endogenous NUDIX genes are increased as compared to an unmodified control. In some embodiments, the engineered plant having increased NUDIX enzyme activity has a one or more modified endogenous NUDIX genes.

In some embodiments, an insertion, deletion, or indel in a modified endogenous NUDIX gene can range in size from 1-50 or more base pairs. In some embodiments, an insertion, deletion, or indel in a modified endogenous NUDIX gene can be 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more.

In some embodiments, the modified NUDIX gene or NUDIX gene product encoding polynucleotide has 1-500 or more mutated or substituted bases or base pairs In some embodiments, the modified NUDIX gene or gene product encoding polynucleotide has 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more mutated or substituted bases or base pairs.

Methods and techniques for introducing mutations, insertions, deletions, substitutions, or any combination thereof are generally known in the art and described elsewhere herein. Such methods and techniques include, but are not limited to, recombinant engineering and genome editing via programmable nucleases (e.g., TALENs, Zinc Finger nuclease, CRISPR-Cas systems, CRISPR-Cas based systems (e.g., CRISPR-associated transposases (CAST), and Primer Editors, and/or the like). See e.g., Malzahn et al., Cell Biosci. 2017 Apr. 24; Molla et al., Nat Plants. 2021. PMID: 34518669k 7:21. doi: 10.1186/s13578-017-0148-4. eCollection 2017; Zhang et al., Prog Mol Biol Transl Sci. 2017; 149:133-150. doi: 10.1016/bs.pmbts.2017.03.008; Sprink et al., Curr Opin Biotechnol. 2015 April; 32:47-53. doi: 10.1016/j.copbio.2014.11.010; Razzaq et al., Int J Mol Sci. 2019 Aug. 19; 20(16):4045. doi: 10.3390/ijms20164045; Sanagala et al., Sanagala R, et al. J Genet Eng Biotechnol. 2017. PMID: 306476; Norman et al., Front Plant Sci. 2016. PMID: 27917188; Molka et al., Strecker et al., Science. 2019. 365(6448):48-53; and Nandy et al., J Biosci. 2020. PMID: 32020912, which can be adapted for use with the present disclosure. In view of at least the reference sequences in the context of the disclosure herein, one of ordinary skill in the art can apply any one or more of these methods and techniques to generate engineered plants having one or more modified endogenous NUDIX genes and/or gene products. In some embodiments, such as when the engineered plant is generated using a CRISPR-Cas system or CRISPR-based system, the engineered plant comprises a CRISPR-Cas system or component(s) thereof.

### Engineered Plants Having a Modified Epigenome

In some embodiments the engineered has an modified epigenome. Epigenome modification refers to the modification of an epigenetic landscape local to an endogenous genomic site of interest. In the context of the instant description, the epigenetic landscape modified is local to the genomic site of one or more endogenous NUDIX genes. Without being bound by theory, by modifying the epigenome local to the genomic of one or more endogenous NUDIX genes. The term “local to” as used in this context herein includes the site of the gene within the genome as well as the region that is in proximity to the gene such that modification of the epigenome in that region can modify (increase or decrease) the accessibility of the genomic site of the gene or regulatory region thereof. In some embodiments, modifying the epigenetic landscape local to the genomic site of the one or more NUDIX genes increases expression of the one or more NUDIX genes. Without being bound by theory, the modification(s) to the epigenetic landscape local to the genomic site of the one or more NUDIX genes increases accessibility to the one or more NUDIX genes such that transcription is increased. In some embodiments, the modification of the epigenetic landscape local to the genomic site of the one or more NUDIX genes is DNA demethylation, histone demethylation, histone acetylation, or any combination thereof. In some embodiments, the engineered plant having increased NUDIX enzyme activity has a modified epigenetic landscape local to the genomic site of one or more endogenous NUDIX genes.

In some embodiments, DNA methylation of one or more of the NUDIX genes, regions thereof, or regulatory region(s) thereof is decreased by 0.01-100%. In some embodiments DNA methylation of one or more of the NUDIX genes, regions thereof, or regulatory region(s) thereof is decreased by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

In some embodiments, histone methylation at an epigenomic site local to one or more of the NUDIX genes, regions thereof, or regulatory region(s) thereof is decreased by 0.01-100%. In some embodiments, histone methylation at an epigenomic site local to one or more of the NUDIX genes, regions thereof, or regulatory region(s) thereof is decreased by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99% 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

In some embodiments, histone acetylation at an epigenomic site local to one or more of the NUDIX genes, regions thereof, or regulatory region(s) thereof is increased by 0.01-100 fold or more. In some embodiments, histone acetylation at an epigenomic site local to one or more of the NUDIX genes, regions thereof, or regulatory region(s) thereof is increased by 0.01, to/or 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 fold or more.

In some embodiments, histone acetylation at an epigenomic site local to one or more of the NUDIX genes, regions thereof, or regulatory region(s) thereof is increased by about 1 to 1000 percent or more. In some embodiments, histone acetylation at an epigenomic site local to one or more of the NUDIX genes, regions thereof, or regulatory region(s) thereof is increased by about 1%, to/or 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more.

Epigenome editing can be accomplished utilizing a programmable nuclease system or other approach to provide targeted epigenomic modification and/or delivery of a demethylase, acetylase, and/or acetyl transferase to the epigenomic site local to the endogenous genomic site of interest (e.g., an endogenous NUDIX gene or regulator region thereof). Methods and techniques of epigenomic modification are generally known in the art as is described in e.g., Thakore et al., Nat Methods. 2016 February; 13(2):127-37. doi: 10.1038/nmeth.3733; Nakamura et al., Nat Cell Biol. 2021 January; 23(1):11-22. doi: 10.1038/s41556-020-00620-7; Brocken et al., Curr Issues Mol Biol. 2018; 26:15-32. doi: 10.21775/cimb.026.015; Holtzman and Gersbach. Annu Rev Genomics Hum Genet. 2018 Aug. 31; 19:43-71. doi: 10.1146/annurev-genom-083117-021632; Rots and Jeltsch. Methods Mol Biol. 2018; 1767:3-18. doi: 10.1007/978-1-4939-7774-1_1; Waryah et al., Methods Mol Biol. 2018; 1767:19-63. doi: 10.1007/978-1-4939-7774-1_2; Gjaltema and Rots. Curr Opin Chem Biol. 2020 August; 57:75-81. doi: 10.1016/j.cbpa.2020.04.020; Enriquex, P., Yale J Biol Med. 2016 Dec. 23; 89(4):471-48; Goell and Hilton. Trends Biotechnol. 2021 July; 39(7):678-691. doi: 10.1016/j.tibtech.2020.10.012; Pei et al., Brief Funct Genomics. 2020 May 20; 19(3):215-228. doi: 10.1093/bfgp/elz035; Zhao et al., Int J Mol Sci. 2020 Feb. 3; 21(3):998. doi: 10.3390/ijms21030998; Gomez et al., Trends Genet. 2019 July; 35(7):527-541. doi: 10.1016/j.tig.2019.04.007; Lau et al., Transgenic Res. 2018 December; 27(6):489-509. doi: 10.1007/s11248-018-0096-8; Kungulovski et al., Trends Genet. 2016 February; 32(2):101-113. doi: 10.1016/j.tig.2015.12.001; Chakravarti et al., Curr Drug Targets. 2022; 23(8):836-853. doi: 10.2174/1389450123666220117105531; Laufer et al., Epigenetics Chromatin. 2015 Sep. 17; 8:34. doi: 10.1186/s13072-015-0023-7; Bozorg et al., Adv Biomed Res. 2019 Aug. 21; 8:49. doi: 10.4103/abr.abr_41_19. eCollection 2019; Moradpour and Abdulah et al., Plant Biotechnol J. 2020 January; 18(1):32-44. doi: 10.1111/pbi.13232. Epub 2019; Kubik et al., Chembiochem. 2016 Jun. 2; 17(11):975-80. doi: 10.1002/cbic.201600072. Epub 2016 Apr. 20; Zhan et al., J Integr Plant Biol. 2021 January; 63(1):3-33. doi: 10.1111/jipb.13063; Dubois and Roudier. Epigenomes. 2021 Aug. 24; 5(3):17. doi: 10.3390/epigenomes5030017; Dhakate et al., Front Genet. 2022 Aug. 23; 13:876987. doi: 10.3389/fgene.2022.876987. eCollection 2022; Selma et al., Transgenic Res. 2021 August; 30(4):381-400. doi: 10.1007/s11248-021-00252-z; Chennakesavulu et al., Plant Cell Rep. 2022 March; 41(3):815-831. doi: 10.1007/s00299-021-02681-w; and Zhang et al., Nat Plants. 2019 August; 5(8):778-794. doi: 10.1038/s41477-019-0461-5, which can be adapted for use with the present disclosure, particularly to generate the engineered plants having modified epigenomes as described herein. In view of at least the reference sequences in the context of the disclosure herein, one of ordinary skill in the art can apply any one or more of these methods and techniques to generate engineered plants having one or more modified epigenome at or local to NUDIX genes and/or encoding polynucleotides. In some embodiments, such as when the engineered plant is generated using a CRISPR-Cas system or CRISPR-based system, the engineered plant comprises a CRISPR-Cas system or component(s) thereof.

### Engineered Plants Having Modified NUDIX Gene Products

As previously described in some embodiments, the engineered plant has increased amount of one or more NUDIX gene products as compared to a suitable control. In some embodiments, the one or more NUDIX gene products is mRNA, a polypeptide, or both. In some embodiments, the NUDIX gene product is modified as compared to a suitable control. In some embodiments, the suitable control is the unmodified NUDIX gene product.

In some embodiments, the NUDIX gene product is (a) a modified NUDIX mRNA, wherein the mRNA has been modified as compared to an unmodified control to increase protein translation, mRNA stability, or both; (b) a modified NUDIX polypeptide, wherein the polypeptide has been modified as compared to an unmodified control to increase protein stability; or both (a) and (b).

mRNA Modifications

In some embodiments, the modified NUDIX mRNA comprises (a) one or more mutations, insertions, deletions, substitutions, or any combination thereof such that translation and/or stability of the NUDIX mRNA is increased; (b) one or more nucleic acid modifications that increases translation and/or mRNA stability; or both (a) and (b).

In some embodiments, the modifications are genetically encoded in DNA that encodes the mRNA such that when transcribed, the one or more modifications are present in the transcribed mRNA so as to produce the modified mRNA. In other embodiments, the mRNA can be directly modified, such as by a programmable nuclease. Methods of using programmable nuclease systems, such as CRISPR-Cas based systems, and other systems to modify RNA are generally known in the art. See e.g., Porto et al., Nat Rev Drug Discov. 2020 December; 19(12): 839-859. doi: 10.1038/s41573-020-0084-6; Rees et al., Nat Rev Genet. 2018 December; 19(12): 770-788. doi: 10.1038/s41576-018-0059-1; Xu et al., Mol Cell. 2022 Jan. 20; 82(2): 389-403. doi: 10.1016/j.molcel.2021.10.010; Li et al., J Zhejiang Univ Sci B. 2021 Apr. 15; 22(4): 253-284. doi: 10.1631/jzus.B2100009; Zeballos and Gaj. Trends Biotechnol. 2021 July; 39(7): 692-705. doi: 10.1016/j.tibtech.2020.10.010; Lo et al., Front Genet. 2022 Jan. 28; 13:834413. doi: 10.3389/fgene.2022.834413. eCollection 2022; Li et al., Funct Integr Genomics. 2022 December; 22(6): 1089-1103. doi: 10.1007/s10142-022-00910-3; Khosravi and Jantsch. RNA Biol. 2021 Oct. 15; 18(sup1): 41-50. doi: 10.1080/15476286.2021.1983288; Kavuri et al., Cells. 2022 Aug. 27; 11(17): 2665. doi: 10.3390/cells11172665, which can be adapted for use with the present disclosure, particularly to generate the engineered plants having modified NUDIX mRNA.

In some embodiments, an insertion, deletion, or indel in a modified endogenous NUDIX gene can range in size from 1-50 or more base pairs. In some embodiments, an insertion, deletion, or indel in a modified endogenous NUDIX gene can be 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more.

In some embodiments, the modified NUDIX RNA has 1-500 or more mutated or substituted bases or base pairs In some embodiments, the modified NUDIX RNA has 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more mutated or substituted bases or base pairs.

In some embodiments, the NUDIX RNA, such as mRNA contains one or more modifications that can modulate stability of the NUDIX RNA. Without being bound by theory some embodiments, where the modification increases stability the translation of a NUDIX RNA, such as an mRNA, is increased, which can increase the amount of a NUDIX polypeptide. Without being bound by theory some embodiments, where the modification decreases stability the translation of a NUDIX RNA, such as an mRNA, is decreased, which can decrease the amount of a NUDIX polypeptide. In some embodiments, the modifications that modulate the stability of the NUDIX RNA are genetically encoded or otherwise introduced into the sequence of the mRNA. In some embodiments, the modifications that modulate the stability of the NUDIX RNA are chemical modifications or synthetic bases that can be coupled to or otherwise integrated into the NUDIX RNA.

In some embodiments, the polynucleotides of a NUDIX RNA are structurally modified and/or chemically modified. As used in this context herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, the NUDIX RNA polynucleotide, e.g., an mRNA, described herein comprises at least one chemical modification. In some embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracil in of the NUDIX RNA, such in the open reading frame of the NUDIX encoding polynucleotide, have a chemical modification.

In some embodiments, the NUDIX mRNA polynucleotide includes a stabilization element. In some embodiments, the stabilization element is a histone stem-loop. In some embodiments, the stabilization element is a nucleic acid sequence having increased GC content relative to wild type sequence.

In some embodiments, the NUDIX mRNA comprises and/or encodes one or more 5′ terminal cap (or cap structure), 3′ terminal cap, 5′ untranslated region, 3′ untranslated region, a tailing region, or any combination thereof. In some embodiments, the capping region of the NUDIX mRNA region may be from 1 to 10, e.g., 2-9, 3-8, 4-7, 1-5, 5-10, or at least 2, or 10 or fewer nucleotides in length. In some embodiments, the cap is absent. In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine. In some embodiments, the 5′ terminal cap is 7 mG (5′) ppp (5′) NlmpNp, m7GpppG cap, N⁷-methylguanine. In some embodiments, the 3′ terminal cap is a 3′-O-methyl-m7GpppG.

In some embodiments, the 3′-UTR is an alpha-globin 3′-UTR. In some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a poly A tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

Other RNA modifications for mRNAs and production of mRNA can be as described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference and can be adapted for use with the present invention.

### Polypeptide Modifications

In some embodiments, the one or more modified gene products is one or more modified NUDIX polypeptide. In some embodiments, at least one of the one or more modified NUDIX polypeptides has increased activity as compared to an unmodified control. In some embodiments, the engineered plant having increased NUDIX enzyme activity has one or more modified NUDIX polypeptides. In some embodiments, one or more modified NUDIX polypeptides comprises one or more amino acid mutations, insertions, deletions, substitutions, or any combination thereof such that activity of the one or more modified NUDIX polypeptides is increased as compared to a control; (b) comprises one or more post-translational modifications such that activity of the one or more modified NUDIX polypeptides is increased as compared to a control.

The modified NUDIX polypeptide can be any one of NUDIX 1-27 that contains 1 or more mutations, insertions, deletions, indels, substitutions, or any combination thereof of one or more continuous or discontinuous amino acids. In some embodiments, an N- and/or C-terminal region is truncated by 1-50 or more amino acids.

In some embodiments, the modification is a post-translational modification. The post translational modification can be reversible or irreversible. The post translational modifications can modulate one or more activities or functions of the NUDIX polypeptide, such as protein lifespan, protein-protein interactions, cell to cell and/or cell to cell-matrix interactions, molecular trafficking, receptor activation and/or ligand binding, protein solubility, protein folding, protein localization and/or any combination thereof. Exemplary post translational modifications include, but are not limited to glycosylation, methylation, phosphorylation, acetylation, succinylation, malonylation, sumolation, S-nitrosylation, glutathionylation, amidation, hydroxylation, palmitolation, pyrrolidone carboxcylic acid, glutarylation, gamma-carboxyglutamic acid, crotonylation, oxidation, myristoylation, sulfantion, formylation, and citrullination. See e.g., Ramazi and Zahiri. Database, Volume 2021, 2021, baab012, https://doi.org/10.1093/database/baab012 for additional exemplary post translational modifications. In some embodiments, the amino acid sequence of a NUDIX polypeptide is modified to influence the post-translational modification. For example, in some embodiments, the NUDIX polypeptide is modified so as to increase or decrease the number of residues capable of being post translationally modified by any one or more post translational modifications.

In some embodiments, the NUDIX polypeptide contains one or more signaling and/or trafficking polypeptides. Exemplary signaling and trafficking sequences can be genetically encoded and are described in greater detail elsewhere herein. In some embodiments, the NUDIX polypeptide contains or is otherwise operatively coupled to one or more reporter polypeptides and/or polynucleotides. Exemplary reporter polypeptides and/or polynucleotides are described in greater detail elsewhere herein.

### Exemplary Engineered Plants

Also described herein are engineered plants that can contain one or more of the engineered cells expressing a NUDIX gene and/or gene product described herein. In some embodiments, the engineered plant is an engineered monocotyledonous plant or an engineered dicotyledonous plant. In some embodiments the engineered dicotyledonous plant belongs to the order Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Brassicales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, Brassicales, or Asterales.

In some embodiments, the engineered monocotyledonous plant belongs to the order Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, Orchidales, Pinales, Ginkogoales, Cycadales, Araucariales, Cupressales or Gnetales.

In some embodiments, the engineered plant is a species of Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Thlaspi, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, or Pseudotsuga.

In some embodiments, the engineered plant is a fern. In some embodiments, the engineered plant is a moss. In some embodiments, the engineered plant is a liverwort. In some embodiments, the engineered plant is of the phyla Bryophyta, Pterophyta, or Lycopodiophita. In some embodiments, the engineered plant is from the order Sphagnales, Jungermanniales, Marchantiles, Polytrichales, Hypnobryales, Dicranales, Polypodiales, Equisetales, or Selaginellales. In some embodiments, the engineered plant is from the genus Sphagnum, Bazzania, Marchantia, Conocephalum, Riccia, Polytrichum, Pleurozium, Dicranum, Nephrolepis, Equistum, or Selaginella. In some embodiments, the engineered plant is Marchantia polymorpha.

In some embodiments, the engineered plant is a grain crop plant (e.g., wheat, maize, rice, millet, barley), a fruit crop plant (e.g., tomato, apple, pear, strawberry, orange), forage crops (e.g., alfalfa), a root vegetable crop plant (e.g., carrot, potato, sugar beets, yam), a leafy vegetable crop plant (e.g., lettuce, spinach); a flowering crop plant (e.g., petunia, rose, chrysanthemum), a conifers or pine tree (e.g., pine fir, spruce); a plant used in phytoremediation (e.g., heavy metal accumulating plants); an oil crop plant (e.g., sunflower, rape seed), or a plant typically used for experimental purposes (e.g., Arabidopsis).

In some embodiments, the engineered plant is an angiosperm or a gymnosperm plant.

In some embodiments, the engineered plant is acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pennycress, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, or zucchini.

In some embodiments, the engineered plant is a turfgrass. In some embodiments, the engineered plant is of the order Cyperales. In some embodiments, the engineered plant is from the family of Poaceae. In some embodiments, the engineered plant is of the genus Achnatherum P. Beauv. (needlegrass), Achnella Barkworth (ricegrass), Acrachne Chiov. (goosegrass), Acroceras Stapf (acroceras), Aegilops L. (goatgrass), Aegopogon Humb. & Bonpl. ex Willd. (relaxgrass), Aeluropus Trin. (Indian walnut), x Agroelymus E. G. Camus ex A. Camus (agroelymus), x Agropogon Fourn. (agropogon), Agropyron Gaertn. (wheatgrass), Agrostis L. (bentgrass), Aira L. (hairgrass), Allolepis Söderst. & Decker (Texas salt), Alloteropsis J. Presl (summergrass), Alopecurus L. (foxtail), Amblyopyrum Eig (amblyopyrum), x Ammocalamagrostis P. Fourn., Ammophila Host (beachgrass), Ampelodesmos Link (Mauritanian grass), Amphibromus Nees (wallaby grass), Amphicarpum Kunth (maidencane), Ancistrachne S. T. Blake, Andropogon L. (bluestem), Anthaenantia P. Beauv. (silkyscale), Anthephora Schreb. (oldfield grass), Anthoxanthum L. (hornwort), Apera Adans. (silkybent), Apluda L. (Mauritian grass), Arctagrostis Griseb. (polargrass), x Arctodupontia Tzvelev (arctodupontia), Arctophila Rupr. ex Andersson (pendantgrass), Genus Aristida L. (threeawn), Arrhenatherum P. Beauv. (oatgrass), Arthraxon P. Beauv. (carpetgrass), Arthrostylidium Rupr. (climbing bamboo), Arundinaria Michx. (cane), Arundinella Raddi (rabo de gato), Arundo L. (giant reed), Astrebla F. Muell. ex Benth., Austrostipa S. W. L. Jacobs & J. Everett, Avellinia Parl., Avena L. (oat), Avenula (Dumort.) Dumort. (oatgrass), Axonopus P. Beauv. (carpetgrass), Bambusa Schreb. (bamboo), Beckmannia Host (sloughgrass), Blepharidachne Hack. (desertgrass), Blepharoneuron Nash (dropseed), Borinda Stapleton (borinda), Bothriochloa Kuntze (beardgrass), Bouteloua Lag. (grama), Brachiaria (Trin.) Griseb. (signalgrass), Brachyachne Stapf, Brachyelytrum P. Beauv. (shorthusk), Brachypodium P. Beauv. (false brome), Brachypodium distachyon, Briza L. (quakinggrass), Bromidium Nees & Meyen (tropical bent), Bromus L. (brome), Calamagrostis Adans. (reedgrass), x Calammophila Brand (calammophila), Calamovilfa (A. Gray) Hack. ex Scribn. & Southworth (sandreed), Calyptochloa C. E. Hubb., Castellia Tineo, Catabrosa P. Beauv. (whorlgrass), Catapodium Link (ferngrass), Cathestecum J. Presl (false grama), Celtica F. M. Vázquez & Barkworth, Cenchrus L. (sandbur), Centotheca Desv., Centropodia (R. Br.) Rchb., Chasmanthium Link (woodoats), Chloris Sw. (windmill grass), Chrysopogon Trin. (false beardgrass), Chusquea Kunth (chusquea bamboo), Cinna L. (woodreed), Cladoraphis Franch. (bristly lovegrass), Cleistachne Benth., Cleistogenes Keng, Coelorachis Brongn. (jointtail grass), Coix L. (Job's tears), Coleanthus Seidel (mossgrass), Cortaderia Stapf (pampas grass), Corynephorus P. Beauv. (clubawn grass), Cottea Kunth (cotta grass), Crypsis Aiton (pricklegrass), Ctenium Panzer (toothache grass), Cutandia Willk. (Memphisgrass), Cymbopogon Spreng. (lemon grass), Cynodon Rich. (Bermudagrass), Cynosurus L. (dogstail grass), Cyrtococcum Stapf, Dactylis L. (orchardgrass), Dactyloctenium Willd. (crowfoot grass), Danthonia DC. (oatgrass), Danthoniopsis Stapf, Dasyochloa Willd. ex Rydb. (woollygrass), Dasypyrum (Coss. & Durieu) T. Dur. (mosquitograss), Dendrocalamus Nees, Deschampsia P. Beauv. (hairgrass), Desmostachya (Stapf) Stapf, Diarrhena P. Beauv. (beakgrain), Dichanthelium (Hitchc. & Chase) Gould (rosette grass), Dichanthium Willem. (bluestem), Dichelachne Endl. (Plumegrass), Diectomis Kunth (foldedleaf grass), Digitaria Haller (crabgrass), Dimeria R. Br., Dinebra Jacq. (viper grass), Dissanthelium Trin. (Catalina grass), Dissochondrus (Hillebr.) Kuntze (false brittlegrass), Distichlis Raf. (saltgrass), x Dupoa J. Cay. & S. J. Darbyshire (dupoa), Dupontia R. Br. (tundragrass), Echinochloa P. Beauv. (cockspur grass), Echinopogon P. Beauv., Ectrosia R. Br. (ectrosia), Ehrharta Thunb. (veldtgrass), Eleusine Gaertn. (goosegrass), Elionurus Humb. & Bonpl. ex Willd. (balsamscale grass), x Elyhordeum Mansf. ex Zizin & Petrowa (barley), x Elyleymus Baum (wildrye), Elymus L. (wildrye), Elytrigia Desv., Enneapogon Desv. ex P. Beauv. (feather pappusgrass), Enteropogon Nees (umbrellagrass), Entolasia Stapf (entolasia) Eragrostis von Wolf (lovegrass), Eremochloa Büse (centipede grass), Eremopyrum (Ledeb.) Jaubert & Spach (false wheatgrass), Eriachne R. Br., Eriochloa Kunth (cupgrass), Eriochrysis P. Beauv. (moco de pavo), Erioneuron Nash (woollygrass), Euclasta Franch. (mock bluestem), Eulalia Trin., Eulaliopsis Honda (sabaigrass), Eustachys Desv. (fingergrass), Festuca L. (fescue), x Festulolium Asch. & Graebn. (Festulolium), Fingerhuthia Nees (Zulu fescue), Garnotia Brongn. (lawngrass), Gastridium P. Beauv. (nit grass), Gaudinia P. Beauv. (fragile oat), Gigantochloa Kurz ex Munro (gigantochloa), Glyceria R. Br. (mannagrass), Gymnopogon P. Beauv. (skeletongrass), Gynerium Willd. ex P. Beauv. (wildcane), Hackelochloa Kuntze (pitscale grass), Hainardia Greuter (barbgrass), Hakonechloa Makino ex Honda (Hakone grass), Helictotrichon Besser ex Schult. & Schult. f. (alpine oatgrass), Hemarthria R. Br. (jointgrass), Hesperostipa (Elias) Barkworth (needle and thread), Heteranthelium Hochst. ex Jaub. & Spach, Heteropogon Pers. (tanglehead), Hierochloe R. Br. (sweetgrass), Hilaria Kunth (curly-mesquite), Holcus L. (velvetgrass), Homolepis Chase (panicgrass), Homopholis C. E. Hubb., Hordelymus (Jess.) Harz, Hordeum L. (barley), Hygroryza Nees (watergrass), Hymenachne P. Beauv. (marsh grass), Hyparrhenia Andersson ex Fourn. (thatching grass), Hypogynium Nees (West Indian bluestem), Ichnanthus P. Beauv. (bedgrass), Imperata Cirillo (satintail), Isachne R. Br. (bloodgrass), Ischaemum L. (murainagrass), Iseilema Andersson, Ixophorus Schltdl. (Central America grass), Jarava Ruiz & Pav. (rice grass), Kalinia H. L. Bell & Columbus (kalinia grass), Karroochloa Conert & Türpe (South African oatgrass), Koeleria Pers. (Junegrass), Lagurus L. (harestail grass), Lamarckia Moench (goldentop grass), Lasiacis (Griseb.) Hitchc. (smallcane), Lasiurus Boiss., Leersia Sw. (cutgrass), Leptochloa P. Beauv. (sprangletop), Leptochloopsis Yates (limestone grass), Leptocoryphium Nees (lanilla), Lepturus R. Br. (thintail), Leucopoa Griseb. (spike fescue), x Leydeum Barkworth (hybrid ryegrass), Leymus Hochst. (wildrye), Limnodea L. H. Dewey (Ozark grass), Lithachne P. Beauv. (diente de perro), Loliolum Krecz. & Bobr., Lolium L. (ryegrass), Lophatherum Brongn. (lophatherum), Loudetia Hochst., Luziola Juss. (watergrass), Lycurus Kunth—(wolfstail), Lygeum Loefl. ex L. (lygeum), Melica L. (melicgrass), Melinis P. Beauv. (stinkgrass), Merxmuellera Conert, Mibora Adans. (sandgrass), Microchloa R. Br. (smallgrass), Microlaena R. Br. (weeping grass), Micropyrum Link, Microstegium Nees (browntop), Milium L. (milletgrass), Miscanthus Andersson (silvergrass), Mnesithea Kunth (jointtail grass), Molinia Schrank (moorgrass), Monanthochloe Engelm. (shoregrass), Muhlenbergia Schreb. (muhly), Munroa Torr., orth. cons. (false buffalograss), Nardus L. (matgrass), Nassella (Trin.) Desv. (needlegrass), Neeragrostis Bush (creeping lovegrass), Neololeba Widjaja (neololeba), Neostapfia Burtt Davy (Colusagrass), Neyraudia Hook. f. (neyraudia), Ochthochloa Edgew., Olyra L. (carrycillo), Ophiuros C. F. Gaertn., Oplismenus P. Beauv. (basketgrass), Orcuttia Vasey (Orcutt grass), Oryza L. (rice), Oryzopsis Michx. (ricegrass), Ottochloa Dandy, Oxychloris M. Lazarides, Oxytenanthera Munro (oxytenanthera), Panicum L. (panicgrass), Pappophorum Schreb. (pappusgrass), Parapholis C. E. Hubbard (sicklegrass), Pascopyrum Á. Löve (wheatgrass), Paspalidium Stapf (watercrown grass), Paspalum L. (crowngrass), Patis Ohwi (ricegrass), Pennisetum Rich. ex Pers. (fountaingrass), Perotis Aiton, Phalaris L. (canarygrass), Phanopyrum (Raf.) Nash (savannah-panicgrass), Pharus L. (stalkgrass), Phippsia (Trin.) R. Br. (icegrass), Phleum L. (timothy), Phragmites Adans. (reed), Phyllostachys Siebold & Zucc. (bamboo), Piptatheropsis Romasch., P. M. Peterson & R. J. Soreng (ricegrass), Piptatherum P. Beauv. (ricegrass), Piptochaetium J. Presl (speargrass), Plectrachne Henrard, Pleioblastus Nakai (dwarf bamboo), Pleuraphis Torr. (galleta grass), Pleuropogon R. Br. (semaphoregrass), Poa L. (bluegrass), Pogonatherum P. Beauv., Polypogon Desf. (rabbitsfoot grass), Polytoca R. Br., Polytrias Hack. (Java grass), Psathyrostachys Nevski (wildrye), x Pseudelymus Barkworth & D. R. Dewey (foxtail wheatgrass), Pseudoroegneria (Nevski) Á. Löve (wheatgrass), Pseudosasa Makino ex Nakai (arrow bamboo), Psilurus Trin., Ptilagrostis Griseb. (false needlegrass), Puccinellia Parl. (alkaligrass), x Pucciphippsia Tzvelev (pucciphippsia), Redfieldia Vasey (blowout grass), Reimarochloa Hitchc. (reimar grass), Rostraria Trin. (hairgrass), Rottboellia L. f. (itchgrass), Rytidosperma Steud. (wallaby grass), Saccharum L. (sugarcane), Sacciolepis Nash (cupscale grass), Sasa Makino & Shib. (broadleaf bamboo), x Schedolium Holub (fescue ryegrass), Schedonnardus Steud. (tumblegrass), Schedonorus P. Beauv. (fescue), Schismus P. Beauv. (Mediterranean grass), Schizachne Hack. (false melic), Schizachyrium Nees (little bluestem), Schizostachyum Nees (Polynesian 'ohe P), Schmidtia Moench, Sclerochloa P. Beauv. (hardgrass), Scleropogon Phil. (burrograss), Sclerostachya A. Camus, Scolochloa Link (rivergrass), Scribneria Hack. (Scribner's grass), Secale L. (rye), Sehima Forssk., Genus Semiarundinaria Makino, Sesleria Scop., Setaria P. Beauv. (bristlegrass), Setaria viridis (e.g. of the Andropogoneae tribe), Setariopsis Scribn. ex Millsp. (setariopsis), Shibataea Makino ex Nakai, Sinocalamus McClure (wideleaf bamboo), Snowdenia C. E. Hubb., Sorghastrum Nash (Indiangrass), Sorghum Moench (sorghum), Spartina Schreb. (cordgrass), Sphenopholis Scribn. (wedgescale), Spinifex L., Spodiopogon Trin., Sporobolus R. Br. (dropseed), Steinchisma Raf. (gaping grass), Stenotaphrum Trin. (St. Augustine grass), Stipa L., Stipagrostis Nees, Swallenia Söderst. & Decker (dunegrass), Taeniatherum Nevski (medusahead), Tetrachne Nees, Tetrapogon Desf., Thaumastochloa C. E. Hubb., Themeda Forssk. (kangaroo grass), Thinopyrum Á. Löve (wheatgrass), Thuarea Pers. (Kuroiwa grass), Thyridolepis S. T. Blake, Thysanolaena Nees (tiger grass), Torreyochloa Church (false mannagrass), Trachypogon Nees (crinkleawn grass), Tragus Haller (bur grass), Tribolium Desv. (tribolium), Trichloris Fourn. ex Benth. (false Rhodes grass), Tricholaena Schrad., Trichoneura Andersson (Silveus' grass), Tridens Roem. & Schult. (tridens), Triodia R. Br., Triplasis P. Beauv. (sandgrass), Tripogon Roem. & Schult. (fiveminute grass), Tripsacum L. (gamagrass), Triraphis R. Br. (needlegrass), Trisetum Pers. (oatgrass), x Triticosecale Wittm. ex A. Camus (triticale), Triticum L. (wheat), Tuctoria J. Reeder (spiralgrass), Uniola L. (seaoats), Urochloa P. Beauv. (signalgrass), Vahlodea Fr. (hairgrass), Vaseyochloa Hitchc. (Texasgrass), Ventenata Koeler (North Africa grass), Vetiveria Bory (vetivergrass), Vossia Wall. & Griffith (hippo grass), Vulpia C. C. Gmel. (fescue), Whiteochloa C. E. Hubb., Willkommia Hack. (willkommia), Zea L. (corn), Zingeria P. A. Smirn., Zizania L. (wildrice), Zizaniopsis Döll & Asch. (cutgrass), P. australis, or Zoysia Willd. (lawngrass).

In some embodiments, the engineered plant is of the genus Striga. In some embodiments, the engineered plant is witchweed (e.g. Striga hermonthica).

In some embodiments, the engineered plant is of the genus Cardamine. In some embodiments, the engineered plant is Cardamine hirsute (hairy bittercress).

In some embodiments, the engineered plant is an algae.

In some embodiments, the engineered plant is an algae from the phyla Rhodophyta (red algae), Chlorophyta (green algae), Phaeophyta (brown algae), Bacillariophyta (diatoms), Eustigmatophyta, a dinoflagellates, or the prokaryotic phylum Cyanobacteria (blue-green algae).

In some embodiments, the engineered algae the species of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, or Trichodesmium.

In some embodiments, the engineered cell, the engineered plant, or both include a NUDIX gene and/or NUDIX gene product encoding polynucleotide stably integrated into the genome of the engineered cell.

In some embodiments, the engineered cell, the engineered plant, or both include a NUDIX gene and/or NUDIX gene product encoding polynucleotide that is transiently expressed in the engineered cell, engineered plant, or both.

### Methods of Generating NUDIX Overexpressing Plants

Also described herein are methods of modifying a plant cell such that it contains and optionally expresses a NUDIX polypeptide and/or NUDIX gene and/or NUDIX gene product encoding polynucleotide and/or vector or vector system containing a NUDIX gene product encoding polynucleotide, or a combination thereof. In some embodiments, the method includes modifying a plant cell such that the plant cell contains and optionally expresses a heterologous or non-native NUDIX polypeptide, a vector or vector system containing a non-native or heterologous NUDIX gene or NUDIX gene product encoding polynucleotide, or a combination thereof. In some embodiments, modifying includes delivering a polynucleotide having a sequence that is about 70-100% (e.g., 70 to/or 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to (a) any one of SEQ ID NO: 1-27, (b) any one of SEQ ID NO: 4, 12, 13, 14, 16, 17, 18, and 21, or (c) any one of SEQ ID NO: 4, 12, 13, 16, and 21 or a vector or vector system thereof to the cell; delivering a polypeptide having a sequence that is about 70-100% (e.g., 70, to/or 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) identical to (a) any one of SEQ ID NO: 28-54, (b) any one of SEQ ID NO: 31, 39, 40, 41, 43, 44, 45, and 48, or (c) any one of SEQ ID NO: 31, 39, 40, 43, and 48 to the cell; or both. In some embodiments, the NUDIX polypeptide, NUDIX gene, and/or NUDIX gene product is not DDP1. In some embodiments, the NUDIX polypeptide is not a DDP1 polypeptide sequence that is 70-100% identical to SEQ ID NO: 101. In some embodiments, the NUDIX polypeptide is not a DDP1 polypeptide sequence that is 70-100% identical to a coding sequence available at GenBank Accession No. AY558436).

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

“Plant” as used herein encompasses any plant tissue or part of the plant of the invention. In some embodiments, said part is selected from the group of a plant cell, a somatic embryo, a pollen, a gametophyte, an ovule, an inflorescence, a leaf, a seedling, a stem, a callus, a stolon, a microtuber, a root, a shoot, a seed, a fruit and a spore. Further encompassed are T1 generation plants produced from the seeds of the transformed plant (T0). Any suitable method can be used to confirm and detect the modification made in the plant. Such methods are generally known in the art. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

A part of a plant, e.g., a “plant tissue” can be engineered to include a NUDIX gene product encoding polynucleotide, vector, and/or polypeptide described elsewhere herein to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processingcolored substances (e.g., the β-glucuronidase, luciferase, B or C1 genes).

Any suitable method may be used to deliver the transgene to the plant, plant cell, and/or plant cell population. Such transformation techniques are generally known in the art. Example methods and techniques include those in U.S. Pat. No. 6,603,061—Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety.

In some embodiments, where transient expression is desired transgene DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

A method of generating engineered cells and/or plants can include transformation of one or more cells. The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, roots, and shoots. A plant tissue also refers to any clone of such a plant, seed, progeny, propagule whether generated sexually or asexually, and descendants of any of these, such as cuttings or seed. The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the NUDIX transgene or other NUDIX genetic or epigenetic modification system is expressed. Vectors and vector systems are described in greater detail elsewhere herein.

The term plant also encompasses progeny of the plant. The term “progeny”, such as the progeny of a transgenic (or engineered) plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

Also described herein are gametes, seeds, germplasm, embryos, either zygotic or somatic, progeny or hybrids of plants comprising the genetic modification (e.g., inclusion and/or expression of a NUDIX encoding polynucleotide or modified NUDIX polynucleotide), which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

### Stable Integration in the Genome of Plants and Plant Cells

In particular embodiments, the polynucleotides encoding NUDIX gene product are introduced for stable integration into the genome of a plant cell. In these embodiments, the design of the transformation vector or the expression system can be adjusted depending on for when, where and under what conditions the NUDIX gene product encoding polynucleotides are expressed. Suitable vectors and delivery are described in greater detail elsewhere herein.

In particular embodiments, the NUDIX gene product encoding polynucleotides are stably introduced into the genomic DNA of a plant cell. In particular embodiments, the NUDIX gene product encoding polynucleotides are introduced for stable integration into the DNA of a plant organelle such as, but not limited to a plastid, mitochondrion or a chloroplast. In some embodiments, the expression system for stable integration into the genome of a plant cell can contain one or more of the following elements: a promoter element that can be used to express NUDIX gene product encoding polynucleotide(s) in a plant cell; a 5′ untranslated region to enhance expression; an intron element to further enhance expression in certain cells, such as monocot cells; a multiple-cloning site to provide convenient restriction sites for inserting the polynucleotide modifying agent(s) or a system thereof and other desired elements; and a 3′ untranslated region to provide for efficient termination of the expressed transcript. The elements of the expression system may be on one or more expression constructs which are either circular such as a plasmid or transformation vector, or non-circular such as linear double stranded DNA.

DNA construct(s) containing the components of the systems, and, where applicable, template sequence may be introduced into the genome of a plant, plant part, or plant cell by a variety of conventional techniques. The process generally comprises the steps of selecting a suitable host cell or host tissue, introducing the construct(s) into the host cell or host tissue.

In particular embodiments, the DNA construct may be introduced into the plant cell using techniques such as but not limited to electroporation, microinjection, aerosol beam injection of plant cell protoplasts, or the DNA constructs can be introduced directly to plant tissue using biolistic methods, such as DNA particle bombardment (see also Fu et al., Transgenic Res. 2000 February; 9(1):11-9). The basis of particle bombardment is the acceleration of particles coated with gene/s of interest toward cells, resulting in the penetration of the protoplasm by the particles and typically stable integration into the genome. (see e.g. Klein et al, Nature (1987), Klein et ah, Bio/Technology (1992), Casas et ah, Proc. Natl. Acad. Sci. USA (1993).).

In particular embodiments, the DNA constructs containing components of the systems may be introduced into the plant by Agrobacterium-mediated transformation. The DNA constructs may be combined with suitable T-DNA flanking regions and introduced into a conventional Agrobacterium tumefaciens host vector. The foreign DNA can be incorporated into the genome of plants by infecting the plants or by incubating plant protoplasts with Agrobacterium bacteria, containing one or more Ti (tumor-inducing) plasmids. (see e.g. Fraley et al., (1985), Rogers et al., (1987) and U.S. Pat. No. 5,563,055).

### Transient Expression of in Plants and Plant Cells

In some embodiments, the NUDIX gene product encoding polynucleotides can be transiently expressed in the plant cell. In these embodiments, the system can ensure NUDIX gene product expression and any Pi accumulation can further be controlled. As the expression of the necessary components of the NUDIX gene product encoding polynucleotide(s) is transient, plants regenerated from such plant cells typically contain no foreign DNA.

In particular embodiments, the NUDIX gene product encoding polynucleotides can be transiently introduced in the plant cells using a plant viral vector (Scholthof et al. 1996, Annu Rev Phytopathol. 1996; 34:299-323). In further particular embodiments, said viral vector is a vector from a DNA virus. For example, geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). In other particular embodiments, said viral vector is a vector from an RNA virus. For example, tobravirus (e.g., tobacco rattle virus, tobacco mosaic virus), potexvirus (e.g., potato virus X), or hordeivirus (e.g., barley stripe mosaic virus). The replicating genomes of plant viruses are non-integrative vectors. Other suitable vectors are described elsewhere herein

In particular embodiments, the vector used for transient expression of constructs in plants is for instance a pEAQ vector, which is tailored for Agrobacterium-mediated transient expression (Sainsbury F. et al., Plant Biotechnol J. 2009 September; 7(7):682-93) in the protoplast. Precise targeting of genomic locations was demonstrated using a modified Cabbage Leaf Curl virus (CaLCuV) vector to express gRNAs in stable transgenic plants expressing a CRISPR enzyme (Scientific Reports 5, Article number: 14926 (2015), doi:10.1038/srep14926).

In particular embodiments, double-stranded DNA fragments encoding NUDIX gene product can be transiently introduced into the plant cell. In such embodiments, the introduced double-stranded DNA fragments are provided in sufficient quantity to modify the cell but do not persist after a contemplated period of time has passed or after one or more cell divisions. Methods for direct DNA transfer in plants are known by the skilled artisan (see for instance Davey et al. Plant Mol Biol. 1989 September; 13(3):273-85.)

In other embodiments, an RNA polynucleotide encoding the NUDIX gene product is introduced into the plant cell, which is then translated and processed by the host cell generating the protein in sufficient quantity accumulate Pi but which does not persist after a contemplated period of time has passed or after one or more cell divisions. Methods for introducing mRNA to plant protoplasts for transient expression are known by the skilled artisan (see for instance in Gallie, Plant Cell Reports (1993), 13; 119-122).

In some embodiments, a combination of the different methods described above can be used.

### Translocation to and or Expression in Specific Plant Organelles

The system may comprise elements for translocation to and/or expression in a specific plant organelle. In some embodiments, a tissue specific promoter can be included in the expression construct. In some embodiments, a tissue localization or organelle localization sequence or signal can be incorporated into the expression constructs. Such promoters and localization signals are described in greater detail elsewhere herein and/or will be appreciated by one of ordinary skill in the art.

### Chloroplast Targeting

In some embodiments, the engineered plants can be engineered to contain modified chloroplast genes or to ensure expression in the chloroplast. In some embodiments, chloroplast transformation methods or compartmentalization of the NUDIX gene product encoding polynucleotides and/or polypeptides to the chloroplast. For instance, the introduction of genetic modifications in the plastid genome can reduce biosafety issues such as gene flow through pollen.

Methods of chloroplast transformation are known in the art and include Particle bombardment, PEG treatment, and microinjection. Additionally, methods involving the translocation of transformation cassettes from the nuclear genome to the plastid can be used as described in WO2010061186.

In some embodiments, one or more of the NUDIX gene product encoding polynucleotides can be targeted to the plant chloroplast. This can be achieved by incorporating in the expression construct a sequence encoding a chloroplast transit peptide (CTP) or plastid transit peptide, operably linked to the 5′ region of the sequence encoding the Cas protein. The CTP is removed in a processing step during translocation into the chloroplast. Chloroplast targeting of expressed proteins is well known to the skilled artisan (see for instance Protein Transport into Chloroplasts, 2010, Annual Review of Plant Biology, Vol. 61:157-180). In such embodiments it is also can be desirable to target the guide RNA to the plant chloroplast. Methods and constructs which can be used for translocating guide RNA into the chloroplast by means of a chloroplast localization sequence are described, for instance, in US20040142476, incorporated herein by reference. Such variations of constructs can be incorporated into the expression systems of the invention to efficiently translocate the NUDIX gene product encoding polynucleotides.

### Methods of Using the Engineered Plants

The engineered NUDIX overexpressing plants described herein can be propagated, grown, harvested, and/or cultivated for any purpose such as food or commodity production and/or fertilizer. In some embodiments, a method can include cultivating a plant as described elsewhere herein.

### Methods of Soil Remediation

The engineered plants described herein can be propagated, grown, and/or cultivated for soil remediation. As described and demonstrated elsewhere herein, the NUDIX gene and gene product overexpressing plants can be capable of sequestering Pi from the soil and accumulating it in greater amounts as compared to a non-NUDIX or wild-type NUDIX expressing control plant. Thus, in this way, growth of a NUDIX overexpressing plant can facilitate reducing soil levels of Pi. In some embodiments, the NUDIX overexpressing plant is grown in a soil that has a high level of Pi. In some embodiments, the NUDIX overexpressing plant has one or more NUDIX genes or gene product encoding polynucleotides under control of an inducible promoter. In some embodiments, expression of one or more NUDIX genes is not induced until after the engineered NUDIX plant capable of overexpressing a NUDIX gene and/or gene product has reached a desired growth stage, util after a fruit or other component of the plant has been harvested, and/or until after a set period of time after planting, germinating, and/or sprouting.

In some embodiments, NUDIX overexpression is not induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days post planting, germinating, and/or sprouting. In some embodiments, NUDIX over expression is not induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks post planting, germinating, and/or sprouting. In some embodiments, NUDIX overexpression is not induced for 1, 2, 3, 4, 5, or more years post planting, germinating, and/or sprouting.

In some embodiments, NUDIX overexpression is induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, NUDIX overexpression is induced for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks. In some embodiments, NUDIX overexpression is induced for 1, 2, 3, 4, 5, or more years.

Products Produced from the NUDIX Overexpressing Plants

In some embodiments, the NUDIX overexpressing plants and/or parts (e.g., fruits, nuts, seeds, grains, etc.) are harvested. In some embodiments, one or more parts or the whole plant is harvested for fertilizer. In some embodiments, after one or more parts of the NUDIX overexpressing plant is harvested for a non-fertilizer product, the rest is harvested for fertilizer production. Once a NUDIX overexpressing plant has accumulated the Pi, it can be harvested and be used in any suitable form as a fertilizer. In this way, sequestered Pi can be reapplied when desired as a fertilizer and thus serve to recycle phosphorous.

In some embodiments, the harvested NUDIX overexpressing plants can be applied as a fresh-biomass fertilizer. Such fresh biomass can be chopped, shredded, pulverized, and/or otherwise mechanically broken down prior to application.

In other embodiments, the harvested NUDIX overexpressing plants can be further processed, such as carbonized, prior to applying as a fertilizer. In some embodiments, the harvested NUDIX overexpressing plants can be processed into biochar. “Biochar” as used herein is a term of art and refers to a charcoal-like byproduct of the process of pyrolysis, or the anaerobic (meaning without oxygen) thermal decomposition of organic material, particularly plant material in the present context. The biochar can be applied as a fertilizer for plant cultivation.

The cycle can be continued by growing NUDIX overexpressing plants in soil fertilized with DPP1 fresh biomass and/or biochar.

Thus, also described herein is biochar produced from NUDIX overexpressing plants that have accumulated/sequestered Pi from the soil in which they were grown. In some embodiments, the biochar has a slower phosphorus release rate than biochar produced from a wild-type plant (or non-NUDIX expressing or wild-type NUDIX expressing control plant). In some embodiments, the biochar produced from NUDIX overexpressing plants that have accumulated/sequestered Pi from the soil in which they were grown releases phosphorous at a rate 1-1000 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 times slower than biochar produced from a wild-type plant (or non-NUDIX expressing or wild-type NUDIX control plant). In some embodiments, the biochar produced from NUDIX overexpressing plants that have accumulated/sequestered Pi from the soil in which they were grown releases phosphorous about twice as slow (e.g., 50% of the rate) as biochar produced from a wild-type plant (or non-NUDIX expressing or wild-type NUDIX control plant).

In some embodiments, the biochar is combined with one or more other nutrients, soil fortifiers, soils, mulches, binders, or other soil additives.

Methods of Fertilizing Using the Engineered NUDIX Overexpressing Plants and/or Fertilizers Produced Therefrom

Also described herein is methods of using biochar and/or fresh biomass produced from NUDIX overexpressing plants that have accumulated/sequestered Pi from the soil in which they were grown as a fertilizer. In some embodiments, biochar and/or fresh biomass produced from NUDIX overexpressing plants that have accumulated/sequestered Pi from the soil in which they were grown is applied to an area in which plants will be grown or are actively growing as a source of phosphorus for growth of the plants.

In some embodiments, biochar and/or fresh biomass is applied 1-10 (e.g., 1, 2, 3, 4, 5, 6, 7, 8, 9, 10) or more times to an area in which plants will be grown or are growing in a year.

The amount of phosphorous and other nutrient qualities can be measured by any suitable methods and/or techniques. Such methods and/or techniques will be appreciated by one of ordinary skill in the art and/or are described elsewhere herein.

The amount of fresh biomass or biochar applied to any given area, will depend on, for example, the plant to which it is being applied, the starting soil nutrient load, the amount allowed as per nutrient management plan and/or local, state, or federal limit imposed on the particular area, the specific Pi present in the biomass and/or biochar being applied, and the like. Other relevant factors will be appreciated by one of ordinary skill in the art in view of this disclosure.

Further embodiments are illustrated in the following Examples which are given for illustrative purposes only and are not intended to limit the scope of the invention.

### EXAMPLES

Now having described the embodiments of the present disclosure, in general, the following examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

NUDIX (Nucleoside Diphosphate compounds linked to a moiety X) enzymes are a highly diverse set of pyrophosphatases found in all domains of life. They share a highly conserved motif of 23 amino acids known as the Nudix box or MutT: GX₅EX₇REUXEEXGU where X is any amino acid; U is a hydrophobic residue) (Sheikh et al., 1998; Xu et al., 2002; Kraszewska, 2008). The Nudix box motif is essential for NUDIX hydrolysis of a variety of substrates (Gunawardana et al., 2009; Márquez-Moñino et al., 2021). Structurally, this sequence is found in a loop-helix-loop motif within the NUDIX enzyme (McLennan, 2006). NUDIX catalytic activity is dependent on the two underlined glutamates, REUXEE (SEQ ID NO: 62), for hydrolysis activity and binding to metal cofactors (Mildvan et al., 2005; McLennan, 2006). NUDIX enzymes have been found to hydrolyze a wide range of substrates. These include nucleotide-based molecules such as dinucleotides, dNTPs, along with capped mRNAs, diadenosine polyphosphates (ApₙAs), and other modified nucleotides, as well as non-nucleotide-based molecules: inositol pyrophosphates (PP-InsPs), polyphosphates (polyP), 5-phosphoribosyl 1-diphosphate, thiamine pyrophosphate, and dihydroneopterin triphosphate (McLennan, 2006; Kraszewska, 2008). While the structures of multiple NUDIX enzymes have been resolved across organisms (Massiah et al., 2003; Zong et al., 2021; Márquez-Moñino et al., 2021; Srouji et al., 2017), there remain many unknowns within the plant NUDIX enzyme family.

A total of 27 NUDIX enzymes have been identified in Arabidopsis thaliana (Kraszewska, 2008; Williams, 2015). Of these 27 enzymes, only the structures of NUDIX1 (Liu et al., 2018) and NUDIX7 (Tang et al., 2015) have been resolved. Further, the enzymatic activities of about half of these enzymes have been empirically explored but the roles of many of these NUDIX enzymes remain unelucidated (Kraszewska, 2008). Of what has been explored with regard to the plant NUDIX enzymes, the role of these enzymes in inositol pyrophosphate (PP-InsP) signaling in plants lacks clarity. To date, only one study empirically addresses PP-InsP enzyme activity and explores this for the NUDIX13 enzyme (Olejnik et al., 2007). An objective of this Example is to elucidate the NUDIX enzyme(s) involved in PP-InsP signaling. Applicant divided the 27 predicted NUDIX enzymes into 9 groups based on their phylogenic relationships to one another and with the Saccharomyces cerevisiae enzyme, DDP1 (FIG. 1).

The substrates for each group were either empirically (Ogawa et al., 2008; Olejnik et al., 2007) or bioinformatically assessed (Gunawardana et al., 2009; The UniProt Consortium, 2021). Of these groups, group 9, consisting of NUDIX4, -12, -13, -16, -17, -18, and -21, is predicted to share the closest relationship with (FIG. 1). An extensive bioinformatics analysis predicts that NUDIX enzymes from group 9 have diphosphoinositol polyphosphate phosphohydrolase (DIPP) activity (Gunawardana et al., 2009). A separate bioinformatic study shows that homology search of DDP1 and diphosphoinositol polyphosphate phosphohydrolase (DIPP) from Homo sapiens with 29 putative Arabidopsis NUDIX amino acid sequences also identified group 9 NUDIX enzymes to share the highest homology (Williams, 2015). The number above each branch FIG. 1 is the bootstrap support percentage of n=1000 bootstrap replications. This phylogenetic tree was rooted on DDP1. Divided into 9 groups based on structural annotations made in (Gunawardana et al., 2009). Blue boxes, as represented in greyscale, show annotations based on (Gunawardana et al., 2009), orange boxes, as represented in greyscale, show empirically verified substrates from (Ogawa et al., 2008), purple boxes, as represented in greyscale, show overlap between Gunawardana et al annotations and Ogawa et al verified substrates, the green box, as represented in greyscale, shows annotations from UniProt (The UniProt Consortium, 2021), and the gray box shows empirically verified substrates from (Olejnik et al., 2007).

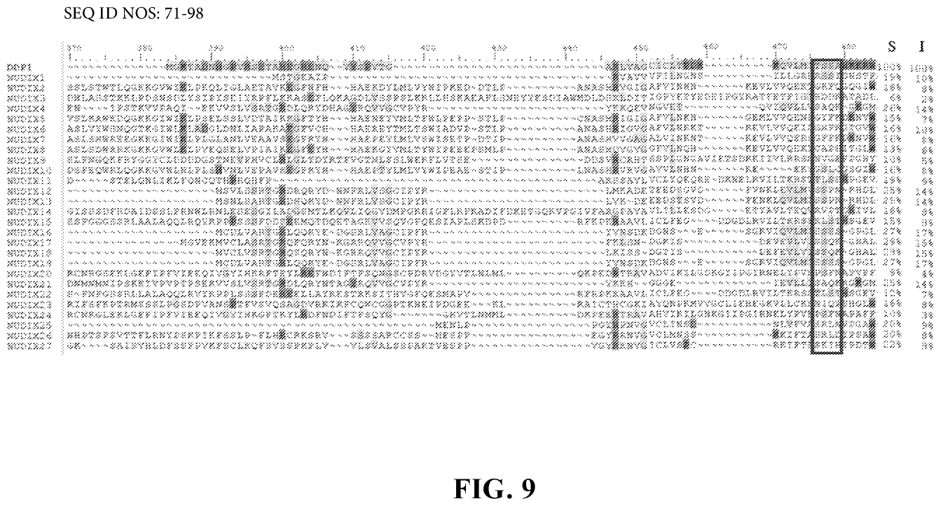

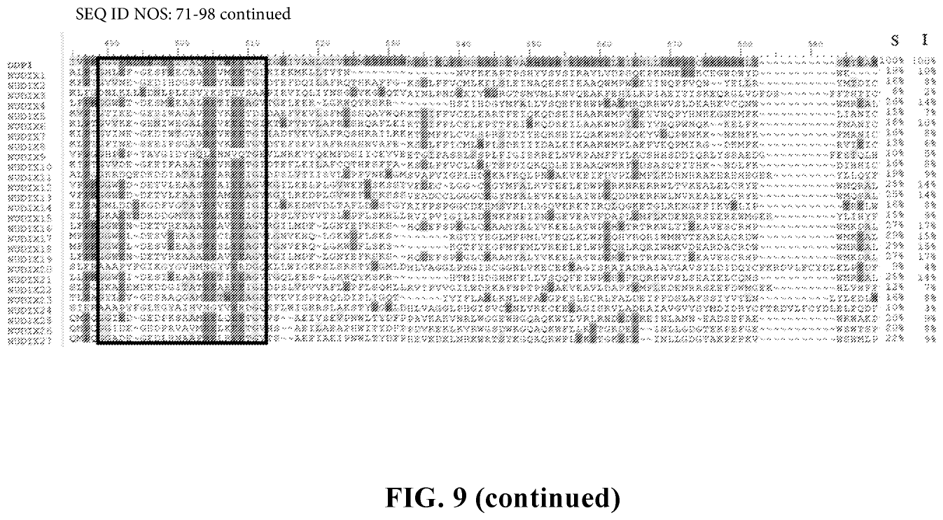

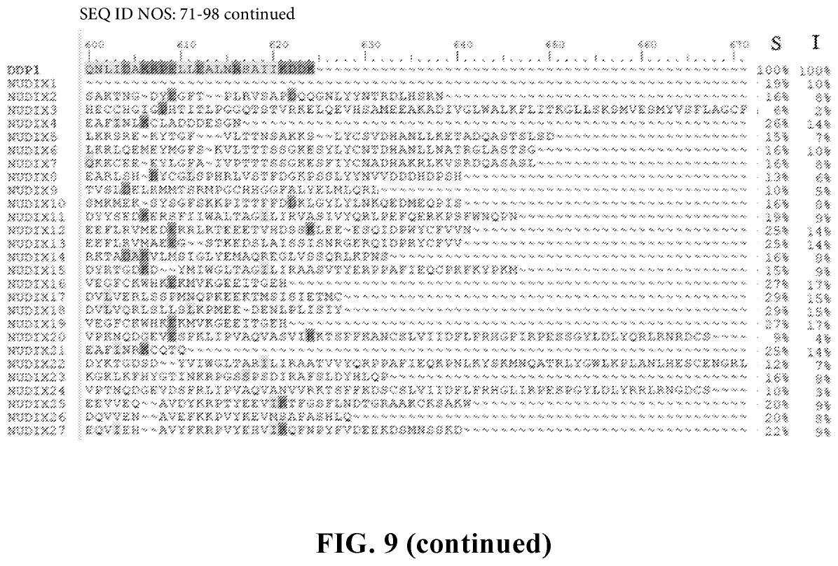

Of group 9 NUDIX enzymes, only NUDIX13 was assessed for PP-InsP hydrolysis in vitro (Olejnik et al., 2007) and it was not found to hydrolyze PP-InsP₅ under their conditions. A recent crystallography study suggested that the 52-SSA motif upstream of the Nudix box in DDP1 allows the active site to accommodate PP-InsPs as a double mutant for these serine residues yielded a nonstable DDP1 enzyme (Márquez-Moñino et al., 2021). Notably, 5 of the 7 NUDIX enzymes within group 9 (NUDIX12, -13, -16, -17, and -18) contain a conserved SS motif upstream of the Nudix box (FIG. 9), suggesting there could be potential for these enzymes to accommodate PP-InsPs within the catalytic pocket.

Based on phylogenetic groupings, bioinformatic annotations, and limited in vitro data, Applicant prioritized the group 9 NUDIX enzymes in this study. An object of this Example was to identify native NUDIX enzymes that are involved in PP-InsP signaling and degradation in planta. To assess this, Applicant utilized two distinct approaches to explore the potential roles of these enzymes in PP-InsP hydrolysis. First, Applicant characterized Arabidopsis gain-of-function plants overexpressing a subset of NUDIX enzymes from group 9. Applicant selected NUDIX12 and NUDIX13 as a subset of group 9 enzymes for this study. While NUDIX13 was not found to hydrolyze PP-InsP₅ in Olejnik et al, this provides the ideal control as to whether this enzyme can manipulate PP-InsP signaling in vivo. NUDIX12 was also included given its close relationship to NUDIX13. Substrate preferences of all group 9 NUDIX enzymes were also queried through a series of in silico docking assays using NUDIX homology models.

### NUDIX12 and NUDIX13 Decrease PP-InsPs in Arabidopsis Seedlings

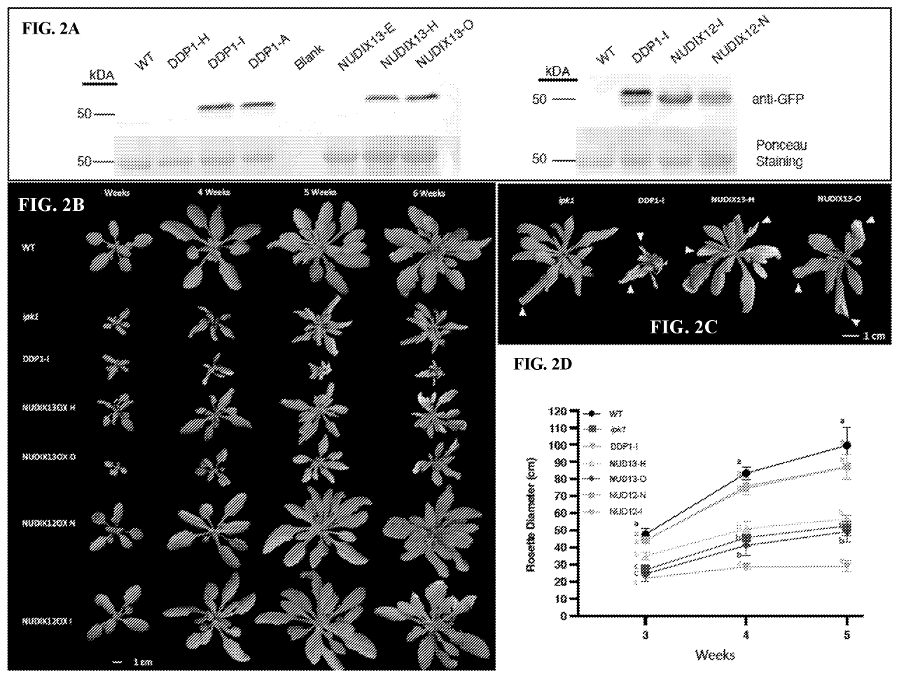

To assess whether group 9 NUDIX enzymes can impact PP-InsP signaling in planta, Applicant selected a subset of group 9 enzymes, specifically NUDIX12 (At1g12880) and NUDIX13 (At3g26690). Transgenic lines that contain the CaMV 35S promoter driving expression of NUDIX12 or NUDIX13 fused to a C-terminal GFP tag were constructed in the Arabidopsis Col-0 background. NUDIX12 and NUDIX13 OX lines selected for this study were characterized based on fusion protein expression by protein blotting with an anti-GFP antibody. Based on previous results with Applicant's DDP1 OX transgenics, Applicant prioritized lines that had higher accumulation of fusion protein (FIG. 2A-2B). Specifically, NUDIX12-I, NUDIX12-N, NUDIX13-H, and NUDIX13-O were selected as all four transgenic lines expressed abundant levels of protein. Interestingly, a preliminary analysis of growth phenotypes revealed that NUDIX13 lines had smaller rosette diameters exhibited leaf tip chlorosis and necrosis phenotypes whereas NUDIX12 OX lines showed no visible growth differences from WT (FIG. 2C-2D). Leaf tip necrosis and chlorosis are an indication of Pi toxicity and are prominently manifested in DDP1 OX transgenic leaves (Aung et al., 2006. This phenotype suggests that overexpression of NUDIX13, but not NUDIX12, could induce a perturbance in PP-InsP accumulation leading to elevated Pi-accumulation and manifestation of Pi-linked phenotypes.

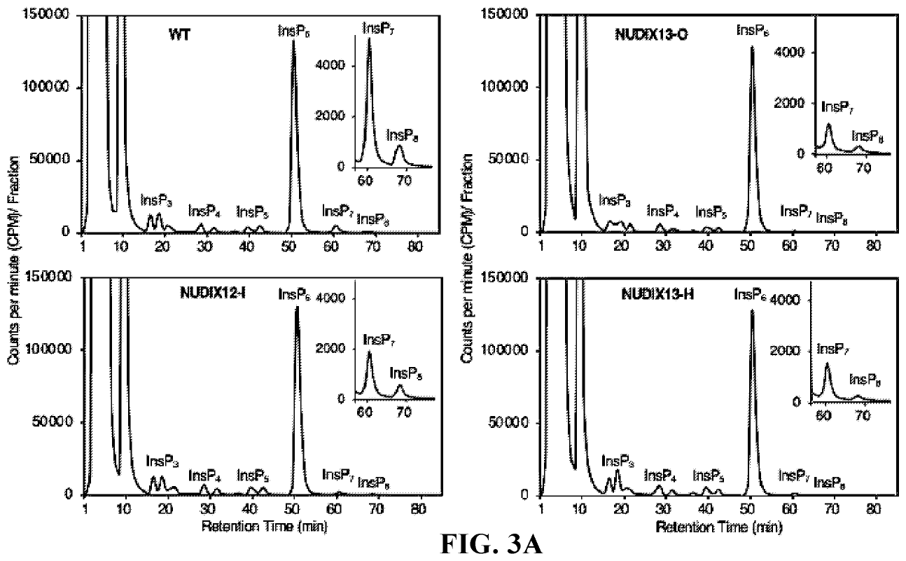

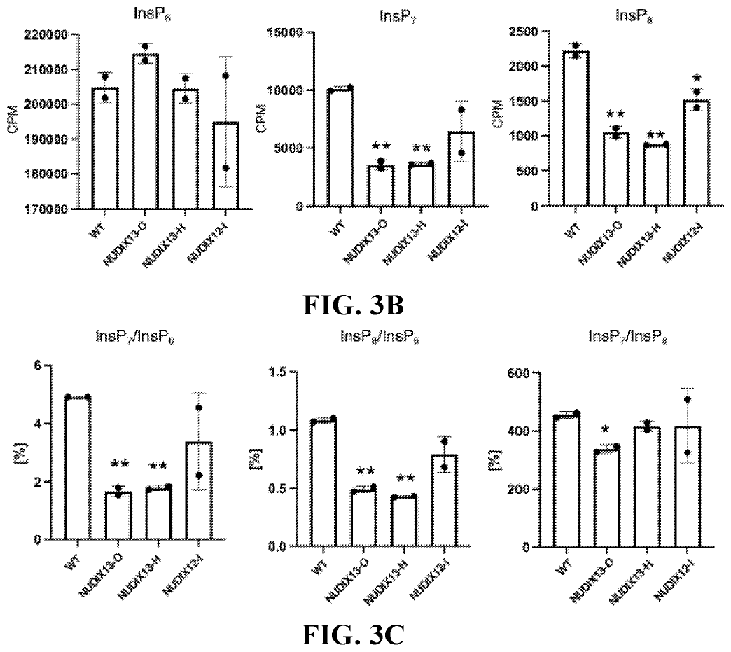

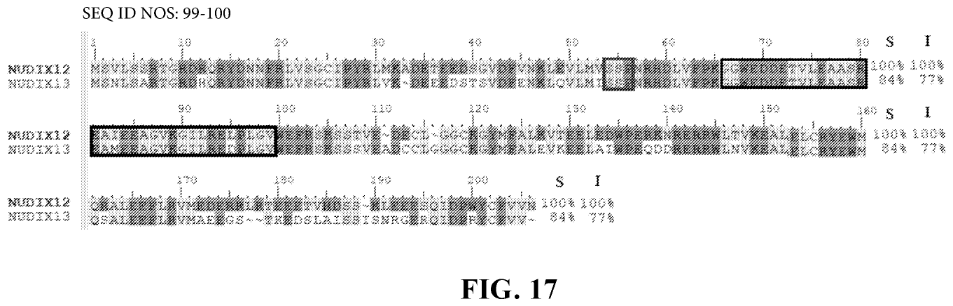

Applicant measured InsPs and PP-InsPs in WT as well as all NUDIX12 and NUDIX13 lines to query impacts on PP-InsPs. InsPs and PP-InsPs were extracted and quantified from 2-week-old seedlings using a previously described in vivo myo-[³H] inositol radiolabeling method followed by high-performance liquid chromatography (HPLC) analysis (Desai et al., 2014; Adepoju et al., 2019). Notably, NUDIX13-O and NUDIX13-H plants exhibited significant decreases in InsP₇ and InsP₈ pools compared to WT plants (FIGS. 3A-3C and 17). NUDIX12-I lines also showed decreased InsP₇ and InsP₈ levels however only the decrease in InsP₈ was significant as compared to WT (FIG. 3A-3B and FIG. 17). There was no significant difference in InsP₆ levels in any transgenics overexpressing NUDIX enzymes. When expressed as ratios, NUDIX13-O and NUDIX13-H had significantly lower InsP₇/InsP₆ and InsP₈/InsP₆ ratios compared to WT, whereas there was no significant change in NUDIX12-I ratios (FIG. 3C). Interestingly, NUDIX13-O lines also had a significant change in InsP₇/InsP₈ ratios compared to WT. Based on these ratios and quantification of InsP species as compared to WT pools, the decrease in InsP₇ and InsP₈ appear to be a proportional decrease in all three transgenic lines (FIG. 3C and Table 3). Comparing the average percent of InsP₃-InsP₈ species in all lines compared to WT revealed that there were no changes in any InsP species except for InsP₇ and InsP₈ (Table 3). NUDIX13-O and NUDIX13-H respectively accumulated 35±4% and 36±0% InsP₇ of the total WT InsP₇ pool and 48±4% and 39±2% of the total WT InsP₈ pool. NUDIX12-I also had decreased PP-InsPs with 64±19% of the total WT InsP₇ pool and 69±7% of the total WT InsP₈ pool. This data suggests that expression of both NUDIX13 and NUDIX12 in Arabidopsis impacts PP-InsPs, with NUDIX13 having a significantly higher impact on PP-InsP signaling than NUDIX12. Given all four lines queried had accumulated similar levels of GFP-tagged protein, this indicates that a NUDIX13 gain-of-function has a greater impact on perturbing PP-InsPs in Arabidopsis, as compared to NUDIX12 gain-of-function. For Table 3 averages were taken from all replicates shown in FIG. 2A-2D and FIG. 9, n=2 biological replicates per genotype. Values that represent 50% or less of the total WT pool are designated with an *; values between 51-75% of the total WT pool are designated with an **.

TABLE 3
The average percent of total, individual InsP species compared
to the total WT pool expressed as a percentage.

| | NUDIX13-O | | NUDIX13-H | | NUDIX12-I | |
|---|---|---|---|---|---|---|
| | Average | Std | Average | Std | Average | Std |
| | % | Dev | % | Dev | % | Dev |
| InsP₃ | 91% | ±2% | 109% | ±8% | 111% | ±6% |
| InsP₄ | 96% | ±4% | 105% | ±13% | 124% | ±9% |
| InsP₅ | 92% | ±0% | 102% | ±9% | 122% | ±10% |
| InsP₆ | 105% | ±3% | 100% | ±3% | 95% | ±5% |
| InsP₇ | 35%* | ±4% | 36%* | ±0% | 64%** | ±19% |
| InsP₈ | 48%* | ±4% | 39%* | ±2% | 69%** | ±7% |

NUDIX13 OX Shares Similarities with DDP1 OX and Mutants with Perturbed PP-InsPs

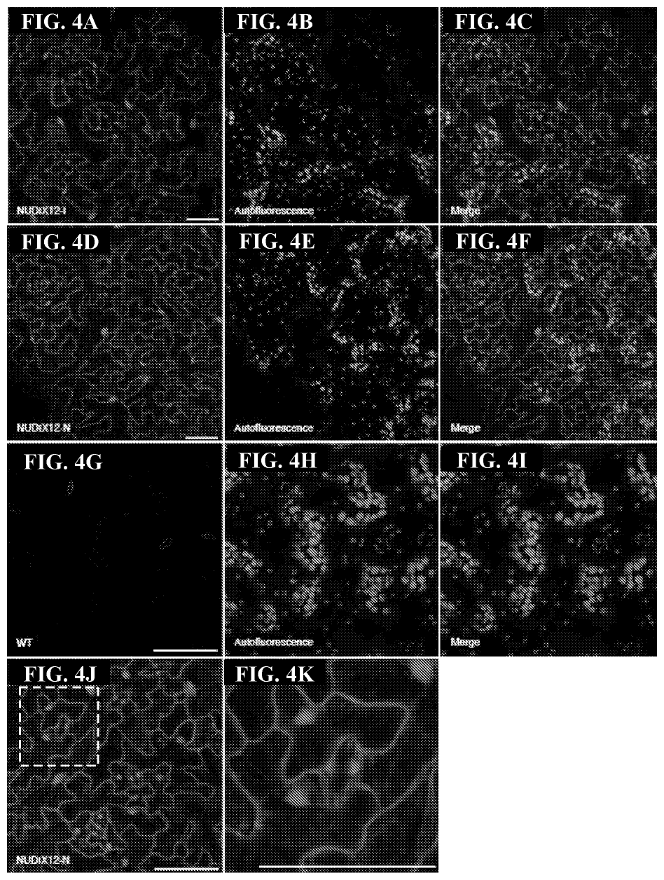

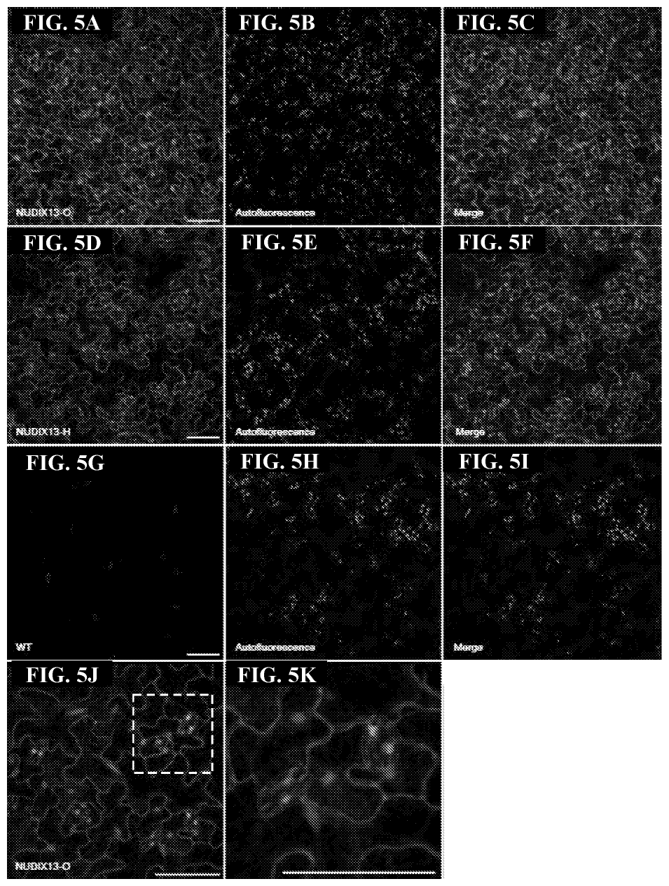

Given the reduction in PP-InsPs, Applicant explored whether NUDIX12 and NUDIX13 shared other common phenotypes with Arabidopsis plants containing perturbed PP-InsPs. Specifically, Applicant gauged whether NUDIX12 and NUDIX13 lines were similar to DDP1 OX in terms of subcellular compartmentalization and in Pi accumulation. To determine whether NUDIX12 and NUDIX13 are located in similar subcellular compartments as DDP1 and other native PP-InsP synthesis enzymes, Applicant examined subcellular compartmentalization in Applicant's stable Arabidopsis gain-of-function lines. While the native location(s) of PP-InsPs has yet to be determined in vivo, most PP-InsP synthesis enzymes and ectopically-expressed DDP1 localize to the cytoplasm and nuclei (Adepoju et al., 2019. This was especially important to determine based on previous studies showing different NUDIX13 subcellular localization patterns (Olejnik et al., 2007; Williams, 2015). NUDIX13 is predicted to be a mitochondrial enzyme and Olejnik et al demonstrates that NUDIX13-GFP localizes to the mitochondria in Arabidopsis protoplasts and yeast (Olejnik et al., 2007). However, an extensive confocal study shows transient expression of NUDIX12-GFP and NUDIX13-GFP in Nicotiana benthamiana localizes to leaf cell nuclei and in the cytoplasm (Williams, 2015). To reconcile these differences, stable NUDIX12-GFP and NUDIX13-GFP expression from intact Arabidopsis leaf cells was visualized using confocal microscopy. These data shows that both constructs localize to the cytoplasm and nuclei of Arabidopsis leaf epidermal cells (FIG. 4A-4K and FIG. 5A-5K). As expected, GFP signal was abundant in both NUDIX12 and NUDIX13 OX lines whereas nearly undetectable in WT (FIG. 3A-3C and FIG. 4A-4K). NUDIX12-GFP and NUDIX13-GFP expression was also high in guard cell nuclei (FIG. 4J-4K and FIG. 5J-5K). These results indicate that NUDIX12 and NUDIX13 stably localize to the same compartments as DDP1-GFP and other PP-InsP kinases (Williams, 2015; Adepoju et al., 2019).

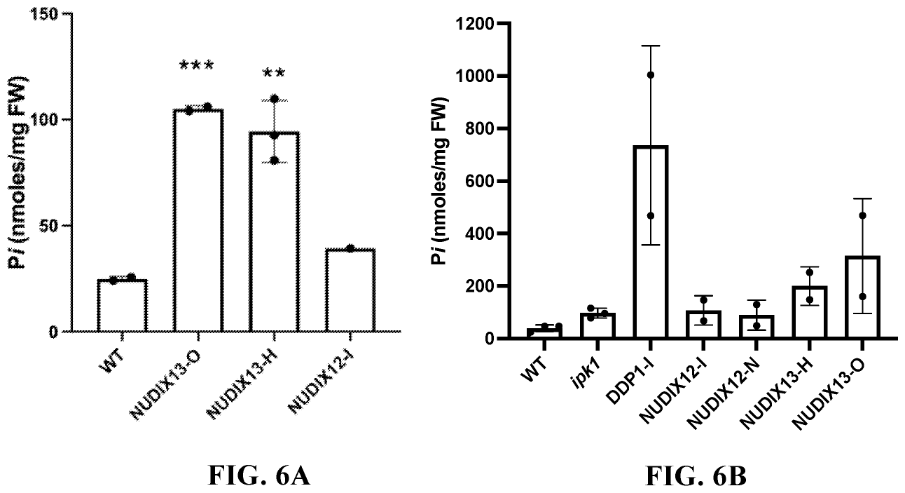

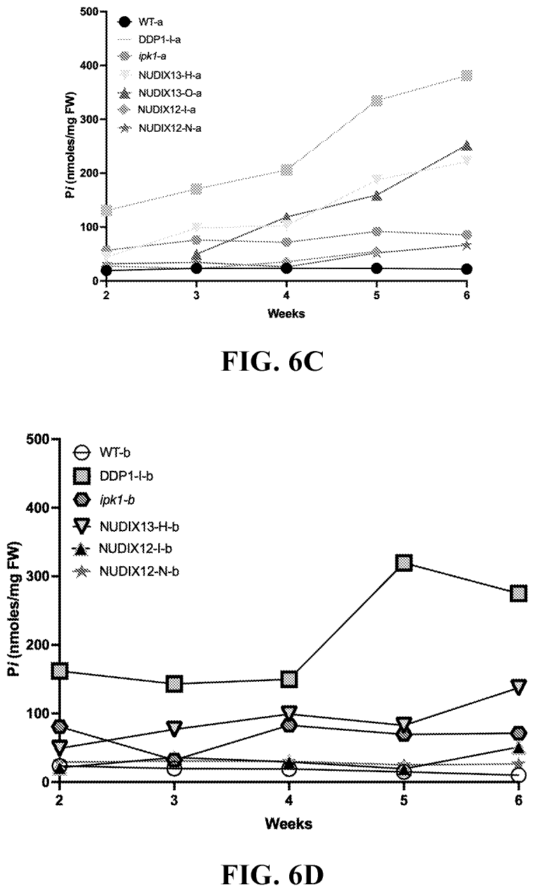

As elevated Pi accumulation is a hallmark of an elevated Phosphate Starvation Response, and is associated with a large reduction in PP-InsPs (Kuo et al., 2018; Zhu et al., 2018; Land et al., 2021), Applicant also measured Pi levels in leaf tissue from 2-6-week-old NUDIX12-I and NUDIX13 OX plants and in 6-week-old cauline leaf tissue (FIG. 6A-C). Both NUDIX13 OX lines accumulated approximately 3.4-5.6 times more Pi compared to WT plants after ˜4 weeks of growth and approximately 6.5-12 times more Pi compared to WT plants after 6 weeks (FIG. 6A, 6C-D). Applicant preliminarily quantified Pi in a single NUDIX12-I replicate and found that there was a slight increase in Pi accumulation compared to WT, however this increase is likely not significant. Preliminary data submitted by the applicant also suggests that both NUDIX13 OX lines accumulate Pi over time whereas NUDIX12 OX lines do not (FIG. 6C-D). Cauline leaf Pi accumulation preliminary data shows NUDIX13 OX lines had about 5-7 times more and NUDIX12 OX lines had about 2 times more Pi accumulation on average compared to WT cauline leaves.

### Group 9 NUDIX Enzymes Vary in Binding Capacity

Applicant used a molecular modeling approach to explore the substrate specificity of NUDIX13 and rationalize the protein structure-function relationships likely associated with substrate specificity. As the structure of NUDIX13 has yet to be resolved through protein crystallography, Applicant utilized a homology modeling and molecular docking process. The goal of this work was to determine whether PP-InsPs could effectively bind within the NUDIX13 catalytic pocket during molecular docking simulations. Applicant included all other NUDIX enzymes in group 9 based on their proximity to DDP1 on the phylogenetic tree (FIG. 1). Since not all NUDIX enzymes in group 9 contain the shared conserved “SS” region predicted to facilitate PP-InsP binding, adding all group 9 NUDIX enzymes could provide further clarity on differences in ligand binding between enzymes. The resolved crystal structure DDP1 (PDB ID: 7AUL) served as the positive control based on its experimentally verified enzymatic activity as well as the similarity in phenotypes of DDP1 OX and NUDIX13 OX Arabidopsis transgenics.

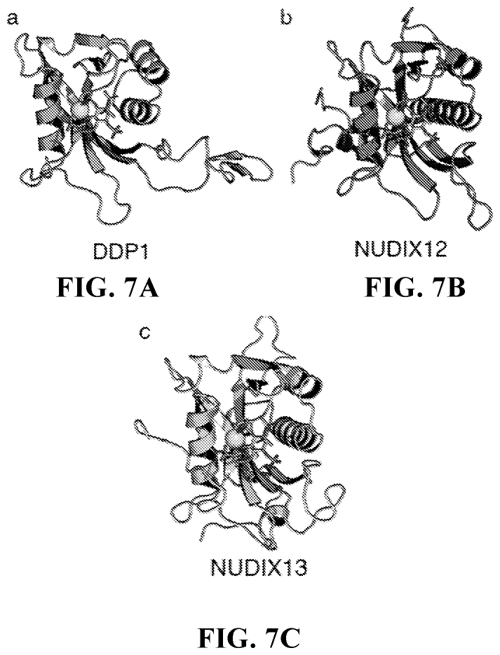

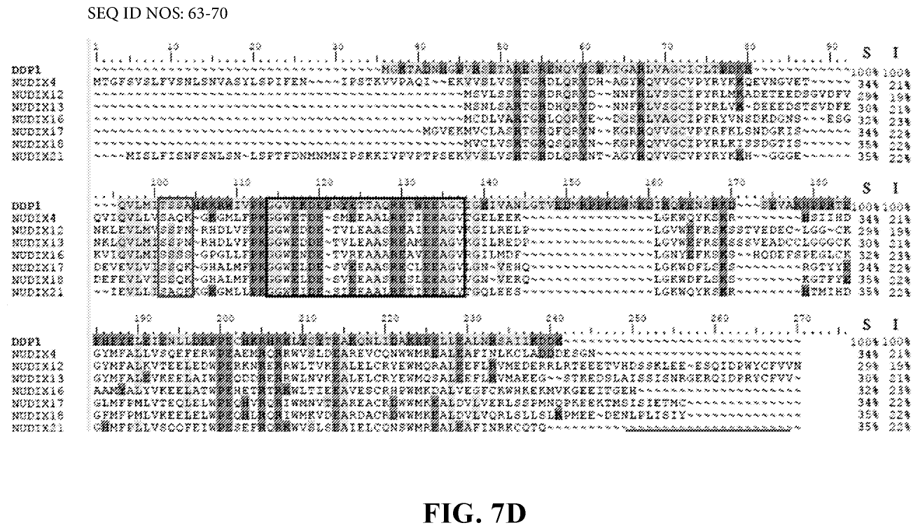

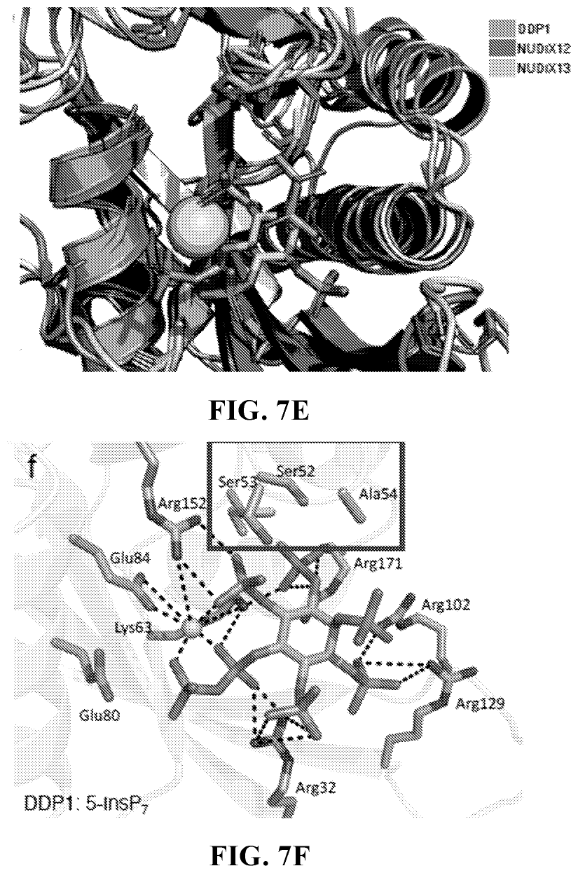

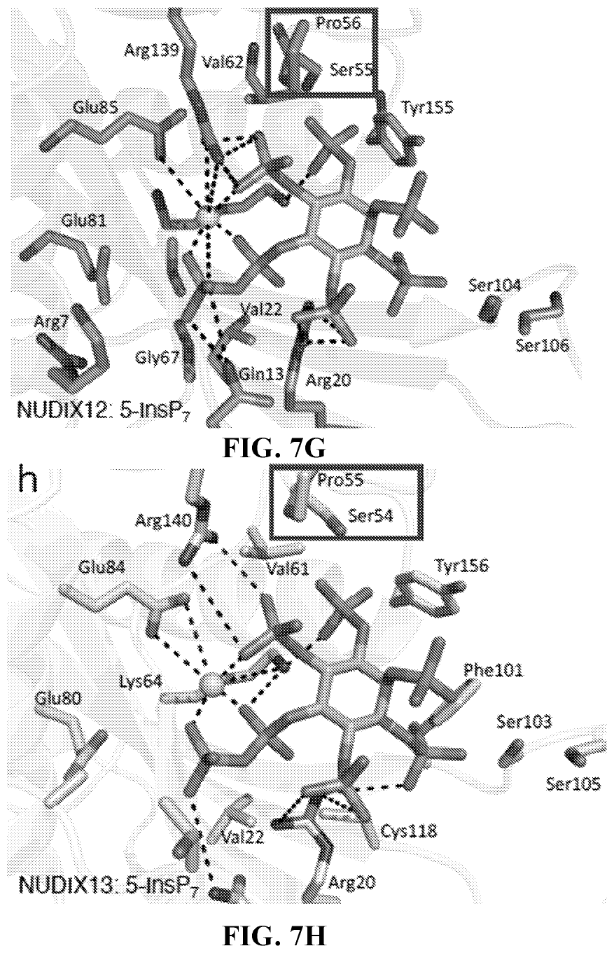

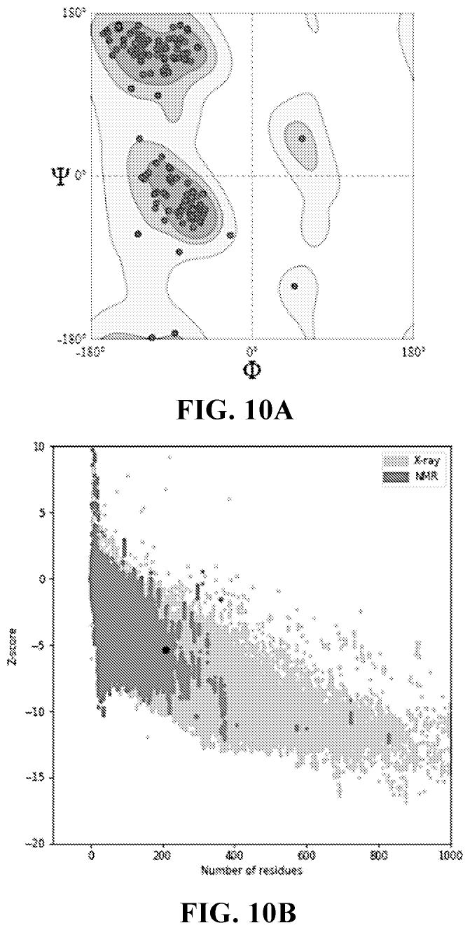

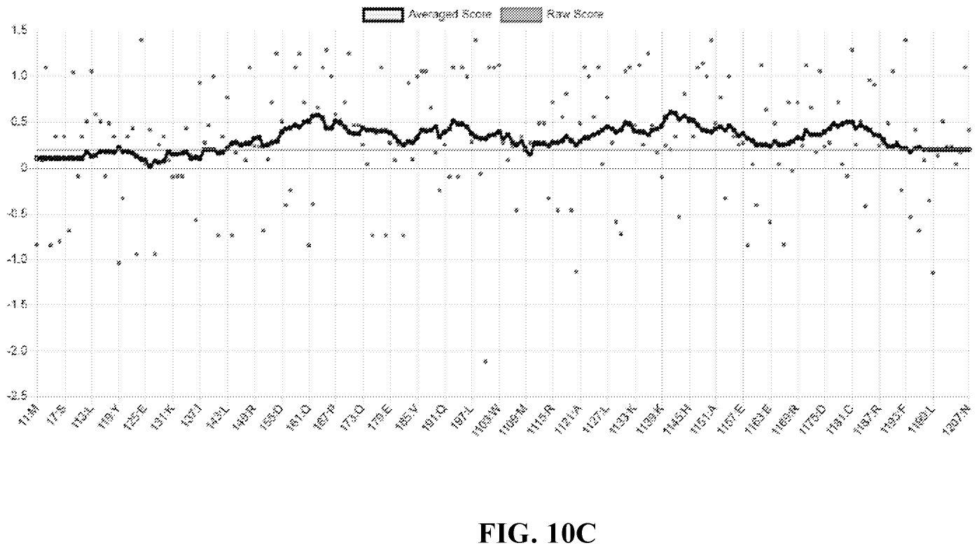

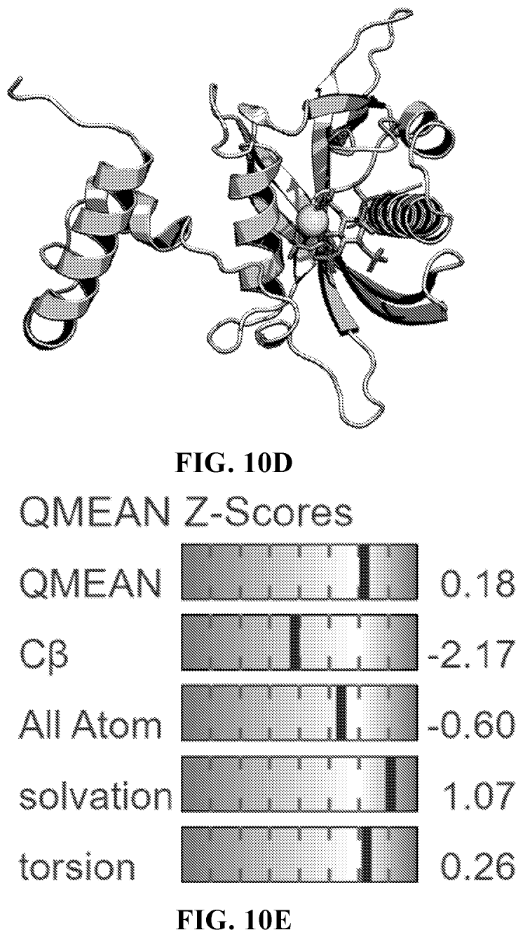

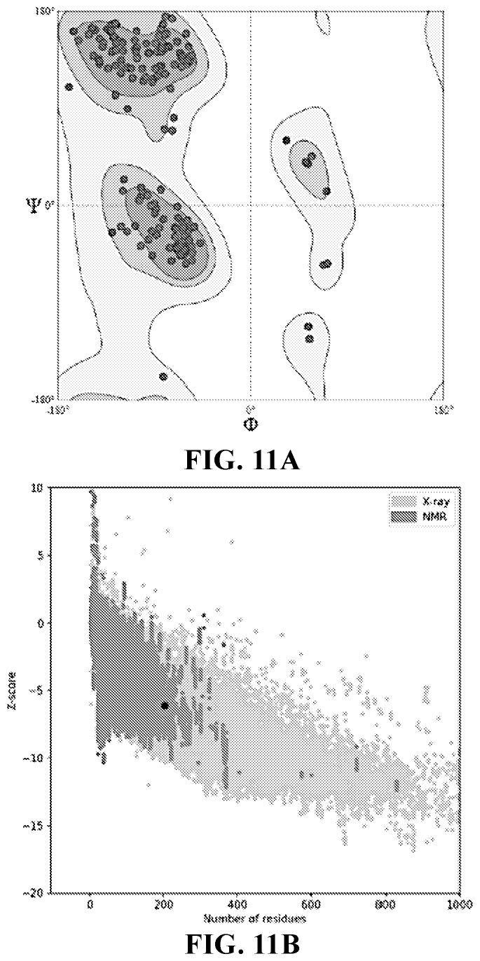

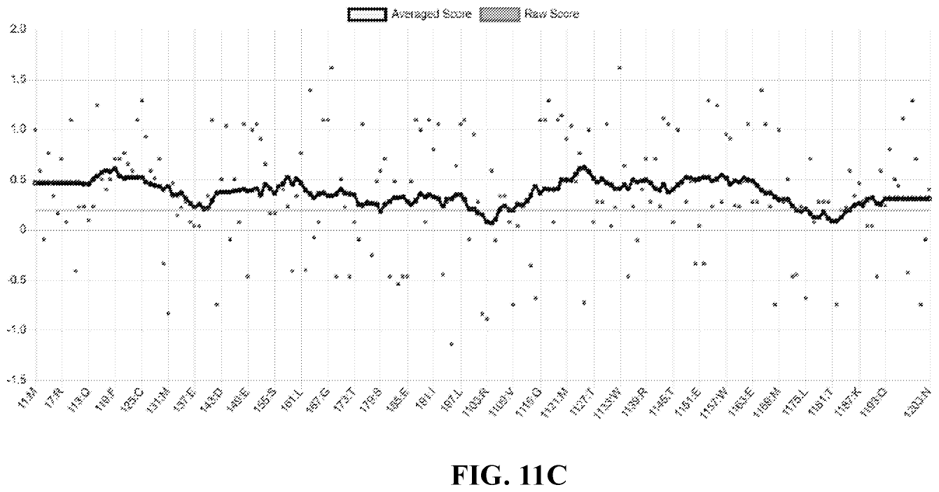

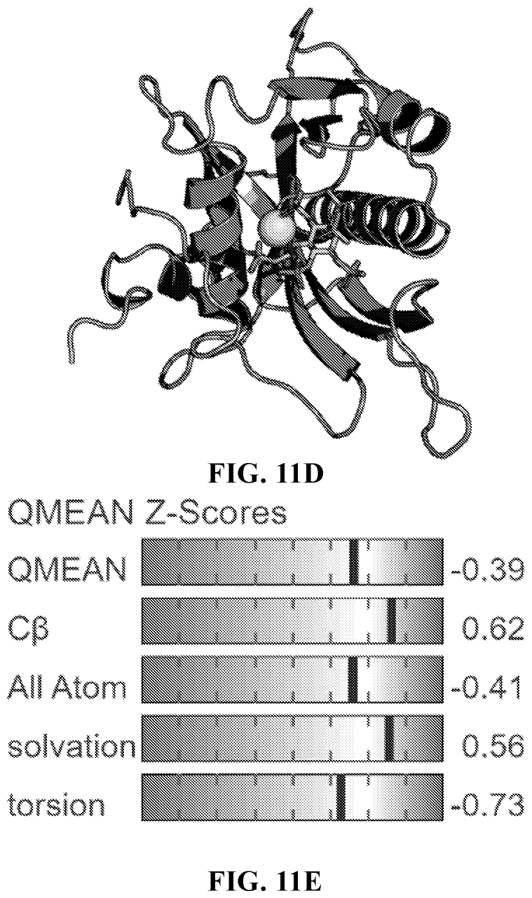

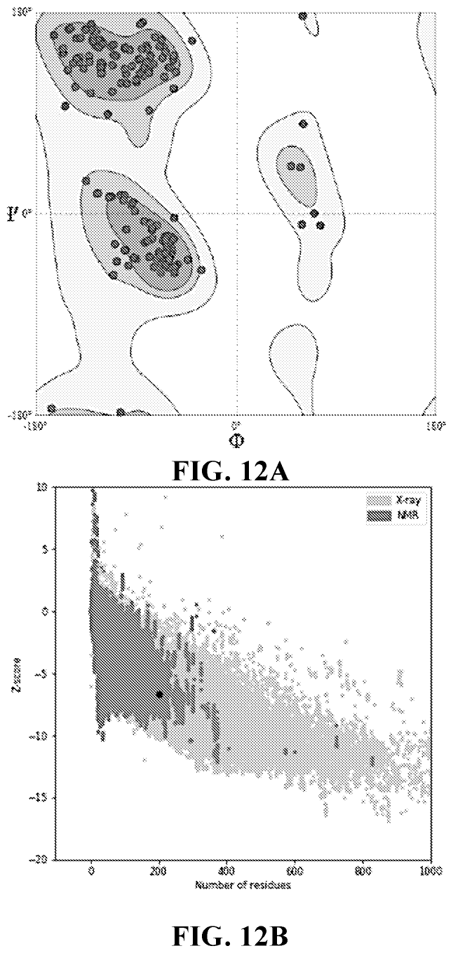

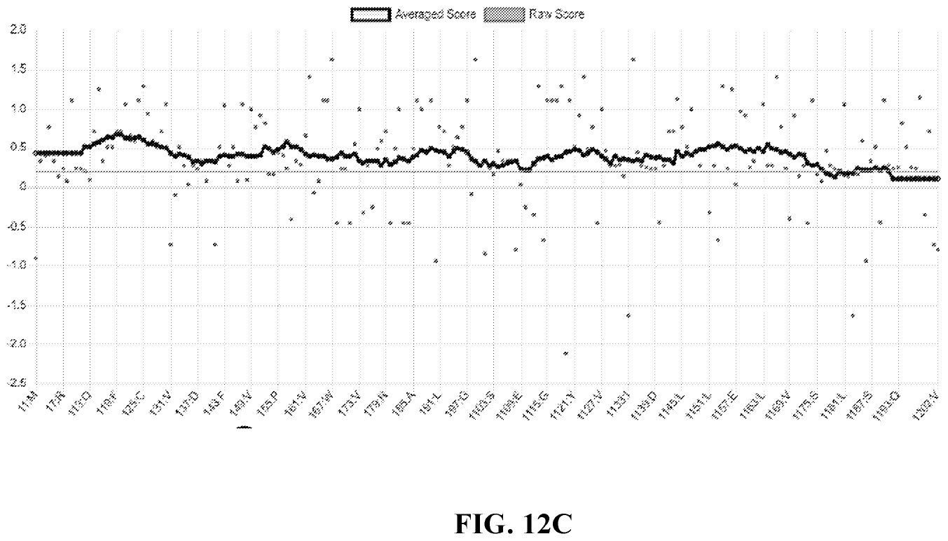

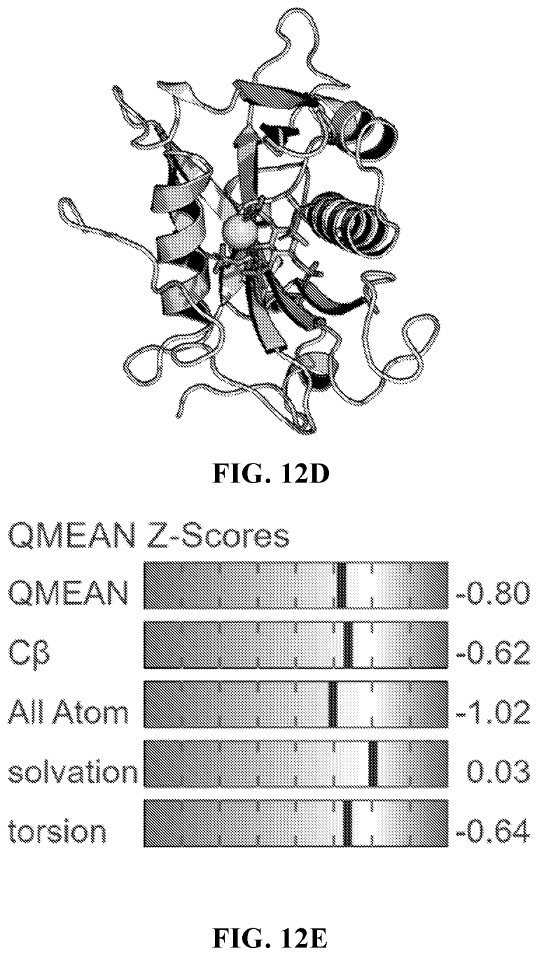

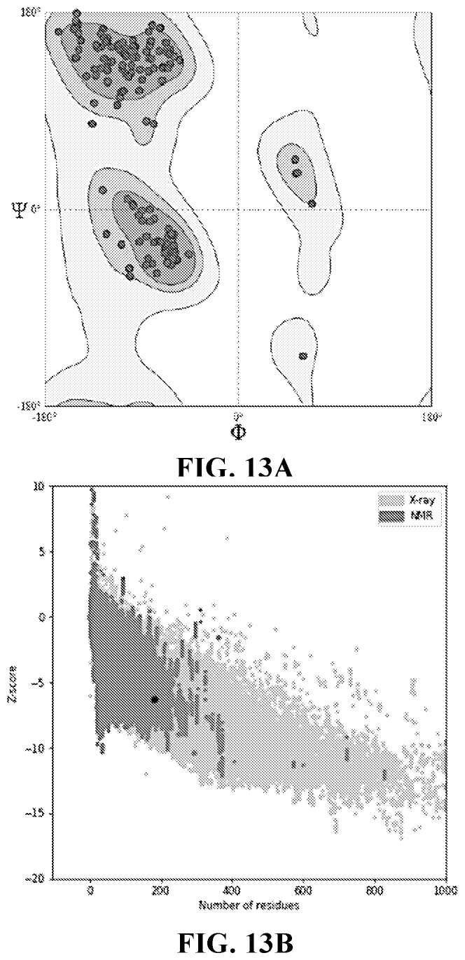

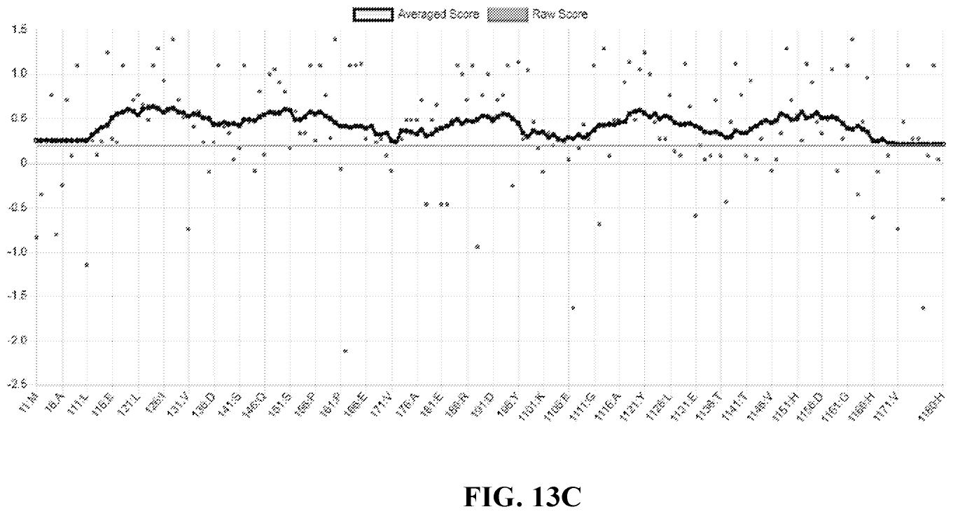

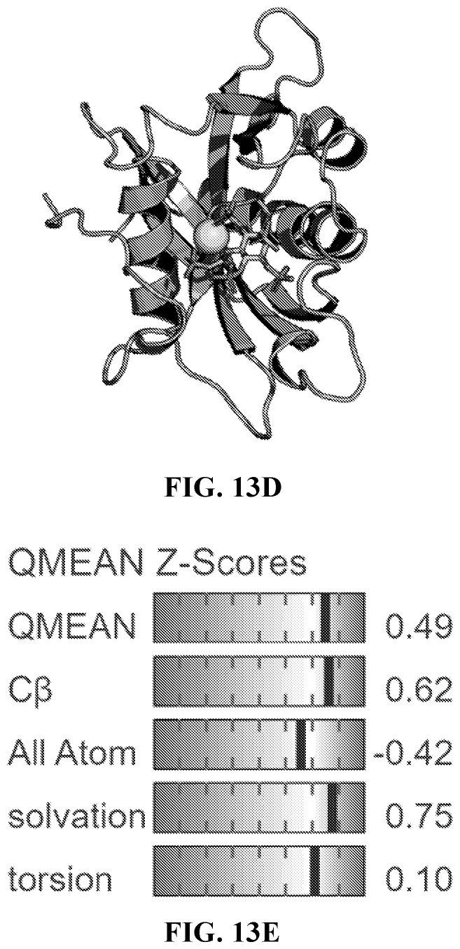

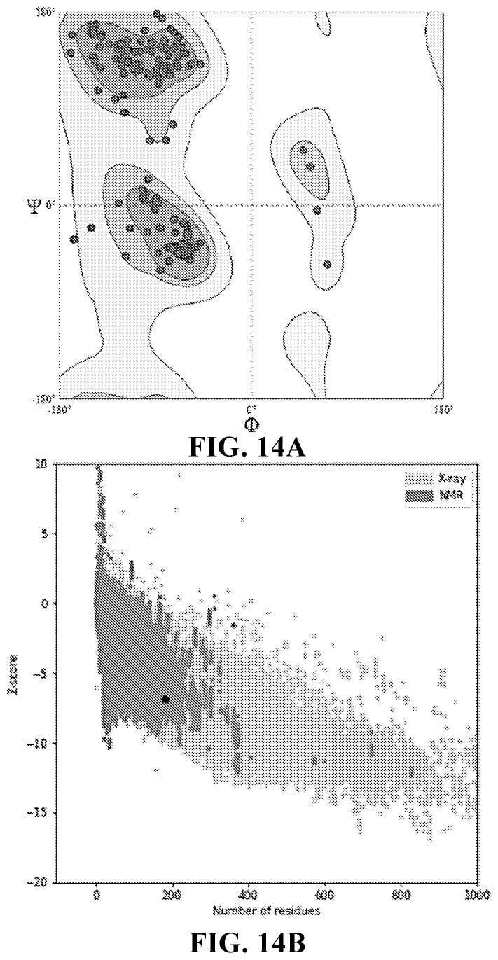

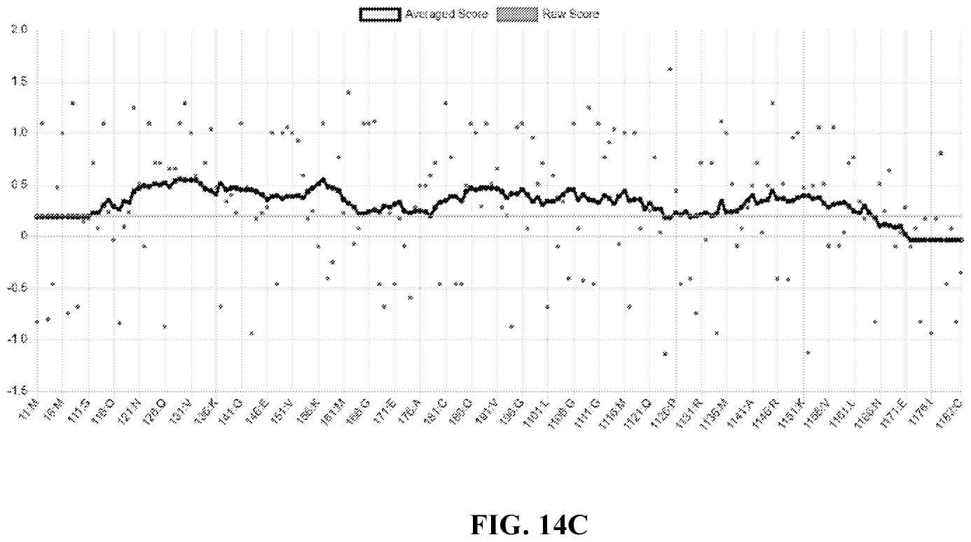

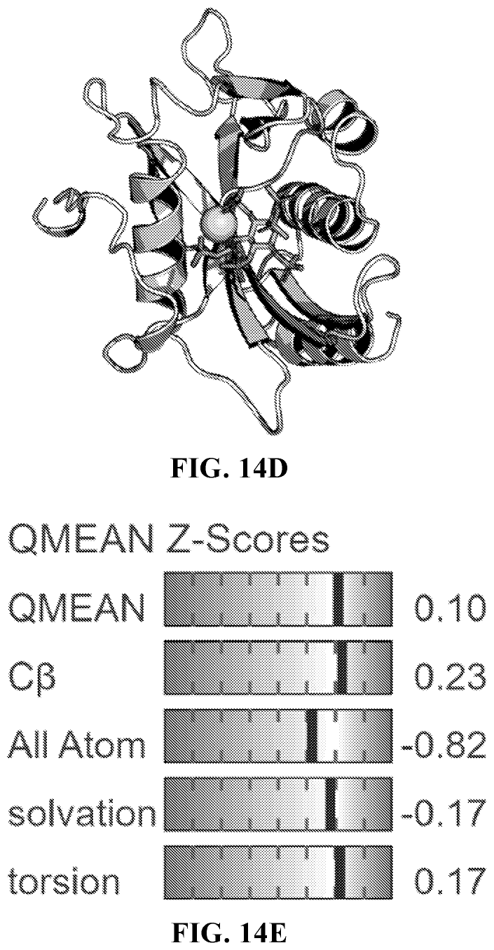

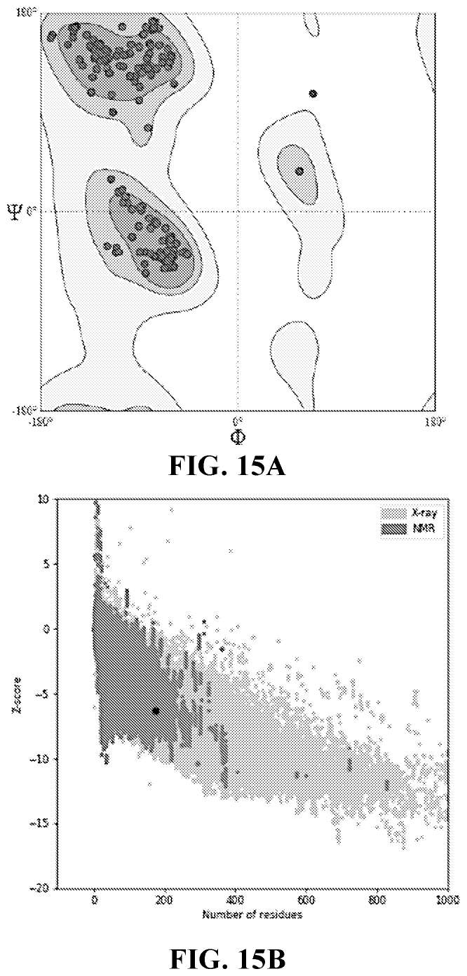

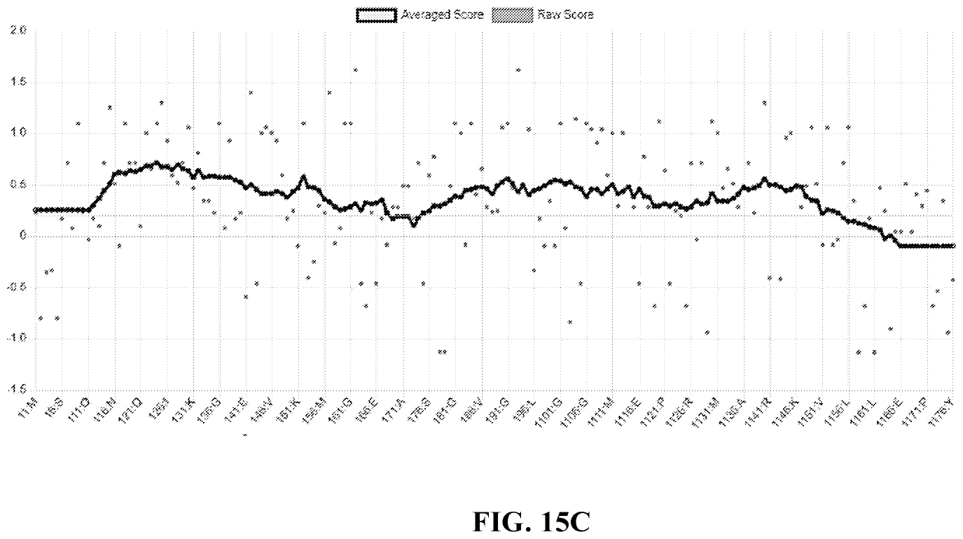

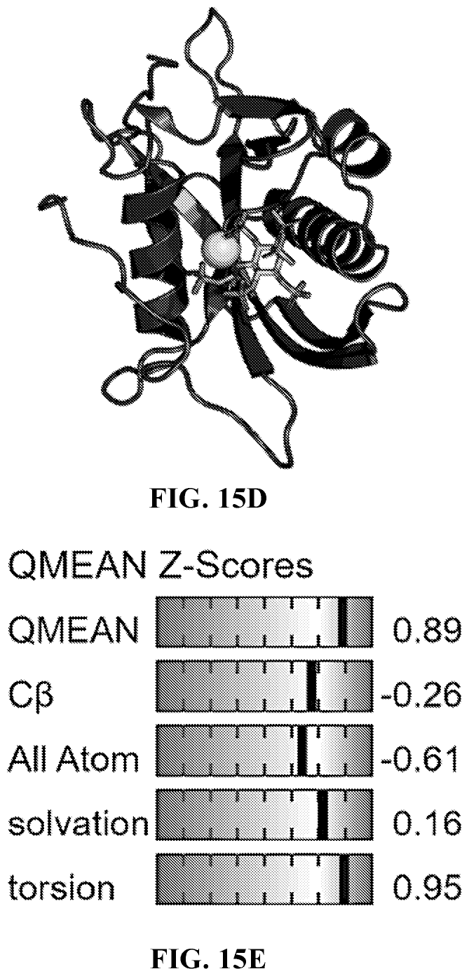

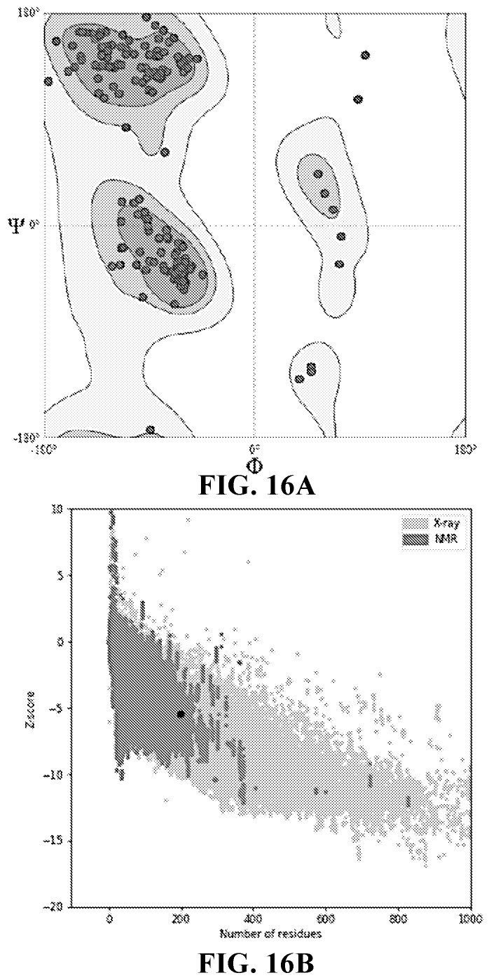

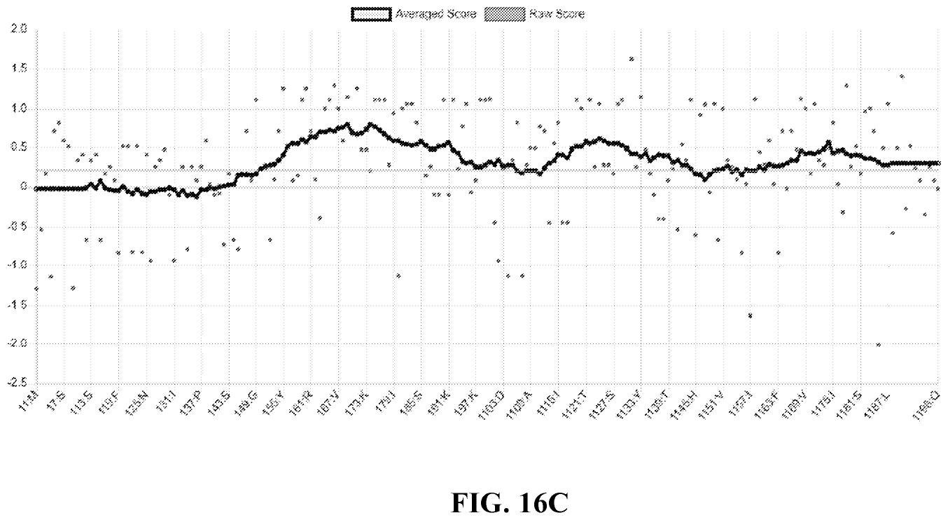

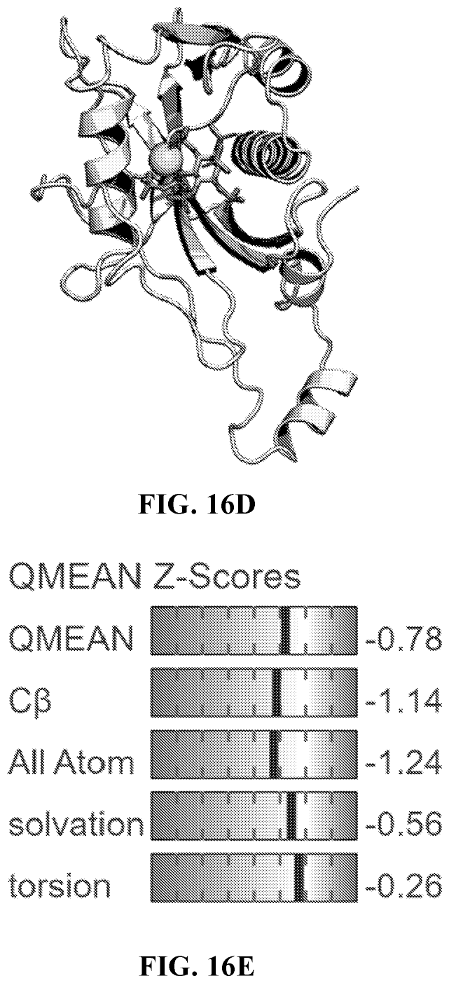

Seven total NUDIX enzymes homology models were generated using a protein structural predication server, RobeTTa fold (Baek et al., 2021) (FIGS. 7A-7H, 10A-10E, 11A-11E, 12A-12E, 13A-13E, 14A-14E, 15A-15E, and 16A-16E). Homology model quality was assessed using a series of structural validation techniques which include metrics to assess torsional energies (Kiefer et al., 2009), similarity to other resolved NMR and crystal structures (Wiederstein and Sippl, 2007), as well as the position of model side chains (Bowie et al., 1991) (FIGS. 10A-10E, 11A-11E, 12A-12E, 13A-13E, 14A-14E, 15A-15E, and 16A-16E). All NUDIX homology models had similar structures and binding pockets compared to crystallized DDP1 (FIGS. 7A-7H, 10A-10E, 11A-11E, 12A-12E, 13A-13E, 14A-14E, 15A-15E, and 16A-16E). Similarities in binding pockets were expected based on the conserved amino acids within the NUDIX box across all assessed enzymes (FIG. 7D) NUDIX13 shared 30% AA similarity and 21% AA identity with DDP1 (FIG. 7D). Pairwise sequence alignment of NUDIX12 and NUDIX13 show that these enzymes contain 84% AA similarity and 77% amino acid identity (FIG. 17). While the similarity between NUDIX12 and NUDIX13 is not surprising based on how close together these enzymes are located on the phylogenetic tree (FIG. 1), it is interesting given that PP-InsP signaling is more greatly impacted in Arabidopsis NUDIX13 OX than NUDIX12 OX transgenics. Homology models overlays indicate similarities and differences in structure (FIG. 7E) and interacting amino acids within the catalytic pocket (FIG. 7F-7H).

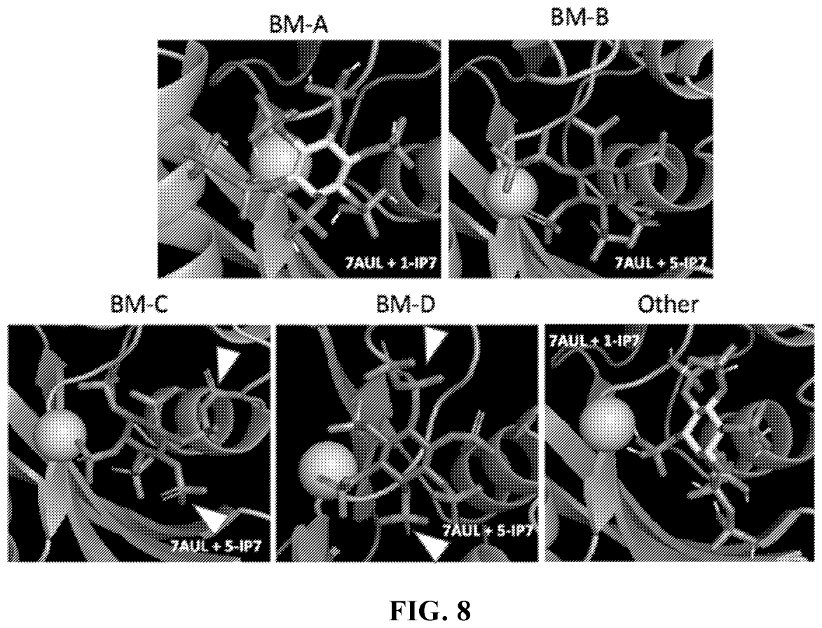

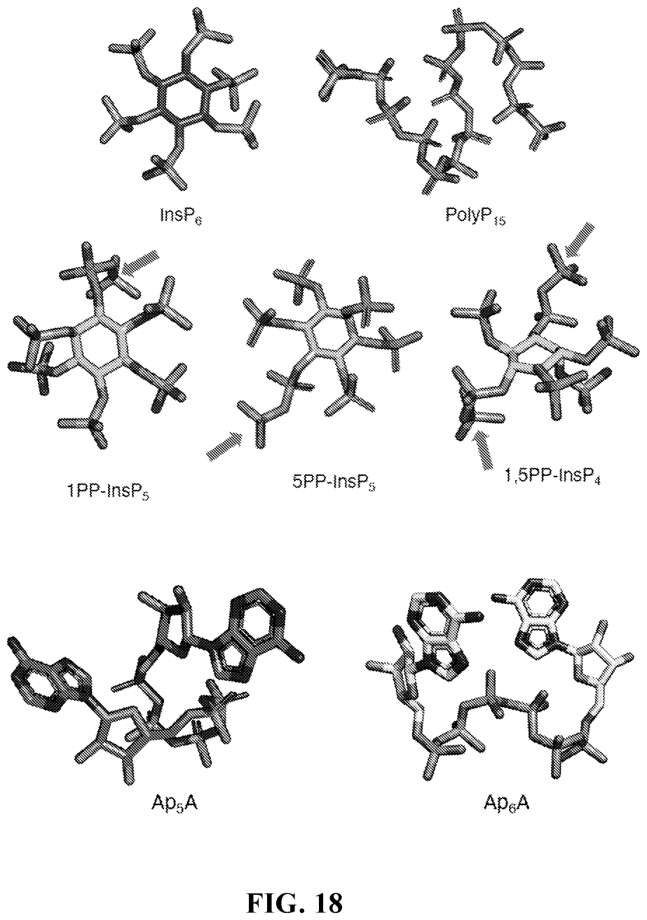

Ligands 1,5P2-InsP₄, 1PP-InsP₅, 5PP-InsP₅, and InsP₆ were docked into all group 9 NUDIX enzymes and DDP1 (FIG. 18). InsP₆ was used as a negative control. Applicant also assessed docking of ligand polyP₁₅ (FIG. 18). These ligand groups' analyses were added to the analysis based on the fact that DDP1 uses them as substrates. A short-chain polyP molecule of 15 Pi moieties (polyP₁₅) was used as longer-chain polyP molecules are not as reliable for docking experiments as are short-chain polyP molecules (Ferrucci et al., 2021). Notably, all of these molecules are highly symmetrical and can bind in multiple different positions within the binding pocket (FIG. 8). The binding modes with 1-InsP₇ and 5-InsP₇ will be defined as follows: “BM-A” with the PPi below the Mg²⁺ ion, “BM-B” with the PPi above the Mg²⁺ ion, “BM-C” with the PPi on one of the spots on the inositol ring farthest from the Mg²⁺ ion, “BM-D” with the PPi on the middle positions of inositol ring, and “other” where the InsP₇ molecule is not in a productive position for hydrolysis. Notably, Márquez-Moñino et al also observed different binding modes of PP-InsPs within in the DDP1 catalytic pocket in their crystallography study with DDP1 and noted some were more productive than others for hydrolysis (Márquez-Moñino et al., 2021). Additionally, Applicant was able to replicate the pose of 5-InsP₇ in DDP1 with redocking (RMSD) and observe similar data as presented in the structural work of Márquez-Moñino (Márquez-Moñino et al., 2021) (see Experimental Procedures section in this Example).

Based on the predicted free-energy molecular mechanics generalized Born model and solvent accessibility method (MMGBSA), polyP₁₅ appeared to bind more favorably than PP-InsPs and InsP₆ for all group 9 enzymes (Tables 4 and 6). Since InsP₆ is the negative control, Applicant selected the predicted free-energy MMGBSA from DDP1:InsP₆ (−9.3 kcal/mol) as the cutoff for favorable binding. The data indicates that group 9 enzymes with the conserved SS region (NUDIX12, 13, 16, 17, and 18) are not predicted to be efficient at binding to PP-InsPs. Intriguingly, NUDIX12 and NUDIX13 had approximately the same predicted free-energy MMGBSA for all ligands tested. NUDIX16 was more similar to DDP1 with average predicted free-energy MMGBSA for InsP₈ and 5-InsP₇ above the cutoff, except 1-InsP₇ was right below the cutoff at −8.6 kcal/mol and InsP₆ was above at −10.9 kcal/mol. NUDIX17 and NUDIX18 predicted free-energy of binding values were very close to NUDIX12 and NUDIX13 except they had the highest favorability for polyP₁₅ compared to DDP1. NUDIX17 and NUDIX18 also had mostly positive predicted free-energy of binding values for all PP-InsPs and InsP₆ except NUDIX18 with 1-InsP₇ at −11.3 kcal/mol. Interestingly, the group 9 two enzymes without the SS region (NUDIX4 and NUDIX21) appeared to be the most similar to DDP 1 in terms of predicted ligand preferences, with NUDIX4 being the closest. NUDIX21 also had predicted binding free-energy values similar to DDP1 for all ligands with exception of InsP₈ at −8.3 kcal/mol.

Table 4 shows the average predicted free-energy of binding for each enzyme and ligand pair. Values are the average of the top three predicted free-energy of binding calculations on ligand/enzyme complexes generated MM-GBSA unless otherwise noted with a † as not all binding modes were energetically favorable or productive. Units for all averaged values are kcal/mol. Values greater than −9.5 are marked with an * (this cutoff is selected based as DDP1:InsP₆ binding, the negative control, is −9.3 kcal/mol). Standard deviations of all averaged predicted free-energies are included in an extensive table in Table 6.

Values in Table 6 are the average of the top three predicted free-energy of binding calculations on ligand/enzyme complexes generated MM-GBSA unless otherwise noted with a † as not all binding modes were energetically favorable or productive. Units for all averaged values are kcal/mol. Values less than −9.5 are designated with an * and bolded (this cutoff is selected based as DDP1:InsP₆ binding, the negative control, is −9.3 kcal/mol. The lower the value, the more energetically favorable the predicted free-energy of binding).

Table 5 provides oligonucleotides used in Example 1.

TABLE 4
Average predicted free-energy of binding
for each enzyme and ligand pair.

| | DDP1 (Ref.) | NUDIX4 | NUDIX12 | NUDIX13 |
|---|---|---|---|---|
| PolyP15 | −49.3 | −56.3* | −65.4* | −50.0* |
| InsP8 | −32.5 | −21.4* | −7.5 | −1.4† |
| 1-InsP7 | −22.7 | −17.6* | −1.2 | 3.4† |
| 5-InsP7 | −22.4 | −17.6* | −0.5† | 4.6† |
| InsP6 | −9.3 | −7.9 | −0.5† | 10.7† |

| | NUDIX16 | NUDIX17 | NUDIX18 | NUDIX 21 |
|---|---|---|---|---|
| PolyP15 | −57.3* | −79.5* | −62.6* | −66.8* |
| InsP8 | −17.6* | 10.8† | −3.5 | −8.3 |
| 1-InsP7 | −8.6 | 1.1† | −11.3* | −11.8*† |
| 5-InsP7 | −12.8* | 0.8† | −2.9 | −11.8*† |
| InsP6 | −10.9* | 7.5† | 6.3 | −7.1 |

TABLE 5
Oligonucleotide primers used in Example 1

| | | | Sequence (5′→3′) | Product |
|---|---|---|---|---|
| Gene Name | Organism | Primer | (SEQ ID NOS: 56-61) | Size |
| DDP1 | S. | Forward | CACCATGGGCAAAACCGCGGATAAT | 560 bp |
| (YOR136w) | cerevisiae | Reverse | TTTGTCGTCTTTAATGATAGCAGACCT | |
| NUDIX12 | A. | Forward | CACCATGTCGGTTCTTTCTTCTCGGACA | 612 bp |
| (At1g12880) | thaliana | Reverse | CTAGTTAACTACAAAACAGTACCA | |
| NUDIX13 | A. | Forward | CACCATGTCGAATCTTTCTGCAAGAACA | 609 bp |
| (At3g26690) | thaliana | Reverse | TTAGACTACAAAGCAGTAGCGAGG | |

TABLE 6
Predicted free-energy of binding averages and standard
deviations for each enzyme and ligand pair

| | DDP1 | | | | | | | |
|---|---|---|---|---|---|---|---|---|
| | (Reference) | | NUDIX4 | | NUDIX12 | | NUDIX13 | |
| | | Std | | Std | | Std | | Std |
| | Average | Dev | Average | Dev | Average | Dev | Average | Dev |
| Poly(P)15 | −49.3 | ±5.8 | −56.3 | ±8.4 | −65.4 | ±7.1 | −50.0 | ±4.6 |
| InsP8 | −32.5 | ±4.1 | −21.4 | ±4.7 | −7.5 | ±0.9 | −1.4† | 0.0 |
| 1-InsP7 | −22.4 | ±6.1 | −17.6 | ±1.4 | −0.5 | ±1.1 | 4.6† | 0.2 |
| 5-InsP7 | −22.7 | ±2.7 | −17.6 | ±3.6 | −1.2† | ±0.8 | 3.4† | 1.2 |
| InsP6 | −9.3* | ±2.1* | −7.9 | ±1.5 | −0.5† | ±0.7 | 10.7† | 0.0 |
| | NUDIX16 | | NUDX17 | | NUDIX18 | | NUDIX21 | |
| | | Std | | Std | | Std | | Std |
| | Average | Dev | Average | Dev | Average | Dev | Average | Dev |
| Poly(P)15 | −57.3 | 7.0 | −79.5 | 6.7 | −62.6 | 13.2 | −66.8 | 3.9 |
| InsP8 | −17.6 | 9.2 | 10.8† | 0.0 | −3.5 | 1.8 | −8.3 | 4.0 |
| 1-InsP7 | −12.8 | 5.2 | 0.8† | 2.1 | −2.9 | 0.2 | −11.8† | 4.5 |
| 5-InsP7 | −8.6 | 3.3 | 1.1† | 1.7 | −11.3 | 0.9 | −11.8† | 4.5 |
| InsP6 | −10.9 | 9.6 | 7.5† | 2.5 | 6.3 | 0.5 | −7.1 | 0.7 |

### DISCUSSION

### NUDIX13 Overexpression Perturbs PP-InsP Signaling

Stable overexpression of NUDIX13 in Arabidopsis decreases PP-InsP levels and phenocopies the impact of DDP1 overexpression in plants. Specifically, data in this Chapter shows that NUDIX13-GFP localizes to the same compartment as DDP1-GFP, and both NUDIX13 and DDP1 stimulate an increase in Pi accumulation. NUDIX12 overexpression also decreases PP-InsP levels, however, NUDIX13 overexpression more significantly impacts PP-InsP accumulation and signaling as compared to NUDIX12 (FIGS. 3A-3C, 17, 12A-12E and Table 3). This is an especially interesting result given the close evolutionary relationship and amino acid identity shared by NUDIX12 and NUDIX13 (FIGS. 1 and 9). Based on the westerns and subcellular localization data, NUDIX12-I and NUDIX12-N lines accumulate similar levels of GFP-tagged protein (FIG. 2A-2D) within the same subcellular compartments (FIGS. 4A-4K and 5A-5K) (Williams, 2015) as the NUDIX13 OX lines, but do not show the same Pi-linked phenotypes (FIGS. 2A-2D and 6A-D). Taken together, this strongly suggests that NUDIX13 has a greater impact on PP-InsP signaling in a gain-of-function as compared to NUDIX12.

Applicant's work characterizing NUDIX13 OX and DDP1 transgenic phenotypes along with that of ipk1 mutants (Kuo et al., 2014; Kuo et al., 2018) and vih1-2vih2-4 mutants (Zhu et al., 2019) suggests an important threshold for InsP₇ and InsP₈ reduction to impact Pi sensing. Arabidopsis transgenics expressing high levels of DDP1 and NUDIX13 lead to a similar and proportional decrease in PP-InsPs and severe Pi-linked phenotypes (FIGS. 3A-3C, 6A-6D and Table 3). This idea of a threshold for Pi sensing is further supported by plants overexpressing NUDIX12, which had 64±19% of the total WT InsP₇ pool and 69±7% of the total WT InsP₈ pool, which did not perturb Pi accumulation. In contrast, InsP profiles from ipk1 plants indicate a similar reduction in PP-InsPs as compared to DDP1 OX transgenics, which is accompanied by changes in Pi-accumulation (Laha et al., 2015; Land et al., 2021). However, it is important to note that changes in DDP1 OX lines are more severe in Pi-linked phenotypes than ipk1 mutants, and ipk1 mutants also have greatly decreased InsP₆ and elevated InsP₅ levels (Stevenson-Paulik et al., 2005; Kuo et al., 2014; Kuo et al., 2018). Notably, characterized vih1-2vih2-4 mutants have no detectable levels of InsP₈ and doubled InsP₇ compared to WT (Zhu et al., 2019), and the growth phenotypes appear to have a more severe growth tradeoff compared to DDP1 OX transgenics.

### PP-InsPs May not be Ideal Substrates for NUDIX13

Molecular docking data suggests that PP-InsPs may not be the ideal substrates for NUDIX13 compared to polyP molecules. This result was surprising the given structural similarity of NUDIX13 homology to DDP1 (FIG. 7A-7H) and similarities in NUDIX13 OX and DDP1 OX gain-of-function phenotypes (FIG. 2A-2D). Another interesting result is that NUDIX12 and NUDIX13 have similar predicted free-energy MMGBSA values, considering their functionality in vivo. Based on the predicted free-energy MMGBSA, polyP is the optimal predicted substrate for DDP1 and all NUDIX enzymes assessed. This result was expected for DDP1 given a past study showing DDP1 is more efficient at hydrolyzing polyP compared to PP-InsPs (Lonetti et al., 2011). However, it is an unexpected finding for the plant NUDIX enzymes as polyP has not been detected in any plant species, to date.

When comparing all the group 9 NUDIX enzymes to DDP1, NUDIX4 and NUDIX16 would be the optimal group 9 NUDIX enzymes to target for hydrolyzing InsP₈ based on the respective predicted free-energy MMGBSA values of −21.4 kcal/mol and −17.6 kcal/mol. This work also highlights that specific NUDIX enzymes may have differing binding preferences for specific InsP₇ isomers. For example, NUDIX18 had a favorable predicted free-binding MMGBSA for 1-InsP₇ (−11.3 kcal/mol), 5-InsP₇ (−2.9 kcal/mol) and InsP₈ (−3.5 kcal/mol). However, NUDIX18:5-InsP₇ and NUDIX18:InsP₈ were unfavorable given the DDP1:InsP₆ cutoff value of −9.3 kcal/mol. This result suggests that NUDIX18 could be involved in hydrolysis of 1-InsP₇ rather than InsP₈ and 5-InsP₇. Additionally, both NUDIX4 and NUDIX21 had identical predicted free-binding MMGBSA values for 1-InsP₇ and 5-InsP₇. Interestingly, DDP1 also showed nearly identical predicted free-binding MMGBSA values for both InsP₇ isomers though in vitro.

DDP1 more effectively binds and hydrolyzes 1-InsP₇ as compared to 5-InsP₇ (Kilari et al., 2013; Márquez-Moñino et al., 2021). Thus, determining the substrate specificity for NUDIX enzymes with InsP₈ and InsP₇ isomers will be important for understanding group 9 NUDIX roles in PP-InsP degradation and signaling.

The molecular modeling data also suggests that NUDIX4 could be the optimal NUDIX enzyme for hydrolyzing PP-InsPs in vivo, rather than NUDIX12 and NUDIX13. There are two possible explanations for the ability of NUDIX13 to decrease PP-InsPs in vivo. First, since NUDIX13 was abundantly overexpressed in Arabidopsis using a strong, constitutive CaMV 35S promoter, it is possible that even if InsP₇ and InsP₈ are not the ideal substrates, that excessive NUDIX13 expression leads to PP-InsP degradation. If this were found to be the case and without being bound by theory, Applicant would expect that NUDIX12 has a weaker preference for hydrolyzing PP-InsPs compared to NUDIX13. The second possible explanation is that NUDIX13 overexpression does not directly hydrolyze PP-InsPs, but rather hydrolyzes another molecule that impacts PP-InsP signaling. There is a paradigm for this type of regulation in yeast, where the PP-InsPs regulate production of polyP, which regulates Pi homeostasis (Eskes et al., 2018). The molecular modeling analysis strongly suggests that polyP, rather than PP-InsPs, is an ideal substrate. The possibility that NUDIX13 hydrolyzes polyP molecule rather than PP-InsPs, while a possibility, seems unlikely as only PP-InsPs, rather than polyP, have been detected in plants (Desai et al., 2014; Laha et al., 2015), and have known synthesis pathways (Desai et al., 2014; Laha et al., 2015; Adepoju et al., 2019; Laha et al., 2019). There have been great efforts made, especially for polyP, to detect these molecules in planta (Lorenzo-Orts et al., 2020; Zhu et al., 2020) however these molecules remain elusive.

### NUDIX13 Functionality in Planta

This Example provides compelling evidence that NUDIX13, and NUDIX12 to a lesser extent, impact PP-InsP signaling in Arabidopsis. The similarities of group 9 NUDIX enzymes to DDP1 would also suggest that at least the other group 9 enzymes may also impact PP-InsP signaling. While NUDIX4 may be the optimal enzyme to hydrolyze PP-InsPs according to the molecular modeling data, previous work shows that NUDIX4 and NUDIX21 localize in transient expression experiments to the chloroplasts rather than the cytoplasm or nuclei (Williams, 2015). This work combines two approaches to preliminarily target a subset of NUDIX enzymes and their involvement in plant PP-InsP signaling.

### Experimental Procedures

### Phylogenetic Analyses and Sequence Alignments

DDP1 and all putative Arabidopsis NUDIX amino acid sequences were aligned using Clustal Omega (Sievers et al., 2011). Phylogenetic trees were constructed in Geneious Prime 2022.0.1 (Geneious, 2022) using RAxML 8.2.11 (Stamatakis, 2014) with protein model GAMMA BLOSUM62 and 1000 bootstraps. All NUDIX and DDP1 amino acid sequences were retrieved from uniprot.org (The UniProt Consortium, 2021) and aligned using Schrödinger-Maestro 2020-2 (Schrödinger, 2021). A pairwise alignment was used to compare NUDIX12 and NUDIX13 and a multiple sequence alignment was used to align group 9 NUDIX enzymes and all 27 NUDIX enzymes with DDP1.

### Cloning and Transformation

NUDIX12 (At1g12880) and NUDIX13 (At3g26690) from Arabidopsis thaliana and DDP1 (YOR163w) from Saccharomyces cerevisiae were PCR amplified for ligation into Gateway pENTR/D-TOPO entry vector. Using the Gateway® LR Clonase™ II kit (Invitrogen Corp., Carlsbad, CA), pENTR clones were recombined with Gateway plant destination vectors pK7FWG2 (C-terminus NUDIX12-GFP, NUDIX13-GFP and DDP1-GFP) (VIB UGent Center for Plant Systems Biology). E. coli transformed with these constructs was selected on antibiotic media. Plasmids purified from resulting colonies were sequencing for verification of correct cloning. Agrobacterium (strain GV3101) was transformed with these constructs and were used for transient Nicotiana benthamiana experiments or to transform Arabidopsis thaliana (ecotype Col-0) plants. benthamiana experiments or to transform Arabidopsis thaliana (ecotype Col-0) plants.

### Plant Growth Conditions

All transgenics and WT Arabidopsis lines were identically grown in concert on soil under long day conditions (16 hours light/8 hours dark, 55% humidity, day/night temperature 23/21° C. and 120 μE light intensity). Seedlings grown under sterile conditions were first sterilized using 100% ethanol for one minute, transferred to a 30% (v/v) Clorox solution for 5 minutes and washed five times with ddH₂O. Seeds were placed in 0.5×MS media+0.2% agar and stratified for 3 days at 4° C. Seeds were either transferred to 0.5×MS solid media plates or multi-well plates containing semi-solid media (0.2% agar). All data was analyzed using JMP Pro and GraphPad Prism.

### Protein Blots of GFP-Fusion Proteins

Protein blots were performed as previously reported (Burnette et al., 2003). Leaf tissue from soil grown plants from was pulverized in liquid nitrogen, homogenized, and separated from cell debris. SDS-bromophenol blue loading dye was added to the extracted proteins and boiled for 5 minutes at 85° C. After a subsequent centrifugation, the supernatant was loaded onto a polyacrylamide gel; equal amounts of protein were added from each sample. For western blotting, a 1:5000 dilution of anti-GFP antibody (Invitrogen Molecular Probes, Eugene, OR) and a 1:2000 dilution of secondary goat anti-rabbit horseradish peroxidase antibody (Bio-Rad Laboratories, Hercules, CA) were used to detect GFP.

### InsP and PP-InsP Profiles of Arabidopsis Seedlings

WT, NUDIX12, and NUDIX13 OX transgenics were grown in semi-solid 0.5×MS media and 0.2% agarose for two weeks, 30 μE light intensity. 24 seedlings of each genotype were transferred to Eppendorf tubes containing 300 μL of 0.5×MS media, 0.2% agar and 50 μCi of [³H] myo-inositol (20 Ci/mmol; American Radiolabeled Chemicals (ARC), St. Louis, MO, USA) for 4 days. InsPs were extracted from seedlings by pulverizing tissue in extraction buffer (1 M Perchloric acid (HC104), 3 mM EDTA, and 0.1 mg/ml InsP₆) and vortexed with glass beads. The pH of the extract was neutralized to pH 6-8 using a neutralization buffer (3 mM EDTA and 1M K₂CO₃). Samples were dried to a volume of 80-100 μL. Negatively charged species were separated via HPLC using a 125×4.6 mm Partisphere SAX-column (Sepax Technologies, Delaware, USA) and an ammonium phosphate elution gradient (Azevedo and Saiardi, 2006). Radioactivity in all collected fractions was quantified using a scintillation counter.

### Subcellular Localization and Imaging

All Arabidopsis and N. benthamiana cells were imaged using a Zeiss LSM 880 confocal microscope (Carl Zeiss) equipped with a 25× C-Apochromat water immersion lens. N. benthamiana plant leaves were infiltrated with transformed with Agrobacterium tumefaciens, as previously described (Kapila et al., 1997). Specifically, Agrobacterium cells were grown overnight, pelleted, and resuspended in MMA (10 mM MES, 10 mM MgCl₂, 200 μM acetosyringone) solution at an OD₆₀₀ of 1.0. After a 2-4 hour incubation period in the dark, N. benthamiana leaves were infiltrated with the Agrobacterium MMA cultures. Leaf sections were imaged after 24, 48, and 72-hours post infiltration. GFP was excited using a 488-nm argon laser and its fluorescence was detected at 500- to 550-nm and YFP was excited using a 488-nm argon laser and its fluorescence was detected at 517- to 561-nm. GFP- and YFP-tagged proteins were colocalized with a set of mCherry tagged organelle markers (Nelson et al., 2007), mCherry was imaged using excitation with a 594-nm laser and fluorescence was detected at 600-650-nm. Chlorophyll autofluorescence was excited using a 594-nm laser and emission above 650 nm was collected.

### Pi Accumulation Assays

Pi was extracted from ˜50 mg from Arabidopsis and pennycress tissue of various ages. Samples were pulverized in liquid nitrogen. A 1:10 ratio of 1% acetic acid was added to each sample, which was vortexed and incubated on ice, and centrifuged. Assays were performed on Pi extracts using a modified micro-titer assay as previously reported (Ames, 1966). 50 μL of the supernatant and 1 mL of working reagent (5% w/v FeH₁₃O₁₁S·7H₂O, 1% w/v (NH₄)2MoO₄, 1N H₂SO₄ aq.) was incubated for an hour. Samples were placed in quartz cuvettes and absorbance at 660 nm was measured using a plate spectrophotometer. All Pi concentrations were calculated based on a standard curve from a set of standards made with known Pi concentrations.

### Homology Modeling and Molecular Docking

Molecular docking experiments were used to explore the binding modes of ligands of group 9 NUDIX enzymes. NUDIX4 (Q9LE73), NUDIX12 (Q93ZY7), NUDIX13 (Q52K88), NUDIX16 (Q9LHK1), NUDIX17 (Q9ZU95), NUDIX18 (Q9LQU5), and NUDIX21 (Q8VY81) sequences were retrieved from uniprot.org (The UniProt Consortium, 2021). Based on amino acid identity and similarities in Arabidopsis NUDIX13 OX and DDP1 OX transgenic phenotypes, the crystal structure of DDP1 (PDB ID: 7AUL and 7AUK) was selected as control structures given their co-crystallization with ligands and cofactors (Márquez-Moñino et al., 2021). Homology models for selected NUDIX enzymes were generated using RobeTTa fold, an open-source protein structural prediction service (Baek et al., 2021). All crystal structures and homology models were additionally energy minimized using Prime with the OPLS3e force field and default parameters in Schrödinger-Maestro 2020-2 (Schrödinger, 2021). All water molecules from crystal structures were removed before energy minimization. A structural validation was performed on each homology model to compare to other experimentally solved protein structures (ProSA), explore favorable positions of amino acid side chains (Verify3D), and assess backbone phi/psi angles (Kiefer et al., 2009). Energy minimized structures of DDP1 and NUDIX enzymes prepared using AutoDock Tools (ADT) (Morris et al., 2009). Autodock Vina was used for docking and pose predictions (Trott and Olson, 2010). Before docking, all energy minimized structures were aligned. The box coordinates (20 Å×22 Å×24 Å) and center (6.667×−16.250×2.139) with a 1.000 Å grid spacing to dock all ligands. These coordinates were based on re-docking experiments with resolved DDP1 crystal structure PDB ID: 7AUL and 5P-InsP₇. The low energy pose of this re-docking experiment was −6.6 kcal/mol with a root-mean-square deviation (RMSD) of 1.79 Å. However, this low energy pose had 5-InsP₇ in BM-4 with the PP group facing away from the active site and Mg²⁺. The lowest energy pose of this re-docking in an optimal binding mode (BM-B) was −6.7 kcal/mol with an RMSD of 5.78. While the RMSD value for this pose is not as favorable as the pose for BM-4, this is more representative given how DDP1 binds to 5-InsP₇ (Márquez-Moñino et al., 2021).

Ligands docked/re-docked in this study include: Poly(P)₁₅ and inositol phosphates (1,5P₂-InsP₄, 1PP-InsP₅, 5PP-InsP₅, and InsP₆). All ligand structural files were obtained from DDP1 crystal structures and PubChem (Márquez-Moñino et al., 2021; Kim et al., 2021). All ligands were docked in DDP1 and NUDIX enzyme binding pockets with an Mg²⁺; location of this Mg²⁺ ion was based and overlayed on its location in structure PDB ID: 7AUL. Nine poses for each model and ligand were generated. As all of the assessed ligands are highly symmetrical, the top three lowest energy poses for each ligand/enzyme combination were selected for further analysis. Predicted free-energy of binding calculations on ligand/enzyme complexes were performed using Molecular Mechanics/Generalized Borne and Surface Area Solvation (MM-GBSA) in Schrödinger Maestro. Residue/ligand interactions were determined using Schrodinger fingerprint analysis. Visualization of results were visualized using PyMOL 2.5.2 (Schrödinger, LLC, 2021).

### REFERENCES FOR EXAMPLE 1

Adepoju, O., Williams, S. P., Craige, B., Cridland, C. A., Sharpe, A. K., Brown, A. M., Land, E., Perera, I. Y., Mena, D., Sobrado, P., and Gillaspy, G. E. (2019). Inositol Trisphosphate Kinase and Diphosphoinositol Pentakisphosphate Kinase Enzymes Constitute the Inositol Pyrophosphate Synthesis Pathway in Plants. bioRxiv: 724914.

Ames, B. N. (1966) Assay of inorganic phosphate, total phosphate and phosphatases. In Methods in Ames, B. N. (1966) Assay of inorganic phosphate, total phosphate and phosphatases. In Methods in Enzymology. Complex Carbohydrates. Academic Press, pp. 115-118.

Aung, K., Lin, S.-I., Wu, C.-C., Huang, Y.-T., Su, C. and Chiou, T.-J. (2006) pho2, a Phosphate Overaccumulator, Is Caused by a Nonsense Mutation in a MicroRNA399 Target Gene. Plant Physiology, 141, 1000-1011.
Baek, M., DiMaio, F., Anishchenko, I., et al. (2021) Accurate prediction of protein structures and interactions using a three-track neural network. Science, 373, 871-876.
Bowie, J. U., Lüthy, R. and Eisenberg, D. (1991) A method to identify protein sequences that fold into a known three-dimensional structure. Science, 253, 164-170.
Burnette, R. N., Gunesekera, B. M. and Gillaspy, G. E. (2003) An Arabidopsis Inositol 5-Phosphatase Gain-of-Function Alters Abscisic Acid Signaling. Plant Physiol., 132, 1011-1019.
Desai, M., Rangarajan, P., Donahue, J. L., et al. (2014) Two inositol hexakisphosphate kinases drive inositol pyrophosphate synthesis in plants. Plant J, 80, 642-653.
Eskes, E., Deprez, M.-A., Wilms, T. and Winderickx, J. (2018) pH homeostasis in yeast; the phosphate perspective. Curr Genet, 64, 155-161.
Ferrucci, V., Kong, D.-Y., Asadzadeh, F., et al. (2021) Long-chain polyphosphates impair SARS-COV-2 infection and replication. Sci Signal, 14, eabe5040.
Geneious, 2022.0.1 (2022) https://geneious.com/.
Gunawardana, D., Likic, V. A. and Gayler, K. R. (2009) A Comprehensive Bioinformatics Analysis of the Nudix Superfamily in Arabidopsis thaliana. Comp Funct Genomics, 2009, 820381.
Kapila, J., De Rycke, R., Van Montagu, M. and Angenon, G. (1997) An Agrobacterium-mediated transient gene expression system for intact leaves. Plant Science, 122, 101-108.
Kiefer, F., Arnold, K., Künzli, M., Bordoli, L. and Schwede, T. (2009) The SWISS-MODEL Repository and associated resources. Nucleic Acids Res, 37, D387-392.
Kilari, R. S., Weaver, J. D., Shears, S. B. and Safrany, S. T. (2013) Understanding inositol pyrophosphate metabolism and function: Kinetic characterization of the DIPPs. FEBS Letters, 587, 3464-3470.
Kim, S., Chen, J., Cheng, T., et al. (2021) PubChem in 2021: new data content and improved web interfaces. Nucleic Acids Research, 49, D1388-D1395.
Kraszewska, E. (2008) The plant Nudix hydrolase family. Acta Biochim Pol, 55, 663-671.
Kuo, H.-F., Chang, T.-Y., Chiang, S.-F., Wang, W.-D., Charng, Y. and Chiou, T.-J. (2014) Arabidopsis inositol pentakisphosphate 2-kinase, AtIPK1, is required for growth and modulates phosphate homeostasis at the transcriptional level. The Plant Journal, 80, 503-515.
Kuo, H.-F., Hsu, Y.-Y., Lin, W.-C., Chen, K.-Y., Munnik, T., Brearley, C. A. and Chiou, T.-J. (2018) Arabidopsis inositol phosphate kinases IPK1 and ITPK1 constitute a metabolic pathway in maintaining phosphate homeostasis. The Plant Journal, 95, 613-630.
Laha, D., Johnen, P., Azevedo, C., et al. (2015) VIH2 Regulates the Synthesis of Inositol Pyrophosphate InsP₈ and Jasmonate-Dependent Defenses in Arabidopsis [OPEN]. Plant Cell, 27, 1082-1097.
Laha, D., Parvin, N., Hofer, A., Giehl, R. F. H., Fernandez-Rebollo, N., Wiren, N. von, Saiardi, A., Jessen, H. J. and Schaaf, G. (2019) Arabidopsis ITPK1 and ITPK2 Have an Evolutionarily Conserved Phytic Acid Kinase Activity. ACS Chem. Biol., 14, 2127-2133.
Land, E. S., Cridland, C. A., Craige, B., Dye, A., Hildreth, S. B., Helm, R. F., Gillaspy, G. E. and Perera, I. Y. (2021) A Role for Inositol Pyrophosphates in the Metabolic Adaptations to Low Phosphate in Arabidopsis. Metabolites, 11, 601.
Liu, J., Guan, Z., Liu, H., Qi, L., Zhang, D., Zou, T. and Yin, P. (2018) Structural Insights into the Substrate Recognition Mechanism of Arabidopsis GPP-Bound NUDX1 for Noncanonical Monoterpene Biosynthesis. Molecular Plant, 11, 218-221.
Lonetti, A., Szijgyarto, Z., Bosch, D., Loss, O., Azevedo, C. and Saiardi, A. (2011) Identification of an Evolutionarily Conserved Family of Inorganic Polyphosphate Endopolyphosphatases. Journal of Biological Chemistry, 286, 31966-31974.
Lorenzo-Orts, L., Couto, D. and Hothorn, M. (2020) Identity and functions of inorganic and inositol polyphosphates in plants. New Phytologist, 225, 637-652.
Losito, O., Szijgyarto, Z., Resnick, A. C. and Saiardi, A. (2009) Inositol Pyrophosphates and Their Unique Metabolic Complexity: Analysis by Gel Electrophoresis J. A. Bauer, ed. PLoS ONE, 4, e5580.
Márquez-Moñino, M. Á., Ortega-Garcia, R., Shipton, M. L., Franco-Echevarria, E., Riley, A. M., Sanz-Aparicio, J., Potter, B. V. L. and González, B. (2021) Multiple substrate recognition by yeast diadenosine and diphosphoinositol polyphosphate phosphohydrolase through phosphate clamping. Science Advances, 7, eabf6744
Massiah, M. A., Saraswat, V., Azurmendi, H. F. and Mildvan, A. S. (2003) Solution structure and NH exchange studies of the MutT pyrophosphohydrolase complexed with Mg(2+) and 8-oxo-dGMP, a tightly bound product. Biochemistry, 42, 10140-10154.
McLennan, A. G. (2006) The Nudix hydrolase superfamily. Cell. Mol. Life Sci., 63, 123-143.
Mildvan, A. S., Xia, Z., Azurmendi, H. F., et al. (2005) Structures and mechanisms of Nudix hydrolases. Arch Biochem Biophys, 433, 129-143.
Morris, G. M., Huey, R., Lindstrom, W., Sanner, M. F., Belew, R. K., Goodsell, D. S. and Olson, A. J. (2009) AutoDock4 and AutoDockTools4: Automated docking with selective receptor flexibility. J Comput Chem, 30, 2785-2791.
Nagy, R., Grob, H., Weder, B., Green, P., Klein, M., Frelet-Barrand, A., Schjoerring, J. K., Brearley, C. and Martinoia, E. (2009) The Arabidopsis ATP-binding Cassette Protein AtMRP5/AtABCC5 Is a High Affinity Inositol Hexakisphosphate Transporter Involved in Guard Cell Signaling and Phytate Storage. Journal of Biological Chemistry, 284, 33614-33622.
Nelson, B. K., Cai, X. and Nebenfuhr, A. (2007) A multicolored set of in vivo organelle markers for co-localization studies in Arabidopsis and other plants. The Plant Journal, 51, 1126-1136.
Ogawa, T., Yoshimura, K., Miyake, H., Ishikawa, K., Ito, D., Tanabe, N. and Shigeoka, S. (2008) Molecular Characterization of Organelle-Type Nudix Hydrolases in Arabidopsis. Plant Physiol, 148, 1412-1424.
Olejnik, K., Murcha, M. W., Whelan, J. and Kraszewska, E. (2007) Cloning and characterization of AtNUDT13, a novel mitochondrial Arabidopsis thaliana Nudix hydrolase specific for long-chain diadenosine polyphosphates. FEBS J, 274, 4877-4885.
Schrödinger (2021) Schrödinger Release 2021-4: Prime, Schrödinger, LLC.
Schrödinger, LLC (2021) The PyMOL Molecular Graphics System, Version 2.5.2.
Sheikh, S., O'Handley, S. F., Dunn, C. A. and Bessman, M. J. (1998) Identification and characterization of the Nudix hydrolase from the Archaeon, Methanococcus jannaschii, as a highly specific ADP-ribose pyrophosphatase. J Biol Chem, 273, 20924-20928.
Sievers, F., Wilm, A., Dineen, D., et al. (2011) Fast, scalable generation of high-quality protein multiple sequence alignments using Clustal Omega. Mol Syst Biol, 7, 539.
Srouji, J. R., Xu, A., Park, A., Kirsch, J. F. and Brenner, S. E. (2017) The evolution of function within the Nudix homology clan. Proteins, 85, 775-811.
Stamatakis, A. (2014) RAxML version 8: a tool for phylogenetic analysis and post-analysis of large phylogenies. Bioinformatics, 30, 1312-1313.
Stevenson-Paulik, J., Bastidas, R. J., Chiou, S.-T., Frye, R. A. and York, J. D. (2005) Generation of phytate-free seeds in Arabidopsis through disruption of inositol polyphosphate kinases. PNAS, 102, 12612-12617.
Tang, Q., Liu, C., Zhong, C. and Ding, J. (2015) Crystal Structures of Arabidopsis thaliana Nudix Hydrolase NUDT7 Reveal a Previously Unobserved Conformation. Molecular Plant, 8, 1557-1559.
The UniProt Consortium (2021) UniProt: the universal protein knowledgebase in 2021. Nucleic Acids Research, 49, D480-D489.
Trott, O. and Olson, A. J. (2010) AutoDock Vina: improving the speed and accuracy of docking with a new scoring function, efficient optimization and multithreading. J Comput Chem, 31, 455-461.
Wiederstein, M. and Sippl, M. J. (2007) ProSA-web: interactive web service for the recognition of errors in three-dimensional structures of proteins. Nucleic Acids Res, 35, W407-410.
Williams, S. P. (2015) Connections Between Inositol Phosphate Signaling and Energy Responses in Plants. Available at: https://vtechworks.lib.vt.edu/handle/10919/77625.
Wilson, M. and Saiardi, A. (2018) Inositol Phosphates Purification Using Titanium Dioxide Beads.
BIO-PROTOCOL, 8. Available at: https://bio-protocol.org/e2959.
Xu, W., Gauss, P., Shen, J., Dunn, C. A. and Bessman, M. J. (2002) The gene e. 1 (nudE.1) of T4 bacteriophage designates a new member of the Nudix hydrolase superfamily active on flavin adenine dinucleotide, adenosine 5′-triphospho-5′-adenosine, and ADP-ribose. J Biol Chem, 277, 23181-23185.
Zhu, J., Lau, K., Puschmann, R., et al. (2019) Two bifunctional inositol pyrophosphate kinases/phosphatases control plant phosphate homeostasis I. T. Baldwin and J. Kleine-Vehn, eds. eLife, 8, e43582.
Zhu, J., Loubéry, S., Broger, L., Zhang, Y., Lorenzo-Orts, L., Utz-Pugin, A., Fernie, A. R., Young-Tae, C. and Hothorn, M. (2020) A genetically validated approach for detecting inorganic polyphosphates in plants. Plant J, 102, 507-516.
Zong, G., Jork, N., Hostachy, S., Fiedler, D., Jessen, H. J., Shears, S. B. and Wang, H. (2021) New structural insights reveal an expanded reaction cycle for inositol pyrophosphate hydrolysis by human DIPP1. FASEB J, 35, e21275

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered embodiments of the disclosed invention. Reference to disclosure in any of the preceding embodiments is applicable to any preceding numbered embodiment and to any combination of any number of preceding embodiments, as recognized by appropriate antecedent disclosure in any combination of preceding embodiments that can be made. The following numbered aspects are provided:
1. An engineered plant comprising (a) increased NUDIX gene expression as compared to a suitable control; (b) increased amount of one or more NUDIX gene products as compared to a suitable control; (c) increased activity of one or more NUDIX enzymes as compared to a suitable control; or (d) any combination of (a) to (c).
2. The engineered plant of aspect 1, wherein the engineered plant comprises one or more modified NUDIX genes and/or gene products thereby producing an engineered plant that overexpresses one or more NUDIX polypeptides as compared to a suitable control.
3. The engineered plant of any one of aspects 1-2, wherein the one or more NUDIX gene products are NUDIX mRNA, NUDIX polypeptide(s), or both.
4. The engineered plant of any one of aspects 1-3, wherein the engineered plant comprising increased NUDIX gene expression (a) comprises one or more exogenous NUDIX genes or portion thereof that encodes a NUDIX polypeptide; (b) comprises one or more modified endogenous NUDIX genes; (c) comprises a modified epigenome thereby increasing NUDIX gene expression; or (d) any combination of (a) to (c).
5. The engineered plant of aspect 4, wherein (a) the one or more modified endogenous NUDIX genes comprise one or more mutations, insertions, deletions, substitutions, or any combination thereof in the coding or non-coding region of the one or more modified endogenous NUDIX genes such that expression of the one or more modified endogenous NUDIX genes are increased as compared to an unmodified control; (b) the modified epigenome comprises decreased DNA methylation of one or more NUDIX genes and/or histone methylation of a histone associated with the one or more NUDIX genes thereby increasing gene expression of the one or more NUDIX genes; (c) the modified epigenome comprises increased histone acetylation of a histone associated with the one or more NUDIX genes thereby increasing gene expression of the one or more NUDIX genes; or (d) any combination of (a) to (c).
6. The engineered plant of any one of aspects 1-3, wherein the engineered plant comprising increased amount of one or more NUDIX gene products as compared to a suitable control comprises (a) a modified NUDIX mRNA, wherein the mRNA has been modified as compared to an unmodified control to increase protein translation, mRNA stability, or both; (b) a modified NUDIX polypeptide, wherein the polypeptide has been modified as compared to an unmodified control to increase protein stability; or both (a) and (b).
7. The engineered plant of aspect 6, wherein the modified NUDIX mRNA comprises (a) one or more mutations, insertions, deletions, substitutions, or any combination thereof such that translation and/or stability of the NUDIX mRNA is increased; (b) one or more nucleic acid modifications that increases translation and/or mRNA stability; or both (a) and (b).
8. The engineered plant of any one of aspects 1-3, wherein the engineered plant comprising increased activity of one or more NUDIX enzymes comprises (a) one or more modified NUDIX polypeptides comprises one or more amino acid mutations, insertions, deletions, substitutions, or any combination thereof such that activity of the one or more modified NUDIX polypeptides is increased as compared to a control; (b) comprises one or more post-translational modifications such that activity of the one or more modified NUDIX polypeptides is increased as compared to a control.
9. The engineered plant of any one of aspects 1-8, wherein the engineered plant comprises a CRISPR-Cas system or component(s) thereof.
10. The engineered plant of any one of aspects 1-9, wherein the NUDIX gene and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof.
11. The engineered plant of aspect 10, wherein the NUDIX gene and/or gene products are selected from NUDIX 4, NUDIX 12, NUDIX 13, NUDIX 14, NUDIX 16, NUDIX 18, NUDIX 17, NUDIX 21, or any combination thereof.
12. The engineered plant of aspect 10, wherein the NUDIX gene and/or gene products are selected from NUDIX 4, NUDIX 12, NUDIX, 13, NUDIX 16, NUDIX 21, or any combination thereof.
13. The engineered plant of aspect 4, wherein the exogenous or endogenous NUDIX gene and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof.
14. The engineered plant of aspect 13, wherein the exogenous or endogenous NUDIX gene and/or gene products are selected from NUDIX 4, NUDIX 12, NUDIX 13, NUDIX 14, NUDIX 16, NUDIX 17, NUDIX 18, NUDIX 21, or any combination thereof.
15. The engineered plant of aspect 13, wherein the exogenous or endogenous NUDIX gene and/or gene products are selected from NUDIX 4, NUDIX 12, NUDIX, 13, NUDIX 16, NUDIX 21, or any combination thereof.
16. A method of producing an engineered plant having increased expression of a NUDIX gene and/or gene product, the method comprising overexpressing a NUDIX gene and/or gene products in one or more cells of the engineered plant.
17. The method of aspect 16, wherein the method comprises (a) expressing one or more exogenous NUDIX genes and/or gene products in one or more cells of the engineered plant; (b) modifying one or more endogenous NUDIX genes and/or gene products in one or more cells of the engineered plant, wherein the one or more modifications increase expression of the one or more modified endogenous NUDIX genes and/or gene products as compared to an unmodified control; or both (a) and (b).
18. The method of any one of aspects 16-17, wherein the one or more NUDIX genes and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof.
19. The method of aspect 18, wherein the one or more NUDIX genes and/or gene products are selected from NUDIX 4, NUDIX 12, NUDIX 13, NUDIX 14, NUDIX 16, NUDIX 17, NUDIX 18, NUDIX 21, or any combination thereof.
20. The method of aspect 18, wherein the one or more NUDIX genes and/or gene products are selected from NUDIX 4, NUDIX 12, NUDIX, 13, NUDIX 16, NUDIX 21, or any combination thereof.
21. A method comprising: growing, propagating, harvesting, and/or cultivating an engineered plant in any one of aspects 1-15.
22. A method of phytoremediation and/or reducing the amount of inorganic phosphate in a soil, water, or other aqueous environment, the method comprising: planting, growing, harvesting, cultivating, or any combination thereof an engineered plant in any one of aspects 1-15 in a soil and/or aqueous environment.
23. The method of aspect 22, wherein the soil and/or aqueous environment is in need of phytoremediation or inorganic phosphate reduction.
24. The method of aspect 22, further comprising producing biochar, fertilizer, or both from the engineered plant.
25. Use of an engineered plant as in any one of aspects 1-15 to reduce the amount of inorganic phosphate in a soil, water, or other aqueous environment in which the engineered plant is grown or cultivated in.
26. Use of the engineered plant of any one of aspects 1-15, 22-23, and 25 to produce biochar, fertilizer, or both.
27. A method of producing biochar, the method comprising: growing, propagating, harvesting, and/or cultivating an engineered plant as in any one of aspects 1-15 in a soil, water, or other aqueous environment, optionally one in need of phytoremediation or reduction of inorganic phosphate; and carbonizing biomass from the engineered plant by a suitable process to form the biochar.
28. A biochar and/or fertilizer produced from an engineered plant as in any one of aspects 1-15.
29. A biochar produced by the method of aspect 27.

1. An engineered plant comprising:
a. increased NUDIX gene expression as compared to a suitable control;
b. increased amount of one or more NUDIX gene products as compared to a suitable control;
c. increased activity of one or more NUDIX enzymes as compared to a suitable control; or
d. any combination of (a) to (c).

2. The engineered plant of claim 1, wherein the engineered plant comprises one or more modified NUDIX genes and/or gene products thereby producing an engineered plant that overexpresses one or more NUDIX polypeptides as compared to a suitable control.

3. The engineered plant of claim 1, wherein the one or more NUDIX gene products are NUDIX mRNA, NUDIX polypeptide(s), or both.

4. The engineered plant of claim 1, wherein the engineered plant comprising increased NUDIX gene expression
a. comprises one or more exogenous NUDIX genes or a portion thereof that encodes a NUDIX polypeptide;
b. comprises one or more modified endogenous NUDIX genes;
c. comprises a modified epigenome thereby increasing NUDIX gene expression; or
d. any combination of (a) to (c).

5. The engineered plant of claim 4, wherein
a. the one or more modified endogenous NUDIX genes comprise one or more mutations, insertions, deletions, substitutions, or any combination thereof in the coding or non-coding region of the one or more modified endogenous NUDIX genes such that expression of the one or more modified endogenous NUDIX genes are increased as compared to an unmodified control;
b. the modified epigenome comprises decreased DNA methylation of one or more NUDIX genes and/or histone methylation of a histone associated with the one or more NUDIX genes thereby increasing gene expression of the one or more NUDIX genes;
c. the modified epigenome comprises increased histone acetylation of a histone associated with the one or more NUDIX genes thereby increasing gene expression of the one or more NUDIX genes; or
d. any combination of (a) to (c).

6. The engineered plant of claim 1, wherein the engineered plant comprising increased amount of one or more NUDIX gene products as compared to a suitable control comprises
(a) a modified NUDIX mRNA, wherein the mRNA has been modified as compared to an unmodified control to increase protein translation, mRNA stability, or both;
(b) a modified NUDIX polypeptide, wherein the polypeptide has been modified as compared to an unmodified control to increase protein stability; or both (a) and (b);
(c) one or more modified NUDIX polypeptides comprises one or more amino acid mutations, insertions, deletions, substitutions, or any combination thereof such that activity of the one or more modified NUDIX polypeptides is increased as compared to a control;
(d) comprises one or more post-translational modifications such that activity of the one or more modified NUDIX polypeptides is increased as compared to a control; or
(e) any combination of (a)-(d).

7. The engineered plant of claim 6, wherein the modified NUDIX mRNA comprises (a) one or more mutations, insertions, deletions, substitutions, or any combination thereof such that translation and/or stability of the NUDIX mRNA is increased; (b) one or more nucleic acid modifications that increases translation and/or mRNA stability; or both (a) and (b).

8. (canceled)

9. The engineered plant of claim 1, wherein the engineered plant comprises a CRISPR-Cas system or component(s) thereof.

10. The engineered plant of claim 1, wherein the NUDIX gene and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof; or
wherein the NUDIX gene and/or gene products are selected from NUDIX 4, NUDIX 12, NUDIX 13, NUDIX 14, NUDIX 16, NUDIX 17, NUDIX 18, NUDIX 21, or any combination thereof; or
wherein the NUDIX gene and/or gene products are selected from NUDIX 4, NUDIX 12, NUDIX, 13, NUDIX 16, NUDIX 21, or any combination thereof.

11. (canceled)

12. (canceled)

13. The engineered plant of claim 4, wherein the exogenous or endogenous NUDIX gene and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof; or
wherein the exogenous or endogenous NUDIX gene and/or gene products are selected from NUDIX 4, NUDIX 12, NUDIX 13, NUDIX 14, NUDIX 16, NUDIX 17, NUDIX 18, NUDIX 21, or any combination thereof; or
wherein the exogenous or endogenous NUDIX gene and/or gene products are selected from NUDIX 4, NUDIX 12, NUDIX, 13, NUDIX 16, NUDIX 21, or any combination thereof.

14. (canceled)

15. (canceled)

16. A method of producing an engineered plant having increased expression of a NUDIX gene and/or gene product, the method comprising overexpressing a NUDIX gene and/or gene product in one or more cells of the engineered plant.

17. The method of claim 16, wherein the method comprises
a. expressing one or more exogenous NUDIX genes and/or gene products in one or more cells of the engineered plant;
b. modifying one or more endogenous NUDIX genes and/or gene products in one or more cells of the engineered plant, wherein the one or more modifications increase expression of the one or more modified endogenous NUDIX genes and/or gene products as compared to an unmodified control;
c. or both (a) and (b).

18. The method of claim 17, wherein the one or more NUDIX genes and/or gene products are selected from NUDIX 1, NUDIX 2, NUDIX 3, NUDIX 4, NUDIX 5, NUDIX 6, NUDIX 7, NUDIX 8, NUDIX 9, NUDIX 10, NUDIX 11, NUDIX 12, NUDIX 13, NUDIX 14, NUDIX 15, NUDIX 16, NUDIX 17, NUDIX 18, NUDIX 19, NUDIX 20, NUDIX 21, NUDIX 22, NUDIX 23, NUDIX 24, NUDIX 25, NUDIX 26, NUDIX 27, or any combination thereof; or
wherein the one or more NUDIX genes and/or gene products are selected from NUDIX 4, NUDIX 12, NUDIX 13, NUDIX 14, NUDIX 16, NUDIX 17, NUDIX 18, NUDIX 21, or any combination thereof; or
wherein the one or more NUDIX genes and/or gene products are selected from NUDIX 4, NUDIX 12, NUDIX, 13, NUDIX 16, NUDIX 21, or any combination thereof.

19. (canceled)

20. (canceled)

21. A method comprising:
growing, propagating, harvesting, and/or cultivating an engineered plant of claim 1.

22. A method of phytoremediation and/or reducing the amount of inorganic phosphate in and/or producing biochar from a soil, water, or other aqueous environment, the method comprising:
planting, growing, harvesting, cultivating, or any combination thereof an engineered plant of claim 1 in a soil and/or aqueous environment.

23. The method of claim 22, wherein the soil and/or aqueous environment is in need of phytoremediation or inorganic phosphate reduction.

24. The method of claim 22, further comprising producing biochar, fertilizer, or both from the engineered plant.

25. (canceled)

26. (canceled)

27. A method of of claim 1, further comprising
carbonizing biomass from the engineered plant by a suitable process to form the biochar, optionally wherein the soil, water, or other aqueous environment is one in need of phytoremediation or reduction of inorganic phosphate.

28. A biochar and/or fertilizer produced from an engineered plant as in claim 1.

29. A biochar produced by the method of claim 27.


SEQUENCE LISTING (101 sequences)

SEQ ID NO: 1 - RNA, 850 nt, Arabidopsis thaliana
  source 1..850: mol_type = mRNA
aaattttttaataaataaaatgaaaacaagtgcaatggaaaaagaaacagcgataattgtgtacagccaatcacaccgacacggcgacacatttgccaacacgtatataaacgcataaccgatacgacatcgttttaaactcaaattaagtcaacaaaactaaaacacgacaagaaagggtttcactgtgttcgataataatgtcgacaggagaagcgataccaagagtcgccgtcgtcgttttcattctcaacggaaactcaatcttattaggtcgccgccgttcctcaatcggcaactccactttcgctcttcccggtggccacctcgaattcggagagagctttgaagaatgtgcagcgagagaagtaatggaggaaacaggtctaaagattgaaaagatgaagcttttgactgttacaaacaatgtcttcaaagaagcaccaacgccatcacactacgtctctgtttcgatacgtgcggtgttggtggatccaagtcaagaaccgaagaatatggaaccagagaagtgtgaaggatgggattggtatgattgggagaatctaccaaagcctttgttttggccacttgagaaattgtttggaagtggtttcaatcctttcactcatggtggtggagactaatagatgtaagagttaatgattgatttgggattgaatgttgcaaattgggcatttggtctagtggtatgattctcgcttagggtgcgagaggtcccgagttcaattctcggaatgcccctctcttttactcttttttattgtatttttggtttctgttttcctaattagtaataaacttatcatgtgtcctatcgattttatttctc

SEQ ID NO: 2 - RNA, 1278 nt, Arabidopsis thaliana
  source 1..1278: mol_type = mRNA
gagaaactattccacgtacgtattaacctttgaccttaaccttcttaaacagaaaatattgaagtttttatgttcttgtgatagaaatatacagtaacagaatattttctttggcctttattgacatattttagaagaaaaatagattacttccttgttttattattatttcactcatcatacgttgtagaaagggaaccgcaaagtgaaaaaacagttgctccaccacgaggaacagatccatgtctgcttctagttcctccacaaatcccatgtcccgtgaggacgcaacaacactgcttccgtctgttcaagacaagtacggtggcgtgatgaccgagatgactcatcccatggatccttctctcttctccacacttctccgatcctctctatctacctggactcttcagggcaaaaagggagtgtggatcaagttgccaaagcagcttattggtcttgctgaaactgctgttaaggagggattctggtttcaccacgcggagaaagactacttgatgcttgtctattggattcccaaagaagacgatacgcttcctgccaatgcatctcaccgtgttggcataggtgcctttgtcattaaccacaacaaagaggtgctggtggttcaagagaagacagggagatttcaaggtcaaggtatctggaagttccccaccggagttgtgaatgagggtgaagacattcatgatggctcagtcagagaggtgaaagaagagaccggagtggatacagaattcgaccaaatattggccttcagacaaacacacaaagcgttctttggaaagtcagatttgttcttcgtgtgcatgttaaagcccctttctttggagatcaacgcacaggagtcagagattgaggcagctcagtggatgccttgggaggaatacataaatcaaccatttgtacaaaattatgagctattgaggtatatgacagacatttgctctgctaaaaccaacggagactacgaaggcttcactcctctccgcgtctctgcacctgatcaacaaggcaacttgtactacaacacccgcgacttacattcgcgcaattgaccttctttctcaacaaacagagctttcctcacagattctaacgaagctggtttctttgctgcctctctatgtaagcattttgacacttgaatcactacataactctcaccaaaacataaaagcatcccaaatgaaaacatttgcattcaatacaatggctttagttacatacaagattcaatgaagcagattcgttagt

SEQ ID NO: 3 - RNA, 2850 nt, Arabidopsis thaliana
  source 1..2850: mol_type = mRNA
caactaacatgaactaactgctgacataatcgtcttaacattaaaaaaaaatgaaatgaatgcatgcattgcatataaatgtacaaatgttggcttgatctcttgacccctacctaagacgtaagaaggtattttagagctgtgaaatgtgtcaagctgagtctcggagtgaaaagggtccggtttagtgaagccaagtaagcaaagccccttgtttttgacggacattatatctcctcctcttctcttctataaattgggcacctttttccaattttacgcacagtcacggagatagatagtaagagagagctatggcggaggagcacttcgacgttctgactaaatccggtgagaagaccggtgtgtccaaacccaggggagaggttcacagagatggagattaccatcgtgctgttcatgtctggatttttgtggagactactcaacagctacttctccaacttcgatctgatgataaagattcttggcctggtcaatgggacatctctagtgctggccatatatcagctggtgatacatcgcttctatctgcacagcgggagcttgaggaagagcttggcgtaaaactcccaaaggatgcatttgaaaagatcttcgtttttctgcaggaatgtgtcacaaacgatggcaaattcataaataatgagttcaatgatgtgtatcttgttactattctgcatccaatcccattagaagcctttactcttcaaaaagaggaagtctctgctgtaaagtacgtaccttacgaagaatacagaaactttctgtctaaggaagatcctgcttatgtgccatatgatgttaatggagagtatggaaaactatttgacataatcagacagaggtgccaagtgaacactgaggcccgtagtttatctctgcagaagcaactccagagatattctccagtcaccctcgaagccaagttgacagaactttctgaagcagatcagaaagcgttgggtctaattgtgaaggctgcaaaaattatggacgatatattctatgagcaggtttggaacagcaatccagccctaagagattggctgaaggatcatgccaatgcatcaaaactggacaaactaaaatgggattacttcacgatcaacaaaagtccatggtcgtctcttgatgaaaatgaagcattcttgagtactgcggattcagctgtaaagttgcttcctggagcaacaaaagccatagctggctggaagggtcttgaatatagagcagcctttccagtgactaaaccacctggtgccaatttctatcctcccgatatggataaaatggagttcaccttatggctcaatggccttacggaagagcaaaagcatgctgcaacaggatttttcagtgttataaaaaggcgaagtgaagctaatttagatgcttctgatcatcttgccagcagcacaaagaagttaccagactctaattccgatctgtatagtattccttattcagagatatatcgacctttcctcaagaaagcttctgaattcttgcagaaagctggggacttggtcagttcgccaagtttgaagaagctactgcacagcaaagctgaagccttcctttcaaatgagtattatgagtcagacatcgcatggatggacttggattcaaagttagatattaccattggtccatatgagacgtacgaggatgaaatctttgggtataaggctacatttgaaaccttcatcggtattcgagatgataaagccacagctgatctgaaactctttggtgacaatctaaagctattggaagataatctcccgcttgagagtgtatacaaatcgacagatgtatctgctgctcctattcgggtcatccagttaatttacaattccggggatgtgaagggtcctcagactgttgcttacaatttgccaaacgatgaaaaaatcgtgaaggatagaggaacatcaatggttatgctcaagaacgtccaagaggcaaagtttgaacatattttaaagcctatagctgaaatcaccatcagcaaggaacagagaggattagttgattttgattctttcttcactcacaccatttgccatgaatgttgccatggcattggtccccataccataacccttcctggtggtcaaacctcaacagtgagaaaggaactacaagaagtccattctgcaatggaagaagcaaaggcagatatagttggtctttgggcactgaagtttctcatcactaaggggttgctttcaaagagtatggtggagagtatgtatgtttcgttcctagctggttgctttagatcgatccggtttggtctcacagaagctcatgggaaagggcaagccttgcaattcaactatttatatgaaaaaggagccttcgtgttccacgaggactctacattctcggttgatttcgctaagatcgaaggtgcagttgagagcctgagccatgagatactaactattcaaggaaaaggtgacaaaaacgcagcaacattgcttctaaataagtactgcactatcaccggaccattgaaaaccgctctggagaatctcgaacgcgttaaggtaccggtggatatatctccgacatttccattagcagaagcattgatgaactgaggaaagtttcagtttcagcaagactctggttttaacttttaataaataaataaaactcagatgatccatcatcataaaccaaatttgggatttaaaagagttatctgaatggtgttgtgttcttatcataataaagttgctacctttacatctttattccatagaagaaatttttgagagctttaagatagaatgttatcatcatcactaagaaata

SEQ ID NO: 4 - RNA, 1136 nt, Arabidopsis thaliana
  source 1..1136: mol_type = mRNA
aaaaccaataacgctcttaacaaattcacatctccctctctcttatccatttcctctctagacttccttaaatcgattctcccgaattcgagaacagagcctttttttctttaattccaactctttctctcttgttctgaattcgtggttcgtcggtattataaaaaaaaacatgacagggttctctgtgtctctttttgtgtcaaacttgtcaaatgtggcctcgtatttgtcgcccatcttcgagaacatcccctccaccaaggttgttcccgcacagatcgagaaagttgtctctttggtctctcgtaccggtcgcgatttgcagcgttacgatcacgctggctatcgtcaagtcgtcggatgtgtaccgtatagatacaagaaacaagaagtcaatggagttgaaacccaagtaatccaagttcttcttgtcagtgctcaaaagggcaaaggaatgttatttccaaaaggaggttgggagacggatgaatcaatggaggaagctgctttgagagagacgatcgaagaagcgggtgtaacaggagagctcgaagaaaagcttgggaaatggcaatacaaaagcaaaagacatagcataattcacgatgggtatatgtttgctttgcttgtcagtcaagagttcgagcgatggcctgaggcggaaatgagacaacgcagatgggtaagtttggatgaagcaagagaagtatgtcagaattggtggatgagagaagctcttgaagcattcattaacctgaaatgtctagctgacgatgatgaaagtgggaactgaattagtagcactcagaagaaaaatacgatgtttatttagagttggtgaaacagagcttttgacttagataacctctacatggagctgaagcttcctaagggatacagttggtcatagcttctctctgtctattgagaggtgtggaaaagttttgattgttcgatgaaagtcaacttttgtcttctaattttattcattcttttgtattttgatgattcattcattcttttattgtgaataaaatgctagacaggttcctttataaaaacgtttactaacttaatcacacatgtcgctttcatgatcatatgtctaatcattccaaaggattttactgcag

SEQ ID NO: 5 - RNA, 1147 nt, Arabidopsis thaliana
  source 1..1147: mol_type = mRNA
ctaacatttacttttgggttctgttttgattgattgaagcgacatgggtttgacaaaaaactttgcttttcttgtgttttgttgtcaaagatactgtagcatggacggtgaagcttttgaaatctcattacttgatggagaagaagacagatttggaggtactgtggtgaacttgatggaagtggaatccatgactattggtgattttgattcaaagcttgatgtttcacttaaggcttggaaggatcaggggaagaaggggatttggataaagctgcctagtgagctatctagtcttgttgatactgcaattaagaaagggtttacataccaccatgccgagaatgagtatgtgatgcttacattttggcttccagagccacctagcactcttccttgcaatgcttctcatcgtattgggatcggtgcttttgtcctaaacaagaacggagagatgcttgtggttcaggagaatagtggatatttcaaagataaaaatgtctggaaggtgcctaccggtactattaaagagggtgagagtatctgggctggagcagttagggaagtgaaagaagaaactgatattgatgcagagtttgtagaagtcttgtctttcatggaaagtcaccaagcagtgtggcaaagaaaaacggatatatttttcgtctgcgagttggaagcaagaacttttgaaatccagaaacaagattctgagatccatgctgctaagtggatgccggttgaggaatacgtaaaccaaccctatcacaacaaggaggggaacgagatgttcaagttaatagctaacatctgcttaaaaaggtcgcgggagaagtacacgggcttcgttcttactacgaattcagccaaaaagagcctttactgcagcgtcgatcacgccaacctcctgaaggaaacagctgaccaagcctccacctctctctctgactagtcggaaagtgtttctgcttctttgtttcacgtcttgtgtattcaattaaagttcaccagtcttgtgtgtccaaagactggagacttgccattagtaattcaccatggtttccttctaaataaaatgtactgaagaagaaacatttgcttgcgaacggcttttgagaaaggctttgttttggttgatttaattctg

SEQ ID NO: 6 - RNA, 1384 nt, Arabidopsis thaliana
  source 1..1384: mol_type = mRNA
aaatgtgcctctttggttccaaagtcgctctgtaacttaagagagagtgactgttccgttgacagagcaaaatggcccaaacctattgaaaaccccaaaacccttagtttggaatctcatctcattttgcttctgttcttttccatgtaatttcaacaaagtttgaaactttgtttctatagcaactcaattacatggaaattcttaaaagtctactctttaagtcgccctcttggctgaaactcttagcttcttgttataaaacctgaagttcgaaatcattccaataccacaaataactcaactttcttcaagcatggacaatgaagatcaggagtcccttttacttcaaggggttccagataattatggaggtgttaaggtgaacttgacagaaccaatgactattgaggattttgtcccaaagctgagagcttcacttgtgtattggagcaatcaggggacgaaggggatatggttaaagcttgccgatgggcttgacaatcttattgcacctgcaaaagcagaagggtttgtgtgccaccatgccgagagggaatacaccatgcttacatcttggatcgctgatgttcctagtactcttcctgccaatgcgtctcatcgtattggagttggtgcctttgtcctaaacaagaagaccaaagaggtgcttgtggtccaggagattgatggtcatttcaaaggtacaggagtgtggaaactgcctaccggtgttgttaaagagggtgagaatatttgggaaggagcacttagggaagtggaagaagaaactggtattaagacaaaatttgtagaagtcctggctttcagggaaagccaccaagcattcttggaaataaaaacggatatatttttcctctgtgagttagaaccaaccacttttgaaatcaagaaacaagactctgagatcttagctgctaagtggatgccaattgaagaatacgtaaaccaaccctggaaccagaagaaagaactgtttaggttcatggcgaacatctgcttgaagagattgcaggagatggaatacatgggtttctccaaagtccttactacgacttcatctgggaaagagagctatctttactgcaacaccgatcacgccaacctcctcaacgcaacgcgtggcctagcctccacctctggttgaaaaacccttcttcagaacgtgtttatgtttcgttgtttcatgtccattgttcaataaaagcaccagtctcgtgtgtacaacgattggagacttctttatgtaatttaccatcatttcctcactacaacttcatatataatatatttctttggctaccaaaaagaggtatcctttttattctagtttcaataagagtgtgaacccatagatgctgag

SEQ ID NO: 7 - RNA, 1777 nt, Arabidopsis thaliana
  source 1..1777: mol_type = mRNA
ctaacacaaagaataaacgactacaatttataatttttcatttataaaataaaataaaaattggaatatattgtgcggaattcctatatatcagaactcattgatctgctgcttcttctgaaagttcttaagtggttccacgatgtttccattttgttctgacttccgaggccaacgagatgagtccaataaacaaagggcacggaaacattctttttaaattaggtttgtttttttatttttggtttttggtttttcccggaaattgtatgaagtcttcgttacagtttctctctctcaccttctctgtataaagtcttcttgagatcgaaggataacttcaattcaattctctccaaggtacacacagctgatttttgtgataagaagattgattgagatgggtactagagctcagcagattcctttacttgaaggtgagactgataattacgatggtgttactgtaaccatggtggaacctatggattctgaggtttttactgaaagtcttagggcttctctttcgcattggagagaagaggggaagaagggaatttggataaagctgcctcttggattggctaatcttgtggaggctgcagttagtgaaggatttagatatcaccacgcggagcctgagtacttgatgcttgtatcttggatctctgaaactcctgatacaatcccagccaatgcttctcatgttgtaggtgctggtgctttggtcatcaacaaaaatactaaagaggtcctcgttgtccaggagaggagtgggtttttcaaagataaaaatgtgtggaagctgcctactggtgttatcaacgagggcgaggatatatggactggagtagctagggaagtggaagaagaaactggaattattgcagattttgtcgaagtactggctttcaggcaaagccacaaagccatcttaaaaaagaaaacagatatgtttttcctgtgtgtcttaagtccgcgctcttacgatattactgaacaaaaatctgagatcttgcaagctaagtggatgccgatccaagagtatgtagaccaaccatggaacaagaagaacgagatgttcaagttcatggctaacatttgccaaaagaagtgtgaggaagaatacttgggattcgccattgtgccaactaccacatcatctggtaaagagagctttatctactgcaatgcggatcatgccaagcgccttaaagtatcgcgtgaccaagcctctgcttctctctgaaaacattctcatctgattctgtttgatctcggcatgtgtgtgtccagtgtgtacaaaacaggaaactttcttctatattattccgattctacaaaattgcgtctaaatatttaagtttataccatctctatgagccactgtaaaacttgccatcgacttctagtctatttccagtgatggtttgattctcccaacgcttacgtgctttttaaagatatgaggcttaagagtgttaccatacacttggtttattcttccaaaatatcttctcgtaaccggttttaagttctcagctgtatcgtctcttgaccatttactacactggtaaatggtgtgaatccaagaaagtgttaatgccattaaaaaggttcacattactttgtatatgtacaccaatttttgcgactcgtttgtactgtgtgcctaacgtgaaaaagataaggcatatatgggctttagccccaaaaaaactttataggccccatattaaaattatagtgtatttatttttccagaaatagaaaattt

SEQ ID NO: 8 - RNA, 1522 nt, Arabidopsis thaliana
  source 1..1522: mol_type = mRNA
aaccactcgatccactttaaaccacacattataagtgtgcctcaaaaaaatctaacattactctctcaacacatctctctttcttctccatttccttcttctctttcattctcttatttaagatttaaatctgctcaaatgcttgtaaacaaatttcaagttctttaaacgatttccttttcaagaaaaatccttctccttcttctttttaccacatagatcttcttgatctacaatggattctgtttctctttctgaggttactgtcattaaaggaacaacacatttgggttttatgcacagtttcagacagccattttgtggtgtcaaaatatctccaaagttctatttatccaaagttgatggaccaaaggcaatatcttcgagcagcaacacaaagagtcaatttgtttatggaggaggaagcatagctgcaacttcagattcaggttataagatgaatggagtaaacctaaaaagcagaacattgatgagttctgcagttaaagaaagatcattacttgatgcttatgatgatgagtatggtggagtaatagtagatcatggaaaattaccatcaaacccttatgctttcgcttctatgcttcgagcttctctatccgattggagaagaaagggaaagaaaggagtttggttaaagttacctgtggaacaatcagaattagtcccaatagctataaaggaaggttttgagtatcatcatgcagagaaaggatatgtaatgttaacatattggataccagaggaggaacctagtatgcttcctgcaaatgcttcacatcaagttggtgttggaggttttgtattaaatcaacataaagaggtgcttgtggtacaagaaaagtattgtgctccttcgattactggtctatggaagttaccaacagggtttattaatgaatctgaagagattttctctggtgctgtaagagaagtcaaggaagaaactggggtagatacagagttctcagaggttatagctttcagacatgctcacaacgttgcatttgagaaatctgatctgttcttcatctgtatgttgagaccactctctgataagataatcatcgatgctcttgagatcaaagccgcaaagtggatgccattggctgaatttgtggagcaaccgatgataagaggagacaaaatgtttaaaagagtgattgaaatatgcgaggcgagattaagccatcggtactgcggtctttctcctcatcgacttgtctctacttttgatggcaaaccttcttctctctattacaacgttgttgatgatgatcatgatccttcccactccaattgtagcactgagttttatagatagacgggtaaaacagaaccaaatcaagtggttcgattctgtatatacttattctcatgtaatgtatgttgttatttgatactctctcttcagtatagtaaatagtaatagatcctctgcaaaatccatgtagcatcgaaccggtactctcagtagactatacaataaaaagatctgttc

SEQ ID NO: 9 - RNA, 2100 nt, Arabidopsis thaliana
  source 1..2100: mol_type = mRNA
ataatagtaatcattcacaaatccaaaccaaaaatacctaaacttactctcaaatcaaatatatatatgcttctttcaaaatgttctttcaactattctcacttctcttcttaactctatttgttacttccgttacttccagccgtttcatctacgactttgatcctgtcccatcatctgccaccacgccacttgaaactaacccagttggcttcagcggagccgtcatcaaagtcggggcaaatatggccgtcagtggaagtcagattccgttagctagccttttgatacgtaatggcgccattttgaccagtaacaaagacgaagtccctctcattcaccatcaccatatcagtggtacaatatccaacataatcaaatgatcagctcgttggtacaactagtcaacaaaaccataatttttaaggaattgtttttctcaatacatgcaacgttttcttctagagtgaattcattatcgacttggaagattgaacatgtatcatcaagagttgtatctgtaatcatatcagttgacagtttgaatggtcttattgaactaaggggtgttaacaaagccaaagcaagtagatttatgctccaatgagcttattaagatagaaataaactagggacttgaatagctcatgcagagctttattgtgcaaagaagtaaagagaggaagaacaacttatcaaaagattaaagcgttgacaaaaatataaaatagatttaagcttcggcccattagttcccttcttgggattaatggtttatttatcggcccatttagtgatttcagcccattaatggtttcattcctctctcatttttttctttctgtgttcgagcttcttccatggcgaaactagcagaagagatgaatcctgaaggttcacgataccagcttcttctctcatgtccttctggcctctctccgtctcaggtctcagtggatttctcgaaatcgcatgatcggattcctcgtcaagatcctggtctagaagattctatttcacaggtttgggagcagagatcacagggaaattcatcattgttcaatggccaaaaatttaggtatggaggatattgtttagatgatgatgatggtagtaccaatgaagtgccacacgtttgtcttcgtcttggcctgacagattacaggacttttgtaggaacgaatttgagttctctgtgggagaagttccttgttacatcagaagatgactctgttagatgtcgacacacctcaagtccattgggaaatggtgcagttatcgaaacttctgataagaagattattgtgttacgtcggagtaataatgttggggaatttccaggacactatgtattccctgggggccatccagagccaacagcagtaggtattgattatcatcagcttgaaaacaatgttcaaaccggtgaggttttaaacaagaaggtaactcaagaaatgtttgatagcattatctgtgaagttgtcgaagaaactggaataccagcatcatcacttagctcgcctctcttcatcggtatatctcgaagggagttgaatgtaagaccagctatgtttttctacctcaagtgtagtcatcattcagatgacattcaaagattatactctagcgctgaagatgggttcgagtcaacacagctccacactgtctcattggatgagctgaagatgatgacatcgagaatgccaggttgccatcatggcggattcgctctctacgaactgatgcttcaacgtctcaagaacactaaggaaacgtctttaatagcaacgtaagaggaagatacttaaactttacacctagttgtgaattattgtttgctaatgatcatgtcattttcgagtaacttttattagattaaccgtattaatttgtaaaatccatgataatgagtaactatcatgtaattcagggatagaaagctctaagttatgttggataacaggttcatttgttcacatatgtaaatggtaagggtttcgtttctgtagaacctacaacatggggaatatgaaagcttacaaatctcacgcaaataccattatgattcatgcgtttaccctgttttggatgaaaatt

SEQ ID NO: 10 - RNA, 1437 nt, Arabidopsis thaliana
  source 1..1437: mol_type = mRNA
cgtgattgttctccttttgaaatcatttgcttcgttgaaaccacactctccttttactttatcttctccttttcgttgaatacttgaaatcttccaatcttcctccaaattttttcagagtttgaagcatgtcagaccaagaggctcctctgcgtaatggagtagaacacaagattttcgaagtacttccgtttgtggatgatgattacggtggagtcatcgtggagatgaaaactcctatggatactaagaatttcgttgcagcgcttagagattcattcgagcagtggcgcttacagggtaaaaagggagtctggttgaacttgcctctctcacacgtaaacctcgtggaacccgcggttaaggaaggtttccggtatcaccatgctgagccaacttacctgatgctagtttattggattccggaagccgagagtactattcctttgaatgcttcccatcgtgttcgtgtaggtgctgttgtcttgaaccataacaaagaggaaaaatacggttcattatgcggatcaggtatctggaagatcccaacaggagttgttgacgagggtgaggagattttcgccgcagccattagagaagtaaaagaagaaacaggcatcgacactgagtttttggagattctagctttctgccaaacccacgagtcattttttgcaaagtcagatttattcttcgtttgtctcttgagacctacctccttcgatattcaaaaacaggacttagagatcgaagcagctcaatggatgcggttcgaggactctgcctctcaacctatcacacacaagaacgatctcttcaaagatattcaccacatttgctcaatgaaaatggagaagagctatagtggtttttcaaaaaaacctattacaactttctttgacgacaagttaggttatctctacttgaataaacaagaagatatggaacaacctatttcttaaacttcttccaagaacatcgtcattaagagatcatattctgatctaaaacaatcaatcgatctgaatgaaaaacaaatgcatgagtttcagatttgttatcctaaaagagatgctgaattcttctccagcaagcaaaaatacaaatacagtaagaagagagggagcttgtgacgaggaggaattaagaagtgaagatcttgtttttatttgatgatttgaaatcactagaatccactaatacaataattaaataaataaatacatacataccgaagcacaattatttgattgcactcaaatgaatcattgtcttcaagactccttcatccatttttctttaacgttccattctaaactcgactctttgaatgttgatgtatttgttttaagtaattcattttgattattccatcattaattaccactttttctaataacttggaaattgatgttgatgtgctttgtcttccagttt

SEQ ID NO: 11 - RNA, 866 nt, Arabidopsis thaliana
  source 1..866: mol_type = mRNA
aaataaagatatggatctgttactaaaacaaagtcgctttattcagatctgaagaagaagaagaagaagaaacatagttgaagtctctgttaatatgtcttcaacaacaacagattctacagaattgcaaaatctgatcaaactgtttcaaaattgccaaactcatcctcgtcaacattttcctgcaaaatcttcagctgtattagtttgtctataccaagaacaaagagaagacaaaaacgagctacgcgtaatcttaaccaaacgatctactactctctcttctcatcctggggaagtagcattgcctggaggaaaaagagaccaagaagataaagatgatatagctactgcgttgagagaagctcgtgaagaaatcggtttagatccttctcttgttacaatcatttctgttcttgaaccatttgttaacaagaagggaatgtcggtagcaccagttatcggttttctacacgataagaaagcgtttaaacagttaccaaatccagctgaagtggaagagatctttgatgtgccattagagatgttcctcaaggacagaaacaggcgagcagaggaacgagagcatgaaggagagagatatcttcttcaatactttgattactattccgaagataaagagagaagctttatcatatgggcactcactgctggtattctgatcagagttgcctccattgtttatcagagattaccggagtttcaagaacgtaaaccgagtttctggaatcagcctaactgattcttgacttgatctgattgagaaaattgaaacttttatgtataataagcgatacagattgaatcttctttaaatgtgtttcaagaatctatctttactctaa

SEQ ID NO: 12 - RNA, 1268 nt, Arabidopsis thaliana
  source 1..1268: mol_type = mRNA
caaaaataatttatttattttcatgtataaatattaaaactgattatatagagagacaggtgatagttagcagtgaatgctcgttggtacaactcctcattcttctccgcaaaaatagcaagttatttaacaaacaaaaaaattcatataaacgattttatttttttgtgacttttgagattcgttaattctggaaaatttgtttattttcaaaacaagaatatggattcgtcataaaagctaggctatgatcaacatgtttcatacaaacaagaacagggcttttcaagaaagtagcattctctgtttctgttgtctcaatcccttttgattttttttgtgttggcagattcatcaaatgtcggttctttcttctcggacaggacgagatcgtcaacgttacgacaacaacttccgtctcgtttctggatgcattccttataggctgatgaaagctgacgaaactgaagaagactcaggtgttgactttgtaaacaaacttgaagttcttatggtttcatctcctaatcgtcatgatcttgtcttcccaaagggaggatgggaagatgatgagactgttcttgaagctgcttctcgtgaagccattgaagaagcaggagttaaaggaatacttagagaacttcctttaggagtttgggaatttagaagcaaaagtagcacagtggaggatgaatgtttaggtggatgtaaaggatatatgtttgcattaaaagttaccgaagaactcgaggattggccagagcgaaagaatcgtgaacgaagatggttgactgttaaagaagcgttggagctttgtcggtatgagtggatgcagagagcacttgaagagtttttgagagtaatggaagatgaaagaagattgagaacagaggaagagacagtgcatgattcttcaaagctagaggaggagagtcaaatagatccttggtactgttttgtagttaactagtttttcatattctttagctaacgtaaacaaattgaaaaaatgagaagtaaagtgactgcaacacatgtgacatgatataatctcaaatcatctgtcttgaatcttgatatctgtctgtttgaagtgataaagatagagttgtctttatatgcaattgggacaccaaatgcaaaaagacaaaggacatagaagaaattacctaatcacacacaaatcaatgcttaaggcttgacttcaaaatcaactgaatacacttaagaatccaaatccctaagcttatgtcacatgtttagttagg

SEQ ID NO: 13 - RNA, 1157 nt, Arabidopsis thaliana
  source 1..1157: mol_type = mRNA
cttattatatatgtccccgcaaaaatctcatctttttccttcttccttccttcctatatgtgccaacaacaaaccaaaaccgttcctttctttttccatttttcttgtttttttttgttttgtttttggagttgtgtttcacctgttgttggttctttagttcgtcttctgggttaaacaaagaagatcggttctgtttttatttttattttgaaacaagaagaagaaagcaagctacgatgatgtttcttgctgacgccaatagataaagacacgacttttacaaagcttaggacctgcattctcaccgtaattagctcttcttctcaatttctccaaataaatttgtgtgtttttaatgtgtgtgttgcaggttatcaaaaatgtcgaatctttctgcaagaacaggacgagaccatcaacgttatgacaacaacttccgtcttgtttctggatgcattccgtataggctggtgaaagatgaagaagaggactcaacaagtgttgactttgaaaacaagcttcaagttcttatgatttcatctcctaatcgtcacgatcttgtcttcccaaagggaggatgggaagatgatgaaactgttcttgaagctgcttctcgtgaagctatggaagaagcaggagttaaaggaatactaagagaagatccattaggagtttgggaatttagaagtaagagtagcagtgtggaagctgattgctgcttaggtggtggatgcaaaggttatatgtttgcgttagaggtgaaggaagaacttgcgatatggcctgagcaagatgaccgcgagagaagatggctgaatgtaaaagaagccttggagctgtgtcggtatgagtggatgcagagcgctcttgaagagtttctacgagtaatggcagaagaaggaagcacaaaggaagactctcttgccatttccagcatttcaaatcgaggagagcgtcaaattgatcctcgctactgctttgtagtctaactagtttcgtttctctaggcgtatcaaacctctctctcaattgttatatattatggaaagacttcttctgtaaacaaagttaaaacatgagaagcaaatgctttcagaccctgtgacttgaatgtgtttctcataagcaaaagatatatagagattatattaact

SEQ ID NO: 14 - RNA, 1155 nt, Arabidopsis thaliana
  source 1..1155: mol_type = mRNA
gtgatttgcttgctttcacgtttttcattgatggctggctttactcttcttccctcgcggttactcgcgtttccttcacgcgctttacctcgtcgccttcatcaccaccatgcaaagctgattcttcgctgcaaaatgtcttcttcttcttcctctcttactcaatccatcactctcccgagccaacccaacgagcctgtccttgtctctgcaaccgctggaatctcttcttccgatttcagggatgcgattgattcatctctgtttaggaattggctaaggaatttggaatcagagagtggaattttagctgatggttcaatgacattaaagcaagttcttatccagggagttgatatgtttggcaaaagaattggatttctcaaattcaaagcggatattttcgacaaggaaactggtcaaaaggttccaggtattgtatttgcacgaggaccagctgtagctgtgcttattctcttggagtcagatggtgagacttatgcggttctcactgaacaggttcgggttcctactgggaagattgttctggaattacctgctggaatgttggatgatgataaaggtgactttgttggtactgcagttcgcgaggtcgaagaggagattggtataaaactgaaaaaagaagatatggttgatctcactgcttttcttgacccatctactggttaccggatcttcccttctcctggaggctgtgatgaagagatgagcgtgtttctttacagaggacaagtcgaaaaggaaacaattagacagttgcaaggcaaagagacaggactccgtgaacacggtgagttcatcaaagtccgactcatcccatatagagagctctggcgcaaaacagctgacgctaaggttcttatgagcattggtctctatgaaatggctcagcgagagggtcttgtgtccagccagagactgaaacccaactcttgatctcctacaattctctgaaatatacaataactggattcagtcaaccagatgagatagattgagattagacgttcttatgttgtaaaaggagtttttagataaatgttatcgctttataacttggtaccatgacaacttgggaacttatctttagaacaaaagattttgcaaacccaatttagggtttaggtttcc

SEQ ID NO: 15 - RNA, 1440 nt, Arabidopsis thaliana
  source 1..1440: mol_type = mRNA
aaaaaaggagaaacacgtggacactcacacgaggaggtgtctgattttgtctaattaaatttgagttttttttttcttctttgaaatcatatgattgatctacacagaatccatttccgcaaatcggattcatgtttttgctttatcgtaggcttccttcatttgcacgaacaacaacaacaacccttctctgcaaatctatggagcctgcgataacagcgacttcttcttcttctttcggtggtggctcttctcgtctcgccgctttagcccagcaacttcgccaatacaagcccccaccttcttcatcgttcgatgactccgaggagatgcagacagatcaggagaccgctgggaaagtcgtttctcaggttgggtttcaggaatctattgctcctctttcgaaagaccctgataggtttaaacccaagagagccgctgtgttgatctgtctctttgaaggagatgatggtgatttgcgtgttattcttactaagagatcttccaaattgtctactcactctggagaagtttcattgccaggtggtaaagcggaggaggatgataaagatgatgggatgactgctaccagagaggctgaggaagagattggattggacccttctcttgttgatgttgttacttctcttgaaccatttctgtctaagcatcttcttagagtaattcctgtgataggcatcttgagggacaaaaataaattcaatccgataccaaatcctggggaagtggaagctgtgtttgatgcacccttggaaatgttccttaaggatgagaatcgaagatctgaagagagagagtggatgggtgaaaagtatttgatccattactttgactacagaacaggagataaggattatatgatatggggtttaactgctgggattttgatcagagctgcatctgtgacttatgaaagaccacctgcttttatcgagcagtgcccgaagtttaagtaccctaaaatggtagaaaaacatacttgtatgccttaatgaacataatttttcgaatgatgctttcctagactagtcttcttccactatttgataacttgtaccagacaacaagacaataacattccatacaactgattctctgtaccagtgaatggctgaatgcagaacttggctacttgcttatatatatgcagacgcagtagctgaagctccaattcatttcctgctggggcatctcataacaggcttcatttctgagttgttcccacaggatgctcaatggtgattcagagaaaagttggcttgttatgattctaggcaaacatcaccctcaattgtgaatttggaggaatatttgtactcagtatttaccttcaaacgctcatttttcattgtatacttgttatcttctatttgtatactgaaccaatttcaaagcaaacttgtcttatgaggcttatga

SEQ ID NO: 16 - RNA, 808 nt, Arabidopsis thaliana
  source 1..808: mol_type = mRNA
atagacagacacacccaaaccctagatctaaccccagccgtcagatcagattgatcgggcaatcgcttctagaccgttggaatcaagcaatggtagagcagcggtacgaggatggctctcgtctcgtcgccgggtgtattccgtttaggtatgtaaattctgacaaagatggtaattctgaatctgggaaagtgattcaagtgttgatgatcagctcgtctagtggacctggacttttgtttcccaagggaggatgggagaatgatgagacagtcagggaggctgcagcgagggaagcagtggaagaagcaggagtccgtgggattcttatggattttttggggaattatgagttcaagagcaaatcacaccaagatgagtttagcccagaaggtttatgtaaagcggcaatgtacgcgttgtatgtgaaagaagagctagcgacgtggccggaacatgagacgagaacacgtaaatggctgacaattgaagaagcggtagagagttgcaggcacccttggatgaaggacgcattggtggaaggattctgtaaatggcataaggagaagatggttaaaggagaggagataactggtgaacattgaagcaaatgtgcataaacctctgagcgttggtttcattcctttcgattttgtttttacctttttgtttttgtttttgaagtcttatgcatggataatctcttgtgactgcgaaacgtaatagtttcgaagatttaaagttcaattattgccctattttcacatagctgtcttcaccattttcaataattatggaaaatgacagc

SEQ ID NO: 17 - RNA, 1470 nt, Arabidopsis thaliana
  source 1..1470: mol_type = mRNA
gcttctcgaccaatgatgaaaattccaagaaaagaaagaaacattcataattgtttttttttcaaacaaaaaaaaagagcggccgttttattataaaaagaagataagctattggaggttgatacaacgctttttgtctcatctccttcttcttctccttctcaaatcttttttttcaggcgtaacgaatcgaatcataaacacttacgcagaaacaaaaactaaactttggatcttttaaccgaccatagagattcgtgggttcttcttagtttcgacagattcttgatccgttttagttgttacgctcatctttgcttttatacatcagacccttctcttctcttccttctttatctaagaaacattagggttctttgaaatcttgctaaaaaaaatgggtgttgagaaaatggtgtgtttagcttctcgtacgggtcgtcagtttcagagatacaacaaaggtcgtcgtcaagtcgttggatgtgttccttacagattcaagctatctaatgatggtaaaataagtgatgaagtagaagttcttgttatctcttcccaaaaaggccacgccttaatgttccccaagggtggttgggagcttgatgaatctgtagaagaagctgcatcaagagaatgtcttgaagaagctggagttcttggcaacgttgagcaccaacttggaaaatgggattttttaagcaaaagcagaggaacatactatgaaggactaatgttccctatgcttgttacagaacaacttgagctttggcctgaacaacatgttaggcaaagaatctggatgaatgtaaccgaagcaagagaagcttgtagagattggtggatgaaagaagcattagatgttttggttgagagactttcatcaccaatgaatcaaccaaaggaagagaagactatgtcaatctctattgaaacaatgtgttgatgatgaagctgatgattgctaaattgctgattgttgatgggagtttttatgagctaatatggatctctgtaggtattaaatagatgggtctcaattagttattgagacggctaaaaaaatctatttcgaaaagccgatgcagagagcatggacaaagaaaaagatggtagagttttaagttttggatcagaaagattaacatcatgtgatttaagaagtcttgtgtttgtgtttataatgtttctgcaagaacttcttaagatatgattaagagggaagatgataaacttagagtgcaagcaaaacctgtataattgtgtttccctctgtttatttttcctctctctttgaatttgcaatggagacatttgatttcatcatgatgcttttgtgattcagagatacttaaattatgcaagtagatgatacgtggcgtgatgtgattgatttacaagaaacaaagcaagaataagttactctaatcatatgaaaaaaaaaacatgtcattttcaaaatttattatg

SEQ ID NO: 18 - RNA, 1329 nt, Arabidopsis thaliana
  source 1..1329: mol_type = mRNA
agagcaaaaaagagtttagaaaatgtatgtcgtttttctataaaaaggataagccgcaaggaggcaaaagacaacgctttttgtcttatctcaaatcttttgttcactcaccccatcataacttcataactcctatccaatctttgcttataactattatcaaacaattcttaacttcgatctctaacctgcaaaaacattcattacactcttcagatcctccttccctttagccgccaaagcaaaaccgtcatcgatctctcttcttttatacagtcaaaaattagattcagattcttcaagaactctgtttttctagtagcatggtgtgtttggtctcccgtacgggtcgtcaatcccaaagatacaacaagggacggcgtcaagtcgtcgggtgtattccatacagactcaagatttcgagtgatggcacaattagcgacgaatttgaggttcttgttatttcatcacaaaagggtcacgctctgatgttcccaaagggtggttgggagcttgatgaatctgtagaagaagctgcgtcaagagaatctctagaagaagctggagttgttggaaacgttgagagacaacttggaaaatgggatttcttaagcaaaagcaaaggaacattctatgaaggattcatgttcccaatgcttgtgaaagaagagcttgagctttggcctgaacaacatcttcgtcaaagaatttggatgaaagttgatgaagcaagagacgcttgtagagattggtggatgaaggaagcgttagatgttttggtccaaaggctttctttactatcattgaagcctatggaagaagacgagaaccttccactgatctctatctactgatcaaaaaaaaaacaatcagacgtgttcttgatcactttgtatataacatcttacgcctgcctttccatgaagagtctactctactgctgcaggaaaagtatgtacagtgaaactttaggtgtgaacttggccgagatttgtttggatttcggtttggtttaacaaaaaggttttgtaattgttgtcatgatgtatccgcaactgcttaggatgggaggaagaagacagaagcaaagagtgcaaacaaaaaactgtacaataacgtttcctctctttgcatctctcctcatcttcaaatgaatctatgatctaaatgaaactaataaagagaatttacagaaaatcatattcacaagactattatacaacttaattctgttttcttaactgcatatatttgttattttgaatttcatgataagttaaaaaagaaaaaaagaacccaacagctactaaatacgaatatatgctattt

SEQ ID NO: 19 - RNA, 1562 nt, Arabidopsis thaliana
  source 1..1562: mol_type = mRNA
ctcatttctcactctgtgtgggagcctaaacaatggggagacgaaaccagagatgcttgctctcttcctctcttcttcttcgtatccaactctctctttcctctccagatcagtaactctaaacttagccagaacaacaacactctcagctctcacaatgtcgatgaacctaaagactcatgccttcgcaggcaatcccttgaagtcgaaaactccgaaatctaccgatccattctcacccacctccgctttcgaatctctgaaaaccctaattccagtaatccctaatcactcaacaccttcccctgattttaaagtccttccctttagcaagggtcgtccactggtgttctcaagcggtggagatgctaatacgacgccgatttggcatctgggttgggtcagcttggctgattgtaaggttttgttagcaagttgtggggttgatctgaatgaggactcactcgtgtatttaggacccaagttggaagaagatttggtgtattgggctgttgatttggcagaggatggtttcgtttctgaattgggaggtaggaagttgtgttttgttgagcttcgaaccctaatggtagctgctgattgggcggatcaacgtgctatggatgagttggctattgccggcaatgccagggcgttacttgaatggcacaacgtgtcacaattttgtggatcatgtggaagtaaaacttttccaaaggaagcaggaaggagaaaacaatgctcagacgaaacatgcagaaagagggtctatccccgtgttgatccggtggttataatgttagttattgatcgagaaaatgatcgcgcacttttaagcaggcaatctagatatgtaccgagaatgtggagttgtttagctggatttattgagccaggggaaagcttagaagaggctgtgaggcgggaaacatgggaagagacaggcattgaagtaggagatgttgtataccacagctctcagccttggcctgtgggaccaagtagcatgccatgccaattgatgctcggtttctttgctttcgccaagacgctcgacataaacgtagacaaagaagagttagaagatgctcaatggcacagtagagaagaggtgaagaaagcattggcggttgcagaatacaggaaggctcaaagaacagcagctgcaaaggtagagcagatctgtaaaggtgtggagagaagccagagtctatcaactgatttcaatcttgaaagcggtgagcttgctcctatgtttatcccggggccatttgccatcgcgcaccacctgatctcagcatgggtaaatcaggctcctgacgatgttcactcaaaacaacaagcgggtgtctctctctcaagtttgtagctagaaagatgaacttagatgcttagaaaccaaacgtttgctatggcttctatgcttattttgaattgctgagtcctaagtgtgtggacttcgctcgaagttatttctttgcaacgacgatttagatcaataaaaattaatcaaagattgacctcaataaatttgagttttatagttcgtttttctttagtgg

SEQ ID NO: 20 - RNA, 1938 nt, Arabidopsis thaliana
  source 1..1938: mol_type = mRNA
cacgtttatcaaattcataaataaaaactactactactatttaactataaatatttttccaaacaacacaaaataatagcaataggacgtatactaatagagtaataggtgggagcaattgttagattgttaccaatcaaacgagccgtgaccaaaactatagcgcggaaagacaaaactatttaacgaaaacgatattgccatttgcgtttgttaataatcactacttaccagtgctttttaccgaacagtaccgagccgtgactaaaaatcaagcatttcaatatcgagtatactcgattccgattttggtcatccgtatataataactcttgatttccaaactcgacacagccatcgaaaatggcatctgggttttgctcactggctctaactgtcaccacctccttattctcgtcccacgcgattacacggcgcgttctacccattctacggtggcgttccagttctatgtccctttctccgctgcgtcattcccgcgctttatcagctgcaacaactgttccgataagctcatccttcacgtgggacgacgtaatcgagaccggtcgggcagagtacaactcttccgatctcaccggatttttcgagaaaatcaaccgctgcaatcgcggatccgaaaagttaggtgagttcattccatttgttatagaggaacaaatcgttggttatattcataaaagatttacggagtacttgagggagtttcatgatatctttacattttcacaaaatggttcttgtcctgaccgtgttgatggctatgtaacgcttaatctgatgcttcaaaagcctgaagatagaaccagagcagttgcagatgtgatcaaaatattgggtgacaaaggaatcattccaggaatacgtaatgagctgtatcctgtgaaaccatcgtttaatgccccggtctttttttctctagagcgtgctgctgctccttatttcggaattaagggttacggagttcacatgaatgggtatgtagaaagagatggacagaaattactgtggataggtaagagaagtctctcaaaatccacgtatccaggaatgcttgatcatctggttgctggaggattgcctcacgggattagctgcggtggaaatctagtaaaggaatgtgaagaggaagctgggatttccagagccattgctgatagggcaatagcggttggtgctgtctcatatttggacatcgatcagtactgtttcaaacgtgatgtgctgttttgttatgatttggaactccctgaagattttgttcccaaaaatcaagatggagaagttgagagcttcaagttgattccagtagcccaagttgctagtgtgattaagaaaactagtttcttcaaggcgaactgttcccttgtcattattgacttcttgtttcggcacgggtttatcagaccagaatcatcgggttacttggatctataccaacgcctgaggaatagagattgctcatagggatgttactatctgatgattagtctgctgggtcacttcaggtgccttcatgaacataatgaagtccaaagaatacaattttttatacagagagcgagcaaaacaaactcacgtttagagttgtacattttagctcctgttctgttctagactatgagactacgagggaaaatgcttctctattagttgtgatttctggtttgagctaaccggaagctaatgagctcacatggaattgaaggctagcttagtgaattgttggaaacctagccgagattaggggatcacacactgtaattgcttaactagtaaatcgcactgtgatcattatcttctttctcgatcaatatacaagtttctcgattaatgttccatactttgactgtgaaatcatggttgatgcaaagatcgcaacattctctctacggatcagctctaatgtgagccttg

SEQ ID NO: 21 - RNA, 1097 nt, Arabidopsis thaliana
  source 1..1097: mol_type = mRNA
gtgagattttccatataaagacgctcgcaaacccaatcctctttcacaaatcacaaatcacatctcttcatcttcctcggtttatccaaatcctcattcagacttcatcaaatcgtttcttgcttccgatccgaaatttgcggcgggaaaagaagataacatcaacaaaaaaatgatttctctattcatctcaaacttttcaaacttatcaaacttatctccgacctttgataacatgaacatgaacatcccatcgaagaagatcgtaccggtaccgacaccgagtgagaaagttgtctctttggtatctcgaaccggacgagatctccaacgttacaacaccgcagggtatcgtcaagtcgtcggatgtgtaccatatagatacaagaaacacggaggaggagaaattgaagtgcttttgataagtgcacagaagaaagggaaaggaatgttattaccaaaaggaggttgggagattgatgaatcaatagaagaagctgctttaagagagacaattgaagaagctggtgttacgggtcagcttgaagaatcacttgggaaatggcaatacaaaagcaaaagacatactatgattcatgatggtcatatgtttcctttgcttgttagtcagcagtttgagatatggcctgaatctgaattcagacaacgcaaatgggtttctttgtctgaagcgattgagttgtgtcagaattcgtggatgagagaagctttggaagctttcattaaccggaaatgccagacccaataaggataatgtagcatctctgggaacaaagagctcttcatttggaagcagatgcatataattgaagagcaggtcctcaagtttagtgttctgatttgattcattcatttgttaatgatgttagtagactaattgataataactgattgttctgaatgattgtcttcacaacatgtaatgtgtacatatataaaatggatcaatctcatagatgtaataacgacttggtcttcacttttgatttcttatgattttcaatttgttggtgttattttttgaaaaaaatgcagattaggcggtgatgaataattattgacgagaccaaggaaaa

SEQ ID NO: 22 - RNA, 2051 nt, Arabidopsis thaliana
  source 1..2051: mol_type = mRNA
ttggtctgcctttcaagcgagccttggctagcggacaactgttgtaattcactgcaattccatgaagtcgggagcatcagccgcgtcgccaacggcgaaaagcttcaattttggatcttccagactcttagccttggcgcagcagctgcgtgtatacaagccgccgctttcgtcatccttcgacgagagggaagagctgcttgcgtacaaggaaagcaccaggaaatcgataacccacgttgggtttcaggaatctatggctccggtgaggttcagaccgaagaaagctgctgtcttgatatgtctcttcgaaggagacgatggtgatttgcgtgtgattcttactaagaggtcttccacattgtctactcactcaggagaagtttctttgccaggtggtaaagcagaagagcatgataaggatgatgggattactgcaactagagaggcagaggaagagattggattagacccttcgcttgttgatgttgttgctttcctcgaaccatttctatctcagcatctgcttagagtaattcctgtggtaggaatattatgggacagaaaagcattcaatccaacaccaaatcctgcagaagtggaagccgtgcttgatgcaccctttgaaatgtttctgaaggatgagaaccggagatccgaggagtttgattggatgggagaaaagcatttggtccacttctttgattacaaaacaggagatagtgattatgttatatggggcttaactgcaagaatcttgattagagctgcaacagtggtttatcaacgaccacctgctttcattgaacagaaacctaacttgaagtactccaaaatgaaccaggctactcgtttatatggatggctgaagccgctggcaaatttacatgaatcttgtgaaaatggaagattactaggtcatgtcccagcatactgcttgcgttatctagttggaatatcatcaagatgatcgtgtttgtgcatatctcaatcgtttgtgctgaaagacaagctcattcttggaaagtctttcagcatgctttttggatgactctggtatcggaaatcaacagatttaagttatctaattcatctgatgcaagagcaaaggttaaaaagctaaagggaaggctgctgcgtagatccatcttctagatgctttgtttagctctttcatgttctaatgtctcaagagccactgatgcaggtaataggtttagagccatttctgattgatttgcatctaggaaggtggttttgattccgtgaaactaagccagttgatctgatgacaaggatacaatttggatgtggttttcctagtggacctttaccacataatcaaatattggatgtgctccttgaaaacatgggtagctcccgttttgcaagaagtgtgttttttggtgaaaccccaaccatttgacctgaaagtaaagctcgttcttgaaaaatctgccagcaggtgggtagtgcttgtgctgctttctgatgttttccggtttttctggtggaacaaattaatacatctgatgcgaaagaaaagctcgttcttgccttcttggagaatctggcagcatagcgctttgtcatggtcaatgtgtgtctgatattatctggttggaaatctcaatcatatgacctgatagcaaaggagcatttgacttcagttaaaagagctacagacacgctgttctaataggtctgagatgaaattccgtgtaactttggtctcctgaagaagccaagtggtcgataaagccatcactcgtcttatggaacaaaggagaatgcctaaatataatgaatcaacgttgccaaaactaagatggatgataaaccttataagttagggatcaaattcaataaaaaataaacaatgagaaaagtaaaacagtacatgtatgaagtgacacacgagtgaaaaagaagactaaatcgtcttattgtactccaaacattgatcctctacatttctttaggttatatgtaattatgcatgctacaacttaagcatattgtcgtaatttagtgatatattggagccacat

SEQ ID NO: 23 - RNA, 3110 nt, Arabidopsis thaliana
  source 1..3110: mol_type = mRNA
attgccctgtgattctttaatgtttgttttctctttagtttacaagtacatgtgactctataacactcactcagctctgcttttgtgtctgaaccggttaatttggtcaaattaaaccggttgttgggtcttagtgaaaccaataatgccggacctttggctaagttaaagagtcacttcattcaacaaatctcctcaaatgtcaaatttttggaacgaaatagattggccatcgagagaggagcttcattgacaaagagtcctatagccggaagtgaagaaatttccggtgcgtataatgctaaaggccgtccagatcttgggttggtcatcagggttaaccatctcacagagattgaccaagactcgcaagtcctctaccgtctctttcatctcgtcatctctcaatctctcctctgtcacttcgagctctcctcggagaatcttctctttcaagccaaccagaatgtcttcctcacttcccgggtctgacccagttgcaaattccccgactttcgtctctgttcaatcagctggagatgttcgtaagataaagttctgtcaatggtgtggaggaccaaccaagcatgaaatacctgacggtgaagagaaattgagagctatttgcacacattgcggcaaaatcgcttatcagaatcctaagatggttgttggttgccttatagagcatgaaggaaaagttttactttgcaaacgtaacattcaaccttcacacggtttatggactcttcctgctggttatctagaagttggagaatcagctgcacaaggagcaatgagggaaacttgggaagaagcaggagctactgtggaagttatttcaccttttgctcaacttgatattcctcttattggccaaacttatgttatattcttagccaaactgaagaacctgcattttgcacctggtcctgaatcattggagtgccgtctttttgcactagatgagataccgtttgattccttggctttttcatctatatatgttaccttaaatttgtacttggaagatctgaaaaaagggaaactaaagtttcactatggtacgataaataaaaggcctggaagtagtccttcagatattcgcgcctttagtcttgattaccatctgcaaccgtaactcaagtttacagaagcagcttctggattggctttgttcttccatgtcgagtcttttcatttcgcaactcgatgttactaaaagaaaaggaatgatgaagttgcttatgtgagagattccttggacagatgagaaagaactcggaagaagataatttggacagagttttgttctgtatttgtggtggagaaataggtttggatgacttttaggtctctcatgtcagattttatggctgaacaatgtgtatctttatctgtttctggtttcagttgcctcaaatgtaaaaggaaaaaaatcatatgtggatgcaacatgttgatcaatctttgtgtaattaagtcaccgtgttactgttacttcaaggtcttcagtgaaagaaagataaaccaaatagataggatatatttattcattgccctgtgattctttaatgtttgttttctctttagtttacaagtacatgtgactctataacactcactcagctctgcttttgtgtctgaaccggttaatttggtcaaattaaaccggttgttgggtcttagtgaaaccaataatgccggacctttggctaagttaaagagtcacttcattcaacaaatctcctcaaatgtcaaatttttggaacgaaatagattggccatcgagagaggagcttcattgacaaagagtcctatagccggaagtgaagaaatttccggtgcgtataatgctaaaggccgtccagatcttgggttggtcatcagggttaaccatctcacagagattgaccaagactcgcaagtcctctaccgtctctttcatctcgtcatctctcaatctctcctctgtcacttcgagctctcctcggagaatcttctctttcaagccaaccagaatgtcttcctcacttcccgggtctgacccagttgcaaattccccgactttcgtctctgttcaatcagctggagatgttcgtaagataaagttctgtcaatggtgtggaggaccaaccaagcatgaaatacctgacggtgaagagaaattgagagctatttgcacacattgcggcaaaatcgcttatcagaatcctaagatggttgttggttgccttatagagcatgaaggaaaagttttactttgcaaacgtaacattcaaccttcacacggtttatggactcttcctgctggttatctagaagttggagaatcagctgcacaaggagcaatgagggaaacttgggaagaagcaggagctactgtggaagttatttcaccttttgctcaacttgatattcctcttattggccaaacttatgttatattcttagccaaactgaagaacctgcattttgcacctggtcctgaatcattggagtgccgtctttttgcactagatgagataccgtttgattccttggctttttcatctatatatgttaccttaaatttgtacttggaagatctgaaaaaagggaaactaaagtttcactatggtacgataaataaaaggcctggaagtagtccttcagatattcgcgcctttagtcttgattaccatctgcaaccgtaactcaagtttacagaagcagcttctggattggctttgttcttccatgtcgagtcttttcatttcgcaactcgatgttactaaaagaaaaggaatgatgaagttgcttatgtgagagattccttggacagatgagaaagaactcggaagaagataatttggacagagttttgttctgtatttgtggtggagaaataggtttggatgacttttaggtctctcatgtcagattttatggctgaacaatgtgtatctttatctgtttctggtttcagttgcctcaaatgtaaaaggaaaaaaatcatatgtggatgcaacatgttgatcaatctttgtgtaattaagtcaccgtgttactgttacttcaaggtcttcagtgaaagaaagataaaccaaatagataggatatatttattc

SEQ ID NO: 24 - RNA, 1357 nt, Arabidopsis thaliana
  source 1..1357: mol_type = mRNA
gagattttttttcaaaaaacttaatcatctgtatttataacgccttgcatcccaagctagaacagcattgtgtctcctccacagacatggcatctgcattttgctcactttgtcccactcccacctccttattctcttcccacgcgcttatacccactctacagtggcgttcgagttcgagttcgaggtctcctccgctacatatttcccgcgttttatcagttgaaactgttcctttaagcccatcattcacctggaacgatgtttttgagaacagtcgaaaagaatacgtgcctcagaactcctccgatctcaccggatttctcgagaaagtcgaccgctgtaatcgtggattagagaagttaggtgagttcattccatttgttatagaggaacaaatcgttggttatattcacaagggatttacaaagtacttgagggactttaatgatatctttacattttcacaatatggtggccatgtaacgcttaacatgatgcttgacaagcctgaagaaagaaccagagcagttgcacatgtgatcaaaatattgggtaacaaaggaatcatccctgggatacgaaatgagctatatcctgtgaagccatcgtttaatgctcctgcctttttttctatagagcgtgctgctgctccttattttggattgaagggttacgcaattcatgtgaatgggtatgtagaaagagatggacaaaaatttctatggataggtaaaagaagtctagcaaaatccacttatccaggaaaacttgatcatctggttgctggaggattgcctcacgggattagcgtttgtgagaatctagtaaaggaatgcgaagaggaagctgggatttccaaagtcttggctgatagggcgattgcggtcggtgttgtttcctacatggatatcgatcggtactgtttcacacgtgatgtgctgttttgttatgatttggaactccctcaagattttgttcccacaaatcaagatggagaagttgacagcttcaggttgattccagtcgctcaagttgctaatgtggttcggaagactagtttttttaaggacagttgttccctggtcattattgacttcttgtttcggcacgggttaatcagaccagagtcaccgggttacttggatctataccgacgcctgaggaatggagattgctcataaggacgttactttctatctaatgttttaaagaaaaagctcactctctaataaattaatattagatactctgactatgtaataagactcatgcatgtatataatattcagagtcttcatgtgccttcatgaacatatatataccaatcaaggaagttataaaacttcatttatac

SEQ ID NO: 25 - RNA, 847 nt, Arabidopsis thaliana
  source 1..847: mol_type = mRNA
acattagaagttaaaacaaattttgtcaactaatttgaaaattacaaccaggtcatataagtataaaaaaacaaaaaaaaaacaaaacacacacacacacacatgttcttcgttgctgcttcgttgggttaaaaacttttcttttgtttcttgctttttgcaaagtcaaagataacaaataaataagaaaaatggagaatctaccacctggttatcgtcccaatgttggtgtttgtctaatcaactcggataatctggtatttgtagcttctagattgaatgttccaggagcatggcagatgccacagggaggcattgaagatggggaggatccaaagtcagcagccatgagagagttacaagaagaaactggtgttgtttcagctgaaatcgtctctgaggtcccaaattggttgacatatgattttccaccagcagtaaaagcaaaagttaaccgtctttggggcggtgaatggcatggtcaagctcagaaatggtatttagtgagactgaggaacgatgaggacgagaaagagatcaatctagcgaacaacgaagcggattcagagtttgcggagtggaaatgggcgaagccagaagaagtggtagagcaagcagtggattacaaaaggccaacctatgaagaagtcatcaagacttttggttcgttcttaaacgacacaggaagagctgctaaatgtaaatcagccaagtggtgatataagataccaagaaccaatgtttgtctttttattttttttgttaactgttgtttttctttttttgggcggttctttttagtagaacccttttggaatgcaaaagctttctctaggggttttttact

SEQ ID NO: 26 - RNA, 910 nt, Arabidopsis thaliana
  source 1..910: mol_type = mRNA
gtgtagtctttgtacgtaatattcatgatgagtcaggggcataacggtccagttacttattaatttcccgaatacgtggaaaaataggttttggctcgaaatggcactgtaccgaccccttctcttgcatcatccaacttctccgtctgtaactacgtttctacgaaattacccctcgaaaccgatcaaattctccagcctgcccttccttcatcgctgccgtaagtctcgggtctcctcctcctccgctcggtgttgttcttccatggaatctcctcctgaaggatacagaagaaatgtcggcgtctgcctcatgaattcttccaaaaagattttcactgcctcgaggttggatattcccagtgcttggcaaatgcctcagggtggaatcgacgaaggtgaagatccaagagttgcagtcatgagagagcttaaagaagagactggagttcactctgctgagattctcgctgaggcacctcactggataacctatgacttccctcctgatgttagggagaagcttaaggtccgatggggatccgattggaagggtcaagcacagaaatggtttcttctgaaatttactgggaaagatgaagaaatcaatcttcttggcgatggtactgagaaacctgagtttggtgaatggtcctggacttctcctgatcaagtcgttgaaaatgcagtggagttcaagaagccagtgtacaaggaagtcatgtcagctttcgcttctcatctccagtagcaaaataaatcgaaaactaaactctctgttttaagtttttcgtaagaaactctgagaataagctcttcttgacatatttgctcttttttgaacaatctttgaatcaaaatacaattcttgttccctctcttgataaaagtaaagtgtgagatcctgtca

SEQ ID NO: 27 - RNA, 1035 nt, Arabidopsis thaliana
  source 1..1035: mol_type = mRNA
gtctaagcgggtaagggtagtgtcgtaataatatattttatcactagtgagaaaattgaaaccaatcaccgattgttctgagccggacgaaggagaggcagccgcctaagaagttgccgtcgtgcgccatggccgtgaaggcttctggattcattgggaaatcagcaatctctgttcatctagatttctcttcgtttcctgtgaaattttcatgcttgaaacagttttctgtatcttcaccaaagccactggtcgttttatccgttgccttatcgtcgccggcaagaacagttgagtctccgcctgttggttaccggaagaatgttgggatttgtctcgttagcccatgtagaaagatttttacggcgtcaaagattcacattccggatacatggcagatgcctcagggtggtgctgatgagggagaagatttgaggaacgctgcctttagagaactgagagaagaaacaggggttacttcggcagaattcattgcggagataccaaactggctgacctatgattttccccgagaagtaaaggacaaactaaaccggaaatggcgaacaagttacaaaggccaagctcagaagtggtttctctttaagttcacgggcaaggaagaagagataaatctccttggagacggtactgcaaaaccagagtttaaggtgtggtcgtggatgctaccggaacaagttattgaacatgctgtatatttcaagagacctgtttatgagcatgtcataaagcaattcaacccttattttgtggatgaagaaaaggattccatgaactcgtctaaggattgaatgtgcacaaagattcaccaagtgaatactgattagaagcaaatcaatcaattcattatactggtttctcttgaatctcatcaaaaataaaaattcatgaatctcagacattgatcttgtagtttgatgctatatataaatcctcacgaattaaattcacgaactaaattaccacattaataaataattaatttacccataaattacaacattatgctgatac

SEQ ID NO: 28 - protein, 147 aa, Arabidopsis thaliana
MSTGEAIPRVAVVVFILNGNSILLGRRRSSIGNSTFALPGGHLEFGESFEECAAREVMEETGLKIEKMKLLTVTNNVFKEAPTPSHYVSVSIRAVLVDPSQEPKNMEPEKCEGWDWYDWENLPKPLFWPLEKLFGSGFNPFTHGGGD

SEQ ID NO: 29 - protein, 278 aa, Arabidopsis thaliana
MSASSSSTNPMSREDATTLLPSVQDKYGGVMTEMTHPMDPSLFSTLLRSSLSTWTLQGKKGVWIKLPKQLIGLAETAVKEGFWFHHAEKDYLMLVYWIPKEDDTLPANASHRVGIGAFVINHNKEVLVVQEKTGRFQGQGIWKFPTGVVNEGEDIHDGSVREVKEETGVDTEFDQILAFRQTHKAFFGKSDLFFVCMLKPLSLEINAQESEIEAAQWMPWEEYINQPFVQNYELLRYMTDICSAKTNGDYEGFTPLRVSAPDQQGNLYYNTRDLHSRN

SEQ ID NO: 30 - protein, 772 aa, Arabidopsis thaliana
MAEEHFDVLTKSGEKTGVSKPRGEVHRDGDYHRAVHVWIFVETTQQLLLQLRSDDKDSWPGQWDISSAGHISAGDTSLLSAQRELEEELGVKLPKDAFEKIFVFLQECVTNDGKFINNEFNDVYLVTILHPIPLEAFTLQKEEVSAVKYVPYEEYRNFLSKEDPAYVPYDVNGEYGKLFDIIRQRCQVNTEARSLSLQKQLQRYSPVTLEAKLTELSEADQKALGLIVKAAKIMDDIFYEQVWNSNPALRDWLKDHANASKLDKLKWDYFTINKSPWSSLDENEAFLSTADSAVKLLPGATKAIAGWKGLEYRAAFPVTKPPGANFYPPDMDKMEFTLWLNGLTEEQKHAATGFFSVIKRRSEANLDASDHLASSTKKLPDSNSDLYSIPYSEIYRPFLKKASEFLQKAGDLVSSPSLKKLLHSKAEAFLSNEYYESDIAWMDLDSKLDITIGPYETYEDEIFGYKATFETFIGIRDDKATADLKLFGDNLKLLEDNLPLESVYKSTDVSAAPIRVIQLIYNSGDVKGPQTVAYNLPNDEKIVKDRGTSMVMLKNVQEAKFEHILKPIAEITISKEQRGLVDFDSFFTHTICHECCHGIGPHTITLPGGQTSTVRKELQEVHSAMEEAKADIVGLWALKFLITKGLLSKSMVESMYVSFLAGCFRSIRFGLTEAHGKGQALQFNYLYEKGAFVFHEDSTFSVDFAKIEGAVESLSHEILTIQGKGDKNAATLLLNKYCTITGPLKTALENLERVKVPVDISPTFPLAEALMN

SEQ ID NO: 31 - protein, 207 aa, Arabidopsis thaliana
MTGFSVSLFVSNLSNVASYLSPIFENIPSTKVVPAQIEKVVSLVSRTGRDLQRYDHAGYRQVVGCVPYRYKKQEVNGVETQVIQVLLVSAQKGKGMLFPKGGWETDESMEEAALRETIEEAGVTGELEEKLGKWQYKSKRHSIIHDGYMFALLVSQEFERWPEAEMRQRRWVSLDEAREVCQNWWMREALEAFINLKCLADDDESGN

SEQ ID NO: 32 - protein, 302 aa, Arabidopsis thaliana
MGLTKNFAFLVFCCQRYCSMDGEAFEISLLDGEEDRFGGTVVNLMEVESMTIGDFDSKLDVSLKAWKDQGKKGIWIKLPSELSSLVDTAIKKGFTYHHAENEYVMLTFWLPEPPSTLPCNASHRIGIGAFVLNKNGEMLVVQENSGYFKDKNVWKVPTGTIKEGESIWAGAVREVKEETDIDAEFVEVLSFMESHQAVWQRKTDIFFVCELEARTFEIQKQDSEIHAAKWMPVEEYVNQPYHNKEGNEMFKLIANICLKRSREKYTGFVLTTNSAKKSLYCSVDHANLLKETADQASTSLSD

SEQ ID NO: 33 - protein, 283 aa, Arabidopsis thaliana
MDNEDQESLLLQGVPDNYGGVKVNLTEPMTIEDFVPKLRASLVYWSNQGTKGIWLKLADGLDNLIAPAKAEGFVCHHAEREYTMLTSWIADVPSTLPANASHRIGVGAFVLNKKTKEVLVVQEIDGHFKGTGVWKLPTGVVKEGENIWEGALREVEEETGIKTKFVEVLAFRESHQAFLEIKTDIFFLCELEPTTFEIKKQDSEILAAKWMPIEEYVNQPWNQKKELFRFMANICLKRLQEMEYMGFSKVLTTTSSGKESYLYCNTDHANLLNATRGLASTSG

SEQ ID NO: 34 - protein, 282 aa, Arabidopsis thaliana
MGTRAQQIPLLEGETDNYDGVTVTMVEPMDSEVFTESLRASLSHWREEGKKGIWIKLPLGLANLVEAAVSEGFRYHHAEPEYLMLVSWISETPDTIPANASHVVGAGALVINKNTKEVLVVQERSGFFKDKNVWKLPTGVINEGEDIWTGVAREVEEETGIIADFVEVLAFRQSHKAILKKKTDMFFLCVLSPRSYDITEQKSEILQAKWMPIQEYVDQPWNKKNEMFKFMANICQKKCEEEYLGFAIVPTTTSSGKESFIYCNADHAKRLKVSRDQASASL

SEQ ID NO: 35 - protein, 369 aa, Arabidopsis thaliana
MDSVSLSEVTVIKGTTHLGFMHSFRQPFCGVKISPKFYLSKVDGPKAISSSSNTKSQFVYGGGSIAATSDSGYKMNGVNLKSRTLMSSAVKERSLLDAYDDEYGGVIVDHGKLPSNPYAFASMLRASLSDWRRKGKKGVWLKLPVEQSELVPIAIKEGFEYHHAEKGYVMLTYWIPEEEPSMLPANASHQVGVGGFVLNQHKEVLVVQEKYCAPSITGLWKLPTGFINESEEIFSGAVREVKEETGVDTEFSEVIAFRHAHNVAFEKSDLFFICMLRPLSDKIIIDALEIKAAKWMPLAEFVEQPMIRGDKMFKRVIEICEARLSHRYCGLSPHRLVSTFDGKPSSLYYNVVDDDHDPSHSNCSTEFYR

SEQ ID NO: 36 - protein, 311 aa, Arabidopsis thaliana
MAKLAEEMNPEGSRYQLLLSCPSGLSPSQVSVDFSKSHDRIPRQDPGLEDSISQVWEQRSQGNSSLFNGQKFRYGGYCLDDDDGSTNEVPHVCLRLGLTDYRTFVGTNLSSLWEKFLVTSEDDSVRCRHTSSPLGNGAVIETSDKKIIVLRRSNNVGEFPGHYVFPGGHPEPTAVGIDYHQLENNVQTGEVLNKKVTQEMFDSIICEVVEETGIPASSLSSPLFIGISRRELNVRPAMFFYLKCSHHSDDIQRLYSSAEDGFESTQLHTVSLDELKMMTSRMPGCHHGGFALYELMLQRLKNTKETSLIAT

SEQ ID NO: 37 - protein, 277 aa, Arabidopsis thaliana
MSDQEAPLRNGVEHKIFEVLPFVDDDYGGVIVEMKTPMDTKNFVAALRDSFEQWRLQGKKGVWLNLPLSHVNLVEPAVKEGFRYHHAEPTYLMLVYWIPEAESTIPLNASHRVRVGAVVLNHNKEEKYGSLCGSGIWKIPTGVVDEGEEIFAAAIREVKEETGIDTEFLEILAFCQTHESFFAKSDLFFVCLLRPTSFDIQKQDLEIEAAQWMRFEDSASQPITHKNDLFKDIHHICSMKMEKSYSGFSKKPITTFFDDKLGYLYLNKQEDMEQPIS

SEQ ID NO: 38 - protein, 222 aa, Arabidopsis thaliana
MSSTTTDSTELQNLIKLFQNCQTHPRQHFPAKSSAVLVCLYQEQREDKNELRVILTKRSTTLSSHPGEVALPGGKRDQEDKDDIATALREAREEIGLDPSLVTIISVLEPFVNKKGMSVAPVIGFLHDKKAFKQLPNPAEVEEIFDVPLEMFLKDRNRRAEEREHEGERYLLQYFDYYSEDKERSFIIWALTAGILIRVASIVYQRLPEFQERKPSFWNQPN

SEQ ID NO: 39 - protein, 203 aa, Arabidopsis thaliana
MSVLSSRTGRDRQRYDNNFRLVSGCIPYRLMKADETEEDSGVDFVNKLEVLMVSSPNRHDLVFPKGGWEDDETVLEAASREAIEEAGVKGILRELPLGVWEFRSKSSTVEDECLGGCKGYMFALKVTEELEDWPERKNRERRWLTVKEALELCRYEWMQRALEEFLRVMEDERRLRTEEETVHDSSKLEEESQIDPWYCFVVN

SEQ ID NO: 40 - protein, 202 aa, Arabidopsis thaliana
MSNLSARTGRDHQRYDNNFRLVSGCIPYRLVKDEEEDSTSVDFENKLQVLMISSPNRHDLVFPKGGWEDDETVLEAASREAMEEAGVKGILREDPLGVWEFRSKSSSVEADCCLGGGCKGYMFALEVKEELAIWPEQDDRERRWLNVKEALELCRYEWMQSALEEFLRVMAEEGSTKEDSLAISSISNRGERQIDPRYCFVV

SEQ ID NO: 41 - protein, 309 aa, Arabidopsis thaliana
MAGFTLLPSRLLAFPSRALPRRLHHHHAKLILRCKMSSSSSSLTQSITLPSQPNEPVLVSATAGISSSDFRDAIDSSLFRNWLRNLESESGILADGSMTLKQVLIQGVDMFGKRIGFLKFKADIFDKETGQKVPGIVFARGPAVAVLILLESDGETYAVLTEQVRVPTGKIVLELPAGMLDDDKGDFVGTAVREVEEEIGIKLKKEDMVDLTAFLDPSTGYRIFPSPGGCDEEMSVFLYRGQVEKETIRQLQGKETGLREHGEFIKVRLIPYRELWRKTADAKVLMSIGLYEMAQREGLVSSQRLKPNS

SEQ ID NO: 42 - protein, 293 aa, Arabidopsis thaliana
MFLLYRRLPSFARTTTTTLLCKSMEPAITATSSSSFGGGSSRLAALAQQLRQYKPPPSSSFDDSEEMQTDQETAGKVVSQVGFQESIAPLSKDPDRFKPKRAAVLICLFEGDDGDLRVILTKRSSKLSTHSGEVSLPGGKAEEDDKDDGMTATREAEEEIGLDPSLVDVVTSLEPFLSKHLLRVIPVIGILRDKNKFNPIPNPGEVEAVFDAPLEMFLKDENRRSEEREWMGEKYLIHYFDYRTGDKDYMIWGLTAGILIRAASVTYERPPAFIEQCPKFKYPKMVEKHTCMP

SEQ ID NO: 43 - protein, 171 aa, Arabidopsis thaliana
MVEQRYEDGSRLVAGCIPFRYVNSDKDGNSESGKVIQVLMISSSSGPGLLFPKGGWENDETVREAAAREAVEEAGVRGILMDFLGNYEFKSKSHQDEFSPEGLCKAAMYALYVKEELATWPEHETRTRKWLTIEEAVESCRHPWMKDALVEGFCKWHKEKMVKGEEITGEH

SEQ ID NO: 44 - protein, 182 aa, Arabidopsis thaliana
MGVEKMVCLASRTGRQFQRYNKGRRQVVGCVPYRFKLSNDGKISDEVEVLVISSQKGHALMFPKGGWELDESVEEAASRECLEEAGVLGNVEHQLGKWDFLSKSRGTYYEGLMFPMLVTEQLELWPEQHVRQRIWMNVTEAREACRDWWMKEALDVLVERLSSPMNQPKEEKTMSISIETMC

SEQ ID NO: 45 - protein, 176 aa, Arabidopsis thaliana
MVCLVSRTGRQSQRYNKGRRQVVGCIPYRLKISSDGTISDEFEVLVISSQKGHALMFPKGGWELDESVEEAASRESLEEAGVVGNVERQLGKWDFLSKSKGTFYEGFMFPMLVKEELELWPEQHLRQRIWMKVDEARDACRDWWMKEALDVLVQRLSLLSLKPMEEDENLPLISIY

SEQ ID NO: 46 - protein, 438 aa, Arabidopsis thaliana
MLALFLSSSSYPTLSFLSRSVTLNLARTTTLSALTMSMNLKTHAFAGNPLKSKTPKSTDPFSPTSAFESLKTLIPVIPNHSTPSPDFKVLPFSKGRPLVFSSGGDANTTPIWHLGWVSLADCKVLLASCGVDLNEDSLVYLGPKLEEDLVYWAVDLAEDGFVSELGGRKLCFVELRTLMVAADWADQRAMDELAIAGNARALLEWHNVSQFCGSCGSKTFPKEAGRRKQCSDETCRKRVYPRVDPVVIMLVIDRENDRALLSRQSRYVPRMWSCLAGFIEPGESLEEAVRRETWEETGIEVGDVVYHSSQPWPVGPSSMPCQLMLGFFAFAKTLDINVDKEELEDAQWHSREEVKKALAVAEYRKAQRTAAAKVEQICKGVERSQSLSTDFNLESGELAPMFIPGPFAIAHHLISAWVNQAPDDVHSKQQAGVSLSSL

SEQ ID NO: 47 - protein, 374 aa, Arabidopsis thaliana
MASGFCSLALTVTTSLFSSHAITRRVLPILRWRSSSMSLSPLRHSRALSAATTVPISSSFTWDDVIETGRAEYNSSDLTGFFEKINRCNRGSEKLGEFIPFVIEEQIVGYIHKRFTEYLREFHDIFTFSQNGSCPDRVDGYVTLNLMLQKPEDRTRAVADVIKILGDKGIIPGIRNELYPVKPSFNAPVFFSLERAAAPYFGIKGYGVHMNGYVERDGQKLLWIGKRSLSKSTYPGMLDHLVAGGLPHGISCGGNLVKECEEEAGISRAIADRAIAVGAVSYLDIDQYCFKRDVLFCYDLELPEDFVPKNQDGEVESFKLIPVAQVASVIKKTSFFKANCSLVIIDFLFRHGFIRPESSGYLDLYQRLRNRDCS

SEQ ID NO: 48 - protein, 198 aa, Arabidopsis thaliana
MISLFISNFSNLSNLSPTFDNMNMNIPSKKIVPVPTPSEKVVSLVSRTGRDLQRYNTAGYRQVVGCVPYRYKKHGGGEIEVLLISAQKKGKGMLLPKGGWEIDESIEEAALRETIEEAGVTGQLEESLGKWQYKSKRHTMIHDGHMFPLLVSQQFEIWPESEFRQRKWVSLSEAIELCQNSWMREALEAFINRKCQTQ

SEQ ID NO: 49 - protein, 302 aa, Arabidopsis thaliana
MKSGASAASPTAKSFNFGSSRLLALAQQLRVYKPPLSSSFDEREELLAYKESTRKSITHVGFQESMAPVRFRPKKAAVLICLFEGDDGDLRVILTKRSSTLSTHSGEVSLPGGKAEEHDKDDGITATREAEEEIGLDPSLVDVVAFLEPFLSQHLLRVIPVVGILWDRKAFNPTPNPAEVEAVLDAPFEMFLKDENRRSEEFDWMGEKHLVHFFDYKTGDSDYVIWGLTARILIRAATVVYQRPPAFIEQKPNLKYSKMNQATRLYGWLKPLANLHESCENGRLLGHVPAYCLRYLVGISSR

SEQ ID NO: 50 - protein, 280 aa, Arabidopsis thaliana
MLKAVQILGWSSGLTISQRLTKTRKSSTVSFISSSLNLSSVTSSSPRRIFSFKPTRMSSSLPGSDPVANSPTFVSVQSAGDVRKIKFCQWCGGPTKHEIPDGEEKLRAICTHCGKIAYQNPKMVVGCLIEHEGKVLLCKRNIQPSHGLWTLPAGYLEVGESAAQGAMRETWEEAGATVEVISPFAQLDIPLIGQTYVIFLAKLKNLHFAPGPESLECRLFALDEIPFDSLAFSSIYVTLNLYLEDLKKGKLKFHYGTINKRPGSSPSDIRAFSLDYHLQP

SEQ ID NO: 51 - protein, 365 aa, Arabidopsis thaliana
MASAFCSLCPTPTSLFSSHALIPTLQWRSSSSSRSPPLHISRVLSVETVPLSPSFTWNDVFENSRKEYVPQNSSDLTGFLEKVDRCNRGLEKLGEFIPFVIEEQIVGYIHKGFTKYLRDFNDIFTFSQYGGHVTLNMMLDKPEERTRAVAHVIKILGNKGIIPGIRNELYPVKPSFNAPAFFSIERAAAPYFGLKGYAIHVNGYVERDGQKFLWIGKRSLAKSTYPGKLDHLVAGGLPHGISVCENLVKECEEEAGISKVLADRAIAVGVVSYMDIDRYCFTRDVLFCYDLELPQDFVPTNQDGEVDSFRLIPVAQVANVVRKTSFFKDSCSLVIIDFLFRHGLIRPESPGYLDLYRRLRNGDCS

SEQ ID NO: 52 - protein, 175 aa, Arabidopsis thaliana
MENLPPGYRPNVGVCLINSDNLVFVASRLNVPGAWQMPQGGIEDGEDPKSAAMRELQEETGVVSAEIVSEVPNWLTYDFPPAVKAKVNRLWGGEWHGQAQKWYLVRLRNDEDEKEINLANNEADSEFAEWKWAKPEEVVEQAVDYKRPTYEEVIKTFGSFLNDTGRAAKCKSAKW

SEQ ID NO: 53 - protein, 216 aa, Arabidopsis thaliana
MALYRPLLLHHPTSPSVTTFLRNYPSKPIKFSSLPFLHRCRKSRVSSSSARCCSSMESPPEGYRRNVGVCLMNSSKKIFTASRLDIPSAWQMPQGGIDEGEDPRVAVMRELKEETGVHSAEILAEAPHWITYDFPPDVREKLKVRWGSDWKGQAQKWFLLKFTGKDEEINLLGDGTEKPEFGEWSWTSPDQVVENAVEFKKPVYKEVMSAFASHLQ

SEQ ID NO: 54 - protein, 227 aa, Arabidopsis thaliana
MAVKASGFIGKSAISVHLDFSSFPVKFSCLKQFSVSSPKPLVVLSVALSSPARTVESPPVGYRKNVGICLVSPCRKIFTASKIHIPDTWQMPQGGADEGEDLRNAAFRELREETGVTSAEFIAEIPNWLTYDFPREVKDKLNRKWRTSYKGQAQKWFLFKFTGKEEEINLLGDGTAKPEFKVWSWMLPEQVIEHAVYFKRPVYEHVIKQFNPYFVDEEKDSMNSSKD

SEQ ID NO: 55 - protein, 23 aa, Synthetic construct
  SITE 2..6: note = X is any amino acid residue
  SITE 8..14: note = X is any amino acid residue
  SITE 17: note = X is A, G, V, I, F, P, M, or W
  SITE 18: note = X is any amino acid residue
  SITE 21: note = X is any amino acid residue
  SITE 23: note = X is A, G, V, I, F, P, M, or W
GXXXXXEXXXXXXXREXXEEXGX

SEQ ID NO: 56 - DNA, 25 bp, Saccharomyces cerevisiae
  source 1..25: mol_type = other DNA
caccatgggcaaaaccgcggataat

SEQ ID NO: 57 - DNA, 27 bp, Saccharomyces cerevisiae
  source 1..27: mol_type = other DNA
tttgtcgtctttaatgatagcagacct

SEQ ID NO: 58 - DNA, 28 bp, Arabidopsis thaliana
  source 1..28: mol_type = other DNA
caccatgtcggttctttcttctcggaca

SEQ ID NO: 59 - DNA, 24 bp, Arabidopsis thaliana
  source 1..24: mol_type = other DNA
ctagttaactacaaaacagtacca

SEQ ID NO: 60 - DNA, 28 bp, Arabidopsis thaliana
  source 1..28: mol_type = other DNA
caccatgtcgaatctttctgcaagaaca

SEQ ID NO: 61 - DNA, 24 bp, Arabidopsis thaliana
  source 1..24: mol_type = other DNA
ttagactacaaagcagtagcgagg

SEQ ID NO: 62 - protein, 6 aa, synthetic construct
  SITE 3: note = X is A, G, V, I, F, P, M, or W
  SITE 4: note = X is any amino acid residue
REXXEE

SEQ ID NO: 63 - protein, 188 aa, Saccharomyces cerevisiae
MGKTADNHGPVRSETAREGRENQVYSPVTGARLVAGCICLTPDKKQVLMITSSAHKKRWIVPKGGVEKDEPNYETTAQRETWEEAGCIGKIVANLGTVEDMRPPKDWNKDIKQFENSRKDSEVAKHPPRTEFHFYELEIENLLDKFPECHKRHRKLYSYTEAKQNLIDAKRPELLEALNRSAIIKDDK

SEQ ID NO: 64 - protein, 207 aa, Arabidopsis thaliana; identical to SEQ ID NO: 31

SEQ ID NO: 65 - protein, 203 aa, Arabidopsis thaliana; identical to SEQ ID NO: 39

SEQ ID NO: 66 - protein, 202 aa, Arabidopsis thaliana; identical to SEQ ID NO: 40

SEQ ID NO: 67 - protein, 180 aa, Arabidopsis thaliana
MCDLVARTGRLQQRYEDGSRLVAGCIPFRYVNSDKDGNSESGKVIQVLMISSSSGPGLLFPKGGWENDETVREAAAREAVEEAGVRGILMDFLGNYEFKSKSHQDEFSPEGLCKAAMYALYVKEELATWPEHETRTRKWLTIEEAVESCRHPWMKDALVEGFCKWHKEKMVKGEEITGEH

SEQ ID NO: 68 - protein, 182 aa, Arabidopsis thaliana; identical to SEQ ID NO: 44

SEQ ID NO: 69 - protein, 176 aa, Arabidopsis thaliana; identical to SEQ ID NO: 45

SEQ ID NO: 70 - protein, 198 aa, Arabidopsis thaliana; identical to SEQ ID NO: 48

SEQ ID NO: 71 - protein, 188 aa, Saccharomyces cerevisiae; identical to SEQ ID NO: 63

SEQ ID NO: 72 - protein, 120 aa, Arabidopsis thaliana
MSTGEAIPRVAVVVFILNGNSILLGRERSSIGNSTFALPGGHLEFGESFEECAAREVMEETGLKIEKMKLLTVTNNVFKEAPTPSHYVSVSIRAVLVDPSQEPKNMEPEKCEGWDWYDWE

SEQ ID NO: 73 - protein, 230 aa, Arabidopsis thaliana
SSLSTWTLQGKKGVWIKLPKQLIGLAETAVKEGFWFHHAEKDYLMLVYWIPKEDDTLPANASHRVGIGAFVINHNKEVLVVQEKTGRFQGQGIWKFPTGVVNEGEDIHDGSVREVKEETGVDTEFDQILAFRQTHKAFFGKSDLFFVCMLKPLSLEINAQESEIEAAQWMPWEEYINQPFVQNYELLRYMTDICSAKTNGDYEGFTPLRVSAPDQQGNLYYNTRDLHSRN

SEQ ID NO: 74 - protein, 295 aa, Arabidopsis thaliana
DHLASSTKKLPDSNSDLYSIPYSEIYRPFLKKASEFLQKAGDLVSSPSLKKLLHSKAEAFLSNEYYESDIAWMDLDSKLDITIGPYETYEDEIFGYKATFETFIGIRDDKATADLKLFGDNLKLLEDNLPLESVYKSTDVSAAPIRVIQLIYNSGDVKGPQTVAYNLPNDEKIVKDRGTSMVMLKNVQEAKFEHILKPIAEITISKEQRGLVDFDSFFTHTICHECCHGIGPHTITLPGGQTSTVRKELQEVHSAMEEAKADIVGLWALKFLITKGLLSKSMVESMYVSFLAGCF

SEQ ID NO: 75 - protein, 183 aa, Arabidopsis thaliana
ENIPSTKVVPAQIEKVVSLVSRTGRDLQRYDHAGYRQVVGCVPYRYKKQEVNGVETQVIQVLLVSAQKGKGMLFPKGGWETDESMEEAALRETIEEAGVTGELEEKLGKWQYKSKRHSIIHDGYMFALLVSQEFERWPEAEMRQRRWVSLDEAREVCQNWWMREALEAFINLKCLADDDESGN

SEQ ID NO: 76 - protein, 242 aa, Arabidopsis thaliana
VSLKAWKDQGKKGIWIKLPSELSSLVDTAIKKGFTYHHAENEYVMLTFWLPEPPSTLPCNASHRIGIGAFVLNKNGEMLVVQENSGYFKQKNVWKVPTGTIKEGESIWAGAVREVKEETDIDAEFVEVLSFMESHQAVWQRKTDIFFVCELEARTFEIQKQDSEIHAAKWMPVEEYVNQPYHNKEGNEMFKLIANICLKRSREKYTGFVLTTNSAKKSLYCSVDHANLLKETADQASTSLSD

SEQ ID NO: 77 - protein, 244 aa, Arabidopsis thaliana
ASLVYWSNQGTKGIWLKLADGLDNLIAPAKAEGFVCHHAEREYTMLTSWIADVPSTLPANASHRIGVGAFVLNKKTKEVLVVQEIDGHFKGTGVWKLPTGVVKEGENIWEGALREVEEETGIKTKFVEVLAFRESHQAFLEIKTDIFFLCELEPTTFEIKKQDSEILAAKWMPIEEYVNQPWNQKKELFRFMANICLKRLQEMEYMGFSKVLTTTSSGKESYLYCNTDHANLLNATRGLASTSG

SEQ ID NO: 78 - protein, 243 aa, Arabidopsis thaliana
ASLSHWREEGKKGIWIKLPLGLANLVEAAVSEGFRYHHAEPEYLMLVSWISETPDTIPANASHVVGAGALVINKNTKEVLVVQERSGFFKDKNVWKLPTGVINEGEDIWTGVAREVEEETGIIADFVEVLAFRQSHKAILKKKTDMFFLCVLSPRSYDITEQKSEILQAKWMPIQEYVDQPWNKKNEMFKFMANICQKKCEEEYLGFAIVPTTTSSGKESFIYCNADHAKRLKVSRDQASASL

SEQ ID NO: 79 - protein, 235 aa, Arabidopsis thaliana
ASLSDWRRKGKKGVWLKLPVEQSELVPIAIKEGFEYHHAEKGYVMLTYWIPEEEPSMLPANASHQVGVGGFVLNQHKEVLVVQEKYCAPSITGLWKLPTGFINESEEIFSGAVREVKEETGVDTEFSEVIAFRHAHNVAFESKDLFFICMLRPLSDKIIIDALEIKAAKWMPLAEFVEQPMIRGDKMFKRVIEICEARLSHRYCGLSPHRLVSTFDGKPSSLYYNVVDDDHDPSH

SEQ ID NO: 80 - protein, 236 aa, Arabidopsis thaliana
SLFNGQKFRYGGYCLDDDDGSTNEVPHVCLRLGLTDYRTFVGTNLSSLWEKFLVTSEDDSVRCRHTSSPLGNGAVIETSDKKIIVLRRSNNVGEFPGHYVFPGGHPEPTAVGIDYHQLENNVQTGEVLNKKVTQEMFDSIICEVVEETGIPASSLSSPLFIGISRRELNVRPAMFFYLKCSHHSDDIQRLYSSAEDGFESTQLHTVSLDELKMMTSRMPGCHHGGFALYELMLQRL

SEQ ID NO: 81 - protein, 229 aa, Arabidopsis thaliana
DSFEQWRLQGKKGVWLNLPLSHVNLVEPAVKEGFRYHHAEPTYLMLVYWIPEAESTIPLNASHRVRVGAVVLNHNKEEKYGSLCGSGIWKIPTGVVDEGEEIFAAAIREVKEETGIDTEFLEILAFCQTHESFFAKSDLFFVCLLRPTSFDIQKQDLEIEAAQWMRFEDSASQPITHKNDLFKDIHHICSMKMEKSYSGFSKKPITTFFDDKLGYLYLNKQEDMEQPIS

SEQ ID NO: 82 - protein, 216 aa, Arabidopsis thaliana
DSTELQNLIKLFQNCQTHPRQHFPAKSSAVLVCLYQEQREDKNELRVILTKRSTTLSSHPGEVALPGGKRDQEDKDDIATALREAREEIGLDPSLVTIISVLEPFVNKKGMSVAPVIGFLHDKKAFKQLPNPAEVEEIFDVPLEMFLKDRNRRAEEREHEGERYLLQYFDYYSEDKERSFIIWALTAGILIRVASIVYQRLPEFQERKPSFWNQPN

SEQ ID NO: 83 - protein, 203 aa, Arabidopsis thaliana; identical to SEQ ID NO: 39

SEQ ID NO: 84 - protein, 202 aa, Arabidopsis thaliana; identical to SEQ ID NO: 40

SEQ ID NO: 85 - protein, 246 aa, Arabidopsis thaliana
GISSSDFRDAIDSSLFRNWLRNLESESGILADGSMTLKQVLIQGVDMFGKRIGFLKFKADIFDKETGQKVPGIVFARGPAVAVLILLESDGETYAVLTEQVRVPTGKIVLELPAGMLDDDKGDFVGTAVREVEEEIGIKLKKEDMVDLTAFLDPSTGYRIFPSPGGCDEEMSVFLYRGQVEKETIRQLQGKETGLREHGEFIKVRLIPYRELWRKTADAKVLMSIGLYEMAQREGLVSSQRLKPNS

SEQ ID NO: 86 - protein, 252 aa, Arabidopsis thaliana
SSFGGGSSRLAALAQQLRQYKPPPSSSFDDSEEMQTDQETAGKVVSQVGFQESIAPLSKDPDRFKPKRAAVLICLFEGDDGDLRVILTKRSSKLSTHSGEVSLPGGKAEEDDKDDGMTATREAEEEIGLDPSLVDVVTSLEPFLSKHLLRVIPVIGILRDKNKFNPIPNPGEVEAVFDAPLEMFLKDENRRSEEREWMGEKYLIHYFDYRTGDKDYMIWGLTAGILIRAASVTYERPPAFIEQCPKFKYPKM

SEQ ID NO: 87 - protein, 180 aa, Arabidopsis thaliana; identical to SEQ ID NO: 67

SEQ ID NO: 88 - protein, 182 aa, Arabidopsis thaliana; identical to SEQ ID NO: 44

SEQ ID NO: 89 - protein, 176 aa, Arabidopsis thaliana; identical to SEQ ID NO: 45

SEQ ID NO: 90 - protein, 180 aa, Arabidopsis thaliana; identical to SEQ ID NO: 67

SEQ ID NO: 91 - protein, 288 aa, Arabidopsis thaliana
RCNRGSEKLGEFIPFVIEEQIVGYIHKRFTEYLREFHDIFTFSQNGSCPDRVDGYVTLNLMLQKPEDRTRAVADVIKILGDKGIIPGIRNELYPVHPSFNAPVFFSLERAAAPYFGIKGYGVHMNGYVERDGCKLLWIGKRSLSKSTYPGMLDHLVAGGLPHGISCGGNLVKECEEEAGISRAIADRAIAVGAVSYLDIDQYCFKRDVLFCYDLELPEDFVPKNQDGEVESFKLIPVAQVASVIKKTSFFKANCSLVIIDFLFRHGFIRPESSGYLDLYQRLRNRDCS

SEQ ID NO: 92 - protein, 179 aa, Arabidopsis thaliana
DNMNMNIPSKKIVPVPTPSEKVVSLVSRTGRDLQRYNTAGYRQVVGCVPYRYKKHGGGEIEVLLISAQKKGKGMLLPKGGWEIDESIEEAALRETIEEAGVTGQLEESLGKWQYKSKRHTMIHDGHMFPLLVSQQFEIWPESEFRQRKWVSLSEAIELCQNSWMREALEAFINRKCQTQ

SEQ ID NO: 93 - protein, 271 aa, Arabidopsis thaliana
SFNFGSSRLLALAQQLRVYKPPLSSSFDEREELLAYKESTRKSITHVGFQESMAPVRFRPKKAAVLICLFEGDDGDLRVILTKRSSTLSTHSGEVSLPGGKAEEHDKDDGITATREAEEEIGLDPSLVDVVAFLEPFLSQHLLRVIPVVGILWDRKAFNPTPNPAEVEAVLDAPFEMFLKDENRRSEEFDWMGEKHLVHFFDYKTGDSDYVIWGLTARILIRAATVVYQRPPAFIEQKPNLKYSKMNQATRLYGWLKPLANLHESCENGRL

SEQ ID NO: 94 - protein, 233 aa, Arabidopsis thaliana
RIFSFKPTRMSSSLPGSDPVANSPTFVSVQSAGDVRKIKFCQWCGGPTKHEIPDGEEKLRAICTHCGKIAYQNPKMVVGCLIEHEGKVLLCKRNIQPSHGLWTLPAGYLEVGESAAQGAMRETWEEAGATVEVISPFAQLDIPLIGQTYVIFLAKLKNLHFAPGPESLECRLFALDEIPFDSLAFSSIYVTLNLYLEDLKKGKLKFHYGTINKRPGSSPSDIRAFSLDYHLQP

SEQ ID NO: 95 - protein, 281 aa, Arabidopsis thaliana
RCNRGLEKLGEFIPFVIEEQIVGYIHKGFTKYLRDFNDIFTFSQYGGHVTLNMMLDKPEERTRAVAHVIKILGNKGIIPGIRNELYPVHPSFNAPAFFSIERAAAPYFGLKGYAIHVNGYVERDGQKFLWIGKRSLAKSTYPGKLDHLVAGGLPHGISVCENLVKECEEEAGISKVLADRAIAVGVVSYMDIDRYCFTRDVLFCYDLELPQDFVPTNQDGEVDSFRLIPVAQVANVVRKTSFFKDSCSLVIIDFLFRHGLIRPESPGYLDLYRRLRNGDCS

SEQ ID NO: 96 - protein, 175 aa, Arabidopsis thaliana; identical to SEQ ID NO: 52

SEQ ID NO: 97 - protein, 206 aa, Arabidopsis thaliana
HHPTSPSVTTFLRNYPSKPIKFSSLPFLHRCRKSRVSSSSARCCSSMESPPEGYRRNVGVCLMNSSKKIFTASRLDIPSAWQMPQGGIDEGEDPRVAVMRELKEETGVHSAEILAEAPHWITYDFPPDVREKLKVRWGSDWKGQAKWFLLKFTGKDEEINLLGDGTEKPEFGEWSWTSPDQVVENAVEFKKPVYKEVMSAFASHLQ

SEQ ID NO: 98 - protein, 218 aa, Arabidopsis thaliana
GKSAISVHLDFSSFPVKFSCLKQFSVSSPKPLVVLSVALSSPARTVESPPVGYRKNVGICLVSPCRKIFTASKIHIPDTWQMPQGGADEGEDLRNAAFRELREETGVTSAEFIAEIPNWLTYDFPREVKDKLNRKWRTSYKGQAQKWFLFKFTGKEEEINLLGDGTAKPEFKVWSWMLPEQVIEHAVYFKRPVYEHVIKQFNPYFVDEEKDSMNSSKD

SEQ ID NO: 99 - protein, 203 aa, Arabidopsis thaliana; identical to SEQ ID NO: 39

SEQ ID NO: 100 - protein, 202 aa, Arabidopsis thaliana; identical to SEQ ID NO: 40

SEQ ID NO: 101 - protein, 188 aa, Saccharomyces cerevisiae; identical to SEQ ID NO: 63